(19) United States
(12) Patent Application Publication
(10) Pub. No.: US 2025/0257343 A1
(43) Pub. Date: Aug. 14, 2025

(54) NUCLEIC ACID-BASED THERAPEUTICS

(71) Applicant: Factor Bioscience Inc., Cambridge, MA (US)

(72) Inventors: Matthew ANGEL, Cambridge, MA (US); Christopher ROHDE, Cambridge, MA (US); Simon MOORE, Cambridge, MA (US); Franklin KOSTAS, Cambridge, MA (US); Jasmine HARRIS, Cambridge, MA (US)

(21) Appl. No.: 19/028,477

(22) Filed: Jan. 17, 2025

Related U.S. Application Data
(63) Continuation of application No. 17/041,787, filed on Sep. 25, 2020.
(60) Provisional application No. 62/648,785, filed on Mar. 27, 2018.
(60) Provisional application No. 62/758,437, filed on Nov. 9, 2018.

(51) Int. Cl. C12N 9/22; A61K 9/127; A61K 48/00; A61P 25/00; A61P 37/06; C07K 14/50; C07K 14/54

(52) U.S. Cl. CPC C12N 9/22; A61K 9/127; A61P 25/00; A61P 37/06; C07K 14/503; C07K 14/54; A61K 48/00; C12N 2310/20; C12N 2800/80

(57) ABSTRACT

The present invention relates in part to nucleic acids, including nucleic acids encoding proteins, therapeutics and cosmetics comprising nucleic acids, methods for delivering nucleic acids to cells, tissues, organs, and patients, methods for inducing cells to express proteins using nucleic acids, methods, kits and devices for transfecting, gene editing, and reprogramming cells, and cells, organisms, therapeutics, and cosmetics produced using these methods, kits, and devices.

### PRIORITY

This application is a continuation of U.S. application Ser. No. 17/041,787, filed Sep. 25, 2020, which is a U.S. National Phase Application of International Application No. PCT/US2019/024443, filed Mar. 27, 2019, which claims priority to U.S. Provisional Patent Application No. 62/648,785, filed Mar. 27, 2018 and to U.S. Provisional Patent Application No. 62/758,437, filed Nov. 9, 2018. The entire contents of the aforementioned patent applications are incorporated herein by reference in their entirety.

### DESCRIPTION OF THE TEXT FILE SUBMITTED ELECTRONICALLY

The instant application contains a Sequence Listing which has been submitted in XML format via EFS-Web and is hereby incorporated by reference in its entirety. Said XML copy, created on Jan. 21, 2025, is named “61057-711301.xml” and is 8,449,860 bytes in size.

### FIELD OF THE INVENTION

The present invention relates, in part, to methods, compositions, and products for producing and delivering nucleic acids to cells, tissues, organs, and patients, methods for expressing proteins in cells, tissues, organs, and patients, and cells, therapeutics, and cosmetics produced using these methods, compositions, and products.

### BACKGROUND

Ribonucleic acid (RNA) is ubiquitous in both prokaryotic and eukaryotic cells, where it encodes genetic information in the form of messenger RNA, binds and transports amino acids in the form of transfer RNA, assembles amino acids into proteins in the form of ribosomal RNA, and performs numerous other functions including gene expression regulation in the forms of microRNA and long non-coding RNA. RNA can be produced synthetically by methods including direct chemical synthesis and in vitro transcription, and can be administered to patients for therapeutic use.

However, previously described synthetic RNA molecules are unstable and trigger a potent innate-immune response in human cells. In addition, methods for efficient non-viral delivery of nucleic acids to patients, organs, tissues, and cells in vivo have not been previously described. The many drawbacks of existing synthetic RNA technologies and methods for delivery of nucleic acids make them undesirable for therapeutic or cosmetic use.

Accordingly, there remains a need for improved methods and compositions for the production and delivery of nucleic acids to cells, tissues, organs, and patients.

### SUMMARY OF THE INVENTION

The present invention provides, in part, compositions, methods, articles, and devices for delivering nucleic acids to cells, tissues, organs, and patients, methods for inducing cells to express proteins, methods, articles, and devices for producing these compositions, methods, articles, and devices, and compositions and articles, including cells, organisms, cosmetics and therapeutics, produced using these compositions, methods, articles, and devices. Unlike previously reported methods, certain embodiments of the present invention provide small doses of nucleic acids to achieve significant and lasting protein expression in humans.

An aspect of the present invention is a method for treating a disease or disorder caused by a mutation in a gene, the method comprising administering to a subject in need thereof and comprising the mutation in the gene an effective amount of a synthetic RNA encoding a gene-editing protein capable of creating a single-strand or double-strand break in the gene, wherein the single-strand or double-strand break causes persistent altered splicing of the gene.

In embodiments, the altered splicing results in expression of a truncated protein which lacks at least the polypeptide sequence corresponding to an exon containing the mutation.

In embodiments, the single-strand or double-strand break removes a splice acceptor site or produces a non-functional splice acceptor site in or near an exon of the gene or removes a splice donor site or produces a non-functional splice donor site in or near an exon of the gene.

In embodiments, the gene-editing protein creates a non-functional splice acceptor site that is within about 1 kb or about 0.5 kb or about 0.1 kb of the exon.

In embodiments, the mutation causes altered splicing of the gene and the single-strand or double-strand break causes the expression of a functional gene product.

In embodiments, the mutation inactivates a splice acceptor site or a splice donor site and the single-strand or double-strand break restores a functional exon.

In any of the preceding embodiments and aspect, the single-strand or double-strand break is within about 1 kb or about 0.5 kb or about 0.1 kb of the exon.

In any of the preceding embodiments and aspect, the non-functional splice acceptor site causes excision of the exon when a pre-mRNA comprising the exon is processed into mRNA.

In embodiments, the gene-editing protein creates a non-functional splice donor site in an intron that is within about 1 kb or about 0.5 kb or about 0.1 kb of the exon.

In any of the preceding embodiments and aspect, the non-functional splice donor site causes excision of the exon when a pre-mRNA comprising the exon is processed into mRNA.

In any of the preceding embodiments and aspect, wherein the exon comprises a mutation.

In embodiments, the mutation is a nonsense mutation, a frame shift mutation, or a mutation that introduces a premature stop codon.

In any of the preceding embodiments and aspect, wherein the mRNA is translated into a truncated protein which retains a function of the full-length protein.

In any of the preceding embodiments and aspect, wherein the exon encodes a polypeptide sequence comprising a peptide splice site.

In embodiments, the mRNA is translated into a polypeptide which lacks the peptide splice site.

In embodiments, the cleavage site is a protease cleavage site or a caspase cleavage site.

In any of the preceding embodiments and aspect, wherein the exon encodes a polypeptide sequence comprising a cleavage site.

In embodiments, the mRNA is translated into a polypeptide which lacks the cleavage site.

In any of the preceding embodiments and aspect, the truncated protein possesses a function of the wild-type protein.

In any of the preceding embodiments and aspect, the gene-editing protein is selected from a TALEN, a meganuclease, a nuclease, a zinc finger nuclease, a CRISPR-associated protein, CRISPR/Cas9, Cas9, xCas9, Cas12a (Cpf1), Cas13a, Cas14, CasX, CasY, a Class 1 Cas protein, a Class 2 Cas protein, and MAD7.

In any of the preceding embodiments and aspect, the gene-editing protein comprises: (a) a DNA-binding domain comprising a plurality of repeat sequences and at least one of the repeat sequences comprises the amino acid sequence: LTPvQWAIAwxyzGHGG (SEQ ID NO: 629), wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 630) or GGKQALETVQRLLPVLCQA (SEQ ID NO: 631); and (b) a nuclease domain comprising a catalytic domain of a nuclease.

In embodiments, the nuclease domain is capable of forming a dimer with another nuclease domain.

In any of the preceding embodiments and aspect, the nuclease domain comprises the catalytic domain of a protein comprising the amino acid sequence of SEQ ID NO: 632.

In any of the preceding embodiments and aspect, at least one of the repeat sequences comprising the amino acid sequence LTPvQWAIAwxyzGHGG (SEQ ID NO: 629) is between 36 and 39 amino acids long.

In any of the preceding embodiments and aspect, the gene is selected from ABCA4, ADAMTS-13, APP, ATP6AP2, CEP290, COL17A1, COL4A3, COL4A4, COL4A5, COL6A1, COL6A2, COL6A3, COL7A1, DMD, DMD, FUS, FXN, GABRG2, HNRPDL, HTT, IKBKAP, ITGA6, ITGB4, LAMA3, LAMB3, LAMC2, LMNA, LMNA, LMNA, LMNA, LMNB1, MAPT, PINK1, PRPF6, RBM20, RNU4ATAC, SMN1, SNRNP200, TARDP, TCF4, TTN, U2AF1, USH2A, and USH2A.

In any of the preceding embodiments and aspect, a gene, the sequence identifier (SEQ ID NO) for its NCBI Reference Sequence, a mutation or mutations therein, the intron or introns that are associated with diseases, and/or the exon or exons that are associated with diseases which can be treated by the method is selected from the list Table 2.

In any of the preceding embodiments and aspect, the disease or disorder is selected from Alport Syndrome, Alport Syndrome, Alport Syndrome, Alzheimer's disease, Amyotrophic lateral sclerosis (ALS), Autosomal dominant leukodystrophy (ADLD), Becker muscular dystrophy (BMD), Bethlem myopathy and Ullrich scleroatonic muscular dystrophy, Dilated cardiomyopathy (DCM), Duchenne muscular dystrophy, Dystrophic Epidermolysis Bullosa, Early-onset Parkinson disease (PD), Epidermolysis Bullosa (EB), Familial dysautonomia (FD), Familial partial lipodystrophy type 2 (FPLD2), Febrile seizures (FS); childhood absence epilepsy (CAE), generalized epilepsy with febrile seizures plus (GEFS+), and Dravet syndrome (DS)/severe myoclonic epilepsy in infancy (SMEI), Friedreich ataxia, Frontotemporal dementia with parkinsonism chromosome 17 (FTDP-17), Fuchs endothelial corneal dystrophy (FECD), Huntington's Disease, Hutchinson-Gilford progeria syndrome (HGPS), Junctional Epidermolysis Bullosa, Leber's congenital amaurosis (LCA), Limb girdle muscular dystrophy type 1B (LGMD1B), Limb-girdle muscular dystrophy 1G (LGMD1G), Microcephalic osteodysplastic primordial dwarfism type 1 (MOPD I), Myelodysplastic syndromes (MDS), Retinitis pigmentosa (adRP), Spinal muscular atrophy (SMA), Stargardt disease, Thrombotic thrombocytopenic purpura (TTP), Ushers syndrome type I, Ushers syndrome type II, Various myopathies and dystrophies, a wound, and X-linked parkinsonism with spasticity (XPDS).

In any of the preceding embodiments and aspect, a single administration of the effective amount of the synthetic RNA encoding the gene-editing protein causes persistent altered RNA splicing of the gene.

An aspect of the present invention is a method for treating a neurodegenerative disease or central nervous system injury comprising administering to a subject in need thereof a synthetic RNA encoding a neurotrophic agent, a gene-editing protein, or an enzyme that cleaves a dysfunctional, an abnormally folding, and/or a disease-causing protein, wherein the neurotrophic agent, the gene-editing protein, or the enzyme treats the neurodegenerative disease or central nervous system injury.

In embodiments, the neurodegenerative disease is selected from: a motor neuron disease, a polyglutamine disease, a prion disease, a spinocerebellar ataxia, a trinucleotide repeat disorder, Alzheimer's disease, amyotrophic lateral sclerosis (ALS), ataxia telangiectasia, ataxia-oculomotor apraxia, Batten disease, Cockayne syndrome, dementia, familial encephalopathy, Huntington's disease, Lewy-body dementia, multiple system atrophy, Parkinson's disease, spinocerebellar ataxia type 1, spongiform encephalopathy, and xeroderma pigmentosum.

In embodiments, the central nervous system injury is selected from: concussion, diffuse axonal injury, diffuse brain injury, focal brain injury, hemorrhage, seizure, stroke, traumatic brain injury, traumatic encephalopathy, and traumatic head injury.

In any of the preceding embodiments and aspects, wherein the administering is by intravenous injection or infusion; intra-arterial injection or infusion; intrathecal injection or infusion; intracerebral injection or infusion; injection or infusion into a ventricle, including a lateral ventricle; injection or infusion into the hippocampus; injection or infusion into the striatum; or injection or infusion into one or more of: the putamen, the caudate nucleus, the substantia nigra, the cortex, the third ventricle, the spinal cord, or the basal ganglia.

In any of the preceding embodiments and aspects, wherein the synthetic RNA encodes a neurotrophic agent.

In embodiments, the neurotrophic agent is a neurotrophic protein selected from nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF).

In embodiments, the neurotrophic protein is NGF and comprising the sequence of SEQ ID NO: 254, the neurotrophic protein is BDNF and comprising the sequence of SEQ ID NO: 561, the neurotrophic protein is NT-3 and comprising the sequence of SEQ ID NO: 255, the neurotrophic protein is NT-4 and comprising the sequence of SEQ ID NO: 256, the neurotrophic protein is CNTF and comprising the sequence of SEQ ID NO: 786, or the neurotrophic protein is GDNF family of ligands and comprising the sequence of SEQ ID NO: 787-793.

In embodiments, the synthetic RNA encodes a gene-editing protein that targets a safe harbor locus.

In embodiments, the synthetic RNA encodes a gene-editing protein that targets one or more of: AAVS1, CCR5, the human orthologue of the mouse Rosa26 locus.

In embodiments, the gene-editing protein inserts a functional copy of a gene into the subject's cells.

In embodiments, the inserted functional copy of a gene does not cause alterations of the subject's cell's genome which pose a risk to the subject.

In embodiments, the gene encodes a neurotrophic agent.

In any of the preceding embodiments and aspects, the gene encodes nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF).

In embodiments, the NGF comprises the sequence of SEQ ID NO: 254, the BDNF comprises the sequence of SEQ ID NO: 561, the NT-3 comprises the sequence of SEQ ID NO: 255, the NT-4 comprises the sequence of SEQ ID NO: 256, the CNTF comprises the sequence of SEQ ID NO: 786, or the GDNF family of ligands comprises the sequence of SEQ ID NO: 787-793.

In any of the preceding embodiments and aspects, the gene is inserted downstream of one or more of: a simple promoter, a constitutive promoter, a strong promoter, an endogenous promoter, tissue-specific promoter, cell type-specific promoter, or a drug-inducible promoter.

In any of the preceding embodiments and aspects, the method induces neurogenesis.

In any of the preceding embodiments and aspects, the synthetic RNA encodes an enzyme that cleaves a dysfunctional, abnormally folding, and/or a disease-causing protein.

In embodiments, the dysfunctional, abnormally folding, and/or disease-causing protein forms a glial scar.

In any of the preceding embodiments and aspects, the dysfunctional, abnormally folding, and/or disease-causing protein is amyloid, tau, alpha-synuclein, or huntingtin.

In any of the preceding embodiments and aspects, the administering is by intravenous injection or infusion; intra-arterial injection or infusion; intrathecal injection or infusion; intracerebral injection or infusion; injection or infusion into a ventricle, including a lateral ventricle; injection or infusion into the hippocampus; injection or infusion into the striatum; or injection or infusion into one or more of: the putamen, the caudate nucleus, the substantia nigra, the cortex, the third ventricle, the spinal cord, or the basal ganglia.

In any of the preceding embodiments and aspects, the administering is directly to a target tissue.

In embodiments, the administering is directly to a site of disease or injury.

In any of the preceding embodiments and aspects, the synthetic RNA is not encapsulated in a viral particle.

In any of the preceding embodiments and aspects, the synthetic RNA is formulated in a liposome or lipid particle.

An aspect of the present invention is a method for treating and/or reducing pain comprising administering to a subject in need thereof an effective amount of a synthetic RNA encoding a gene-editing protein capable of creating a single-strand or double-strand break in a voltage-gated sodium channel type 1 (NaV1) gene, wherein the administering is directed to the central nervous system (CNS) or the peripheral nervous system (PNS).

In embodiments, the NaV1 is selected from NaV1.3, NaV1.7, NaV1.8, and NaV1.9.

In embodiments, the NaV1.3 is encoded by the SCN3A gene comprising the sequence of SEQ ID NO: 671, the NaV1.7 is encoded by the SC9N9A gene comprising the sequence of SEQ ID NO: 662, the NaV1.8 is encoded by the SCN10A gene comprising the sequences of SEQ ID NO: 672, and the NaV1.9 is encoded by the SCN11A gene comprising the sequences of SEQ ID NO: 673.

In any of the preceding embodiments and aspects, the administering is directed to neurons and/or glial cells of the CNS or PNS.

In any of the preceding embodiments and aspects, the administering is by intraganglionic injection, injection to the peripheral or central nerve roots, or injection in proximity to the dorsal root ganglion or nerve root.

In any of the preceding embodiments and aspects, the administering is directed into the parenchyma or the cerebrospinal spinal fluid of the central nervous system.

In any of the preceding embodiments and aspects, the synthetic RNA encoding a gene-editing protein is administered systemically and its penetrance to the CNS or PNS is increased by encapsulation in a viral or non-viral particle, by electrical stimulation, by acoustical stimulation, and/or by co-administration with a drug.

In any of the preceding embodiments and aspects, the RNA comprises or encodes a transport signal that directs the RNA or a protein product to a neuron's cell body or to a distal portion of the neuron.

In any of the preceding embodiments and aspects, the synthetic RNA encoding a gene-editing protein decreases expression of a wild-type or a mutant form of NaV 1.3, NaV 1.7, NaV 1.8, or NaV 1.9.

In any of the preceding embodiments and aspects, the synthetic RNA encoding a gene-editing protein increases expression of a wild-type or a mutant form of NaV 1.3, NaV 1.7, NaV 1.8, or NaV 1.9.

In any of the preceding embodiments and aspects, the synthetic RNA encoding a gene-editing protein increases enkephalins and/or glutamic acid decarboxylases in mesenchymal stem cells, thereby treating and/or reducing pain.

In any of the preceding embodiments and aspects, the methods further comprise administering electrical stimulation, a drug, and/or a cell therapy to increase efficacy.

In any of the preceding embodiments and aspects, the gene-editing protein is selected from a TALEN, a meganuclease, a nuclease, a zinc finger nuclease, a CRISPR-associated protein, CRISPR/Cas9, Cas9, xCas9, Cas12a (Cpf1), Cas13a, Cas14, CasX, CasY, a Class 1 Cas protein, a Class 2 Cas protein, and MAD7.

In any of the preceding embodiments and aspects, the gene-editing protein comprises: (a) a DNA-binding domain comprising a plurality of repeat sequences and at least one of the repeat sequences comprises the amino acid sequence: LTPvQWAIAwxyzGHGG (SEQ ID NO: 629), wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 630) or GGKQALETVQRLLPVLCQA (SEQ ID NO: 631); and (b) a nuclease domain comprising a catalytic domain of a nuclease.

In embodiments, the nuclease domain is capable of forming a dimer with another nuclease domain.

In any of the preceding embodiments and aspects, the nuclease domain comprises the catalytic domain of a protein comprising the amino acid sequence of SEQ ID NO: 632.

In any of the preceding embodiments and aspects, at least one of the repeat sequences comprising the amino acid sequence LTPvQWAIAwxyzGHGG (SEQ ID NO: 629) is between 36 and 39 amino acids long.

In any of the preceding embodiments and aspects, the pain is post-surgical and/or chronic pain.

In any of the preceding embodiments and aspects, the synthetic RNA comprises one or more non-canonical nucleotides.

In embodiments, the one or more non-canonical nucleotides avoids substantial cellular toxicity.

In any of the preceding embodiments and aspects, the non-canonical nucleotides have one or more substitutions at positions selected from the 2C, 4C, and 5C positions for a pyrimidine, or selected from the 6C, 7N and 8C positions for a purine.

In any of the preceding embodiments and aspects, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In any of the preceding embodiments and aspects, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 10% of guanine residues are non-canonical nucleotides, and the non-canonical nucleotide is optionally 7-deazaguanosine.

In any of the preceding embodiments and aspects, the synthetic RNA comprises no more than about 50% 7-deazaguanosine in place of guanosine residues.

In any of the preceding embodiments and aspects, the synthetic RNA does not comprise non-canonical nucleotides in place of adenosine residues.

In any of the preceding embodiments and aspects, the synthetic RNA comprises a 5′ cap structure.

In any of the preceding embodiments and aspects, the synthetic RNA comprises a Kozak consensus sequence.

In any of the preceding embodiments and aspects, the synthetic RNA comprises a 5′-UTR which comprises a sequence that increases RNA stability in vivo, and the 5′-UTR optionally comprises an alpha-globin or beta-globin 5′-UTR.

In any of the preceding embodiments and aspects, the synthetic RNA comprises a 3′-UTR which comprises a sequence that increases RNA stability in vivo, and the 3′-UTR optionally comprises an alpha-globin or beta-globin 3′-UTR.

In any of the preceding embodiments and aspects, the synthetic RNA comprises a 5′-UTR which comprises a microRNA binding site that modulates RNA stability in a cell type-specific manner.

In any of the preceding embodiments and aspects, the synthetic RNA comprises a 3′-UTR which comprises a microRNA binding site that modulates RNA stability in a cell type-specific manner.

In any of the preceding embodiments and aspects, the synthetic RNA comprises a 3′ poly(A) tail.

In any of the preceding embodiments and aspects, the synthetic RNA comprises a 3′ poly(A) tail which comprises from about 20 nucleotides to about 250 nucleotides.

In any of the preceding embodiments and aspects, the synthetic RNA comprises about 200 nucleotides to about 5000 nucleotides.

In any of the preceding embodiments and aspects, the synthetic RNA comprises from about 500 to about 2000 nucleotides, or about 500 to about 1500 nucleotides, or about 500 to about 1000 nucleotides.

In any of the preceding embodiments and aspects, the synthetic RNA is prepared by in vitro transcription.

In any of the preceding embodiments and aspects, the effective amount of the synthetic RNA is administered as one or more injections each injection comprising about 10 ng to about 5000 ng of RNA.

In any of the preceding embodiments and aspects, the effective amount of the synthetic RNA is administered as one or more injections each injection comprising no more than about 10 ng, or no more than about 20 ng, or no more than about 50 ng, or no more than about 100 ng, or no more than about 200 ng, or no more than about 300 ng, or no more than about 400 ng, or no more than about 500 ng, or no more than about 600 ng, or no more than about 700 ng, or no more than about 800 ng, or no more than about 900 ng, or no more than about 1000 ng, or no more than about 1100 ng, or no more than about 1200 ng, or no more than about 1300 ng, or no more than about 1400 ng, or no more than about 1500 ng, or no more than about 1600 ng, or no more than about 1700 ng, or no more than about 1800 ng, or no more than about 1900 ng, or no more than about 2000 ng, or no more than about 3000 ng, or no more than about 4000 ng, or no more than about 5000 ng of RNA.

In any of the preceding embodiments and aspects, the effective amount of the synthetic RNA is administered as one or more injections each injection comprising about 10 ng, or about 20 ng, or about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng of RNA.

In any of the preceding embodiments and aspects, the effective amount of the synthetic RNA comprises one or more lipids and/or polymers to enhance uptake of RNA by cells.

In any of the preceding embodiments and aspects, the effective amount of the synthetic RNA comprises a cationic liposome and/or cationic polymer formulation.

In embodiments, a lipid and/or a polymer of the cationic liposome formulation is selected from Table 1.

An aspect of the present invention is a method of polynucleotide delivery to the central nervous system, comprising a synthetic polynucleotide formulated with a liposome comprising one or more lipids selected from Table 1.

In embodiments, the polynucleotide is a synthetic RNA.

In any of the preceding embodiments and aspects, the liposome comprises 1,2-dioleoyl-3-dimethylammonium-propane (DODAP).

In embodiments, the liposome further comprises one or more helper lipids, optionally selected from dioleoyl phosphatidyl ethanolamine (DOPE), 1,2-distearoyl-sn-glycero-3-phosphocholine (DSPC), and cholesterol.

In any of the preceding embodiments and aspects, the liposome further comprises a PEGylated lipid.

In any of the preceding embodiments and aspects, the subject in need is a human.

In any of the preceding embodiments and aspects, the effective amount of the synthetic RNA is administered about weekly, for at least 2 weeks.

In any of the preceding embodiments and aspects, the effective amount of the synthetic RNA is administered about every other week for at least one month.

In any of the preceding embodiments and aspects, the effective amount of the synthetic RNA is administered monthly or about every other month.

In any of the preceding embodiments and aspects, the effective amount of the synthetic RNA is administered for at least two months, or at least 4 months, or at least 6 months, or at least 9 months, or at least one year.

In any of the preceding embodiments and aspects, the synthetic RNA comprises 5-methoxyuridine.

An aspect of the present invention is a composition comprising an effective amount of the synthetic RNA used in the method of any one of the above aspects or embodiments.

In embodiments, having an injection volume of less than about 1 mL, less than about 0.5 mL, less than about 0.2 mL, less than about 0.1 mL, less than about 0.05 mL, less than about 0.02 mL, less than about 0.01 mL, less than about 0.005 mL, less than about 0.002 mL, or less than about 0.001 mL.

An aspect of the present invention is a pharmaceutical composition, comprising the composition of any of the preceding embodiments and aspects and a pharmaceutically-acceptable excipient.

In embodiments, use of the composition of any of the preceding embodiments and aspects, or the pharmaceutical composition of any of the preceding embodiments and aspects in the treatment of a disease or disorder described herein.

In embodiments, use of the composition of any of the preceding embodiments and aspects, or the pharmaceutical composition of any of the preceding embodiments and aspects in the manufacture of a medicament for the treatment of a disease or disorder described herein.

An aspect of the present invention is a composition comprising a synthetic RNA used in the method of any one of the preceding embodiments and aspects and formulated with one or more lipids and/or polymers selected from Table 1.

An aspect of the present invention is a composition comprising a DNA template comprising: (a) a sequence encoding a protein, (b) a tail region comprising deoxyadenosine nucleotides and one or more other nucleotides, and (c) a restriction enzyme binding site.

In embodiments, the one or more other nucleotides comprises deoxyguanosine residues.

In embodiments, the tail region comprises about 1%, about 2%, about 5%, about 10%, about 15%, about 20%, about 25%, about 30%, about 35%, about 40%, about 45%, or about 50% deoxyguanosine residues.

In embodiments, the tail region comprises more than 50% deoxyguanosine residues.

In embodiments, the one or more other nucleotides comprises deoxycytidine residues.

In embodiments, the tail region comprises about 1%, about 2%, about 5%, about 10%, about 15%, about 20%, about 25%, about 30%, about 35%, about 40%, about 45%, or about 50% deoxycytidine residues.

In embodiments, the tail region comprises more than 50% deoxycytidine residues.

In embodiments, the one or more other nucleotides comprises deoxythymidine residues.

In embodiments, the tail region comprises about 1%, about 2%, about 5%, about 10%, about 15%, about 20%, about 25%, about 30%, about 35%, about 40%, about 45%, or about 50% deoxythymidine residues.

In embodiments, the tail region comprises more than 50% deoxythymidine residues.

In embodiments, the one or more other nucleotides comprise deoxyguanosine residues and deoxycytidine residues.

In any of the preceding embodiments and aspects, the tail region comprises about 99%, about 98%, about 95%, about 90%, about 85%, about 80%, about 75%, about 70%, about 65%, about 60%, about 55%, or about 50% deoxyadenosine residues.

In any of the preceding embodiments and aspects, the tail region comprises fewer than 50% deoxyadenosine residues.

In any of the preceding embodiments and aspects, the length of the tail region is between about 80 base pairs and about 120 base pairs, about 120 base pairs and about 160 base pairs, about 160 base pairs and about 200 base pairs, about 200 base pairs and about 240 base pairs, about 240 base pairs and about 280 base pairs, or about 280 base pairs and about 320 base pairs.

In any of the preceding embodiments and aspects, the length of the tail region is greater than 320 base pairs.

An aspect of the present invention is a composition comprising a synthetic RNA comprising: (a) a sequence encoding a protein, and (b) a tail region comprising adenosine nucleotides and one or more other nucleotides.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues.

In embodiments, the tail region comprises more than 50% guanosine residues.

In embodiments, the one or more other nucleotides comprises cytidine residues.

In embodiments, the tail region comprises about 1%, about 2%, about 5%, about 10%, about 15%, about 20%, about 25%, about 30%, about 35%, about 40%, about 45%, or about 50% cytidine residues.

In embodiments, the tail region comprises more than 50% cytidine residues.

In embodiments, the one or more other nucleotides comprises uridine residues.

In embodiments, the tail region comprises about 1%, about 2%, about 5%, about 10%, about 15%, about 20%, about 25%, about 30%, about 35%, about 40%, about 45%, or about 50% uridine residues.

In embodiments, the tail region comprises more than 50% uridine residues.

In embodiments, the one or more other nucleotides comprise guanosine residues and cytidine residues.

In any of the preceding embodiments and aspects, the tail region comprises about 99%, about 98%, about 95%, about 90%, about 85%, about 80%, about 75%, about 70%, about 65%, about 60%, about 55%, or about 50% adenosine residues.

In any of the preceding embodiments and aspects, the tail region comprises fewer than 50% adenosine residues.

In any of the preceding embodiments and aspects, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In any of the preceding embodiments and aspects, the length of the tail region is greater than 320 nucleotides.

An aspect of the present invention is a composition comprising a synthetic RNA comprising a 3-untranslated region sequence having at least 90% homology to the 3-untranslated region of a gene selected from: APOBEC3H, CD52, DMC1, EIF3E, GPR160, and RPS24.

In any of the preceding embodiments and aspects, the synthetic RNA further comprises one or more non-canonical nucleotides.

In embodiments, the non-canonical nucleotides have one or more substitutions at positions selected from the 2C, 4C, and 5C positions for a pyrimidine, or selected from the 6C, 7N and 8C positions for a purine.

In any of the preceding embodiments and aspects, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In any of the preceding embodiments and aspects, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 10% of guanine residues are non-canonical nucleotides, and the non-canonical nucleotide is optionally 7-deazaguanosine.

In any of the preceding embodiments and aspects, the synthetic RNA comprises no more than about 50% 7-deazaguanosine in place of guanosine residues.

In any of the preceding embodiments and aspects, the synthetic RNA does not comprise non-canonical nucleotides in place of adenosine residues.

In any of the preceding embodiments and aspects, the synthetic RNA comprises 5-methoxyuridine.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

Any aspect or embodiment disclosed herein can be combined with any other aspect or embodiment as disclosed herein.

### BRIEF DESCRIPTION OF THE DRAWINGS

FIG. 1 depicts intradermal injection of a solution comprising RNA encoding GFP into the ventral forearm of a healthy, 33 year-old, 70 kg, male human subject.

FIG. 2 depicts a region of the ventral forearm of the subject shown in FIG. 1 after treatment with RNA comprising 5-methoxyuridine and encoding GFP (injection sites 1-3) or COL7 (injection site 4). The image was taken immediately following the final injection.

FIG. 3 depicts the region of FIG. 2, 24 hours after injection.

FIG. 4 depicts the results of fluorescent imaging of the region of FIG. 2, using the indicated fluorescent channels. The dose at each injection site is also indicated. Images were taken 24 hours after injection.

FIG. 5 depicts the results of fluorescent imaging of the region of FIG. 2, using the FITC fluorescent channel. The dose at each injection site is indicated. Images were taken 48 hours after injection.

FIG. 6 depicts the results of quantitative fluorescent imaging of the region of FIG. 2, using the FITC fluorescent channel. The horizontal axis indicates time after injection.

FIG. 7A depicts the results of fluorescent imaging of an independent experiment in which a region of the ventral forearm of as the subject shown in FIG. 2 treated with RNA comprising 5-methoxyuridine and encoding GFP. The image was taken 24 hours after injection.

FIG. 7B depicts intradermal injection of RNA encoding GFP, formulated for intradermal injection, into the ventral forearm of the subject in FIG. 1, 48 months following the injection of FIG. 1. The arrow indicates an approximately 1 cm² area of GFP expression at the site of injection.

FIG. 8 depicts primary human neonatal fibroblasts reprogrammed by five transfections with RNA encoding reprogramming proteins. Cells were fixed and stained for Oct4 protein. Nuclei were counterstained with Hoechst 33342.

FIG. 9 depicts primary adult human dermal fibroblasts transfected with RNA encoding green fluorescent protein (“GFP”), prepared and stored as indicated.

FIG. 10 depicts the results of an experiment in which 100,000 primary human neonatal epidermal keratinocytes (animal-protein free) were transfected with 2 μg RNA encoding the indicated gene-editing proteins (1 μg L and 1 μg R). DNA was harvested after 48 h and analyzed for gene editing (A/B indicates RIBOSLICE_A L, RIBOSLICE_B R; RIBOSLICE A indicates repeat sequences comprising the sequence: GHGG, HG, GHGG, HG, etc.; RIBOSLICE B indicates repeat sequences comprising the sequence: HG, GHGG, HG, GHGG, etc.; L targets the sequence: TGCCTGGTCCCTGTCTCCCT (SEQ ID NO: 615); R targets the sequence: TGTCTTCTGGGCAGCATCTC (SEQ ID NO: 616); a target sequence is approximately 75 bp from A1AT [SERPINA1] start codon). “TAL” indicates a control TALEN directed to the target sequence.

FIG. 11 depicts the results of an experiment in which 100,000 primary human neonatal epidermal keratinocytes (animal-protein free) were transfected with 2 μg RNA encoding the indicated gene-editing proteins (1 μg L and 1 μg R). DNA was harvested after 48 h and analyzed for gene editing (A/B indicates RIBOSLICE_A L, RIBOSLICE_B R; RIBOSLICE A indicates repeat sequences comprising the sequence: GHGG, HG, GHGG, HG, etc.; RIBOSLICE B indicates repeat sequences comprising the sequence: HG, GHGG, HG, GHGG, etc.; L targets the sequence: TATTCCCGGGCTCCCAGGCA (SEQ ID NO: 622); R targets the sequence: TCTCCTGGCCTTCCTGCCTC (SEQ ID NO: 612); a target sequence is near the end of exon 73 of COL7A1). “TAL” indicates a control TALEN directed to the target sequence.

FIG. 12 depicts the results of an experiment in which 50,000 primary human neonatal epidermal keratinocytes (HEKn) (animal-protein free) were transfected with 2 μg RNA encoding the indicated gene-editing proteins. DNA was harvested after 48 h and analyzed for gene editing (“Neg” indicates untreated HEKn DNA; “WT” indicates wild type FokI; “EA” indicates enhanced activity FokI (S35P and K58E); “Het” indicates heterodimer (L: Q103E, N113D, I116L, R: E107K, H154R, I155K); “EA/Het” indicates both EA and Het; L targets the sequence: TATTCCCGGGCTCCCAGGCA (SEQ ID NO: 622); R targets the sequence: TCTCCTGGCCTTCCTGCCTC (SEQ ID NO: 612); a target sequence is near the end of exon 73 of COL7A1).

FIG. 13 depicts the results of an experiment in which 100,000 primary human neonatal epidermal keratinocytes (animal-protein free) were transfected with 2 μg RNA encoding the HBB exon 1 TALEN L and HBB exon 1 TALEN R gene-editing proteins (1 μg each). DNA was harvested after 48 h and analyzed for gene editing (T7E1 assay; forward primer: GCCAAGGACAGGTACGGCTGTCATC (SEQ ID NO: 627); reverse primer: CTTGCCATGAGCCTTCACCTTAGGGTTG (SEQ ID NO: 628); product size: 518 nt; predicted band sizes: 300 nt, 218 nt).

FIG. 14 depicts the results of an experiment in which 100,000 primary human neonatal epidermal keratinocytes (animal-protein free) were transfected with 2 μg RNA encoding the PD1 exon 1 TALEN L and PD1 exon 1 TALEN R gene-editing proteins (1 μg each). DNA was harvested after 48 h and analyzed for gene editing (T7E1 assay; forward primer: TCCTCTGTCTCCCTGTCTCTGTCTCTCTCTC (SEQ ID NO: 594); reverse primer: GGACTTGGGCCAGGGGAGGAG (SEQ ID NO: 595); product size: 612 nt; predicted band sizes: 349 nt, 263 nt).

FIG. 15 depicts the encapsulation efficiency of liposomes comprising PEGylated lipids and encapsulating RNA encoding NOVEPOIETIN or GFP.

FIG. 16 depicts the results of an assay in which liposomes comprising 2 μg of RNA were applied dropwise to 50,000 primary human neonatal epidermal keratinocytes (animal-protein free) in one well of a 6-well plate. After 48 hours, gene editing efficiency was assayed using a mutation-specific nuclease (T7E1).

FIG. 17 depicts a SURVEYOR assay using the DNA of primary adult human dermal fibroblasts transfected with RNA TALENs targeting the sequence TGAGCAGAAGTGGCTCAGTG (SEQ ID NO: 467) and TGGCTGTACAGCTACACCCC (SEQ ID NO: 468), located within the COL7A1 gene. The bands present in the +RNA lane indicate editing of a region of the gene that is frequently involved in dystrophic epidermolysis bullosa.

FIG. 18 depicts a SURVEYOR assay using the DNA of primary adult human dermal fibroblasts transfected with RNA TALENs targeting the sequence TTCCACTCCTGCAGGGCCCC (SEQ ID NO: 469) and TCGCCCTTCAGCCCGCGTTC (SEQ ID NO:470), located within the COL7A1 gene. The bands present in the +RNA lane indicate editing of a region of the gene that is frequently involved in dystrophic epidermolysis bullosa.

FIG. 19 shows the immunogenicity of various synthetic RNA constructs in the context of a gene-editing (i.e. unmodified nucleotides “A,G,U,C”; pseudouridine only “psU”; 5-methylcytidine only “5mC”; both pseudouridine and 5-methylcytidine “psU+5mC”; and a negative control “neg”).

FIG. 20 shows the gene-editing activity in cells transfected with various synthetic RNA constructs (i.e. unmodified nucleotides “A,G,U,C”; psuedouridine only “psU”; 5-methylcytidine only “5mC”; both psuedouridine and 5-methylcytidine “psU+5mC”; and a negative control “neg”).

FIG. 21 depicts gene editing of the COL7A1 gene in primary human epidermal keratinocytes transfected with RNA encoding TALENs and a single-stranded DNA repair template (“RT”) of the indicated length. The presence of bands at the locations shown by asterisks (“*”) indicates successful gene editing.

FIG. 22 depicts gene editing (“T7E1”) and correction (“Digestion”) of the COL7A1 gene in primary human epidermal keratinocytes transfected with RNA encoding TALENs and an 80 nt single-stranded DNA repair template (“RT”). The presence of bands at the locations shown by asterisks (“*”) indicates successful gene editing (“T7E1”) and correction (“Digestion”).

FIG. 23 depicts gene correction of the COL7A1 gene in primary human epidermal keratinocytes transfected with RNA encoding TALENs and a single-stranded DNA repair template (“RT”) of the indicated length. The presence of bands at the locations shown by asterisks (“*”) indicates successful gene correction.

FIG. 24 depicts gene editing of the COL7A1 gene in primary human epidermal keratinocytes transfected with RNA encoding TALENs and an 80 nt single-stranded DNA repair template (“RT”) at the indicated ratios of RNA to repair template. The presence of bands at the locations shown by asterisks (“*”) indicates successful gene editing.

FIG. 25 depicts gene correction of the COL7A1 gene in primary human epidermal keratinocytes transfected with RNA encoding TALENs and an 80 nt single-stranded DNA repair template (“RT”) at the indicated ratios of RNA to repair template. The presence of bands at the locations shown by asterisks (“*”) indicates successful gene correction.

FIG. 26 depicts the serum levels of FGF21, IL15, IL6, IL22, and Novepoietin following a single intradermal injection of various RNAs encoding these proteins as described in Example 45. Three rats were analyzed for each RNA tested.

FIG. 27 depicts the results of an experiment in which 50,000 primary human neonatal epidermal keratinocytes (animal-protein free) were transfected with 2 μg RNA encoding the COL7A1 exon 73 spliceMod TALEN L1-4 and COL7A1 exon 73 spliceMod TALEN R1-3 gene-editing proteins (1 μg each). Specific combinations transfected were L1/R1, L2/R1, L2/R2, L3/R3, and L4/R3. DNA was harvested after 48 h and analyzed for gene editing (T7E1 assay; forward primer: GCATCTGCCCTGCGGGAGATC (SEQ ID NO: 478); reverse primer: CCACGTTCTCCTTTCTCTCCCCGTTC (SEQ ID NO: 479); product size: 535 nt; predicted band sizes: 203 nt, 332 nt for L1/R1; 202 nt, 333 nt for L1/R2; 201 nt, 334 nt for L2/R2; 189 nt, 346 nt for L3/R3; and 186 nt, 349 nt for L4/R3).

FIG. 28 depicts the results of an experiment in which 50,000 primary human neonatal epidermal keratinocytes (animal-protein free) were transfected with 2 μg RNA encoding the COL7A1 exon 73 spliceMod TALEN L2, RIBOSLICE L2A, or RIBOSLICE L2B and COL7A1 exon 73 spliceMod TALEN R2, RIBOSLICE R2A or RIBOSLICE R2B gene-editing proteins (1 μg each). Specific combinations transfected were L2A/R2A, L2B/R2B, and L2/R2. DNA was harvested after 48 h and analyzed for gene editing (T7E1 assay; forward primer: GCATCTGCCCTGCGGGAGATC (SEQ ID NO: 478); reverse primer: CCACGTTCTCCTTTCTCTCCCCGTTC (SEQ ID NO: 479); product size: 535 nt; predicted band sizes: 201 nt, 334 nt).

FIG. 29 depicts the results of an experiment in which 50,000 primary human neonatal epidermal keratinocytes (animal-protein free) were transfected with 2 μg RNA encoding the COL7A1 exon 73 spliceMod TALEN L1-4 and COL7A1 exon 73 spliceMod TALEN R1-3 gene-editing proteins (1 μg each). Specific combinations transfected were L1/R1, L1/R2, L2/R2, L3/R3, and L4/R3. RNA was harvested after 48 h and analyzed for presence of exon 73 in the spliced mRNA (RT-PCR; reverse transcription primer: GCTCTCCTGGTAGACCCGGGTTG (SEQ ID NO: 658), amplification forward primer: GGTTGCTGGAAACTGCTGGCATCAAGGCATCTG (SEQ ID NO: 659) amplification reverse primer: CACCCTTGAGTCCAGGGGGTCCCTGTTCTC (SEQ ID NO: 661); full product size: 513 nt; product size without exon 73: 312 nt).

FIG. 30A depicts the results of an experiment in which 50,000 primary human neonatal epidermal keratinocytes (animal-protein free) were transfected with 2 μg RNA encoding the COL7A1 exon 73 spliceMod TALEN L1-4 and COL7A1 exon 73 spliceMod TALEN R1-3 gene-editing proteins (1 μg each). Specific combinations transfected were L1/R1, L2/R1, L2/R2, L3/R3, and L4/R3. RNA was harvested after 48 h and analyzed for presence of exon 73 in the spliced mRNA (RT-PCR; reverse transcription primer: GCTCTCCTGGTAGACCCGGGTTG (SEQ ID NO: 658), amplification forward primer: GCATCTGCCCTGCGGGAGATC (SEQ ID NO: 478) amplification reverse primer: CCACGTTCTCCTTTCTCTCCCCGTTC (SEQ ID NO: 479); full product size: 353 nt; product size without exon 73: 152 nt).

FIG. 30B depicts the results of an experiment in which 50,000 primary human neonatal epidermal keratinocytes (animal-protein free) were transfected with 2 μg RNA encoding the COL7A1 exon 73 splice acceptor-targeting pairs (target sequences: TGTACAGCCACCAGCATTCT (SEQ ID NO: 652) and TCCAGGAAAGCCGATGGGGC (SEQ ID NO: 656)) (1 μg each individual pair component) with mutations in the N-terminus of the protein. 1: I56L, 2: K57R, 3: R61K, 4: A65G, 5: A70G, 6: K57E, 7. K57E and V60A. DNA was harvested after 48 h and analyzed for gene editing (T7E1 assay; forward primer: GCATCTGCCCTGCGGGAGATC (SEQ ID NO: 478), reverse primer: CCACGTTCTCCTTTCTCTCCCCGTTC (SEQ ID NO: 479), product size: 535 nt, predicted band sizes: 202 nt, 333 nt).

FIG. 31 depicts the results of an experiment in which 4 wells, each containing 50,000 primary human neonatal epidermal keratinocytes (animal-protein free) were each transfected with 2 μg RNA encoding the COL7A1 exon 73 spliceMod TALEN L2 and COL7A1 exon 73 spliceMod TALEN R2 gene-editing proteins (1 μg each). RNA was harvested after 1, 3, 5 and 18 days following transfection and analyzed for presence of exon 73 in the spliced mRNA (RT-PCR; reverse transcription primer: GCTCTCCTGGTAGACCCGGGTTG (SEQ ID NO: 658), amplification forward primer: GGTTGCTGGAAACTGCTGGCATCAAGGCATCTG (SEQ ID NO: 659) amplification reverse primer: CACCCTTGAGTCCAGGGGGTCCCTGTTCTC (SEQ ID NO: 661); full product size: 513 nt; product size without exon 73: 312 nt).

FIG. 32 depicts BMP7 protein levels in rats treated with RNAs encoding BMP7 variants as described in Example 42. Error bars indicate SEM (n=6).

FIG. 33 depicts the results of an experiment in which cortical tissue from embryonic day 18 Sprague Dawley rat embryos was transfected with 0.1 μg RNA encoding mRFP. The tissue was examined approximately 16 h after transfection by brightfield and fluorescent microscopy.

FIG. 34 depicts the results of an experiment in which 50,000-100,000 cortical neurons from embryonic day 18 Sprague Dawley rat embryos were cultured on a poly-D-lysine coated 24-well for 6 days and then transfected with 0.05 μg RNA encoding mRFP or 1-5×10¹⁰ viral genomes of AAV2-mRFP. The cells were imaged every hour for the first 18 h and then every 6 h by brightfield and fluorescent microscopy. Error bars show standard deviation.

FIG. 35 depicts the results of an experiment in which 100,000 cortical neurons from embryonic day 18 Sprague Dawley rat embryos were cultured on a poly-D-lysine coated 24-well for 6 days and then transfected with 0.05 μg RNA encoding mRFP. Images show RFP fluorescence. The number at the top left corner of each panel indicates the time after transfection in hours.

FIG. 36 depicts the results of an experiment in which 50,000 primary human neonatal epidermal keratinocytes (animal-protein free) were transfected with 2 μg RNA encoding the HTT TALEN pairs 1/1 (target sequences: TTTGACAAATGAGTGTTTCT (SEQ ID NO: 730) and TCTCCACTGATCTCATCCTT (SEQ ID NO: 731)) and 2/2 (target sequences: TCGCCATTTGACAAATGAGT and TGATCTCATCCTTCACTGAG) (1 μg each individual pair component). DNA was harvested after 10 d and analyzed for gene editing (T7E1 assay; forward primer: AGTGACCACTGCCAACAGCTTCATGTC (SEQ ID NO: 734); reverse primer: GGGTAACAGCTGAATCAGGCCCTTCG (SEQ ID NO: 735); product size: 920 bp; predicted band sizes: 322 bp, 598 bp for pair 1/1; 328 bp, 592 bp for pair 2/2).

FIG. 37 depicts a tissue section of a liver treated in vivo with RNA encoding GFP and stained for the presence of GFP (shown at 40× magnification).

FIG. 38 depicts a tissue section of a spinal cord treated in vivo with RNA encoding GFP and stained for the presence of GFP (shown at 40× magnification).

FIG. 39 depicts a tissue section of a lateral ventricle treated in vivo with a control buffer and stained for the presence of GFP (shown at 10× magnification).

FIG. 40 depicts a tissue section of a lateral ventricle treated in vivo with RNA encoding GFP and stained for the presence of GFP (shown at 4× magnification).

FIG. 41 depicts a tissue section of a lateral ventricle treated in vivo with RNA encoding GFP and stained for the presence of GFP (shown at 10× magnification).

FIG. 42 depicts a tissue section of a lateral ventricle treated in vivo with RNA encoding GFP and stained for the presence of GFP (shown at 4× magnification).

FIG. 43 depicts a tissue section of a lateral ventricle treated in vivo with RNA encoding GFP and stained for the presence of GFP (shown at 10× magnification).

FIG. 44 depicts a tissue section of a lateral ventricle treated in vivo with a control buffer and stained for the presence of GFP (shown at 10× magnification).

FIG. 45 depicts a tissue section of a lateral ventricle treated in vivo with RNA encoding GFP and stained for the presence of GFP (shown at 4× magnification).

FIG. 46 depicts a tissue section of a lateral ventricle treated in vivo with RNA encoding GFP and stained for the presence of GFP (shown at 10× magnification).

FIG. 47 depicts a tissue section of a lateral ventricle treated in vivo with RNA encoding GFP and stained for the presence of GFP (shown at 40× magnification).

FIG. 48 depicts a tissue section of a hippocampus treated in vivo with a control buffer and stained for the presence of GFP (shown at 10× magnification).

FIG. 49 depicts a tissue section of a hippocampus treated in vivo with RNA encoding GFP and stained for the presence of GFP (shown at 10× magnification).

FIG. 50 depicts a tissue section of a hippocampus treated in vivo with RNA encoding GFP and stained for the presence of GFP (shown at 40× magnification).

FIG. 51 depicts a tissue section of a rat ventricle treated in vivo with RNA encoding GFP (formulated as an LNP) and stained for the presence of GFP (shown at 40× magnification).

FIG. 52 depicts a tissue section of a rat ventricle treated in vivo with RNA encoding GFP (formulated as an LNP) and stained for the presence of GFP (shown at 40× magnification).

FIG. 53 depicts a tissue section of a rat ventricle treated in vivo with RNA encoding GFP (formulated as an LNP) and stained for the presence of GFP (shown at 40× magnification).

FIG. 54 depicts a tissue section of a rat ventricle treated in vivo with RNA encoding GFP (formulated as an LNP) and stained for the presence of GFP (shown at 40× magnification).

FIG. 55 depicts a tissue section of a rat ventricle treated in vivo with RNA encoding GFP (formulated as an LNP) and stained for the presence of GFP (shown at 40× magnification).

FIG. 56 depicts a tissue section of a rat ventricle treated in vivo with RNA encoding GFP (formulated as an LNP) and stained for the presence of GFP (shown at 40× magnification).

FIG. 57 depicts the results of an experiment in which 20,000 human neuroblastoma cells (SH-SY5Y) were transfected with 0.5 μg RNA encoding the below-indicated gene-editing proteins (0.25 μg L and 0.25 μg R). DNA was harvested after 48 h and analyzed for gene editing. “Neg” indicates untreated SH-SY5Y DNA, “1” indicates a TALEN pair targeting sequence TCCATCCAGGCCTCTTATGT (SEQ ID NO: 663) and TCTTTTCATCCTGTATATTT (SEQ ID NO: 664), “2” indicates a TALEN pair targeting sequence TGAAAAGATGGCAATGTTGC (SEQ ID NO: 665) and TGTGAAATGGACAAAGCTCT (SEQ ID NO: 666), “3” indicates a TALEN pair targeting sequence TCCCCCAGGACCTCAGAGCT (SEQ ID NO: 667) and TTCAATGAGGGCAAGAGACT (SEQ ID NO: 668). The T7E1 assay yields an expected product size of 725 nt (forward primer: gatggaatcttctcctggtc, SEQ ID NO: 669; reverse primer: aggaatgtccccatagatga, SEQ ID NO: 670). Predicted band sizes are: for pair 1: 495 nt and 230 nt, for pair 2: 544 nt and 181 nt, and for pair 3: 567 nt and 158 nt.

FIG. 58 depicts the results of staining RNA-reprogrammed human pluripotent stem cells (PSC), cells differentiated therefrom into mesenchymal stem cells (at both early passage and late passage), and STEMPRO™ BM Mesenchymal Stem Cells (Thermo Fisher). Cells were stained for pluripotent stem cell markers (Nanog and Sox2) and mesenchymal stem cell markers (CD73 and CD105).

FIG. 59 depicts the results of an experiment in which the telomere length of cells that were reprogrammed using RNA and differentiated into mesenchymal stem cells was measured.

FIG. 60 depicts the proliferative capacity of mesenchymal stem cells differentiated from RNA-reprogrammed human pluripotent stem cells (PSC-MSC) or STEMPRO™ BM Mesenchymal Stem Cells (BM-MSC; Thermo Fisher). Cells were cultured in 6-well plates in MESENCULT™ Proliferation medium (STEMCELL Technologies). Cells were counted and passaged when at or near confluence until senescence. Population doublings were calculated from total cell number.

FIG. 61 depicts the result of an experiment in which human neonatal epidermal keratinocytes were transfected with RNA encoding GFP and comprising the indicated tail.

FIG. 62 depicts the results of an experiment in which human neonatal epidermal keratinocytes were transfected with RNA encoding NOVEPOIETIN and comprising the indicated tail. The concentration of NOVEPOIETIN in the culture medium was measured by ELISA.

FIG. 63 depicts gene editing of the HBB gene in primary human cord blood CD34⁺ cells transfected with RNA encoding TALENs and with the oligonucleotide repair template (“+RNA”). A sample of cells that was not transfected with RNA or repair template is identified as the negative control, “Neg.”. The presence of bands following digestion with T7E1, analyzed by agarose gel electrophoresis, at the locations shown by asterisks (“*”) indicates successful gene editing.

FIG. 64 depicts gene repair of the HBB gene in primary human cord blood CD34⁺ cells transfected with RNA encoding TALENs and with the oligonucleotide repair template (“+RNA”). A sample of cells that was not transfected with RNA or repair template is identified as the negative control, “Neg.”. The presence of bands following digestion with HindIII-HF, analyzed by agarose gel electrophoresis, at the locations shown by asterisks (“*”), indicates successful gene repair.

FIG. 65 depicts the results of an experiment in which 50,000 primary human neonatal epidermal keratinocytes (animal-protein free) were transfected with 2 μg RNA encoding PD1 TALENs.

FIG. 66 depicts the results of an experiment in which 50,000 primary human neonatal epidermal keratinocytes (animal-protein free) were transfected with 2 μg RNA encoding ADORA2A TALENs.

FIG. 67 depicts the results of an experiment in which 50,000 RNA-reprogrammed human pluripotent stem cells were transfected with 2 μg RNA encoding PD1 TALENs.

### DETAILED DESCRIPTION OF THE INVENTION

The present invention is based, in part, on the discovery of a safe and effective dosing strategy for nucleic acid drugs, including RNA, such as RNA comprising non-canonical (or “modified”) nucleotides, in humans. The inventors believe this to be the first report of safe and effective dosing of RNA molecules, including those comprising non-canonical nucleotides, in humans. Despite reports in the art that very large doses of RNA molecules are needed for mammalian dosing, and minimal therapeutic effect is achieved despite high dosing (see, e.g. US Patent Publication No. 2013/0245103), the present inventors have surprisingly managed to dose synthetic RNA in a human and achieve significant target protein expression with minimal immunological or other side effects.

Synthetic mRNA Formulations

In various embodiments, the present invention provides improved doses, formulations, administration, and methods of use of nucleic acid drugs, which include RNA, which may contain non-canonical nucleotides (e.g. a residue other than adenine, guanine, thymine, uracil, and cytosine or the standard nucleoside, nucleotide, deoxynucleoside or deoxynucleotide derivatives thereof). In various embodiments, the RNA comprising non-canonical nucleotides leads to the expression of a protein encoded by the RNA, the protein often being one of therapeutic benefit (sometimes called the “target” or “protein of interest”). Further, this expression of therapeutic protein is achieved with minimal or negligible toxicity.

In various aspects, the present invention is based on the surprising discovery of safe and effective doses and administration parameters of nucleic acid drugs for human subjects. Nucleic acid drugs include a dsDNA molecule, a ssDNA molecule, a RNA molecule, a dsRNA molecule, a ssRNA molecule, a plasmid, an oligonucleotide, a synthetic RNA molecule, a miRNA molecule, an mRNA molecule, and an siRNA molecule. In various embodiments, the RNA comprises non-canonical nucleotides.

In some aspects, there is provided a method for delivering a nucleic acid drug, comprising administering an effective dose of a nucleic acid drug to a human subject in need thereof, wherein the nucleic acid drug comprises a synthetic RNA. In various embodiments, the effective dose is an amount sufficient to substantially increase an amount of a protein encoded by the nucleic acid drug in the human subject. For example, when the nucleic acid drug is a synthetic RNA comprising one or more modified nucleotides, the nucleic acid drug may result in higher protein expression than levels obtainable with a nucleic acid drug that does not comprise one or more modified nucleotides (e.g., RNA comprising the canonical nucleotides A, G, U, and C). In some embodiments, the nucleic acid drug results in about a 2-fold, or about a 3-fold, or about a 4-fold, or about a 5-fold, or about a 10-fold, or about a 15-fold, or about a 20-fold, or about a 25-fold, or about a 30-fold, or about a 35-fold, or about a 40-fold, or about a 45-fold, or about a 50-fold, or about a 100-fold increase in protein expression as compared to levels obtainable with a nucleic acid drug that does not comprise one or more modified nucleotides.

In some embodiments, the nucleic acid drug provides a sustained therapeutic effect that is optionally mediated by a sustained expression of target protein. For instance, in some embodiments, the therapeutic effect is present for over about 1 day, or over about 2 days, or over about 3 days, or over about 4 days, or over about 5 days, or over about 6 days, or over about 7 days, or over about 8 days, or over about 9 days, or over about 10 days, or over about 14 days after administration. In some embodiments, this sustained effect obviates the need for, or reduces the amount of, maintenance doses.

In some embodiments, the nucleic acid drug provides a sustained target protein level. For instance, in some embodiments, the target protein is present (e.g. in measurable amounts, e.g. in the serum of a patient to whom the nucleic acid drug has been administered) for over about 1 day, or over about 2 days, or over about 3 days, or over about 4 days, or over about 5 days, or over about 6 days, or over about 7 days, or over about 8 days, or over about 9 days, or over about 10 days, or over about 14 days after administration. In some embodiments, this sustained effect obviates the need for, or reduces the amount of, maintenance doses.

In various embodiments, the nucleic acid drug provides therapeutic action without sustained presence of the nucleic acid drug itself. In some embodiments, the nucleic acid drug is rapidly metabolized, for instance, within about 6 hours, or about 12 hours, or about 18 hours, or about 24 hours, or about 2 days, or about 3 days, or about 4 days, or about 5 days, or about 1 week from administration.

In various embodiments, the effective dose is an amount that substantially avoids cell toxicity in vivo. In various embodiments, the effective dose is an amount that substantially avoids an immune reaction in a human subject. For example, the immune reaction may be an immune response mediated by the innate immune system. Immune response can be monitored using markers known in the art (e.g. cytokines, interferons, TLRs). In some embodiments, the effective dose obviates the need for treatment of the human subject with immune suppressants agents (e.g. B18R) used to moderate the residual toxicity. Accordingly, in some embodiments, the present methods allow for dosing that provides increased protein expression and reduces toxicity.

In some embodiments, the immune response is reduced by about 10%, about 20%, about 30%, about 40%, about 50%, about 60%, about 70%, about 80%, about 90%, about 95%, about 99%, about 99.9%, or greater than about 99.9% as compared to the immune response induced by a corresponding unmodified nucleic acid. In some embodiments, upregulation of one or more immune response markers is reduced by about 10%, about 20%, about 30%, about 40%, about 50%, about 60%, about 70%, about 80%, about 90%, about 95%, about 99%, about 99.9%, or greater than about 99.9% as compared to the upregulation of the one or more immune response markers induced by a corresponding unmodified nucleic acid. In some embodiments, the immune response marker comprises an mRNA or protein product of an interferon gene, including an interferon alpha gene, IFNB1, TLR3, RARRES3, EIF2AK2, STAT1, STAT2, IFIT1, IFIT2, IFIT3, IFIT5, OAS1, OAS2, OAS3, OASL, ISG20 or a fragment, variant, analogue, or family-member thereof. In some embodiments, the immune response marker comprises an mRNA or protein product of an TNF gene, including an TNF alpha gene, TNFRSF1A; TNFRSF1B; LTBR; TNFRSF4; CD40; FAS; TNFRSF6B; CD27; TNFRSF8; TNFRSF9; TNFRSF10A; TNFRSF10B; TNFRSF10C; TNFRSF10D; TNFRSF11A; TNFRSF11B; TNFRSF12A; TNFRSF13B; TNFRSF13C; TNFRSF14; NGFR; TNFRSF17; TNFRSF18; TNFRSF19; TNFRSF21; TNFRSF25; and EDA2R or a fragment, variant, analogue, or family-member thereof. In some embodiments, the immune response marker comprises an mRNA or protein product of an interleukin gene, including an IL-6 gene, IL-1; IL-2; IL-3; IL-4; IL-5; IL-6; IL-7; IL-8 or CXCL8; IL-9; IL-10; IL-11; IL-12; IL-13; IL-14; IL-15; IL-16; IL-17; IL-18; IL-19; IL-20; IL-21; IL-22; IL-23; IL-24; IL-25; IL-26; IL-27; IL-28; IL-29; IL-30; IL-31; IL-32; IL-33; IL-35; IL-36 or a fragment, variant, analogue, or family-member thereof.

In some embodiments, cell death is about 10%, about 25%, about 50%, about 75%, about 85%, about 90%, about 95%, or over about 95% less than the cell death observed with a corresponding unmodified nucleic acid. Moreover, cell death may affect fewer than about 50%, about 40%, about 30%, about 20%, about 10%, about 5%, about 1%, about 0.1%, about 0.01% or fewer than about 0.01% of cells contacted with the modified nucleic acids.

In some embodiments, there is provided a method for expressing a protein of interest in a population of cells in a mammalian subject, comprising administering a non-viral transfection composition comprising an effective dose of a RNA encoding the protein of interest to said cells, the RNA containing one or more non-canonical nucleotides that avoid substantial cellular toxicity, where the transfection composition is administered in an amount that allows for expression of said protein in said cells for at least about five days (e.g. about 5, or about 6, or about 7, about 8, or about 9, or about 10, or about 14 days) without substantial cellular toxicity. In some embodiments, there is provided a method for expressing a protein of interest in a population of cells in a mammalian subject, comprising administering a non-viral transfection composition comprising an effective dose of a RNA encoding the protein of interest to said cells, the RNA containing one or more non-canonical nucleotides that avoid substantial cellular toxicity, where the transfection composition is administered in an amount that allows for expression of said protein in said cells for at least about six hours (e.g. about six hours, or about 12 hours, or about 1 day, or about 2 days, or about 3 days, or about 4 days, or about 5 days) without substantial cellular toxicity.

In some embodiments, the effective dose of the nucleic acid drug, including synthetic RNA, is about 100 ng to about 2000 ng, or about 200 ng to about 1900 ng, or about 300 ng to about 1800 ng, or about 400 ng to about 1700 ng, or about 500 ng to about 1600 ng, or about 600 ng to about 1500 ng, or about 700 ng to about 1400 ng, or about 800 ng to about 1300 ng, or about 900 ng to about 1200 ng, or about 1000 ng to about 1100 ng, or about 500 ng to about 2000 ng, or about 500 ng to about 1500 ng, or about 500 ng to about 1000 ng, or about 1000 ng to about 1500 ng, or about 1000 ng to about 2000 ng, or about 1500 ng to about 2000 ng, or about 100 ng to about 500 ng, or about 200 ng to about 400 ng, or about 10 ng to about 100 ng, or about 20 ng to about 90 ng, or about 30 ng to about 80 ng, or about 40 ng to about 70 ng, or about 50 ng to about 60 ng.

In some embodiments, the effective dose of the nucleic acid drug, including synthetic RNA, is no more than about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng.

In some embodiments, the effective dose of the nucleic acid drug, including synthetic RNA, is about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng.

In some embodiments, the effective dose of the nucleic acid drug, including synthetic RNA, is about 0.028 pmol, or about 0.05 pmol, or about 0.1 pmol, or about 0.2 pmol, or about 0.3 pmol, or about 0.4 pmol, or about 0.5 pmol, or about 0.6 pmol, or about 0.7 pmol, or about 0.8 pmol, or about 0.9 pmol, or about 1.0 pmol, or about 1.2 pmol, or about 1.4 pmol, or about 1.6 pmol, or about 1.8 pmol, or about 2.0 pmol, or about 2.2 pmol, or about 2.4 pmol, or about 2.6 pmol, or about 2.8 pmol, or about 3.0 pmol, or about 3.2 pmol, or about 3.4 pmol, or about 3.6 pmol, or about 3.8 pmol, or about 4.0 pmol, or about 4.2 pmol, or about 4.4 pmol, or about 4.6 pmol, or about 4.8 pmol, or about 5.0 pmol, or about 5.5 pmol, or about 5.7 pmol.

In some embodiments, the nucleic acid drug, including synthetic RNA, is administered at a concentration of about 0.1 nM, or about 0.25 nM, or about 0.5 nM, or about 0.75 nM, or about 1 nM, or about 2.5 nM, or about 5 nM, or about 7.5 nM, or about 10 nM, or about 20 nM, or about 30 nM, or about 40 nM, or about 50 nM, or about 60 nM, or about 70 nM, or about 80 nM, or about 90 nM, or about 100 nM, or about 110 nM, or about 120 nM, or about 150 nM, or about 175 nM, or about 200 nM.

In some embodiments, the effective dose of the nucleic acid drug is about 350 ng/cm², or about 500 ng/cm², or about 750 ng/cm², or about 1000 ng/cm², or about 2000 ng/cm², or about 3000 ng/cm², or about 4000 ng/cm², or about 5000 ng/cm², or about 6000 ng/cm², or about 7000 ng/cm². In other embodiments, the effective dose is less than about 350 ng/cm². In certain embodiments, the effective dose is about 35 ng/cm², or about 50 ng/cm², or about 75 ng/cm², or about 100 ng/cm², or about 150 ng/cm², or about 200 ng/cm², or about 250 ng/cm², or about 300 ng/cm², or about 350 ng/cm².

In some embodiments, the effective dose of the nucleic acid drug is about 35 ng/cm² to about 7000 ng/cm², or about 50 ng/cm² to about 5000 ng/cm², or about 100 ng/cm² to about 3000 ng/cm², or about 500 ng/cm² to about 2000 ng/cm², or about 750 ng/cm² to about 1500 ng/cm², or about 800 ng/cm² to about 1200 ng/cm², or about 900 ng/cm² to about 1100 ng/cm².

In some embodiments, the effective dose of the nucleic acid drug is about 1 picomole/cm², or about 2 picomoles/cm², or about 3 picomoles/cm², or about 4 picomoles/cm², or about 5 picomoles/cm², or about 6 picomoles/cm², or about 7 picomoles/cm², or about 8 picomoles/cm², or about 9 picomoles/cm², or about 10 picomoles/cm², or about 12 picomoles/cm², or about 14 picomoles/cm², or about 16 picomoles/cm², or about 18 picomoles/cm², or about 20 picomoles/cm². In other embodiments, the effective dose is less than about 1 picomole/cm². In certain embodiments, the effective dose is about 0.1 picomoles/cm², or about 0.2 picomoles/cm², or about 0.3 picomoles/cm², or about 0.4 picomoles/cm², or about 0.5 picomoles/cm², or about 0.6 picomoles/cm², or about 0.7 picomoles/cm², or about 0.8 picomoles/cm², or about 0.9 picomoles/cm², or about 1 picomole/cm².

In some embodiments, the effective dose of the nucleic acid drug is about 0.1 picomoles/cm² to about 20 picomoles/cm², or about 0.2 picomoles/cm² to about 15 picomoles/cm², or about 0.5 picomoles/cm² to about 10 picomoles/cm², or about 0.8 picomoles/cm² to about 8 picomoles/cm², or about 1 picomole/cm² to about 5 picomoles/cm², or about 2 picomoles/cm² to about 4 picomoles/cm².

In various embodiments, the nucleic acid drug, including synthetic RNA, is administered in a pharmaceutically acceptable formulation. In various embodiments, the nucleic acid drug, including synthetic RNA, is formulated for one or more of injection and topical administration. By way of example, the nucleic acid drug, including synthetic RNA, may be formulated for injection to a tissue of interest, e.g. a disease site (by way of non-limiting example, a tumor). In various embodiments, injection includes delivery via a patch. In some embodiments, the delivery is mediated by electrical stimulation. In various embodiments, the nucleic acid drug, including synthetic RNA, is formulated for administration to one or more of the epidermis (optionally selected from the stratum corneum, stratum lucidum, stratum granulosum, stratum spinosum, and stratum germinativum), basement membrane, dermis (optionally selected from the papillary region and the reticular region), subcutis, conjunctiva cornea, sclera, iris, lens, corneal limbus, optic nerve, choroid, ciliary body, anterior segment, anterior chamber, and retina. In various embodiments, the nucleic acid drug, including synthetic RNA, is formulated for one or more of subcutaneous injection, intradermal injection, subdermal injection, intramuscular injection, intraocular injection, intravitreal injection, intra-articular injection, intracardiac injection, intravenous injection, epidural injection, intrathecal injection, intraportal injection, intratumoral injection, and topical administration. In various embodiments, the nucleic acid drug, including synthetic RNA, is formulated for intradermal (ID) injection to one or more of the dermis or epidermis. In various embodiments, the nucleic acid drug, including synthetic RNA, is administered in a manner such that it effects one or more of keratinocytes and fibroblasts (e.g. causes these cells to express one or more therapeutic proteins).

In some embodiments, the formulation comprises liposomes. In certain embodiments, nucleic acids are fully encapsulated within liposomes. In other embodiments, nucleic acids are partially encapsulated within liposomes. In still other embodiments, nucleic acids and liposomes are both present with no encapsulation of the nucleic acids within the liposomes.

Accordingly, the present invention provides various formulations as described herein. Further, in some embodiments, the formulations described herein find use in the various delivery and/or treatment methods of the present invention. For instance, formulations can comprise a vesicle, for instance, a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989). In various embodiments, the formulation comprises an aqueous suspension of liposomes. Illustrative liposome components are set forth in Table 1, and are given by way of example, and not by way of limitation. In various embodiments, one or more, or two or more, or three or more, or four or more, or five or more of the lipids of Table 1 are combined in a formulation.

TABLE 1
Illustrative Biocompatible Lipids and Polymers

3β-[N-(N′,N′-dimethylaminoethane)-carbamoyl]cholesterol (DC-
Cholesterol)
1,2-dioleoyl-3-trimethylammonium-propane (DOTAP/18:1 TAP)
N-(4-carboxybenzyl)-N,N-dimethyl-2,3-bis(oleoyloxy)propan-1-
aminium (DOBAQ)
1,2-dimyristoyl-3-trimethylammonium-propane (14:0 TAP)
1,2-dipalmitoyl-3-trimethylammonium-propane (16:0 TAP)
1,2-stearoyl-3-trimethylammonium-propane (18:0 TAP)
1,2-dioleoyl-3-dimethylammonium-propane (DODAP/18:1 DAP)
1,2-dimyristoyl-3-dimethylammonium-propane (14:0 DAP)
1,2-dipalmitoyl-3-dimethylammonium-propane (16:0 DAP)
1,2-distearoyl-3-dimethylammonium-propane (18:0 DAP)
dimethyldioctadecylammonium (18:0 DDAB)
1,2-dilauroyl-sn-glycero-3-ethylphosphocholine (12:0 EthylPC)
1,2-dimyristoyl-sn-glycero-3-ethylphosphocholine (14:0 EthylPC)
1,2-dimyristoleoyl-sn-glycero-3-ethylphosphocholine (14:1 EthylPC)
1,2-dipalmitoyl-sn-glycero-3-ethylphosphocholine (16:0 EthylPC)
1,2-distearoyl-sn-glycero-3-ethylphosphocholine (18:0 EthylPC)
1,2-dioleoyl-sn-glycero-3-ethylphosphocholine (18:1 EthylPC)
1-palmitoyl-2-oleoyl-sn-glycero-3-ethylphosphocholine (16:1-18:1
EthylPC)
1,2-di-O-octadecenyl-3-trimethylammonium propane (DOTMA)
N1-[2-((1S)-1-[(3-aminopropyl)amino]-4-[di(3-amino-
propyl)amino]butylcarboxamido)ethyl]-
3,4-di[oleyloxy]-benzamide (MVL5)
2,3-dioleyloxy-N-[2-spermine carboxamide]ethyl-N,N-dimethyl-
1-propanammonium trifluoroacetate (DOSPA)
1,3-di-oleoyloxy-2-(6-carboxy-spermyl)-propylamid (DOSPER)
N-[1-(2,3-dimyristyloxy)propyl]-N,N-dimethyl-N-(2-
hydroxyethyl)ammonium bromide (DMRIE)
LIPOFECTAMINE, LIPOFECTAMINE 2000, LIPOFECTAMINE
RNAiMAX, LIPOFECTAMINE 3000,
LIPOFECTAMINE MessengerMAX, TransIT mRNA
dioctadecyl amidoglyceryl spermine (DOGS)
dioleoyl phosphatidyl ethanolamine (DOPE)
1,2-dilinoleyloxy-3-dimethylaminopropane (DLinDMA)
1,2-dilinoleyl-4-(2-dimethylaminoethyl)-[1,3]-dioxolane (DLin-KC2-
DMA)
Heptatriaconta-6,9,28,31-tetraen-19-yl 4-(dimethylamino)butanoate
(DLin-MC3-DMA)
1,2-distearoyl-sn-glycero-3-phosphocholine (18:0 PC DSPC)
1,2-dioleyl-sn-glycero-3-phosphocholine (18:1 PC)
1,2-distearyl-sn-glycero-3-phosphatidyl ethanolamine (DSPE)
1,2-dilinoleyl-3-dimethylammonium-propane (18:2 DAP)
hexadimethrine bromide (Polybrene ™)
DEAE-Dextran
protamine
protamine sulfate
poly-L-lysine
poly-D-lysine
Poly(beta-amino-ester) polymer
polyethyleneimine
block co-polymer comprising one or more of: PEG, PLGA, PPG, PEI,
PLL, PCL,
a PLURONIC

In some embodiments, the liposomes include LIPOFECTAMINE 3000. In some embodiments, the liposomes include one or more lipids described in U.S. Pat. No. 4,897,355 or 7,479,573 or in International Patent Publication No. WO/2015/089487, or in Felgner, P. L. et al. (1987) Proc. Natl. Acad. Sci. USA 84:7413-7417, the entire contents of each is incorporated by reference in their entireties).

In some embodiments, the liposome comprises N-[1-(2,3-dioleoyloxy)propyl]-N,N,N-trimethylammonium chloride (DOTMA). In some embodiments, the liposome comprises dioleoylphosphatidylethanolamine (DOPE).

In one embodiment, the liposomes include one or more polyethylene glycol (PEG) chains, optionally selected from PEG200, PEG300, PEG400, PEG600, PEG800, PEG1000, PEG1500, PEG2000, PEG3000, and PEG4000. In some embodiments, the PEG is PEG2000. In some embodiments, the liposomes include 1,2-distearoyl-sn-glycero-3-phosphoethanolamine (DSPE) or a derivative thereof. In one embodiment, the formulation comprises PEGylated lipid 1,2-distearoyl-sn-glycero-3-phosphoethanolamine-N-[methoxy(polyethylene glycol)-2000](DSPE-PEG); in another embodiment, the formulation comprises 1,2-dimyristoyl-sn-glycero-3-phosphoethanolamine-N-[methoxy(polyethylene glycol)-2000](DMPE-PEG); in yet another embodiment, the formulation comprises 1,2-dimyristoyl-rac-glycero-3-methoxypolyethylene glycol-2000 (DMG-PEG). In further embodiments, the formulation comprises a mixture of PEGylated lipids or free PEG chains.

In some embodiments, the formulation comprises one or more of N-(carbonyl-ethoxypolyethylene glycol 2000)-1,2-distearoyl-sn-glycero-3-phosphoethanolamine (MPEG2000-DSPE), fully hydrogenated phosphatidylcholine, cholesterol, LIPOFECTAMINE 3000, a cationic lipid, a polycationic lipid, and 1,2-distearoyl-sn-glycero-3-phosphoethanolamine-N-[folate(polyethylene glycol)-5000](FA-MPEG5000-DSPE).

In one embodiment, the formulation comprises about 3.2 mg/mL N-(carbonyl-ethoxypolyethylene glycol 2000)-1,2-distearoyl-sn-glycero-3-phosphoethanolamine (MPEG2000-DSPE), about 9.6 mg/mL fully hydrogenated phosphatidylcholine, about 3.2 mg/mL cholesterol, about 2 mg/mL ammonium sulfate, and histidine as a buffer, with about 0.27 mg/mL 1,2-distearoyl-sn-glycero-3-phosphoethanolamine-N-[folate(polyethylene glycol)-5000](FA-MPEG5000-DSPE) added to the lipid mixture. In another embodiment, the nucleic acids are complexed by combining 1 μL of LIPOFECTAMINE 3000 per about 1 μg of nucleic acid and incubating at room temperature for at least about 5 minutes. In one embodiment, the LIPOFECTAMINE 3000 is a solution comprising a lipid at a concentration of about 1 mg/mL. In some embodiments, nucleic acids are encapsulated by combining about 10 μg of the liposome formulation per about 1 μg of nucleic acid and incubating at room temperature for about 5 minutes.

In some embodiments, the formulation comprises one or more nanoparticles. In one embodiment, the nanoparticle is a polymeric nanoparticle. In various embodiments, the formulation comprises one or more of a diblock copolymer, a triblock copolymer, a tetrablock copolymer, and a multiblock copolymer. In various embodiments, the formulation comprises one or more of polymeric nanoparticles comprising a polyethylene glycol (PEG)-modified polylactic acid (PLA) diblock copolymer (PLA-PEG), PEG-polypropylene glycol-PEG-modified PLA-tetrablock copolymer (PLA-PEG-PPG-PEG), and Poly(lactic-co-glycolic acid) copolymer. In another embodiment, the formulation comprises a statistical, or an alternating, or a periodic copolymer, or any other sort of polymer.

In some embodiments, the formulation comprises one or more lipids that are described in WO/2000/027795, the entire contents of which are hereby incorporated by reference.

In some embodiments, the liposome comprises Polybrene™ (hexadimethrine bromide) as described in U.S. Pat. No. 5,627,159, the entire contents of which is incorporated herein by reference.

In various embodiments, the liposome components comprise one or more polymers. Examples of polymer include hexadimethrine bromide (Polybrene™), DEAE-Dextran, protamine, protamine sulfate, poly-L-lysine, or poly-D-lysine.

These polymers may be used in combination with cationic lipids to result in synergistic effects on uptake by cells, stability of the formulation, including serum stability (e.g., stability in vivo), endosomal escape, cell viability, and protein expression.

It has now been discovered that liposomal formulations of synthetic RNA delivered to a lateral ventricle can efficiently transfect the tissue lining the ventricle, including ventricular ependymal cells. Certain embodiments are therefore directed to a method of delivering RNA to the central nervous system. In some embodiments, the formulation specifically targets one or more cell types. In some aspects, a cell-specific targeting ligand is included in the formulation.

In other aspects, one or more cell types are targeted and other cell types are not targeted. By way of non-limiting example, intradermally-injected RNA may be formulated to target transfection of keratinocytes and avoid transfection of fibroblasts.

In some embodiments, the formulation comprises one or more polymers. In various embodiments, the formulation comprises polymers, or polymer nanoparticles, or hybrid lipid-polymer nanoparticles, or mixtures of liposomes and polymer nanoparticles, or mixtures of liposomes and free polymers, or mixtures of free lipids and polymer nanoparticles.

In embodiments, the formulation comprises a poly(beta-amino-ester) polymer.

An aspect of the present invention is a composition comprising an effective amount of the synthetic RNA used in the method of any of the herein-disclosed aspects or embodiments.

In embodiments, having an injection volume of less than about 1 mL, less than about 0.5 mL, less than about 0.2 mL, less than about 0.1 mL, less than about 0.05 mL, less than about 0.02 mL, less than about 0.01 mL, less than about 0.005 mL, less than about 0.002 mL, or less than about 0.001 mL.

An aspect of the present invention is a pharmaceutical composition, comprising the composition of any of the preceding embodiments and aspects and a pharmaceutically-acceptable excipient.

In embodiments, use of the composition of any of the preceding embodiments and aspects, or the pharmaceutical composition of any of the preceding embodiments and aspects in the treatment of a disease or disorder described herein.

In embodiments, use of the composition of any of the preceding embodiments and aspects, or the pharmaceutical composition of any of the preceding embodiments and aspects in the manufacture of a medicament for the treatment of a disease or disorder described herein.

An aspect of the present invention is a composition comprising a synthetic RNA used in the method of any one of the preceding embodiments and aspects and formulated with one or more lipids and/or polymers selected from Table 1.

In some embodiments, liposomes are created using microfluidics. In one aspect, liposomes are manufactured using a Nanoassembir instrument (Precision Nanosystems). In another aspect, syringe pumps are used to mix organic and aqueous solutions at a specified flowrate. Optionally, the ratio of the flowrate of the aqueous solution to that of the organic solution may be selected from about 1:1, about 2:1, about 3:1, about 4:1, about 5:1, about 6:1, about 8:1, or about 10:1. In some embodiments, the organic solution comprises one or more of ethanol, acetonitrile, dimethyl sulfoxide, and chloroform, or a mixture thereof. In other embodiments, other solvents are used.

In some aspects, liposomes are manufactured by dropwise mixture of one solution into another. In various other aspects, liposomes are manufactured with a spray mechanism, or by solvent evaporation, or by sonication, or by extrusion through one or more membranes, or through a process of self-assembly, or by a combination of methods.

In some embodiments, liposomes include lipids selected from one or more of the following categories: cationic lipids; anionic lipids; neutral lipids; multi-valent charged lipids; and zwitterionic lipids. In some cases, a cationic lipid may be used to facilitate a charge-charge interaction with nucleic acids. Several cationic lipids that accomplish this in certain embodiments of the invention are provided among the lipids of Table 1; these are provided for illustration only.

In various embodiments, the formulation comprises a cationic or polycationic lipid, a PEGylated lipid, and/or one or more helper lipids. In some embodiments, the helper lipid is a phospholipid; in other embodiments, the helper lipid is cholesterol; in still other embodiments, both a phospholipid and cholesterol are used as helper lipids. In one embodiment, the phospholipids 18:0 PC, 18:1 PC, 18:2 PE, DSPE, DOPE, 18:2 PE, or a combination thereof are used as helper lipids. In certain embodiments, cholesterol is derived from plant sources. Ifn other embodiments, cholesterol is derived from animal, fungal, bacterial or archaeal sources.

In any of the preceding embodiments and aspects, the effective amount of the synthetic RNA comprises one or more lipids and/or polymers to enhance uptake of RNA by cells.

In any of the preceding embodiments and aspects, the effective amount of the synthetic RNA comprises a cationic liposome and/or cationic polymer formulation.

In embodiments, a lipid and/or a polymer of the cationic liposome formulation is selected from Table 1.

An aspect of the present invention is a method of polynucleotide delivery to the central nervous system, comprising a synthetic polynucleotide formulated with a liposome comprising one or more lipids selected from Table 1.

In embodiments, the polynucleotide is a synthetic RNA.

In any of the preceding embodiments and aspects, the liposome comprises 1,2-dioleoyl-3-dimethylammonium-propane (DODAP).

In embodiments, the liposome further comprises one or more helper lipids, optionally selected from dioleoyl phosphatidyl ethanolamine (DOPE), 1,2-distearoyl-sn-glycero-3-phosphocholine (DSPC), and cholesterol.

In any of the preceding embodiments and aspects, the liposome further comprises a PEGylated lipid.

In any of the preceding embodiments and aspects, the subject in need is a human.

In any of the preceding embodiments and aspects, the effective amount of the synthetic RNA is administered about weekly, for at least 2 weeks.

In any of the preceding embodiments and aspects, the effective amount of the synthetic RNA is administered about every other week for at least one month.

In any of the preceding embodiments and aspects, the effective amount of the synthetic RNA is administered monthly or about every other month.

In any of the preceding embodiments and aspects, the effective amount of the synthetic RNA is administered for at least two months, or at least 4 months, or at least 6 months, or at least 9 months, or at least one year.

In any of the preceding embodiments and aspects, the synthetic RNA comprises 5-methoxyuridine.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal, intraportal or intravenous injection, or by direct injection into diseased, e.g. cancer, tissue. The agents disclosed herein may also be administered by catheter systems. Such compositions would normally be administered as pharmaceutically acceptable compositions as described herein.

Administration of the compositions described herein may be, for example, by injection, topical administration, ophthalmic administration, and intranasal administration. The injection, in some embodiments, may be linked to an electrical force (e.g. electroporation, including with devices that find use in electrochemotherapy (e.g. CLINIPORATOR, IGEA Srl, Carpi [MO], Italy)). The topical administration may be, but is not limited to, a cream, lotion, ointment, gel, spray, solution and the like. The topical administration may further include a penetration enhancer such as, but not limited to, surfactants, fatty acids, bile salts, chelating agents, non-chelating non-surfactants, polyoxyethylene-9-lauryl ether, polyoxyethylene-20-cetyl ether, fatty acids and/or salts in combination with bile acids and/or salts, sodium salt in combination with lauric acid, capric acid and UDCA, and the like. The topical administration may also include a fragrance, a colorant, a sunscreen, an antibacterial, and/or a moisturizer. The compositions described herein may be administered to at least one site such as, but not limited to, forehead, scalp, hair follicles, hair, upper eyelids, lower eyelids, eyebrows, eyelashes, infraorbital area, periorbital areas, temple, nose, nose bridge, cheeks, tongue, nasolabial folds, lips, periobicular areas, jaw line, ears, neck, breast, forearm, upper arm, palm, hand, finger, nails, back, abdomen, sides, buttocks, thigh, calf, feet, toes and the like.

Routes of administration include, for example: intradermal, intramuscular, intraperitoneal, intravenous, subcutaneous, intranasal, epidural, oral, sublingual, intracerebral, intravaginal, transdermal, intraportal, rectally, by inhalation, or topically, particularly to the ears, nose, eyes, or skin. In some embodiments, the administering is effected orally or by parenteral injection.

Upon formulation, solutions may be administered in a manner compatible with the dosage formulation and in such amount as is therapeutically effective, as described herein. The formulations may easily be administered in a variety of dosage forms such as injectable solutions, drug release capsules and the like. For parenteral administration in an aqueous solution, for example, the solution generally is suitably buffered and the liquid diluent first rendered isotonic with, for example, sufficient saline or glucose. Such aqueous solutions may be used, for example, for intravenous, intramuscular, subcutaneous, and intraperitoneal administration. Preferably, sterile aqueous media are employed as is known to those of skill in the art, particularly in light of the present disclosure.

In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose is administered to a surface area of about 4 mm² to about 150 mm² (e.g. about, or no more than about, 4 mm², or about 5 mm², or about 6 mm², or about 7 mm², or about 8 mm², or about 10 mm², or about 20 mm², or about 50 mm², or about 100 mm², or about 150 mm²). In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose administered to a surface area of no more than about 4 mm², or about 5 mm², or about 6 mm², or about 7 mm², or about 8 mm², or about 10 mm², or about 20 mm², or about 50 mm², or about 100 mm², or about 150 mm². In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose administered to a surface area of about 4 mm², or about 5 mm², or about 6 mm², or about 7 mm², or about 8 mm², or about 10 mm², or about 20 mm², or about 50 mm², or about 100 mm², or about 150 mm².

In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose (weight RNA/surface area of injection) is about 35 ng/cm² to about 7000 ng/cm². In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose (weight RNA/surface area of injection) is no more than about 35 ng/cm², or about 50 ng/cm², or about 75 ng/cm², or about 100 ng/cm², or about 125 ng/cm², or about 150 ng/cm², or about 175 ng/cm², or about 200 ng/cm², or about 225 ng/cm², or about 250 ng/cm², or about 500 ng/cm², or about 1000 ng/cm², or about 2000 ng/cm², or about 5000 ng/cm², or about 7000 ng/cm². In various embodiments, the nucleic acid drug, including RNA comprising one or more non-canonical nucleotides, and/or formulations comprising the same, is administered locally, optionally by one or more of subcutaneous injection, intradermal injection, subdermal injection and intramuscular injection, and the effective dose (weight RNA/surface area of injection) is about 35 ng/cm², or about 50 ng/cm², or about 75 ng/cm², or about 100 ng/cm², or about 125 ng/cm², or about 150 ng/cm², or about 175 ng/cm², or about 200 ng/cm², or about 225 ng/cm², or about 250 ng/cm², or about 500 ng/cm², or about 1000 ng/cm², or about 2000 ng/cm², or about 5000 ng/cm², or about 7000 ng/cm².

Pharmaceutical preparations may additionally comprise delivery reagents (a.k.a. “transfection reagents”, a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients (a.k.a. “subjects”) are well known in the art, and are set forth in numerous publications, including, for example, in US Patent Appl. Pub. No. US 2008/0213377, the entirety of which is incorporated herein by reference.

For example, the present compositions can be in the form of pharmaceutically acceptable salts. Such salts include those listed in, for example, J. Pharma. Sci. 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. Non-limiting examples of pharmaceutically acceptable salts include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, tartarate salts, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipients are sterile when administered to a subject.

Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any agent described herein, if desired, can also comprise minor amounts of wetting or emulsifying agents, or pH buffering agents.

Dosage forms suitable for parenteral administration (e.g. subcutaneous, intradermal, subdermal, intramuscular, intravenous, intraperitoneal, intra-articular, and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

In some embodiments, the formulations described herein may comprise albumin and a nucleic acid molecule.

In some embodiments, the invention relates to a cosmetic composition. In one embodiment, the cosmetic composition comprises albumin. In another embodiment, the albumin is treated with an ion-exchange resin or charcoal. In yet another embodiment, the cosmetic composition comprises a nucleic acid molecule. In a further embodiment, the cosmetic composition comprises both albumin and a nucleic acid molecule. Still other embodiments are directed to a cosmetic treatment article comprising a cosmetic composition contained in a device configured to deliver the composition to a patient. Still other embodiments are directed to a device configured to deliver a cosmetic composition to a patient. In one embodiment, the nucleic acid molecule encodes a member of the group: elastin, collagen, tyrosinase, melanocortin 1 receptor, keratin, filaggren, an antibody, and hyaluronan synthase or a biologically active fragment, variant, analogue or family member thereof.

### Non-Canonical Nucleotides

In some embodiments, the present invention provides treatment regimens. The inventors have discovered that the doses and administration described herein can produce a substantial protein expression effect quickly (e.g. in about 6, or about 12, or about 24, or about 36, or about 48 hours). Further, these effects can be sustained for about 7 days, or longer. In some embodiments, the present methods provide for administration of a nucleic acid drug, including RNA comprising one or more non-canonical nucleotides, about weekly to about once every 20 weeks.

In some embodiments, the nucleic acid drug, including RNA comprising one or more non-canonical nucleotides, is administered about weekly, for at least 2 weeks (e.g. 3, or 4, or 5, or 6, or 7, or 8, or 9, or 10 weeks). In some embodiments, the nucleic acid drug, including RNA comprising one or more non-canonical nucleotides, is administered about every other week for at least one month (e.g. 1, or 2, or 3, or 4, or 5, or 6, or 12 months). In some embodiments, the nucleic acid drug, including RNA comprising one or more non-canonical nucleotides, is administered monthly or about every other month. In some embodiments, the nucleic acid drug, including RNA comprising one or more non-canonical nucleotides, is administered is administered for at least two months, or at least 4 months, or at least 6 months, or at least 9 months, or at least one year.

In some embodiments, the nucleic acid drug, including RNA comprising one or more non-canonical nucleotides, is administered about weekly, or about once every 2 weeks, or about once every 3 weeks, or about once every 4 weeks, or about once every 5 weeks, or about once every 6 weeks, or about once every 7 weeks, or about once every 8 weeks, or about once every 9 weeks, or about once every 10 weeks, or about once every 11 weeks, or about once every 12 weeks, or about once every 13 weeks, or about once every 14 weeks, or about once every 15 weeks, or about once every 20 weeks, or about once every 24 weeks.

In some embodiments, the nucleic acid drug, including RNA comprising one or more non-canonical nucleotides, is administered no more than about weekly, or about once every 2 weeks, or about once every 3 weeks, or about once every 4 weeks, or about once every 5 weeks, or about once every 6 weeks, or about once every 7 weeks, or about once every 8 weeks, or about once every 9 weeks, or about once every 10 weeks, or about once every 11 weeks, or about once every 12 weeks, or about once every 13 weeks, or about once every 14 weeks, or about once every 15 weeks, or about once every 20 weeks, or about 24 weeks.

Certain proteins have long half-lives, and can persist in tissues for several hours, days, weeks, months, or years. It has now been discovered that certain methods of treating a patient can result in accumulation of one or more proteins, including, for example, one or more beneficial proteins. Certain embodiments are therefore directed to a method for treating a patient comprising delivering to a patient in a series of doses a nucleic acid encoding one or more proteins. In one embodiment, the nucleic acid comprises RNA comprising one or more non-canonical nucleotides. In another embodiment, a first dose is given at a first time-point. In yet another embodiment, a second dose is given at a second time-point. In a further embodiment, the amount of at least one of the one or more proteins in the patient at the second time-point is greater than the amount of said protein at the first time-point. In a still further embodiment, the method results in the accumulation of said protein in the patient.

In various embodiments, the present invention relates to nucleic acid drugs, which, in various embodiments are RNA comprising one or more non-canonical nucleotides. Certain non-canonical nucleotides, when incorporated into RNA molecules, can reduce the toxicity of the RNA molecules, in part, without wishing to be bound by theory, by interfering with binding of proteins that detect exogenous nucleic acids, for example, protein kinase R, Rig-1 and the oligoadenylate synthetase family of proteins. Non-canonical nucleotides that have been reported to reduce the toxicity of RNA molecules when incorporated therein include pseudouridine, 5-methyluridine, 2-thiouridine, 5-methylcytidine, N6-methyladenosine, and certain combinations thereof. However, the chemical characteristics of non-canonical nucleotides that can enable them to lower the in vivo toxicity of RNA molecules have, until this point, remained unknown.

Furthermore, incorporation of large amounts of most non-canonical nucleotides, for example, 5-methyluridine, 2-thiouridine, 5-methylcytidine, and N6-methyladenosine, can reduce the efficiency with which RNA molecules can be translated into protein, limiting the utility of RNA molecules containing these nucleotides in applications that require protein expression. In addition, while pseudouridine can be completely substituted for uridine in RNA molecules without reducing the efficiency with which the synthetic RNA molecules can be translated into protein, in certain situations, for example, when performing frequent, repeated transfections, synthetic RNA molecules containing only adenosine, guanosine, cytidine, and pseudouridine can exhibit excessive toxicity.

It has now been discovered that, and in some embodiments the invention pertains to, RNA molecules containing one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

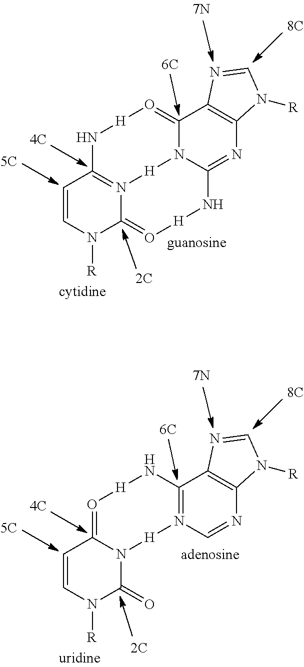

Examples of non-canonical nucleotides that include one or more substitutions at the 2C and/or 4C and/or 5C positions in the case of a pyrimidine or the 6C and/or 7N and/or 8C positions in the case of a purine include, but are not limited to 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, 6-thio-7-deaza-8-azaguanosine, and 5-methoxyuridine.

In some embodiments, the invention relates to one or more non-canonical nucleotides selected from 5-hydroxycytidine, 5-methylcytidine, 5-hydroxymethylcytidine, 5-carboxycytidine, 5-formylcytidine, 5-methoxycytidine, 5-hydroxyuridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine. In some embodiments, at least 50%, or at least 55%, or at least 60%, or at least 65%, or at least 70%, or at least 75%, or at least 80%, or at least 85%, or at least 90%, or at least 95%, or 100% of the non-canonical nucleotides are one or more of 5-hydroxycytidine, 5-methylcytidine, 5-hydroxymethylcytidine, 5-carboxycytidine, 5-formylcytidine, 5-methoxycytidine, 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 50%, or at least about 55%%, or at least 60%, or at least 65%, or at least 70%, or at least 75%, or at least 80%, or at least 85%, or at least 90%, or at least 95%, or 100% of cytidine residues are non-canonical nucleotides selected from 5-hydroxycytidine, 5-methylcytidine, 5-hydroxymethylcytidine, 5-carboxycytidine, 5-formylcytidine, 5-methoxycytidine.

In some embodiments, at least about 20%, or about 30%, or about 40%, or about 50%, or at least about 55%, or at least 60%, or at least 65%, or at least 70%, or at least 75%, or at least 80%, or at least 85%, or at least 90%, or at least 95%, or 100% of uridine residues are non-canonical nucleotides selected from 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% (e.g. 10%, or about 20%, or about 30%, or about 40%, or about 50%) of guanosine residues are non-canonical nucleotides, and the non-canonical nucleotide is optionally 7-deazaguanosine.

In some embodiments, the RNA contains no more than about 50% 7-deazaguanosine in place of guanosine residues.

In some embodiments, the RNA does not contain non-canonical nucleotides in place of adenosine residues.

Note that alternative naming schemes exist for certain non-canonical nucleotides. For example, in certain situations, 5-methylpseudouridine can be referred to as “3-methylpseudouridine” or “N3-methylpseudouridine” or “1-methylpseudouridine” or “N1-methylpseudouridine”.

Nucleotides that contain the prefix “amino” can refer to any nucleotide that contains a nitrogen atom bound to the atom at the stated position of the nucleotide, for example, 5-aminocytidine can refer to 5-aminocytidine, 5-methylaminocytidine, and 5-nitrocytidine. Similarly, nucleotides that contain the prefix “methyl” can refer to any nucleotide that contains a carbon atom bound to the atom at the stated position of the nucleotide, for example, 5-methylcytidine can refer to 5-methylcytidine, 5-ethylcytidine, and 5-hydroxymethylcytidine, nucleotides that contain the prefix “thio” can refer to any nucleotide that contains a sulfur atom bound to the atom at the given position of the nucleotide, and nucleotides that contain the prefix “hydroxy” can refer to any nucleotide that contains an oxygen atom bound to the atom at the given position of the nucleotide, for example, 5-hydroxyuridine can refer to 5-hydroxyuridine and uridine with a methyl group bound to an oxygen atom, wherein the oxygen atom is bound to the atom at the 5C position of the uridine.

Certain embodiments are therefore directed to RNA comprising one or more non-canonical nucleotides, wherein the RNA molecule contains one or more nucleotides that includes one or more substitutions at the 2C and/or 4C and/or 5C positions in the case of a pyrimidine or the 6C and/or 7N and/or 8C positions in the case of a purine. Other embodiments are directed to a therapeutic, wherein the therapeutic contains one or more RNA molecules comprising one or more non-canonical nucleotides, and wherein the one or more RNA molecules comprising one or more non-canonical nucleotides contains one or more nucleotides that includes one or more substitutions at the 2C and/or 4C and/or 5C positions in the case of a pyrimidine or the 6C and/or 7N and/or 8C positions in the case of a purine. In one embodiment, the therapeutic comprises a transfection reagent. In another embodiment, the transfection reagent comprises a cationic lipid, liposome, or micelle. In still another embodiment, the liposome or micelle comprises folate and the therapeutic composition has anti-cancer activity. In another embodiment, the one or more nucleotides includes at least one of pseudouridine, 2-thiouridine, 4-thiouridine, 5-azauridine, 5-hydroxyuridine, 5-methyluridine, 5-aminouridine, 2-thiopseudouridine, 4-thiopseudouridine, 5-hydroxypseudouridine, 5-methylpseudouridine, 5-aminopseudouridine, pseudoisocytidine, N4-methylcytidine, 2-thiocytidine, 5-azacytidine, 5-hydroxycytidine, 5-aminocytidine, 5-methylcytidine, N4-methylpseudoisocytidine, 2-thiopseudoisocytidine, 5-hydroxypseudoisocytidine, 5-aminopseudoisocytidine, 5-methylpseudoisocytidine, 7-deazaadenosine, 7-deazaguanosine, 6-thioguanosine, and 6-thio-7-deazaguanosine. In another embodiment, the one or more nucleotides includes at least one of pseudouridine, 2-thiouridine, 4-thiouridine, 5-azauridine, 5-hydroxyuridine, 5-methyluridine, 5-aminouridine, 2-thiopseudouridine, 4-thiopseudouridine, 5-hydroxypseudouridine, 5-methylpseudouridine, and 5-aminopseudouridine and at least one of pseudoisocytidine, N4-methylcytidine, 2-thiocytidine, 5-azacytidine, 5-hydroxycytidine, 5-aminocytidine, 5-methylcytidine, N4-methylpseudoisocytidine, 2-thiopseudoisocytidine, 5-hydroxypseudoisocytidine, 5-aminopseudoisocytidine, and 5-methylpseudoisocytidine. In still another embodiment, the one or more nucleotides include at least one of pseudouridine, 2-thiouridine, 4-thiouridine, 5-azauridine, 5-hydroxyuridine, 5-methyluridine, 5-aminouridine, 2-thiopseudouridine, 4-thiopseudouridine, 5-hydroxypseudouridine, and 5-methylpseudouridine, 5-aminopseudouridine and at least one of pseudoisocytidine, N4-methylcytidine, 2-thiocytidine, 5-azacytidine, 5-hydroxycytidine, 5-aminocytidine, 5-methylcytidine, N4-methylpseudoisocytidine, 2-thiopseudoisocytidine, 5-hydroxypseudoisocytidine, 5-aminopseudoisocytidine, and 5-methylpseudoisocytidine and at least one of 7-deazaguanosine, 6-thioguanosine, 6-thio-7-deazaguanosine, and 5-methoxyuridine. In yet another embodiment, the one or more nucleotides includes 5-methylcytidine and 7-deazaguanosine. In another embodiment, the one or more nucleotides also includes pseudouridine or 4-thiouridine or 5-methyluridine or 5-aminouridine or 4-thiopseudouridine or 5-methylpseudouridine or 5-aminopseudouridine. In a still another embodiment, the one or more nucleotides also includes 7-deazaadenosine. In another embodiment, the one or more nucleotides includes pseudoisocytidine and 7-deazaguanosine and 4-thiouridine. In yet another embodiment, the one or more nucleotides includes pseudoisocytidine or 7-deazaguanosine and pseudouridine. In still another embodiment, the one or more nucleotides includes 5-methyluridine and 5-methylcytidine and 7-deazaguanosine. In a further embodiment, the one or more nucleotides includes pseudouridine or 5-methylpseudouridine and 5-methylcytidine and 7-deazaguanosine. In another embodiment, the one or more nucleotides includes pseudoisocytidine and 7-deazaguanosine and pseudouridine. In one embodiment, the RNA comprising one or more non-canonical nucleotides is present in vivo.

Certain non-canonical nucleotides can be incorporated more efficiently than other non-canonical nucleotides into RNA molecules by RNA polymerases that are commonly used for in vitro transcription, due in part to the tendency of these certain non-canonical nucleotides to participate in standard base-pairing interactions and base-stacking interactions, and to interact with the RNA polymerase in a manner similar to that in which the corresponding canonical nucleotide interacts with the RNA polymerase. As a result, certain nucleotide mixtures containing one or more non-canonical nucleotides can be beneficial in part because in vitro-transcription reactions containing these nucleotide mixtures can yield a large quantity of RNA. Certain embodiments are therefore directed to a nucleotide mixture containing one or more nucleotides that includes one or more substitutions at the 2C and/or 4C and/or 5C positions in the case of a pyrimidine or the 6C and/or 7N and/or 8C positions in the case of a purine. Nucleotide mixtures include, but are not limited to (numbers preceding each nucleotide indicate an exemplary fraction of the non-canonical nucleotide triphosphate in an in vitro-transcription reaction, for example, 0.2 pseudoisocytidine refers to a reaction containing adenosine-5′-triphosphate, guanosine-5′-triphosphate, uridine-5′-triphosphate, cytidine-5′-triphosphate, and pseudoisocytidine-5′-triphosphate, wherein pseudoisocytidine-5′-triphosphate is present in the reaction at an amount approximately equal to 0.2 times the total amount of pseudoisocytidine-5′-triphosphate+cytidine-5′-triphosphate that is present in the reaction, with amounts measured either on a molar or mass basis, and wherein more than one number preceding a nucleoside indicates a range of exemplary fractions): 1.0 pseudouridine, 0.1-0.8 2-thiouridine, 0.1-0.8 5-methyluridine, 0.2-1.0 5-hydroxyuridine, 0.2-1.0 5-methoxyuridine, 0.1-1.0 5-aminouridine, 0.1-1.0 4-thiouridine, 0.1-1.0 2-thiopseudouridine, 0.1-1.0 4-thiopseudouridine, 0.1-1.0 5-hydroxypseudouridine, 0.2-1 5-methylpseudouridine, 0.2-1.0 5-methoxypseudouridine, 0.1-1.0 5-aminopseudouridine, 0.2-1.0 2-thiocytidine, 0.1-0.8 pseudoisocytidine, 0.2-1.0 5-methylcytidine, 0.2-1.0 5-hydroxycytidine, 0.2-1.0 5-hydroxymethylcytidine, 0.2-1.0 5-methoxycytidine, 0.1-1.0 5-aminocytidine, 0.2-1.0 N4-methylcytidine, 0.2-1.0 5-methylpseudoisocytidine, 0.2-1.0 5-hydroxypseudoisocytidine, 0.2-1.0 5-aminopseudoisocytidine, 0.2-1.0 N4-methylpseudoisocytidine, 0.2-1.0 2-thiopseudoisocytidine, 0.2-1.0 7-deazaguanosine, 0.2-1.0 6-thioguanosine, 0.2-1.0 6-thio-7-deazaguanosine, 0.2-1.0 8-azaguanosine, 0.2-1.0 7-deaza-8-azaguanosine, 0.2-1.0 6-thio-8-azaguanosine, 0.1-0.5 7-deazaadenosine, and 0.1-0.5 N6-methyladenosine.

In various embodiments, the RNA comprising one or more non-canonical nucleotides composition or synthetic polynucleotide composition (e.g., which may be prepared by in vitro transcription) contains substantially or entirely the canonical nucleotide at positions having adenine or “A” in the genetic code. The term “substantially” in this context refers to at least 90%. In these embodiments, the RNA composition or synthetic polynucleotide composition may further contain (e.g., consist of) 7-deazaguanosine at positions with “G” in the genetic code as well as the corresponding canonical nucleotide “G”, and the canonical and non-canonical nucleotide at positions with G may be in the range of 5:1 to 1:5, or in some embodiments in the range of 2:1 to 1:2. In these embodiments, the RNA composition or synthetic polynucleotide composition may further contain (e.g., consist of) one or more (e.g., two, three or four) of 5-hydroxycytidine, 5-methylcytidine, 5-hydroxymethylcytidine, 5-carboxycytidine, 5-formylcytidine, 5-methoxycytidine at positions with “C” in the genetic code as well as the canonical nucleotide “C”, and the canonical and non-canonical nucleotide at positions with C may be in the range of 5:1 to 1:5, or in some embodiments in the range of 2:1 to 1:2. In some embodiments, the level of non-canonical nucleotide at positions of “C” are as described in the preceding paragraph. In these embodiments, the RNA composition or synthetic polynucleotide composition may further contain (e.g., consist of) one or more (e.g., two, three, or four) of 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridineat positions with “U” in the genetic code as well as the canonical nucleotide “U”, and the canonical and non-canonical nucleotide at positions with “U” may be in the range of 5:1 to 1:5, or in some embodiments in the range of 2:1 to 1:2.

In some embodiments, the level of non-canonical nucleotide at positions of “U” are as described in the preceding paragraph.

It has now been discovered that combining certain non-canonical nucleotides can be beneficial in part because the contribution of non-canonical nucleotides to lowering the toxicity of RNA molecules can be additive. Certain embodiments are therefore directed to a nucleotide mixture, wherein the nucleotide mixture contains more than one of the non-canonical nucleotides listed above, for example, the nucleotide mixture contains both pseudoisocytidine and 7-deazaguanosine or the nucleotide mixture contains both N4-methylcytidine and 7-deazaguanosine, etc. In one embodiment, the nucleotide mixture contains more than one of the non-canonical nucleotides listed above, and each of the non-canonical nucleotides is present in the mixture at the fraction listed above, for example, the nucleotide mixture contains 0.1-0.8 pseudoisocytidine and 0.2-1.0 7-deazaguanosine or the nucleotide mixture contains 0.2-1.0 N4-methylcytidine and 0.2-1.0 7-deazaguanosine, etc.

In certain situations, for example, when it may not be necessary or desirable to maximize the yield of an in vitro-transcription reaction, nucleotide fractions other than those given above may be used. The exemplary fractions and ranges of fractions listed above relate to nucleotide-triphosphate solutions of typical purity (greater than 90% purity). Larger fractions of these and other nucleotides can be used by using nucleotide-triphosphate solutions of greater purity, for example, greater than about 95% purity or greater than about 98% purity or greater than about 99% purity or greater than about 99.5% purity, which can be achieved, for example, by purifying the nucleotide triphosphate solution using existing chemical-purification technologies such as high-pressure liquid chromatography (HPLC) or by other means. In one embodiment, nucleotides with multiple isomers are purified to enrich the desired isomer.

Other embodiments are directed to a method for inducing a cell in vivo to express a protein of interest by contacting the cell with a RNA molecule that contains one or more non-canonical nucleotides that includes one or more substitutions at the 2C and/or 4C and/or 5C positions in the case of a pyrimidine or the 6C and/or 7N and/or 8C positions in the case of a purine. Still other embodiments are directed to a method for transfecting, reprogramming, and/or gene-editing a cell in vivo by contacting the cell with a RNA molecule that contains one or more non-canonical nucleotides that includes one or more substitutions at the 2C and/or 4C and/or 5C positions in the case of a pyrimidine or the 6C and/or 7N and/or 8C positions in the case of a purine. In one embodiment, the RNA molecule is produced by in vitro transcription. In one embodiment, the RNA molecule encodes one or more reprogramming factors. In another embodiment, the one or more reprogramming factors includes Oct4 protein. In another embodiment, the cell is also contacted with a RNA molecule that encodes Sox2 protein. In yet another embodiment, the cell is also contacted with a RNA molecule that encodes Klf4 protein. In yet another embodiment, the cell is also contacted with a RNA molecule that encodes c-Myc protein. In yet another embodiment, the cell is also contacted with a RNA molecule that encodes Lin28 protein.

Enzymes such as T7 RNA polymerase may preferentially incorporate canonical nucleotides in an in vitro-transcription reaction containing both canonical and non-canonical nucleotides. As a result, an in vitro-transcription reaction containing a certain fraction of a non-canonical nucleotide may yield RNA containing a different, often lower, fraction of the non-canonical nucleotide than the fraction at which the non-canonical nucleotide was present in the reaction. In certain embodiments, references to nucleotide incorporation fractions (for example, “a synthetic RNA molecule containing 50% pseudoisocytidine” or “0.1-0.8 pseudoisocytidine”) therefore can refer both to RNA molecules containing the stated fraction of the nucleotide, and to RNA molecules synthesized in a reaction containing the stated fraction of the nucleotide (or nucleotide derivative, for example, nucleotide-triphosphate), even though such a reaction may yield RNA containing a different fraction of the nucleotide than the fraction at which the non-canonical nucleotide was present in the reaction.

Different nucleotide sequences can encode the same protein by utilizing alternative codons. In certain embodiments, references to nucleotide incorporation fractions therefore can refer both to RNA molecules containing the stated fraction of the nucleotide, and to RNA molecules encoding the same protein as a different RNA molecule, wherein the different RNA molecule contains the stated fraction of the nucleotide.

It has now been discovered that the non-canonical nucleotide members of the 5-methylcytidine de-methylation pathway, when incorporated into synthetic RNA, can increase the efficiency with which the synthetic RNA can be translated into protein in vivo, and can decrease the toxicity of the synthetic RNA in vivo. These non-canonical nucleotides include, for example: 5-methylcytidine, 5-hydroxymethylcytidine, 5-formylcytidine, and 5-carboxycytidine (a.k.a. “cytidine-5-carboxylic acid”). Certain embodiments are therefore directed to a nucleic acid. In some embodiments, the nucleic acid is present in vivo. In one embodiment, the nucleic acid is a synthetic RNA molecule. In another embodiment, the nucleic acid comprises one or more non-canonical nucleotides. In one embodiment, the nucleic acid comprises one or more non-canonical nucleotide members of the 5-methylcytidine de-methylation pathway. In another embodiment, the nucleic acid comprises at least one of 5-methylcytidine, 5-hydroxymethylcytidine, 5-formylcytidine, and 5-carboxycytidine or a derivative thereof. In a further embodiment, the nucleic acid comprises at least one of pseudouridine, 5-methylpseudouridine, 5-hydroxyuridine, 5-methyluridine, 5-methylcytidine, 5-hydroxymethylcytidine, N4-methylcytidine, N4-acetylcytidine, and 7-deazaguanosine or a derivative thereof.

### 5-methylcytidine De-Methylation Pathway

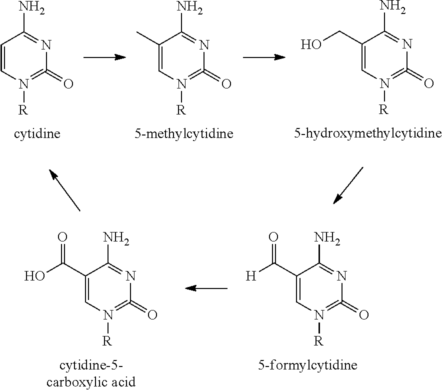

Certain embodiments are directed to a protein. Other embodiments are directed to a nucleic acid that encodes a protein. In one embodiment, the protein is a protein of interest. In another embodiment, the protein is selected from a reprogramming protein and a gene-editing protein. In one embodiment, the nucleic acid is a plasmid. In another embodiment, the nucleic acid is present in a virus or viral vector. In a further embodiment, the virus or viral vector is replication incompetent. In a still further embodiment, the virus or viral vector is replication competent. In one embodiment, the virus or viral vector includes at least one of an adenovirus, a retrovirus, a lentivirus, a herpes virus, an adeno-associated virus or a natural or engineered variant thereof, and an engineered virus.

It has also been discovered that certain combinations of non-canonical nucleotides can be particularly effective at increasing the efficiency with which synthetic RNA can be translated into protein in vivo, and decreasing the toxicity of synthetic RNA in vivo, for example, the combinations: 5-methyluridine and 5-methylcytidine, 5-hydroxyuridine and 5-methylcytidine, 5-hydroxyuridine and 5-hydroxymethylcytidine, 5-methyluridine and 7-deazaguanosine, 5-methylcytidine and 7-deazaguanosine, 5-methyluridine, 5-methylcytidine, and 7-deazaguanosine, and 5-methyluridine, 5-hydroxymethylcytidine, and 7-deazaguanosine. Certain embodiments are therefore directed to a nucleic acid comprising at least two of 5-methyluridine, 5-methylcytidine, 5-hydroxymethylcytidine, and 7-deazaguanosine or one or more derivatives thereof. Other embodiments are directed to a nucleic acid comprising at least three of 5-methyluridine, 5-methylcytidine, 5-hydroxymethylcytidine, and 7-deazaguanosine or one or more derivatives thereof. Other embodiments are directed to a nucleic acid comprising all of 5-methyluridine, 5-methylcytidine, 5-hydroxymethylcytidine, and 7-deazaguanosine or one or more derivatives thereof. In one embodiment, the nucleic acid comprises one or more 5-methyluridine residues, one or more 5-methylcytidine residues, and one or more 7-deazaguanosine residues or one or more 5-methyluridine residues, one or more 5-hydroxymethylcytidine residues, and one or more 7-deazaguanosine residues.

It has been further discovered that synthetic RNA molecules containing certain fractions of certain non-canonical nucleotides and combinations thereof can exhibit particularly high translation efficiency and low toxicity in vivo. Certain embodiments are therefore directed to a nucleic acid comprising at least one of one or more uridine residues, one or more cytidine residues, and one or more guanosine residues, and comprising one or more non-canonical nucleotides. In one embodiment, between about 20% and about 80% of the uridine residues are 5-methyluridine residues. In another embodiment, between about 30% and about 50% of the uridine residues are 5-methyluridine residues. In a further embodiment, about 40% of the uridine residues are 5-methyluridine residues. In one embodiment, between about 60% and about 80% of the cytidine residues are 5-methylcytidine residues. In another embodiment, between about 80% and about 100% of the cytidine residues are 5-methylcytidine residues. In a further embodiment, about 100% of the cytidine residues are 5-methylcytidine residues. In a still further embodiment, between about 20% and about 100% of the cytidine residues are 5-hydroxymethylcytidine residues. In one embodiment, between about 20% and about 80% of the guanosine residues are 7-deazaguanosine residues. In another embodiment, between about 40% and about 60% of the guanosine residues are 7-deazaguanosine residues. In a further embodiment, about 50% of the guanosine residues are 7-deazaguanosine residues. In one embodiment, between about 20% and about 80% or between about 30% and about 60% or about 40% of the cytidine residues are N4-methylcytidine and/or N4-acetylcytidine residues. In another embodiment, each cytidine residue is a 5-methylcytidine residue. In a further embodiment, about 100% of the cytidine residues are 5-methylcytidine residues and/or 5-hydroxymethylcytidine residues and/or N4-methylcytidine residues and/or N4-acetylcytidine residues and/or one or more derivatives thereof. In a still further embodiment, about 40% of the uridine residues are 5-methyluridine residues, between about 20% and about 100% of the cytidine residues are N4-methylcytidine and/or N4-acetylcytidine residues, and about 50% of the guanosine residues are 7-deazaguanosine residues. In one embodiment, about 40% of the uridine residues are 5-methyluridine residues and about 100% of the cytidine residues are 5-methylcytidine residues. In another embodiment, about 40% of the uridine residues are 5-methyluridine residues and about 50% of the guanosine residues are 7-deazaguanosine residues. In a further embodiment, about 100% of the cytidine residues are 5-methylcytidine residues and about 50% of the guanosine residues are 7-deazaguanosine residues. In a further embodiment, about 100% of the uridine residues are 5-hydroxyuridine residues. In one embodiment, about 40% of the uridine residues are 5-methyluridine residues, about 100% of the cytidine residues are 5-methylcytidine residues, and about 50% of the guanosine residues are 7-deazaguanosine residues. In another embodiment, about 40% of the uridine residues are 5-methyluridine residues, between about 20% and about 100% of the cytidine residues are 5-hydroxymethylcytidine residues, and about 50% of the guanosine residues are 7-deazaguanosine residues. In some embodiments, less than 100% of the cytidine residues are 5-methylcytidine residues. In other embodiments, less than 100% of the cytidine residues are 5-hydroxymethylcytidine residues. In one embodiment, each uridine residue in the synthetic RNA molecule is a pseudouridine residue or a 5-methylpseudouridine residue. In another embodiment, about 100% of the uridine residues are pseudouridine residues and/or 5-methylpseudouridine residues. In a further embodiment, about 100% of the uridine residues are pseudouridine residues and/or 5-methylpseudouridine residues, about 100% of the cytidine residues are 5-methylcytidine residues, and about 50% of the guanosine residues are 7-deazaguanosine residues.

Other non-canonical nucleotides that can be used in place of or in combination with 5-methyluridine include, but are not limited to pseudouridine, 5-hydroxyuridine, 5-hydroxypseudouridine, 5-methoxyuridine, 5-methoxypseudouridine, 5-carboxyuridine, 5-carboxypseudouridine, 5-formyluridine, 5-formylpseudouridine, 5-hydroxymethyluridine, 5-hydroxymethylpseudouridine, and 5-methylpseudouridine (“1-methylpseudouridine”, “N1-methylpseudouridine”) or one or more derivatives thereof. Other non-canonical nucleotides that can be used in place of or in combination with 5-methylcytidine and/or 5-hydroxymethylcytidine include, but are not limited to pseudoisocytidine, 5-methylpseudoisocytidine, 5-hydroxymethylcytidine, 5-formylcytidine, 5-carboxycytidine, 5-methoxycytidine, N4-methylcytidine, N4-acetylcytidine or one or more derivatives thereof. In certain embodiments, for example, when performing only a single transfection, injection or delivery or when the cells, tissue, organ or patient being transfected, injected or delivered to are not particularly sensitive to transfection-associated toxicity or innate-immune signaling, the fractions of non-canonical nucleotides can be reduced. Reducing the fraction of non-canonical nucleotides can be beneficial, in part, because reducing the fraction of non-canonical nucleotides can reduce the cost of the nucleic acid. In certain situations, for example, when minimal immunogenicity of the nucleic acid is desired, the fractions of non-canonical nucleotides can be increased.

Enzymes such as T7 RNA polymerase may preferentially incorporate canonical nucleotides in an in vitro-transcription reaction containing both canonical and non-canonical nucleotides. As a result, an in vitro-transcription reaction containing a certain fraction of a non-canonical nucleotide may yield RNA containing a different, often lower, fraction of the non-canonical nucleotide than the fraction at which the non-canonical nucleotide was present in the reaction. In certain embodiments, references to nucleotide incorporation fractions (for example, “50% 5-methyluridine”) therefore can refer both to nucleic acids containing the stated fraction of the nucleotide, and to nucleic acids synthesized in a reaction containing the stated fraction of the nucleotide (or nucleotide derivative, for example, nucleotide-triphosphate), even though such a reaction may yield a nucleic acid containing a different fraction of the nucleotide than the fraction at which the non-canonical nucleotide was present in the reaction. In addition, different nucleotide sequences can encode the same protein by utilizing alternative codons. In certain embodiments, references to nucleotide incorporation fractions therefore can refer both to nucleic acids containing the stated fraction of the nucleotide, and to nucleic acids encoding the same protein as a different nucleic acid, wherein the different nucleic acid contains the stated fraction of the nucleotide.

### Untranslated Regions (UTRs)

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the synthetic RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20 nt and about 250 nt or between about 120 nt and about 150 nt long. In a further embodiment, the 3′-poly(A) tail is about 20 nt, or about 30 nt, or about 40 nt, or about 50 nt, or about 60 nt, or about 70 nt, or about 80 nt, or about 90 nt, or about 100 nt, or about 110 nt, or about 120 nt, or about 130 nt, or about 140 nt, or about 150 nt, or about 160 nt, or about 170 nt, or about 180 nt, or about 190 nt, or about 200 nt, or about 210 nt, or about 220 nt, or about 230 nt, or about 240 nt, or about 250 nt long.

It has now been discovered that poly(A) tails produced by poly(A) polymerase may vary in length depending on reaction conditions including reaction time and enzyme activity, and that an enzymatic tailing reaction may produce a mixture of RNA molecules having poly(A) tails of varied lengths. Certain embodiments are directed to a synthetic RNA molecule containing a tail of about 10, about 20, about 30, about 40, about 50, about 75, about 100, about 125, about 150, about 175, about 200, about 225, about 250, about 275, about 300, about 325, about 350, or about 400, or more than about 400 nucleotides. In one embodiment, the tail is a poly(A) tail. Other embodiments are directed to a tail containing fewer than about 10 nucleotides.

It has now been discovered that synthesizing RNA using a template that encodes a tail can enable increased control over the length of the tail and reduced variability within or among reactions. Certain embodiments are therefore directed to a template encoding a tail. In certain embodiments, the tail contains about 10, about 20, about 30, about 40, about 50, about 75, about 100, about 125, about 150, about 175, about 200, about 225, about 250, about 275, about 300, about 325, about 350, or about 400 nucleotides. Other embodiments are directed to a synthetic RNA molecule synthesized using a template that encodes a tail.

### Exon Skipping

Cells can be induced to “skip” over faulty sections of pre-mRNA molecules by interfering with mRNA splicing. Such “exon skipping” can thus lead to a truncated, yet functional protein despite the presence of a genetic mutation. Exon skipping involves binding an antisense oligonucleotide to a splice site in a pre-mRNA molecule. When the pre-mRNA that is bound by the antisense oligonucleotide is processed into a mature mRNA, the corresponding exon can be “skipped” over, which, for example, can restore a disrupted reading frame caused by the mutation. Exon skipping can allow translation of an internally-deleted, but largely functional protein. However, current exon skipping methods are limited by the transience of the antisense oligonucleotide, and a persistent effect can thus require that the antisense oligonucleotide be continuously present in a patient's cells. Otherwise, if the cell lacks or possesses a sub-optimal amount of the antisense oligonucleotide, mature mRNA comprising a mutation can be processed and ultimately translated into a defective protein. Consequently, current exon skipping methods are limited by the need to provide the antisense oligonucleotide to the patient chronically and/or regularly.

An aspect of the present invention is a method for treating a disease or disorder caused by a mutation in a gene, the method comprising administering to a subject in need thereof and comprising the mutation in the gene an effective amount of a synthetic RNA encoding a gene-editing protein capable of creating a single-strand or double-strand break in the gene, wherein the single-strand or double-strand break causes persistent altered splicing of the gene.

In embodiments, the altered splicing results in expression of a truncated protein which lacks at least the polypeptide sequence corresponding to an exon containing the mutation.

In embodiments, the single-strand or double-strand break removes a splice acceptor site or produces a non-functional splice acceptor site in or near an exon of the gene or removes a splice donor site or produces a non-functional splice donor site in or near an exon of the gene.

In embodiments, the gene-editing protein creates a non-functional splice acceptor site that is within about 1 kb or about 0.5 kb or about 0.1 kb of the exon.

In embodiments, the mutation causes altered splicing of the gene and the single-strand or double-strand break causes the expression of a functional gene product.

In embodiments, the mutation inactivates a splice acceptor site or a splice donor site and the single-strand or double-strand break restores a functional exon.

In any of the preceding embodiments and aspect, the single-strand or double-strand break is within about 1 kb or about 0.5 kb or about 0.1 kb of the exon.

In any of the preceding embodiments and aspect, the non-functional splice acceptor site causes excision of the exon when a pre-mRNA comprising the exon is processed into mRNA.

In embodiments, the gene-editing protein creates a non-functional splice donor site in an intron that is within about 1 kb or about 0.5 kb or about 0.1 kb of the exon.

In any of the preceding embodiments and aspect, the non-functional splice donor site causes excision of the exon when a pre-mRNA comprising the exon is processed into mRNA.

In any of the preceding embodiments and aspect, wherein the exon comprises a mutation.

In embodiments, the mutation is a nonsense mutation, a frame shift mutation, or a mutation that introduces a premature stop codon.

In any of the preceding embodiments and aspect, wherein the mRNA is translated into a truncated protein which retains a function of the full-length protein.

In any of the preceding embodiments and aspect, wherein the exon encodes a polypeptide sequence comprising a peptide splice site.

In embodiments, the mRNA is translated into a polypeptide which lacks the peptide splice site.

In embodiments, the cleavage site is a protease cleavage site or a caspase cleavage site.

In any of the preceding embodiments and aspect, wherein the exon encodes a polypeptide sequence comprising a cleavage site.

In embodiments, the mRNA is translated into a polypeptide which lacks the cleavage site.

In any of the preceding embodiments and aspect, the truncated protein possesses a function of the wild-type protein.

In any of the preceding embodiments and aspect, the gene-editing protein is selected from a TALEN, a meganuclease, a nuclease, a zinc finger nuclease, a CRISPR-associated protein, CRISPR/Cas9, Cas9, xCas9, Cas12a (Cpf1), Cas13a, Cas14, CasX, CasY, a Class 1 Cas protein, a Class 2 Cas protein, and MAD7.

In any of the preceding embodiments and aspect, the gene-editing protein comprises: (a) a DNA-binding domain comprising a plurality of repeat sequences and at least one of the repeat sequences comprises the amino acid sequence: LTPvQWAIAwxyzGHGG (SEQ ID NO: 629), wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 630) or GGKQALETVQRLLPVLCQA (SEQ ID NO: 631); and (b) a nuclease domain comprising a catalytic domain of a nuclease.

In embodiments, the nuclease domain is capable of forming a dimer with another nuclease domain.

In any of the preceding embodiments and aspect, the nuclease domain comprises the catalytic domain of a protein comprising the amino acid sequence of SEQ ID NO: 632.

In any of the preceding embodiments and aspect, at least one of the repeat sequences comprising the amino acid sequence LTPvQWAIAwxyzGHGG (SEQ ID NO: 629) is between 36 and 39 amino acids long.

In any of the preceding embodiments and aspect, the gene is selected from ABCA4, ADAMTS-13, APP, ATP6AP2, CEP290, COL17A1, COL4A3, COL4A4, COL4A5, COL6A1, COL6A2, COL6A3, COL7A1, DMD, DMD, FUS, FXN, GABRG2, HNRPDL, HTT, IKBKAP, ITGA6, ITGB4, LAMA3, LAMB3, LAMC2, LMNA, LMNA, LMNA, LMNA, LMNB1, MAPT, PINK1, PRPF6, RBM20, RNU4ATAC, SMN1, SNRNP200, TARDP, TCF4, TTN, U2AF1, USH2A, and USH2A.

In any of the preceding embodiments and aspect, a gene, the sequence identifier (SEQ ID NO) for its NCBI Reference Sequence, a mutation or mutations therein, the intron or introns that are associated with diseases, and/or the exon or exons that are associated with diseases which can be treated by the method is selected from the list Table 2.

In any of the preceding embodiments and aspect, the disease or disorder is selected from Alport Syndrome, Alport Syndrome, Alport Syndrome, Alzheimer's disease, Amyotrophic lateral sclerosis (ALS), Autosomal dominant leukodystrophy (ADLD), Becker muscular dystrophy (BMD), Bethlem myopathy and Ullrich scleroatonic muscular dystrophy, Dilated cardiomyopathy (DCM), Duchenne muscular dystrophy, Dystrophic Epidermolysis Bullosa, Early-onset Parkinson disease (PD), Epidermolysis Bullosa (EB), Familial dysautonomia (FD), Familial partial lipodystrophy type 2 (FPLD2), Febrile seizures (FS); childhood absence epilepsy (CAE), generalized epilepsy with febrile seizures plus (GEFS+), and Dravet syndrome (DS)/severe myoclonic epilepsy in infancy (SMEI), Friedreich ataxia, Frontotemporal dementia with parkinsonism chromosome 17 (FTDP-17), Fuchs endothelial corneal dystrophy (FECD), Huntington's Disease, Hutchinson-Gilford progeria syndrome (HGPS), Junctional Epidermolysis Bullosa, Leber's congenital amaurosis (LCA), Limb girdle muscular dystrophy type 1B (LGMD1B), Limb-girdle muscular dystrophy 1G (LGMD1G), Microcephalic osteodysplastic primordial dwarfism type 1 (MOPD I), Myelodysplastic syndromes (MDS), Retinitis pigmentosa (adRP), Spinal muscular atrophy (SMA), Stargardt disease, Thrombotic thrombocytopenic purpura (TTP), Ushers syndrome type I, Ushers syndrome type II, Various myopathies and dystrophies, a wound, and X-linked parkinsonism with spasticity (XPDS).

In any of the preceding embodiments and aspect, a single administration of the effective amount of the synthetic RNA encoding the gene-editing protein causes persistent altered RNA splicing of the gene.

Aspects of the present invention are directed to modulating exon splicing, also referred to herein as “altering RNA splicing”.

In contrast to other methods, various embodiments of the present invention modify genomic DNA by introducing a single or double-stranded break in or near an exon to create a non-functional splice acceptor site in or near the exon or a non-functional splice donor site in an intron near the exon. In embodiments, “near an/the exon” means within about 1 kb or about 0.5 kb or about 0.1 kb of the exon. In some embodiments, the exon will be skipped during pre-mRNA processing. In some embodiments, the exon will be skipped without needing to be bound by an antisense oligonucleotide. Thus, in contrast to other methods, some embodiments of the present invention are effective following a single or a few administrations of RNA that express gene-editing proteins that target an exon. In some embodiments, the exon contains a mutation. In some embodiments, the mutation is a disease-causing mutation. In addition, a disease or disorder may be caused by protein splicing which produces a deleterious spliceform. Certain embodiments are therefore directed to produce an mRNA which lacks the exon that encodes a polypeptide sequence comprising a splice site. In certain embodiments, the resulting protein cannot form the deleterious spliceform.

Gene-editing proteins (and nucleic acids encoding gene-editing proteins) of the present invention may thus be used for altering RNA splicing for any genetic disorder that could be treated by exon skipping, e.g., Alport Syndrome, Alzheimer's disease, Bethlem myopathy and Ullrich scleroatonic muscular dystrophy, Duchenne muscular dystrophy, Dystrophic Epidermolysis Bullosa, Friedreich ataxia, Huntington's Disease, Junctional Epidermolysis Bullosa, Leber's congenital amaurosis (LCA), and various myopathies and dystrophies. The following table, Table 2, includes illustrative genes, mutations in the genes, introns, and exons that are associated with diseases which can be treated by modulating exon splicing, as disclosed herein.

TABLE 2

| | | | | SEQ ID | |
|---|---|---|---|---|---|
| | | | | NO: for | |
| | | | NCBI | the NCBI | Mutation/ |
| | | NCBI | Reference | Reference | relevant |
| Gene | Diseases | Gene ID | Sequence | Sequence | gene domain |
| ABCA4 | Stargardt | 24 | NG_009073.1 | 740 | (c.5461-10T > C); |
| | disease | | | | Exon 39 |
| ADAMTS-13 | Thrombotic | 11093 | NG_011934.2 | 741 | |
| | thrombocytopenic | | | | |
| | purpura (TTP) | | | | |
| APP | Alzheimer's | 351 | NG_007376.2 | 742 | exon 17 splicing |
| | disease | | | | which removes |
| | | | | | cleavage site |
| ATP6AP2 | X-linked | 10159 | NG_008874.1 | 743 | (c.345C > T); |
| | parkinsonism | | | | Exon 4 |
| | with spasticity | | | | |
| | (XPDS) | | | | |
| CEP290 | Leber's | 80184 | NG_008417.1 | 744 | Intron 26, |
| | congenital | | | | most common |
| | amaurosis (LCA) | | | | LCA-causing |
| | | | | | mutation |
| | | | | | results in a |
| | | | | | splice-donor |
| | | | | | in intron 26 |
| C0L17A1 | Junctional | 1308 | NG_007069.1 | 745 | |
| | Epidermolysis | | | | |
| | Bullosa | | | | |
| COL4A3 | Alport Syndrome | 1285 | NG_011591.1 | 746 | |
| COL4A4 | Alport Syndrome | 1286 | NG_011592.1 | 747 | |
| COL4A5 | Alport Syndrome | 1287 | NG_011977.2 | 748 | |
| COL6A1 | Bethlem | 1291 | NG_008674.1 | 749 | |
| | myopathy and | | | | |
| | Ullrich | | | | |
| | scleroatonic | | | | |
| | muscular | | | | |
| | dystrophy | | | | |
| COL6A2 | Bethlem | 1292 | NG_008675.1 | 750 | |
| | myopathy and | | | | |
| | Ullrich | | | | |
| | scleroatonic | | | | |
| | muscular | | | | |
| | dystrophy | | | | |
| COL6A3 | Bethlem | 1293 | NG_008676.1 | 751 | |
| | myopathy and | | | | |
| | Ullrich | | | | |
| | scleroatonic | | | | |
| | muscular | | | | |
| | dystrophy | | | | |
| COL7A1 | Dystrophic | 1294 | NG_007065.1 | 752 | |
| | Epidermolysis | | | | |
| | Bullosa | | | | |
| DMD | Duchenne | 1756 | NG_012232.1 | 753 | |
| | muscular | | | | |
| | dystrophy | | | | |
| DMD | Becker | 1756 | NG_012232.1 | 753 | (c.4250T > A); |
| | muscular | | | | Exon 31 |
| | dystrophy (BMD) | | | | |
| FUS | Amyotrophic | 2521 | NG_012889.2 | 755 | (c. 1566C > T), |
| | lateral | | | | (c. 1561T > G) |
| | sclerosis (ALS) | | | | |
| FXN | Friedreich | 2395 | NG_008845.2 | 756 | Intron 1 |
| | ataxia | | | | Target regions |
| | | | | | surround expanded |
| | | | | | GAA repeats |
| GABRG2 | Febrile | 2566 | NG_009290.1 | 757 | |
| | seizures (FS); | | | | |
| | childhood absence | | | | |
| | epilepsy (CAE), | | | | |
| | generalized | | | | |
| | epilepsy with | | | | |
| | febrile | | | | |
| | seizures plus | | | | |
| | (GEFS+), and | | | | |
| | Dravet syndrome | | | | |
| | (DS)/severe | | | | |
| | myoclonic epilepsy | | | | |
| | in infancy (SMEI) | | | | |
| HNRPDL | Limb-girdle | 9987 | NG_029681.1 | 758 | (c. 1667G > A), |
| | muscular | | | | (c.1667G > C) |
| | dystrophy 1G | | | | |
| | (LGMD1G) | | | | |
| HTT | Huntington's | 3064 | NG_009378.1 | 759 | exon 12 splicing - |
| | Disease | | | | Removal of exon 12 |
| | | | | | inhibits caspase-6 |
| | | | | | cleavage of HTT, |
| | | | | | reducing toxicity |
| IKBKAP | Familial | 8518 | NG_008788.1 | 760 | (c.2204 + 6T > C); |
| | dysautonomia | | | | Exon 20 |
| | (FD) | | | | |
| ITGA6 | Junctional | 3694 | NG_042041.1 | 761 | |
| | Epidermolysis | | | | |
| | Bullosa | | | | |
| ITGB4 | Junctional | 3691 | NG_007372.1 | 762 | |
| | Epidermolysis | | | | |
| | Bullosa | | | | |
| LAMA3 | Junctional | 3909 | NG_007853.2 | 763 | |
| | Epidermolysis | | | | |
| | Bullosa | | | | |
| LAMB3 | Junctional | 3914 | NG_007116.1 | 764 | |
| | Epidermolysis | | | | |
| | Bullosa | | | | |
| LAMC2 | Junctional | 3918 | NG_007079.2 | 765 | |
| | Epidermolysis | | | | |
| | Bullosa | | | | |
| LMNA | Dilated | 4000 | NG_008692.2 | 766 | (c.640-10A > G); |
| | cardiomyopathy | | | | Exon 4 |
| | (DCM) | | | | |
| LMNA | Familial partial | 4000 | NG_008692.2 | 766 | (c.1488 + 5G > C); |
| | lipodystrophy | | | | Intron 8 |
| | type 2 (FPLD2) | | | | |
| LMNA | Hutchinson- | 4000 | NG_008692.2 | 766 | (c.1824C > T); |
| | Gilford progeria | | | | Exon 11 |
| | syndrome (HGPS) | | | | |
| LMNA | Limb girdle | 4000 | NG_008692.2 | 766 | (c.1608 + 5G > C); |
| | muscular dystrophy | | | | Intron 9 |
| | type 1B (LGMD1B) | | | | |
| LMNB1 | Autosomal | 4001 | NG_008360.2 | 770 | |
| | dominant | | | | |
| | leukodystrophy | | | | |
| | (ADLD) | | | | |
| MAPT | Frontotemporal | 4137 | NG_007398 | 771 | (c.892A > G); |
| | dementia with | | | | Exon 10 |
| | parkinsonism | | | | |
| | chromosome 17 | | | | |
| | (FTDP-17) | | | | |
| PINK1 | Early-onset | 65018 | NG_008164.1 | 772 | (c.1488 + 1G > A); |
| | Parkinson | | | | Exon 7 |
| | disease (PD) | | | | |
| PRPF6 | Retinitis | 24148 | NG_029719.1 | 773 | (c.2185C > T) |
| | pigmentosa (adRP) | | | | |
| RBM20 | Dilated | 282996 | NG_021177.1 | 774 | (c.1962T > G) |
| | cardiomyopathy | | | | |
| | (DCM) | | | | |
| RNU4ATAC | Microcephalic | 100151683 | NG_029832.1 | 775 | (g.30G > A), |
| | osteodysplastic | | | | (g.50G > A), |
| | primordial | | | | (g.50G > C), |
| | dwarfism type 1 | | | | (g.51G > A), |
| | (MOPD I) | | | | (g.53C > G), |
| | | | | | (g.55G > A), |
| | | | | | (g.111G > A) |
| SMN1 | Spinal muscular | 6606 | NG_008691.1 | 776 | (c.922 + 6T/G) |
| | atrophy (SMA) | | | | |
| SNRNP200 | Retinitis | 23020 | NG_016973.1 | 777 | (c.3260C > T), |
| | pigmentosa (adRP) | | | | (c.3269G > T) |
| TARDP | Amyotrophic | 23435 | NG_008734.1 | 778 | (c.991C > A), |
| | lateral sclerosis | | | | (c.1009A > G) |
| | (ALS) | | | | |
| TCF4 | Fuchs endothelial | 6925 | NG_011716.2 | 779 | |
| | corneal dystrophy | | | | |
| | (FECD) | | | | |
| TTN | Various myopathies | 7273 | NG_011618.3 | 780 | |
| | and dystrophies | | | | |
| U2AF1 | Myelodysplastic | 7307 | NG_029455.1 | 781 | (c.101G > A) |
| | syndromes (MDS) | | | | |
| USH2A | Ushers | 7399 | NG_009497.1 | 782 | Exon 13 |
| | syndrome type I | | | | |
| USH2A | Ushers | 7399 | NG_009497.1 | 782 | PE40 |
| | syndrome type II | | | | (cryptic exon) |

| | | | Exons | | |
|---|---|---|---|---|---|
| | | Exons | amenable | | |
| | | amenable | to single | Exons | Exons |
| | | to single | target site | amenable | amenable |
| | | target site | treatment | to double | to triple |
| | | treatment | correction | target site | target site |
| | | to estore | of frameshift | treatment of | treatment of |
| | | reading | or premature | frameshift or | frameshift or |
| | | frame | stop codon | PSC within | PSC within |
| | | outside of | (PSC) within | one or more | one or more |
| Gene | Diseases | the exon | the exon | exons | exons |
| ABCA4 | Stargardt | | | | |
| | disease | | | | |
| ADAMTS-13 | Thrombotic | | | | |
| | thrombocytopenic | | | | |
| | purpura (TTP) | | | | |
| APP | Alzheimer's | | | | |
| | disease | | | | |
| ATP6AP2 | X-linked | | | | |
| | parkinsonism | | | | |
| | with spasticity | | | | |
| | (XPDS) | | | | |
| CEP290 | Leber's | | | | |
| | congenital | | | | |
| | amaurosis (LCA) | | | | |
| COL17A1 | Junctional | | 2, 3, 4, 5, 6, 7, | | |
| | Epidermolysis | | 8, 9, 10, 11, 12, | | |
| | Bullosa | | 13, 14, 15, 16, 17, | | |
| | | | 18, 19, 20, 21, 22, | | |
| | | | 23, 24, 25, 26, 27, | | |
| | | | 28, 29, 30, 31, 32, | | |
| | | | 33, 34, 35, 36, 37, | | |
| | | | 38, 39, 40, 41, 42, | | |
| | | | 43, 44, 45, 46, 47, | | |
| | | | 48, 49, 50, 51, 52, | | |
| | | | 53, 54, 55 | | |
| COL4A3 | Alport Syndrome | 19, 24, 26, 29, 30, | 2, 3, 4, 5, 6, 7, | (24; 26), (29; 30), | (24; 25; 26) |
| | | 37, 38, 43, 44, 49, | 8, 9, 10, 11, 12, | (37; 38), (43; 44), | |
| | | 50, 51 | 13, 14, 15, 16, 17, | (50; 51), (51; 52) | |
| | | | 18, 20, 21, 22, 23, | | |
| | | | 25, 27, 28, 31, 32, | | |
| | | | 33, 34, 35, 36, 39, | | |
| | | | 40, 41, 42, 45, 46, | | |
| | | | 47, 48 | | |
| COL4A4 | Alport Syndrome | 2, 3, 18, 22, 23, | 4, 5, 6, 7, 8, 9, | (22; 23), (23; 24), | |
| | | 24, 25, 33, 34, 47 | 10, 11, 12, 13, 14, | (24; 25), (33; 34) | |
| | | | 15, 16, 17, 19, 20, | | |
| | | | 21, 26, 27, 28, 29, | | |
| | | | 30, 31, 32, 35,36, | | |
| | | | 37, 38, 39, 40, 41, | | |
| | | | 42, 43, 44, 45, 46 | | |
| COL4A5 | Alport Syndrome | 19, 23, 25, 28, 29, | 2, 3, 4, 5, 6, 7, | (23; 25), (28; 29), | (23; 24; 25) |
| | | 36, 37, 44, 45, 50, | 8, 9, 10, 11, 12, | (36; 37), (44; 45), | |
| | | 51, 52 | 13, 14, 15, 16, 17, | (51; 52), (52; 53) | |
| | | | 18, 20, 21, 22, 24, | | |
| | | | 26, 27, 30, 31, 32, | | |
| | | | 33, 34, 35, 38, 39, | | |
| | | | 40, 41, 42, 43, 46, | | |
| | | | 47, 48, 49 | | |
| COL6A1 | Bethlem | 2, 4, 28, 30, 31, | 3, 5, 6, 7, 8, 9, | (28; 30), (31; 32) | (28; 29; 30) |
| | myopathy and | 32, 33 | 10, 11, 12, 13, 14, | | |
| | Ullrich | | 15, 16, 17, 18, 19, | | |
| | scleroatonic | | 20, 21, 22, 23, 24, | | |
| | muscular | | 25, 26, 27, 29, 34 | | |
| | dystrophy | | | | |
| COL6A2 | Bethlem | 2, 3, 24 | 4, 5, 6, 7, 8, 9, | (2; 3) | |
| | myopathy and | | 10, 11, 12, 13, 14, | | |
| | Ullrich | | 15, 16, 17, 18, 19, | | |
| | scleroatonic | | 20, 21, 22, 23, 25, | | |
| | muscular | | 26, 27 | | |
| | dystrophy | | | | |
| COL6A3 | Bethlem | 2, 12, 13, 14, 34, | 3, 4, 5, 6, 7, 8, | (12; 13), (13; 14), | (34; 35; 36) |
| | myopathy and | 36, 37, 39, 40 | 9, 10, 11, 15, 16, | (34; 36), (36; 37), | |
| | Ullrich | | 17, 18, 19, 20, 21, | (39; 40) | |
| | scleroatonic | | 22, 23, 24, 25, 26, | | |
| | muscular | | 27, 28, 29, 30, 31, | | |
| | dystrophy | | 32, 33, 35, 38, 41, | | |
| | | | 42, 43 | | |
| COL7A1 | Dystrophic | 2, 3, 4, 6, 7, 24, | 5, 8, 9, 10, 11, 12, | (4; 6), (6; 7), | (2; 3; 4), (4; 5; 6), |
| | Epidermolysis | 25, 27, 113 | 13, 14, 15, 16, 17, | (24; 25), (25; 27) | (25; 26; 27) |
| | Bullosa | | 18, 19, 20, 21, 22, | | |
| | | | 23, 26, 28, 29, 30, | | |
| | | | 31, 32, 33, 34, 35, | | |
| | | | 36, 37, 38, 39, 40, | | |
| | | | 41, 42, 43, 44, 45, | | |
| | | | 46, 47, 48, 49, 50, | | |
| | | | 51, 52, 53, 54, 55, | | |
| | | | 56, 57, 58, 59, 60, | | |
| | | | 61, 62, 63, 64, 65, | | |
| | | | 66, 67, 68, 69, 70, | | |
| | | | 71, 72, 73, 74, 75, | | |
| | | | 76, 77, 78, 79, 80, | | |
| | | | 81, 82, 83, 84, 85, | | |
| | | | 86, 87, 88, 89, 90, | | |
| | | | 91, 92, 93, 94, 95, | | |
| | | | 96, 97, 98, 99, 100, | | |
| | | | 101, 102, 103, 104, | | |
| | | | 105, 106, 107, 108, | | |
| | | | 109, 110, 111, 112, | | |
| | | | 114, 115, 116, 117 | | |
| DMD | Duchenne | 2, 6, 7, 8, 11, 12, | 3, 4, 5, 9, 10, 13, | (11; 12), (17; 18), | (6; 7; 8), |
| | muscular | 17, 18, 19, 20, 21, | 14, 15, 16, 23, 24, | (19; 20), (20; 21), | (59; 60; 61), |
| | dystrophy | 22, 43, 44, 45, 46, | 25, 26, 27, 28, 29, | (21; 22), (43; 44), | (63; 64; 65), |
| | | 50, 51, 52, 53, 54, | 30, 31, 32, 33, 34, | (44; 45), (45; 46), | (66; 67; 68), |
| | | 55, 56, 57, 58, 59, | 35, 36, 37, 38, 39, | (50; 51), (51; 52), | (76; 77; 78) |
| | | 61, 62, 63, 65, 66, | 40, 41, 42, 47, 48, | (52; 53), (54; 55), | |
| | | 67, 68, 69, 70, 75, | 49, 60, 64, 71, 72, | (55; 56), (56; 57), | |
| | | 76, 78 | 73, 74, 77 | (58; 59), (59; 61), | |
| | | | | (62; 63), (63; 65), | |
| | | | | (65; 66), (68; 69), | |
| | | | | (69; 70), (76; 78) | |
| DMD | Becker | | | | |
| | muscular | | | | |
| | dystrophy (BMD) | | | | |
| FUS | Amyotrophic | | | | |
| | lateral | | | | |
| | sclerosis (ALS) | | | | |
| FXN | Friedreich | | | | |
| | ataxia | | | | |
| GABRG2 | Febrile | | | | |
| | seizures (FS); | | | | |
| | childhood absence | | | | |
| | epilepsy (CAE), | | | | |
| | generalized | | | | |
| | epilepsy with | | | | |
| | febrile | | | | |
| | seizures plus | | | | |
| | (GEFS+), and | | | | |
| | Dravet syndrome | | | | |
| | (DS)/severe | | | | |
| | myoclonic epilepsy | | | | |
| | in infancy (SMEI) | | | | |
| HNRPDL | Limb-girdle | | | | |
| | muscular | | | | |
| | dystrophy 1G | | | | |
| | (LGMD1G) | | | | |
| HTT | Huntington's | | | | |
| | Disease | | | | |
| IKBKAP | Familial | | | | |
| | dysautonomia | | | | |
| | (FD) | | | | |
| ITGA6 | Junctional | 2, 3, 4, 5, 10, 11, | 6, 7, 8, 9, 13 | (2; 3), (11; 12) | (3; 4; 5) |
| | Epidermolysis | 12, 14 | | | |
| | Bullosa | | | | |
| ITGB4 | Junctional | 2, 3, 5, 6, 7, 12, | 4, 8, 9, 10, 11, | (3; 5), (14; 16), | (3; 4; 5), (5; 6; 7), |
| | Epidermolysis | 13, 14, 16, 18, 19, | 15, 17, 20, 23, 25, | (16; 18), (18; 19), | (12; 13; 14), |
| | Bullosa | 21, 22, 24, 26, 27, | 30, 31, 32, 33, 34, | (19; 21), (26; 27), | (14; 15; 16), |
| | | 28, 29 | 35, 36, 37, 38, 39 | (27; 28), (28; 29) | (16; 17; 18), |
| | | | | | (19; 20; 21) |
| LAMA3 | Junctional | 3, 4, 6, 7, 8, 9, | 2, 5, 10, 11, 12, | (3; 4), (8; 9), | (6; 7; 8), |
| | Epidermolysis | 14, 15, 16, 17, 18, | 13, 34, 35, 36, 37, | (14; 15), (15; 16), | (17; 18; 19), |
| | Bullosa | 19, 20, 21, 22, 23, | 38, 39, 41, 43, 45, | (16; 17), (20; 21), | (18; 19; 20), |
| | | 24, 25, 26, 27, 28, | 47, 49, 53, 54, 56, | (22; 23), (23; 24), | (29; 30; 31), |
| | | 29, 30, 31, 32, 33, | 61, 64, 66, 70, 74 | (24; 25), (25; 26), | (44; 45; 46), |
| | | 40, 42, 44, 46, 48, | | (26; 27), (28; 29), | (46; 47; 48), |
| | | 50, 51, 52, 55, 57, | | (31; 32), (44; 46), | (60; 61; 62), |
| | | 58, 59, 60, 62, 63, | | (46; 48), (50; 51), | (63; 64; 65), |
| | | 65, 67, 68, 69, 71, | | (57; 58), (58; 59), | (65; 66; 67) |
| | | 72, 73 | | (59; 60), (60; 62), | |
| | | | | (62; 63), (63; 65), | |
| | | | | (65; 67), (67; 68), | |
| | | | | (68; 69), (71; 72), | |
| | | | | (72; 73) | |
| LAMB3 | Junctional | 2, 3, 4, 5, 7, 8, | 6, 10, 11, 17, 21 | (3; 4), (4; 5), | (5; 6; 7), |
| | Epidermolysis | 9, 12, 13, 14, 15, | | (5; 7), (7; 8), | (16; 17; 18), |
| | Bullosa | 16, 18, 19, 20, 22 | | (8; 9), (12; 13), | (18; 19; 20) |
| | | | | (14; 15), (16; 18) | |
| LAMC2 | Junctional | 2, 3, 4, 6, 8, 10, | 5, 7, 9, 12, 13, 14, | (4; 6), (6; 8), | (2; 3; 4), (4; 5; 6), |
| | Epidermolysis | 11, 15, 16, 17, 18, | 21, 23, 26 | (8; 10), (10; 11), | (6; 7; 8), (8; 9; |
| | Bullosa | 19, 20, 22, 24, 25, | | (15; 16), (17; 18), | 10), (20; 21; 22), |
| | | 27 | | (18; 19), (20; 22), | (25; 26; 27) |
| | | | | (24; 25), (25; 27), | |
| | | | | (27; 28) | |
| LMNA | Dilated | | | | |
| | cardiomyopathy | | | | |
| | (DCM) | | | | |
| LMNA | Familial partial | | | | |
| | lipodystrophy | | | | |
| | type 2 (FPLD2) | | | | |
| LMNA | Hutchinson- | | | | |
| | Gilford progeria | | | | |
| | syndrome (HGPS) | | | | |
| LMNA | Limb girdle | | | | |
| | muscular dystrophy | | | | |
| | type 1B (LGMD1B) | | | | |
| LMNB1 | Autosomal | | | | |
| | dominant | | | | |
| | leukodystrophy | | | | |
| | (ADLD) | | | | |
| MAPT | Frontotemporal | | | | |
| | dementia with | | | | |
| | parkinsonism | | | | |
| | chromosome 17 | | | | |
| | (FTDP-17) | | | | |
| PINK1 | Early-onset | | | | |
| | Parkinson | | | | |
| | disease (PD) | | | | |
| PRPF6 | Retinitis | | | | |
| | pigmentosa (adRP) | | | | |
| RBM20 | Dilated | | | | |
| | cardiomyopathy | | | | |
| | (DCM) | | | | |
| RNU4ATAC | Microcephalic | | | | |
| | osteodysplastic | | | | |
| | primordial | | | | |
| | dwarfism type 1 | | | | |
| | (MOPD I) | | | | |
| SMN1 | Spinal muscular | | | | |
| | atrophy (SMA) | | | | |
| SNRNP200 | Retinitis | | | | |
| | pigmentosa (adRP) | | | | |
| TARDP | Amyotrophic | | | | |
| | lateral sclerosis | | | | |
| | (ALS) | | | | |
| TCF4 | Fuchs endothelial | | | | |
| | corneal dystrophy | | | | |
| | (FECD) | | | | |
| TTN | Various myopathies | 2, 5, 6, 7, 18, 19, | 3, 4, 8, 9, 10, 11, | (6; 7), (19; 21), | (19; 20; 21), (25; |
| | and dystrophies | 21, 22, 24, 25, 27, | 12, 13, 14, 15, 16, | (25; 27), (27; 28), | 26; 27), (39; 40; 41), |
| | | 28, 39, 40, 41, | 17, 20, 23, 26, 29, | (228; 229), (233; | (241; 242; 243), |
| | | 102, 103, 105, 228, | 30, 31, 32, 33, 34, | 234), (238; 239), | (255; 256; 257), |
| | | 229, 233, 234, 238, | 35, 36, 37, 38, 42, | (239; 240), (240; | (282; 283; 284), |
| | | 239, 240, 241, 243, | 43, 44, 45, 46, 47, | 241), (241; 243), | (284; 285; 286), |
| | | 244, 248, 249, 254, | 48, 49, 50, 51, 52, | (243; 244), (248; | (361; 362; 363) |
| | | 255, 257, 258, 259, | 53, 54, 55, 56, 57, | 249), (254; 255), | |
| | | 260, 263, 264, 267, | 58, 59, 60, 61, 62, | (255; 257), (257; | |
| | | 268, 281, 282, 284, | 63, 64, 65, 66, 67, | 258), (259; 260), | |
| | | 286, 292, 293, 358, | 68, 69, 70, 71, 72, | (263; 264), (267; | |
| | | 359, 360, 361 | 73, 74, 75, 76, 77, | 268), (281; 282), | |
| | | | 78, 79, 80, 81, 82, | (282; 284), (284; | |
| | | | 83, 84, 85, 86, 87, | 286), (292; 293), | |
| | | | 88, 89, 90, 91, 92, | (358; 359), (359; | |
| | | | 93, 94, 95, 96, 97, | 360), (360; 361), | |
| | | | 98, 99, 100, 101, | (361; 363) | |
| | | | 104, 106, 107, 108, | | |
| | | | 109, 110, 111, 112, | | |
| | | | 113, 114, 115, 116, | | |
| | | | 117, 118, 119, 120, | | |
| | | | 121, 122, 123, 124, | | |
| | | | 125, 126, 127, 128, | | |
| | | | 129, 130, 131, 132, | | |
| | | | 133, 134, 135, 136, | | |
| | | | 137, 138, 139, 140, | | |
| | | | 141, 142, 143, 144, | | |
| | | | 145, 146, 147, 148, | | |
| | | | 149, 150, 151, 152, | | |
| | | | 153, 154, 155, 156, | | |
| | | | 157, 158, 159, 160, | | |
| | | | 161, 162, 163, 164, | | |
| | | | 165, 166, 167, 168, | | |
| | | | 169, 170, 171, 172, | | |
| | | | 173, 174, 175, 176, | | |
| | | | 177, 178, 179, 180, | | |
| | | | 181, 182, 183, 184, | | |
| | | | 185, 186, 187, 188, | | |
| | | | 189, 190, 191, 192, | | |
| | | | 193, 194, 195, 196, | | |
| | | | 197, 198, 199, 200, | | |
| | | | 201, 202, 203, 204, | | |
| | | | 205, 206, 207, 208, | | |
| | | | 209, 210, 211, 212, | | |
| | | | 213, 214, 215, 216, | | |
| | | | 217, 218, 219, 220, | | |
| | | | 221, 222, 223, 224, | | |
| | | | 225, 226, 227, 230, | | |
| | | | 231, 232, 235, 236, | | |
| | | | 237, 242, 245, 246, | | |
| | | | 247, 250, 251, 252, | | |
| | | | 253, 256, 261, 262, | | |
| | | | 265, 266, 269, 270, | | |
| | | | 271, 272, 273, 274, | | |
| | | | 275, 276, 277, 278, | | |
| | | | 279, 280, 283, 285, | | |
| | | | 287, 288, 289, 290, | | |
| | | | 291, 294, 295, 296, | | |
| | | | 297, 298, 299, 300, | | |
| | | | 301, 302, 303, 304, | | |
| | | | 305, 306, 307, 308, | | |
| | | | 309, 310, 311, 312, | | |
| | | | 313, 314, 315, 316, | | |
| | | | 317, 318, 319, 320, | | |
| | | | 321, 322, 323, 324, | | |
| | | | 325, 326, 327, 328, | | |
| | | | 329, 330, 331, 332, | | |
| | | | 333, 334, 335, 336, | | |
| | | | 337, 338, 339, 340, | | |
| | | | 341, 342, 343, 344, | | |
| | | | 345, 346, 347, 348, | | |
| | | | 349, 350, 351, 352, | | |
| | | | 353, 354, 355, 356, | | |
| | | | 357, 362 | | |
| U2AF1 | Myelodysplastic | | | | |
| | syndromes (MDS) | | | | |
| USH2A | Ushers | | | | |
| | syndrome type I | | | | |
| USH2A | Ushers | | | | |
| | syndrome type II | | | | |

The data relevant and presently published with respect to the NCBI Gene ID and NCBI Reference Sequence listed in the table above are hereby incorporated by reference in their entireties.

In various embodiments, the present compositions alter RNA splicing of exons associated with a disease or disorder. In various embodiments, the disease or disorder is selected from Alport Syndrome, Alzheimer's disease, Amyotrophic lateral sclerosis (ALS), Autosomal dominant leukodystrophy (ADLD), Becker muscular dystrophy (BMD), Bethlem myopathy and Ullrich scleroatonic muscular dystrophy, Dilated cardiomyopathy (DCM), Duchenne muscular dystrophy, Dystrophic Epidermolysis Bullosa, Early-onset Parkinson disease (PD), Familial dysautonomia (FD), Familial partial lipodystrophy type 2 (FPLD2), Febrile seizures (FS); childhood absence epilepsy (CAE), generalized epilepsy with febrile seizures plus (GEFS+), and Dravet syndrome (DS)/severe myoclonic epilepsy in infancy (SMEI), Friedreich ataxia, Frontotemporal dementia with parkinsonism chromosome 17 (FTDP-17), Fuchs endothelial corneal dystrophy (FECD), Huntington's Disease, Hutchinson-Gilford progeria syndrome (HGPS), Junctional Epidermolysis Bullosa, Leber's congenital amaurosis (LCA), Limb girdle muscular dystrophy type 1B (LGMD1B), Limb-girdle muscular dystrophy 1G (LGMD1G), Microcephalic osteodysplastic primordial dwarfism type 1 (MOPD I), Myelodysplastic syndromes (MDS), Retinitis pigmentosa (adRP), Spinal muscular atrophy (SMA), Stargardt disease, Thrombotic thrombocytopenic purpura (TTP), Ushers syndrome type I, Ushers syndrome type II, X-linked parkinsonism with spasticity (XPDS), and Various myopathies and dystrophies.

### Treating Diseases, Disorders, or Injuries of the Central Nervous System (CNS)

In various embodiments, the present methods and compositions find use in methods of treating, preventing, or ameliorating a disease, disorder, and/or condition. For instance, in some embodiments, the described methods of in vivo delivery, including various effective doses, administration strategies, and formulations are used in a method of treatment.

An aspect of the present invention is a method for treating a neurodegenerative disease or central nervous system injury comprising administering to a subject in need thereof a synthetic RNA encoding a neurotrophic agent, a gene-editing protein, or an enzyme that cleaves a dysfunctional, an abnormally folding, and/or a disease-causing protein, wherein the neurotrophic agent, the gene-editing protein, or the enzyme treats the neurodegenerative disease or central nervous system injury.

In embodiments, the neurodegenerative disease is selected from: a motor neuron disease, a polyglutamine disease, a prion disease, a spinocerebellar ataxia, a trinucleotide repeat disorder, Alzheimer's disease, amyotrophic lateral sclerosis (ALS), ataxia telangiectasia, ataxia-oculomotor apraxia, Batten disease, Cockayne syndrome, dementia, familial encephalopathy, Huntington's disease, Lewy-body dementia, multiple system atrophy, Parkinson's disease, spinocerebellar ataxia type 1, spongiform encephalopathy, and xeroderma pigmentosum.

In embodiments, the central nervous system injury is selected from: concussion, diffuse axonal injury, diffuse brain injury, focal brain injury, hemorrhage, seizure, stroke, traumatic brain injury, traumatic encephalopathy, and traumatic head injury.

In any of the preceding embodiments and aspects, wherein the administering is by intravenous injection or infusion; intra-arterial injection or infusion; intrathecal injection or infusion; intracerebral injection or infusion; injection or infusion into a ventricle, including a lateral ventricle; injection or infusion into the hippocampus; injection or infusion into the striatum; or injection or infusion into one or more of: the putamen, the caudate nucleus, the substantia nigra, the cortex, the third ventricle, the spinal cord, or the basal ganglia.

In any of the preceding embodiments and aspects, wherein the synthetic RNA encodes a neurotrophic agent.

In embodiments, the neurotrophic agent is a neurotrophic protein selected from nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF).

In embodiments, the neurotrophic protein is NGF and comprising the sequence of SEQ ID NO: 254, the neurotrophic protein is BDNF and comprising the sequence of SEQ ID NO: 561, the neurotrophic protein is NT-3 and comprising the sequence of SEQ ID NO: 255, the neurotrophic protein is NT-4 and comprising the sequence of SEQ ID NO: 256, the neurotrophic protein is CNTF and comprising the sequence of SEQ ID NO: 786, or the neurotrophic protein is GDNF family of ligands and comprising the sequence of SEQ ID NO: 787-793.

In embodiments, the synthetic RNA encodes a gene-editing protein that targets a safe harbor locus.

In embodiments, the synthetic RNA encodes a gene-editing protein that targets one or more of: AAVS1, CCR5, the human orthologue of the mouse Rosa26 locus.

In embodiments, the gene-editing protein inserts a functional copy of a gene into the subject's cells.

In embodiments, the inserted functional copy of a gene does not cause alterations of the subject's cell's genome which pose a risk to the subject.

In embodiments, the gene encodes a neurotrophic agent.

In any of the preceding embodiments and aspects, the gene encodes nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF).

In embodiments, the NGF comprises the sequence of SEQ ID NO: 254, the BDNF comprises the sequence of SEQ ID NO: 561, the NT-3 comprises the sequence of SEQ ID NO: 255, the NT-4 comprises the sequence of SEQ ID NO: 256, the CNTF comprises the sequence of SEQ ID NO: 786, or the GDNF family of ligands comprises the sequence of SEQ ID NO: 787-793.

In any of the preceding embodiments and aspects, the gene is inserted downstream of one or more of: a simple promoter, a constitutive promoter, a strong promoter, an endogenous promoter, tissue-specific promoter, cell type-specific promoter, or a drug-inducible promoter.

In any of the preceding embodiments and aspects, the method induces neurogenesis.

In any of the preceding embodiments and aspects, the synthetic RNA encodes an enzyme that cleaves a dysfunctional, abnormally folding, and/or a disease-causing protein.

In embodiments, the dysfunctional, abnormally folding, and/or disease-causing protein forms a glial scar.

In any of the preceding embodiments and aspects, the dysfunctional, abnormally folding, and/or disease-causing protein is amyloid, tau, alpha-synuclein, or huntingtin.

In any of the preceding embodiments and aspects, the administering is by intravenous injection or infusion; intra-arterial injection or infusion; intrathecal injection or infusion; intracerebral injection or infusion; injection or infusion into a ventricle, including a lateral ventricle; injection or infusion into the hippocampus; injection or infusion into the striatum; or injection or infusion into one or more of: the putamen, the caudate nucleus, the substantia nigra, the cortex, the third ventricle, the spinal cord, or the basal ganglia.

In any of the preceding embodiments and aspects, the administering is directly to a target tissue.

In embodiments, the administering is directly to a site of disease or injury.

In any of the preceding embodiments and aspects, the synthetic RNA is not encapsulated in a viral particle.

In any of the preceding embodiments and aspects, the synthetic RNA is formulated in a liposome or lipid particle.

In some aspects, the present invention relates to methods and compositions for treating a neurodegenerative disease or neural injury comprising delivering to a patient a synthetic RNA molecule. In various embodiments, the neurodegenerative disease is selected from Alzheimer's disease, Huntington's disease, Parkinson's disease, Lewy-body dementia, and dementia. In various embodiments, the neural injury is stroke. In various embodiments, the administering is by intrathecal injection or infusion, intracerebral injection, injection into a ventricle, including a lateral ventricle, injection into the hippocampus, injection into the striatum, or injection into one or more of the putamen, the caudate nucleus, the substantia nigra, the cortex, the third ventricle, the spinal cord, or the basal ganglia.

In various embodiments, the synthetic RNA molecule encodes a neurotrophic agent. In various embodiments, the neurotrophic agent is a neurotrophic protein. In various embodiments, the neurotrophic protein is selected from nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF). In various embodiments, the neurotrophic protein is BDNF.

In various embodiments, the synthetic RNA molecule encodes a gene-editing protein that targets a safe harbor locus, wherein the safe harbor locus is capable of accommodating the integration of new genetic material such that the integrated inserted genetic elements function predictably and/or do not cause alterations of the host genome which pose a risk to the host cell or organism. In various embodiments, the method comprises inserting a functional copy of a gene into the patient's cells. In various embodiments, the gene encodes a neurotrophic agent. In various embodiments, the gene encodes nerve growth factor (NGF), brain-derived neurotrophic factor (BDNF), neurotrophin-3 (NT-3), neurotrophin-4 (NT-4), the GDNF family of ligands, and ciliary neurotrophic factor (CNTF). In various embodiments, the gene encodes BDNF. In various embodiments, the gene is inserted downstream of one or more of a simple promoter, a constitutive promoter, a strong promoter, an endogenous promoter, or a drug-inducible promoter. In various embodiments, the drug-inducible promoter is inducible by tetracycline, e.g., a Tet-On or Tet-Off promoter. In various embodiments, the drug-inducible promoter is inducible by doxycycline.

In various embodiments, the present methods and compositions target the Huntingtin (HTT) gene or DNA upstream or downstream of the HTT gene, e.g., for treating Huntington's disease.

In various embodiments, the composition or method induces neurogenesis.

In some aspects, the invention provides methods for obtaining protein expression in a desired tissue. The methods comprising in vivo administering to an animal a composition comprising an mRNA such that the mRNA contacts a cell, is uptaken by the cell, and is expressed by the cell in the desired tissue. The in vivo administering may be intravenous, intra-arterial, or directly to the desired tissue. In embodiments, the mRNA encodes a protein that is absent in the cell or is insufficiently produced by the cell, such that the cell is in or contributes to a disease state.

In embodiments, the mRNA encodes a digestive enzyme that cleaves a dysfunctional protein, abnormally folding, and/or a disease-causing protein. In embodiments, the disease-causing protein forms a glial scar. In embodiments, the abnormally folding and/or disease-causing protein is tau, alpha-synuclein, or huntingtin. In embodiments, the mRNA encodes a gene-editing protein. In embodiments, the mRNA is not encapsulated in a viral particle and/or is formulated in a liposome.

### Treatment of Pain

In various embodiments, the present compositions are used to treat and reduce pain, e.g., post-surgical pain or chronic pain.

An aspect of the present invention is a method for treating and/or reducing pain comprising administering to a subject in need thereof an effective amount of a synthetic RNA encoding a gene-editing protein capable of creating a single-strand or double-strand break in a voltage-gated sodium channel type 1 (NaV1) gene, wherein the administering is directed to the central nervous system (CNS) or the peripheral nervous system (PNS).

In embodiments, the NaV1 is selected from NaV1.3, NaV1.7, NaV1.8, and NaV1.9.

In embodiments, the NaV1.3 is encoded by the SCN3A gene comprising the sequence of SEQ ID NO: 671, the NaV1.7 is encoded by the SC9N9A gene comprising the sequence of SEQ ID NO: 662, the NaV1.8 is encoded by the SCN10A gene comprising the sequences of SEQ ID NO: 672, and the NaV1.9 is encoded by the SCN11A gene comprising the sequences of SEQ ID NO: 673.

In any of the preceding embodiments and aspects, the administering is directed to neurons and/or glial cells of the CNS or PNS.

In any of the preceding embodiments and aspects, the administering is by intraganglionic injection, injection to the peripheral or central nerve roots, or injection in proximity to the dorsal root ganglion or nerve root.

In any of the preceding embodiments and aspects, the administering is directed into the parenchyma or the cerebrospinal spinal fluid of the central nervous system.

In any of the preceding embodiments and aspects, the synthetic RNA encoding a gene-editing protein is administered systemically and its penetrance to the CNS or PNS is increased by encapsulation in a viral or non-viral particle, by electrical stimulation, by acoustical stimulation, and/or by co-administration with a drug.

In any of the preceding embodiments and aspects, the RNA comprises or encodes a transport signal that directs the RNA or a protein product to a neuron's cell body or to a distal portion of the neuron.

In any of the preceding embodiments and aspects, the synthetic RNA encoding a gene-editing protein decreases expression of a wild-type or a mutant form of NaV 1.3, NaV 1.7, NaV 1.8, or NaV 1.9.

In any of the preceding embodiments and aspects, the synthetic RNA encoding a gene-editing protein increases expression of a wild-type or a mutant form of NaV 1.3, NaV 1.7, NaV 1.8, or NaV 1.9.

In any of the preceding embodiments and aspects, the synthetic RNA encoding a gene-editing protein increases enkephalins and/or glutamic acid decarboxylases in mesenchymal stem cells, thereby treating and/or reducing pain.

In any of the preceding embodiments and aspects, the methods further comprise administering electrical stimulation, a drug, and/or a cell therapy to increase efficacy.

In any of the preceding embodiments and aspects, the gene-editing protein is selected from a TALEN, a meganuclease, a nuclease, a zinc finger nuclease, a CRISPR-associated protein, CRISPR/Cas9, Cas9, xCas9, Cas12a (Cpf1), Cas13a, Cas14, CasX, CasY, a Class 1 Cas protein, a Class 2 Cas protein, and MAD7.

In any of the preceding embodiments and aspects, the gene-editing protein comprises: (a) a DNA-binding domain comprising a plurality of repeat sequences and at least one of the repeat sequences comprises the amino acid sequence: LTPvQWAIAwxyzGHGG (SEQ ID NO: 629), wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 630) or GGKQALETVQRLLPVLCQA (SEQ ID NO: 631); and (b) a nuclease domain comprising a catalytic domain of a nuclease.

In embodiments, the nuclease domain is capable of forming a dimer with another nuclease domain.

In any of the preceding embodiments and aspects, the nuclease domain comprises the catalytic domain of a protein comprising the amino acid sequence of SEQ ID NO: 632.

In any of the preceding embodiments and aspects, at least one of the repeat sequences comprising the amino acid sequence LTPvQWAIAwxyzGHGG (SEQ ID NO: 629) is between 36 and 39 amino acids long.

In any of the preceding embodiments and aspects, the pain is post-surgical and/or chronic pain.

In any of the preceding embodiments and aspects, the synthetic RNA comprises one or more non-canonical nucleotides.

In embodiments, the one or more non-canonical nucleotides avoids substantial cellular toxicity.

In any of the preceding embodiments and aspects, the non-canonical nucleotides have one or more substitutions at positions selected from the 2C, 4C, and 5C positions for a pyrimidine, or selected from the 6C, 7N and 8C positions for a purine.

In various embodiments, the present methods and compositions target any proteins associated with pain or treat or reduce pain, e.g., post-surgical pain and chronic pain. In various embodiments, the present invention targets the full-length and/or truncated forms of any of Voltage-gated Sodium channel type 1 (NaV1) proteins. As examples, NaV1.3 (encoded by SCN3A, SEQ ID NO: 671), NaV1.7 (encoded by SC9N9A, SEQ ID NO: 662), NaV1.8 (encoded by SCN10A, SEQ ID NO: 672), and NaV1.9 (encoded by SCN11A, SEQ ID NO: 673). In various embodiments, the present invention targets precursor forms and/or mature forms and/or isoforms of any of the NaV1 proteins disclosed herein.

### Non-Standard Untranslated Regions (UTRs)

It has now been discovered that the inclusion of guanosine nucleotides within the tail can enhance stability and/or translation efficiency of a synthetic RNA molecule. Some embodiments are therefore directed to a synthetic RNA molecule comprising a tail, wherein the tail comprises adenosine nucleotides and one or more other nucleotides.

Other embodiments are directed to a template that encodes a tail, wherein the tail comprises deoxyadenosine nucleotides and one or more other nucleotides. In one embodiment, the tail includes guanosine nucleotides. In another embodiment, the tail includes cytosine nucleotides. In a further embodiment, the tail includes uridine nucleotides. In a still further embodiment, the tail includes one or more chemically modified nucleotides and/or non-canonical nucleotides. In various embodiments, the other nucleotides are incorporated at regularly spaced intervals, or at random intervals, or in pairs or groups of adjacent nucleotides separated by one or more adenosine nucleotides. In one embodiment, the tail includes deoxyguanosine nucleotides. In another embodiment, the tail includes deoxycytosine nucleotides. In a further embodiment, the tail includes deoxyuridine nucleotides. In various embodiments, the other nucleotides are incorporated at regularly spaced intervals, or at random intervals, or in pairs or groups of adjacent nucleotides separated by one or more deoxyadenosine nucleotides.

An aspect of the present invention is a composition comprising a DNA template comprising: (a) a sequence encoding a protein, (b) a tail region comprising deoxyadenosine nucleotides and one or more other nucleotides, and (c) a restriction enzyme binding site.

In embodiments, the one or more other nucleotides comprises deoxyguanosine residues.

In embodiments, the tail region comprises about 1%, about 2%, about 5%, about 10%, about 15%, about 20%, about 25%, about 30%, about 35%, about 40%, about 45%, or about 50% deoxyguanosine residues.

In embodiments, the tail region comprises more than 50% deoxyguanosine residues.

In embodiments, the one or more other nucleotides comprises deoxycytidine residues.

In embodiments, the tail region comprises about 1%, about 2%, about 5%, about 10%, about 15%, about 20%, about 25%, about 30%, about 35%, about 40%, about 45%, or about 50% deoxycytidine residues.

In embodiments, the tail region comprises more than 50% deoxycytidine residues.

In embodiments, the one or more other nucleotides comprises deoxythymidine residues.

In embodiments, the tail region comprises about 1%, about 2%, about 5%, about 10%, about 15%, about 20%, about 25%, about 30%, about 35%, about 40%, about 45%, or about 50% deoxythymidine residues.

In embodiments, the tail region comprises more than 50% deoxythymidine residues.

In embodiments, the one or more other nucleotides comprise deoxyguanosine residues and deoxycytidine residues.

In any of the preceding embodiments and aspects, the tail region comprises about 99%, about 98%, about 95%, about 90%, about 85%, about 80%, about 75%, about 70%, about 65%, about 60%, about 55%, or about 50% deoxyadenosine residues.

In any of the preceding embodiments and aspects, the tail region comprises fewer than 50% deoxyadenosine residues.

In any of the preceding embodiments and aspects, the length of the tail region is between about 80 base pairs and about 120 base pairs, about 120 base pairs and about 160 base pairs, about 160 base pairs and about 200 base pairs, about 200 base pairs and about 240 base pairs, about 240 base pairs and about 280 base pairs, or about 280 base pairs and about 320 base pairs.

In any of the preceding embodiments and aspects, the length of the tail region is greater than 320 base pairs.

An aspect of the present invention is a composition comprising a synthetic RNA comprising: (a) a sequence encoding a protein, and (b) a tail region comprising adenosine nucleotides and one or more other nucleotides.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues.

In embodiments, the tail region comprises more than 50% guanosine residues.

In embodiments, the one or more other nucleotides comprises cytidine residues.

In embodiments, the tail region comprises about 1%, about 2%, about 5%, about 10%, about 15%, about 20%, about 25%, about 30%, about 35%, about 40%, about 45%, or about 50% cytidine residues.

In embodiments, the tail region comprises more than 50% cytidine residues.

In embodiments, the one or more other nucleotides comprises uridine residues.

In embodiments, the tail region comprises about 1%, about 2%, about 5%, about 10%, about 15%, about 20%, about 25%, about 30%, about 35%, about 40%, about 45%, or about 50% uridine residues.

In embodiments, the tail region comprises more than 50% uridine residues.

In embodiments, the one or more other nucleotides comprise guanosine residues and cytidine residues.

In any of the preceding embodiments and aspects, the tail region comprises about 99%, about 98%, about 95%, about 90%, about 85%, about 80%, about 75%, about 70%, about 65%, about 60%, about 55%, or about 50% adenosine residues.

In any of the preceding embodiments and aspects, the tail region comprises fewer than 50% adenosine residues.

In any of the preceding embodiments and aspects, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In any of the preceding embodiments and aspects, the length of the tail region is greater than 320 nucleotides.

An aspect of the present invention is a composition comprising a synthetic RNA comprising a 3-untranslated region sequence having at least 90% homology to the 3-untranslated region of a gene selected from: APOBEC3H, CD52, DMC1, EIF3E, GPR160, and RPS24.

In any of the preceding embodiments and aspects, the synthetic RNA further comprises one or more non-canonical nucleotides.

In embodiments, the non-canonical nucleotides have one or more substitutions at positions selected from the 2C, 4C, and 5C positions for a pyrimidine, or selected from the 6C, 7N and 8C positions for a purine.

In any of the preceding embodiments and aspects, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In any of the preceding embodiments and aspects, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In any of the preceding embodiments and aspects, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In any of the preceding embodiments and aspects, at least about 10% of guanine residues are non-canonical nucleotides, and the non-canonical nucleotide is optionally 7-deazaguanosine.

In any of the preceding embodiments and aspects, the synthetic RNA comprises no more than about 50% 7-deazaguanosine in place of guanosine residues.

In any of the preceding embodiments and aspects, the synthetic RNA does not comprise non-canonical nucleotides in place of adenosine residues.

In any of the preceding embodiments and aspects, the synthetic RNA comprises 5-methoxyuridine.

Certain embodiments are directed to a tail comprising no adenosine or deoxyadenosine nucleotides. In one embodiment, the tail is a poly(G) tail.

In certain embodiments, the tail comprises repeated sequences, wherein each repeated sequence comprises a poly(A) sequence followed by a nucleotide other than adenosine or deoxyadenosine. In some embodiments, the poly(A) tail comprises (A)₁₄G (SEQ ID NO: 710), or (A)₉G (SEQ ID NO: 711), or (A)₄G (SEQ ID NO: 712). In some embodiments, the tail comprises 10 repeats of (A)₁₄G (SEQ ID NO: 710), or 15 repeats of (A)₉G (SEQ ID NO: 711), or 30 repeats of (A)₄G (SEQ ID NO: 712). In other embodiments, the tail comprises (A)₁₃GG (SEQ ID NO: 713), or (A)₁₂GGG (SEQ ID NO: 714), or (A)₈GG (SEQ ID NO: 715), or (A)₇GGG (SEQ ID NO: 716), or (A)₃GG (SEQ ID NO: 717). In other embodiments, the tail comprises 10 repeats of (A)₁₃GG (SEQ ID NO: 713), or 10 repeats of (A)₁₂GGG (SEQ ID NO: 714), or 15 repeats of (A)₈GG (SEQ ID NO: 715), or 15 repeats of (A)₇GGG (SEQ ID NO: 716), or 30 repeats of (A)₃GG (SEQ ID NO: 717).

In other embodiments, the tail contains cytidine or uridine residues, or modified versions thereof, or non-canonical nucleotides.

In some embodiments, the tail comprises about 1%, or about 2%, or about 3%, or about 5%, or about 10%, or about 15%, or about 20%, or about 25%, or about 30%, or about 40%, or about 50%, or about 60%, or about 70%, or about 80%, or about 90%, or greater than about 90% nucleotides other than adenosine or deoxyadenosine, and/or modified and/or non-canonical nucleotides.

In some embodiments, the tail includes non-adenosine nucleotides or non-canonical nucleotides that are incorporated in an enzymatic reaction or a non-enzymatic reaction. In one embodiment, the reaction does not include a template. In another embodiment the enzyme is a non-canonical poly(A) polymerase. In a further embodiment, the enzyme is selected from TENT4A (PAPD7), TENT4B (PAPD5), TENT5A, TENT5B, TENT5C, and TENT5D. In other embodiments, the template is an RNA template, PNA template, LNA template, or a hybrid RNA/DNA template.

Other embodiments are directed to a method for analyzing the length of a tail, a template, or a synthetic RNA molecule comprising isolating isolate the tail or template, or some portion of the RNA or template inclusive of the tail, using restriction digestion. In various embodiments, the analysis is performed by gel electrophoresis, or by capillary electrophoresis, or by high-performance liquid chromatography (HPLC), or by mass spectrometry, or by size-exclusion chromatography (SEC), or by sequencing reactions, or by polymerase chain reactions (PCR), or by other biological, biochemical or biophysical methods.

In certain situations, bacteria or other organisms are used to amplify a plasmid or polynucleotide sequence comprising a template, the sequence of a synthetic RNA molecule, or both. It has now been discovered that the repetitive or homopolymeric nature of the tail may destabilize the plasmid or polynucleotide sequence during amplification and culture growth.

Some embodiments are therefore directed to a method of amplifying a template using modified or engineered bacteria expressing mutations that enhance the stability of repetitive or homopolymeric plasmids or polynucleotides.

In one embodiment, the bacteria are Stable Competent E. coli(New England Biolabs). In another embodiment, the bacteria are telN-expressing E. coli, and the telN-expressing E. coli are transformed with a linear plasmid comprising the template. Still other embodiments are directed to methods for amplifying a template in yeast or other eukaryotes.

It has now been discovered that certain 3′-UTR sequences can modulate stability and translation efficiency of synthetic RNA molecules. Certain embodiments are therefore directed to a synthetic RNA molecule comprising a 3′-UTR sequence that confers stability. Other embodiments are directed to a 3′-UTR sequence that confers high efficiency translation. In one embodiment, the 3′-UTR is selected from APOBEC3H, CD52, DMC1, EIF3E, GPR160, and RPS24. In another embodiment, the synthetic RNA molecule encodes a protein of interest.

In one aspect, the synthetic RNA molecule has an effective dose lower than a synthetic RNA molecule having a 3′-UTR comprising an HBB sequence.

It has now been discovered that in certain situations, a synthetic RNA molecule having a short half-life can be beneficial, for example, in the case of expressing a gene-editing protein, to minimize off-target effects. Certain embodiments are therefore directed to a synthetic RNA molecule with a half-life shorter than about 24 hours, or about 18 hours, or about 12 hours, or about 9 hours, or about 6 hours, or about 3 hours, or about 2, or about 1 hour.

It has now been discovered that a synthetic RNA molecule comprising 3′-UTRs containing one or more microRNA binding sites can enable cell-type specific expression. Certain embodiments are therefore directed to a synthetic RNA molecule comprising a 3′-UTR containing one or microRNA binding sites. In one embodiment, the synthetic RNA molecule is preferentially expressed in stem cells, or erythrocytes, or leukocytes, or platelets, or neurons, or neuroglial cells, or myocytes, or chondrocytes, or osteoclasts, or osteoblasts, or osteocytes, or lining cells, or keratinocytes, or melanocytes, or Langerhans cells, or fibroblasts, or merkel cells, endothelial cells, or epithelial cells, or adipocytes, or gametes. In one embodiment, the synthetic RNA molecule is preferentially expressed in epithelial tissue, or connective tissue, or muscle tissue, or nervous tissue.

It has now been discovered that a synthetic RNA molecule comprising a 3′-UTR containing one or more transfection reagent binding sites can enable high efficiency transfection of cells.

It has now been discovered that a synthetic RNA molecule comprising a 3′-UTR containing one or more predefined sequence elements can enable controlled degradation, for example, by a sequence element that binds to a molecule that is administered to a patient to reduce the in vivo half-life of the synthetic RNA molecule.

Certain embodiments are directed to methods of making nucleic acid drugs, including RNA comprising one or more non-canonical nucleotides. Such methods yield substantially stable RNA.

In various embodiments, the present methods and compositions find use in methods of altering, modifying and/or changing a tissue (e.g. cosmetically).

### Gene Editing for Treating Various Diseases/Indications

Several naturally occurring proteins contain DNA-binding domains that can recognize specific DNA sequences, for example, zinc fingers (ZFs) and transcription activator-like effectors (TALEs). Fusion proteins containing one or more of these DNA-binding domains and the cleavage domain of FokI endonuclease can be used to create a double-strand break in a desired region of DNA in a cell (see, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). Other gene-editing proteins include clustered regularly interspaced short palindromic repeat (CRISPR)-associated proteins.

However, current methods for gene editing cells are inefficient and carry a risk of uncontrolled mutagenesis, making them undesirable for research, therapeutic or cosmetic use. Methods for DNA-free gene editing of somatic cells have not been previously explored, nor have methods for simultaneous or sequential gene editing and reprogramming of somatic cells. In addition, methods for directly gene editing cells in patients (i.e., in vivo) have not been previously explored, and the development of such methods has been limited by a lack of acceptable targets, inefficient delivery, inefficient expression of the gene-editing protein/proteins, inefficient gene editing by the expressed gene-editing protein/proteins, due in part to poor binding of DNA-binding domains, excessive off-target effects, due in part to non-directed dimerization of the FokI cleavage domain and poor specificity of DNA-binding domains, and other factors. Finally, the use of gene editing in anti-bacterial, anti-viral, and anti-cancer treatments has not been previously explored.

In various embodiments, the present methods and compositions include using a nucleic acid drug, including a synthetic RNA, in the diagnosing, treating, preventing or ameliorating of a disease, disorder and/or condition described herein. In various embodiments, the present methods and compositions include using a nucleic acid drug, including a synthetic RNA, in the altering, modifying and/or changing of a tissue (e.g. cosmetically).

Generally speaking, in various embodiments, a synthetic RNA as described herein is administered to a human at specific doses described herein and the synthetic RNA comprises a sequence, sometimes referred to as a target sequence that encodes a protein of interest, which may be a therapeutic protein.

Synthetic RNA comprising only canonical nucleotides can bind to pattern recognition receptors, can be recognized as a pathogen-associated molecular pattern, and can trigger a potent immune response in cells, which can result in translation block, the secretion of inflammatory cytokines, and cell death. It has now been discovered that synthetic RNA comprising certain non-canonical nucleotides can evade detection by the innate immune system, and can be translated at high efficiency into protein, including in humans. It has been further discovered that synthetic RNA comprising at least one of the non-canonical nucleotides described herein, including, for example, a member of the group: 5-methylcytidine, 5-hydroxycytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine can evade detection by the innate immune system, and can be translated at high efficiency into protein, including in humans. Certain embodiments are therefore directed to a method for inducing a cell to express a protein of interest comprising contacting a cell with synthetic RNA. Other embodiments are directed to a method for transfecting a cell with synthetic RNA comprising contacting a cell with a solution comprising one or more synthetic RNA molecules. Still other embodiments are directed to a method for treating a patient comprising administering to the patient synthetic RNA. In one embodiment, the synthetic RNA comprises at least one of the non-canonical nucleotides described herein, including, for example, a member of the group: 5-methylcytidine, 5-hydroxycytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine. In another embodiment, the synthetic RNA encodes a protein of interest. Exemplary RNAs may contain combinations and levels of non-canonical and non-canonical nucleotides as described elsewhere herein, including with respect to the expression of any protein of interest described herein. In yet another embodiment, the method results in the expression of the protein of interest. In a further embodiment, the method results in the expression of the protein of interest in the patient's skin.

Other embodiments are directed to a method for delivering a nucleic acid to a cell in vivo. Still other embodiments are directed to a method for inducing a cell in vivo to express a protein of interest. Still other embodiments are directed to a method for treating a patient. In one embodiment, the method comprises disrupting the stratum corneum. In another embodiment, the method comprises contacting a cell with a nucleic acid. In yet another embodiment, the method results in the cell internalizing the nucleic acid. In a further embodiment, the method results in the cell expressing the protein of interest. In a still further embodiment, the method results in the expression of the protein of interest in the patient. In a still further embodiment, the method results in the amelioration of one or more of the patient's symptoms. In a still further embodiment, the patient is in need of the protein of interest. In a still further embodiment, the patient is deficient in the protein of interest.

Still other embodiments are directed to a method for treating a patient comprising delivering to a patient a composition. In another embodiment, the composition comprises one or more nucleic acid molecules. In yet another embodiment, at least one of the one or more nucleic acid molecules encodes a protein of interest. In some embodiments, the nucleic acid is synthetic RNA. In other embodiments, the method results in the amelioration of one or more of the patient's symptoms. Other embodiments are directed to a method for treating an indication by delivering to a cell or a patient a nucleic acid encoding a protein or a peptide. Still other embodiments are directed to a composition comprising a nucleic acid encoding a protein or a peptide. Indications that can be treated using the methods and compositions of the present invention and proteins and peptides that can be encoded by compositions of the present invention are set forth in Table 3A, Table 3B, and/or Table 3C, and are given by way of example, and not by way of limitation. In one embodiment, the indication is selected from Table 3A, Table 3B, and/or Table 3C. In another embodiment the protein or peptide is selected from Table 3A, Table 3B, and/or Table 3C. In yet another embodiment, the indication and the protein or peptide are selected from the same row of Table 3A, Table 3B, and/or Table 3C. In another embodiment, the protein is a gene-editing protein. In yet another embodiment, the gene-editing protein targets a gene that is at least partly responsible for a disease phenotype. In yet another embodiment, the gene-editing protein targets a gene that encodes a protein selected from Table 3A, Table 3B, and/or Table 3C. In still another embodiment, the gene-editing protein corrects or eliminates, either alone or in combination with one or more other molecules or gene-editing proteins, a mutation that is at least partly responsible for a disease phenotype.

In various embodiments, the present invention contemplates the targeting of the precursor forms and/or mature forms and/or isoforms and/or mutants of any of the proteins disclosed in Table 3A, Table 3B, and/or Table 3C and such proteins. In some embodiments, any of the precursor forms and/or mature forms and/or isoforms and/or mutants have enhanced secretion relative to the corresponding wild type proteins. In some embodiments, any of the precursor forms and/or mature forms and/or isoforms and/or mutants have altered half-lives (e.g. serum, plasma, intracellular)—for instance, longer or shorter half-lives. In some embodiments, this is relative to wild type.

TABLE 3A
Illustrative Indications

| Illustrative | |
|---|---|
| Indication | Illustrative Protein/Peptide |
| Acne | Retinol Dehydrogenase 10 |
| Aging | Elastin, sp\|P15502\|ELN_HUMAN Elastin, (isoform 3), |
| | (SEQ ID NO: 486) |
| Aging | Collagen Type I, P02452\|CO1A1_HUMAN Collagen |
| | alpha-1(I) chain, (SEQ ID NO: 487); P08123\|CO1A2_ |
| | HUMAN Collagen alpha-2(I) chain, (SEQ ID NO: 488) |
| Aging | Collagen Type III, P02461\|CO3A1_HUMAN Collagen |
| | alpha-1(III) chain, (isoform 1), (SEQ ID NO: 489) |
| Aging | Collagen Type VII, Q02388\|CO7A1_HUMAN |
| | Collagen alpha-1(VII) chain, (SEQ ID NO: 490) |
| Aging | Hyaluronan Synthase |
| Aging | Telomerase Reverse Transcriptase |
| Albinism | Tyrosinase, P14679\|TYRO_HUMAN Tyrosinase, |
| | (isoform 1), (SEQ ID NO: 491) |
| Alport | Collagen Type IV; P02462\|CO4A1_HUMAN Collagen |
| Syndrome | alpha-1(IV) chain, (isoform 1), (SEQ ID NO: 492); |
| | P08572\|CO4A2_HUMAN Collagen alpha-2(IV) chain, |
| | (SEQ ID NO: 493); Q01955\|CO4A3_HUMAN Collagen |
| | alpha-3(IV) chain, (isoform 1), (SEQ ID NO: 494); |
| | P53420\|CO4A4_HUMAN Collagen alpha-4(IV) chain, |
| | (SEQ ID NO: 495); P29400\|CO4A5_HUMAN Collagen |
| | alpha-5(IV) chain, (isoform 1), (SEQ ID NO: 496); |
| | Q14031\|CO4A6_HUMAN Collagen alpha-6(IV), |
| | (isoform A), (SEQ ID NO: 497) |
| Anemia | Erythropoietin |
| Atopic | Filaggrin |
| Dermatitis | |
| Cutis Laxa | Elastin, sp\|P15502\|ELN_HUMAN Elastin, (isoform 3), |
| | (SEQ ID NO: 486) |
| Dry Skin | Filaggrin |
| Dystrophic | Collagen Type VII; Q02388\|CO7A1_HUMAN Collagen |
| Epidermo- | alpha-1(VII) chain, (SEQ ID NO: 498) |
| lysis | |
| Bullosa | |
| Ehlers- | Collagen Type V; P20908\|CO5A1_HUMAN Collagen |
| Danlos | alpha-1(V) chain, (SEQ ID NO: 499); P05997\|CO5A2_ |
| Syndrome | HUMAN Collagen alpha-2(V) chain, (SEQ ID NO: 500); |
| | P25940\|CO5A3_HUMAN Collagen alpha-3(V) chain, |
| | (SEQ ID NO: 501) |
| Ehlers- | Collagen Type 1, P02452\|CO1A1_HUMAN Collagen |
| Danlos | alpha-1(I) chain, (SEQ ID NO: 487); P08123\|CO1A2_ |
| Syndrome | HUMAN Collagen alpha-2(I) chain, (SEQ ID NO: 488) |
| Epidermo- | ADAM17, P78536\|ADA17_HUMAN Disintegrin and |
| lysis | metalloproteinase domain-containing protein 17, |
| bullosa, | (isoform A), (SEQ ID NO: 502) |
| lethal | |
| acantho- | |
| lytic | |
| Epidermo- | Collagen Type III, P02461\|CO3A1_HUMAN Collagen |
| lysis | alpha-1(III) chain, (isoform 1), (SEQ ID NO: 489) |
| bullosa, | |
| type IV | |
| Erythro- | Ferrochelatase, P22830\|HEMH_HUMAN Ferrochelatase, |
| poietic | mitochondrial, (isoform 1), (SEQ ID NO: 503) |
| Proto- | |
| porphyria | |
| Eczema | Filaggrin |
| Excess Fat | Thermogenin, P25874\|UCP1_HUMAN Mitochondrial |
| | brown fat uncoupling protein 1, (SEQ ID NO: 504) |
| Excess Fat | Lipase; Lipoprotein lipase, P06858\|LIPL_HUMAN |
| | Lipoprotein lipase, (SEQ ID NO: 516); Hepatic lipase, |
| | P11150\|LIPC_HUMAN Hepatic triacylglycerol lipase, |
| | (SEQ ID NO: 517); Pancreatic lipase, P16233\|LIPP_ |
| | HUMAN Pancreatic triacylglycerol lipase, (SEQ ID NO: |
| | 518); Endothelial lipase, (isoform 1), Q9Y5X9\|LIPE_ |
| | HUMAN Endothelial lipase, (SEQ ID NO: 519); |
| | Lysosomal lipase, P38571\|LICH_HUMAN Lysosomal |
| | acid lipase/cholesteryl ester hydrolase, (isoform 1), |
| | (SEQ ID NO: 520); Hormone sensitive lipase, |
| | Q05469\|LIPS_HUMAN Hormone-sensitive lipas, |
| | (isoform 1), (SEQ ID NO: 521); Gastric lipase, |
| | P07098\|LIPG_HUMAN Gastrict riacylglycerol lipase, |
| | (isoform 1), (SEQ ID NO: 522); Pancreatic Lipase- |
| | Related Protein 1), P54315\|LIPR1_HUMAN Inactive |
| | pancreatic lipase-related protein 1, (isoform 1), (SEQ |
| | ID NO: 523); Pancreatic Lipase-Related Protein 2, |
| | P54317\|LIPR2_HUMAN Pancreatic lipase-related protein |
| | 2, (SEQ ID NO: 524); Carboxyl Ester Lipase, P19835\| |
| | CEL_HUMAN Bile salt-activated lipase, (isoform long), |
| | (SEQ ID NO: 525) |
| Hypo- | ADAM17, P78536\|ADA17_HUMAN Disintegrin and |
| trichosis | metalloproteinase domain-containing protein 17, (isoform |
| | A), (SEQ ID NO: 502) |
| Ichthyosis | Filaggrin |
| Vulgaris | |
| Infections | Genetic Antibiotics (e.g. Anti-Sigma Factors) |
| Inflam- | Desmoglein 2, Q14126\|DSG2_HUMAN Desmoglein-2, |
| matory | (SEQ ID NO: 505) |
| and | |
| Bullous | |
| Skin | |
| Bowel | |
| Syndrome | |
| Keratosis | Retinol Dehydrogenase 10 |
| Pilaris | |
| Oily Skin | Retinol Dehydrogenase 10 |
| Osteo- | Hyaluronan Synthase |
| arthritis | |
| Pemphigus | Plakophilin-1, Q13835\|PKP1_HUMAN Plakophilin-1, |
| Vulgaris | (isoform 2), (SEQ ID NO: 506) |
| Pseudo- | Elastin, sp\|P15502\|ELN_HUMAN Elastin, (isoform 3), |
| xanthoma | (SEQ ID NO: 486) |
| elasticum | |
| Psoriasis | Retinol Dehydrogenase 10 |
| Scar | Tyrosinase, P14679\|TYRO_HUMAN Tyrosinase, |
| Treatment | (isoform 1), (SEQ ID NO: 491) |
| Scarring | Elastin, sp\|P15502\|ELN_HUMAN Elastin, (isoform 3), |
| | (SEQ ID NO: 486) |
| Scarring | Collagen Type 1, P02452\|CO1A1_HUMAN Collagen |
| | alpha-1(I) chain, (SEQ ID NO: 487); P08123\|CO1A2_ |
| | HUMAN Collagen alpha-2(I) chain, (SEQ ID NO: 488) |
| Scarring | Collagen Type III, P02461\|CO3A1_HUMAN Collagen |
| | alpha-1(III) chain, (isoform 1), (SEQ ID NO: 489) |
| Skin | Interferon; Interferon, Alpha 1, P01562\|IFNA1_HUMAN |
| Cancer | Interferon alpha-1/13, (SEQ ID NO: 530); Interferon, |
| | Alpha 2, P01563\|IFNA2_HUMAN Interferon alpha-2, |
| | (SEQ ID NO: 531); Interferon, Alpha 4, P05014\|IFNA4_ |
| | HUMAN Interferon alpha-4, (SEQ ID NO: 532); |
| | Interferon, Alpha 5, P01569\|IFNA5_HUMAN Interferon |
| | alpha-5, (SEQ ID NO: 533), Interferon, Alpha 6, |
| | P05013\|IFNA6_HUMAN Interferon alpha-6, (SEQ ID NO: |
| | 534); Interferon, Alpha 7, P01567\|IFNA7_HUMAN |
| | Interferon alpha-7, (SEQ ID NO: 535); Interferon, Alpha 8, |
| | P32881\|IFNA8_HUMAN Interferon alpha-8, (SEQ ID NO: |
| | 536); Interferon, Alpha 10, P01566\|IFN10_HUMAN |
| | Interferon alpha-10, (SEQ ID NO: 537); Interferon, Alpha |
| | 14, P01570\|IFN14_HUMAN Interferon alpha-14 OS, (SEQ |
| | ID NO: 538); Interferon, Alpha 16, P05015\|IFN16_ |
| | HUMAN Interferon alpha-16, (SEQ ID NO: 539); |
| | Interferon, Alpha 17, P01571\|IFN17_HUMAN Interferon |
| | alpha-17, (SEQ ID NO: 540); Interferon, Alpha 21, |
| | P01568\|IFN21_HUMAN Interferon alpha-21, (SEQ ID |
| | NO: 541); Interferon, Gamma, P01579\|IFNG_HUMAN |
| | Interferon gamma, (SEQ ID NO: 542); Interferon, Beta, |
| | P01574\|IFNB_HUMAN Interferon beta, (SEQ ID NO: |
| | 543); Interferon, Kappa, Q9P0W0\|IFNK_HUMAN |
| | Interferon kappa, (SEQ ID NO: 544); Interferon, |
| | Epsilon, Q86WN2\|IFNE_HUMAN Interferon epsilon, |
| | (SEQ ID NO: 545) |
| Striate | ADAM17, P78536\|ADA17_HUMAN Disintegrin and |
| Palmo- | metalloproteinase domain-containing protein 17, (isoform |
| plantar | A), (SEQ ID NO: 502) |
| Kerato- | |
| derma | |
| Tanning | Tyrosinase, P14679\|TYRO_HUMAN Tyrosinase, (isoform |
| | 1), (SEQ ID NO: 491) |
| Vitiligo | Melanocyte-Stimulating Hormone; Alpha-MSH, P01189\| |
| | 138-150, (SEQ ID NO: 526); , Beta-MSH, P01189\|217-234, |
| | (SEQ ID NO: 527); Gamma-MSH, P01189\|77-87, (SEQ ID |
| | NO: 528); Proopiomelanocortin, P01189\|COLI_HUMAN |
| | Pro-opiomelanocortin, (SEQ ID NO: 529) |
| Vitiligo | Tyrosinase, P14679\|TYRO_HUMAN Tyrosinase, (isoform |
| | 1), (SEQ ID NO: 491) |
| Warts | Interferon; Interferon, Alpha 1, P01562\|IFNA1_HUMAN |
| | Interferon alpha-1/13, (SEQ ID NO: 530); Interferon, Alpha |
| | 2, P01563\|IFNA2_HUMAN Interferon alpha-2, (SEQ ID |
| | NO: 531); Interferon, Alpha 4, P05014\|IFNA4_HUMAN |
| | Interferon alpha-4, (SEQ ID NO: 532); Interferon, Alpha 5, |
| | P01569\|IFNA5_HUMAN Interferon alpha-5, (SEQ ID NO: |
| | 533), Interferon, Alpha 6, P05013\|IFNA6_HUMAN |
| | Interferon alpha-6, (SEQ ID NO: 534); Interferon, Alpha |
| | 7, P01567\|IFNA7_HUMAN Interferon alpha-7, (SEQ ID |
| | NO: 535); Interferon, Alpha 8, P32881\|IFNA8_HUMAN |
| | Interferon alpha-8, (SEQ ID NO: 536); Interferon, Alpha |
| | 10, P01566\|IFN10_HUMAN Interferon alpha-10, (SEQ |
| | ID NO: 537); Interferon, Alpha 14, P01570\|IFN14_ |
| | HUMAN Interferon alpha-14 OS, (SEQ ID NO: 538); |
| | Interferon, Alpha 16, P05015\|IFN16_HUMAN Interferon |
| | alpha-16, (SEQ ID NO: 539); Interferon, Alpha 17, P01571\| |
| | IFN17_HUMAN Interferon alpha-17, (SEQ ID NO: 540); |
| | Interferon, Alpha 21, P01568\|IFN21_HUMAN Interferon |
| | alpha-21, (SEQ ID NO: 541); Interferon, Gamma, |
| | P01579\|IFNG_HUMAN Interferon gamma, |
| | (SEQ ID NO: 542); Interferon, Beta, P01574\|IFNB_ |
| | HUMAN Interferon beta, (SEQ ID NO: 543); |
| | Interferon, Kappa, Q9P0W0\|IFNK_HUMAN Interferon |
| | kappa, (SEQ ID NO: 544); Interferon, Epsilon, |
| | Q86WN2\|IFNE_HUMAN Interferon epsilon, |
| | (SEQ ID NO: 545) |
| Wound | Elastin, sp\|P15502\|ELN_HUMAN Elastin, (isoform 3), |
| Healing | (SEQ ID NO: 486) |
| Wound | Collagen Type 1, P02452\|CO1A1_HUMAN Collagen |
| Healing | alpha-1(I) chain, (SEQ ID NO: 487); P08123\|CO1A2_ |
| | HUMAN Collagen alpha-2(I) chain, (SEQ ID NO: 488) |
| Wound | Collagen Type III, P02461\|CO3A1_HUMAN Collagen |
| Healing | alpha-1(III) chain, (isoform 1), (SEQ ID NO: 489) |
| Xeroderma | DNA Polymerase Eta, Q9Y253\|POLH_HUMAN DNA |
| Pigment- | polymerase eta, (isoform 1), (SEQ ID NO: 507) |
| osum | |

TABLE 3B
Illustrative Proteins and Illustrative Peptides
Protein/Peptide Illustrative Identifier Reference

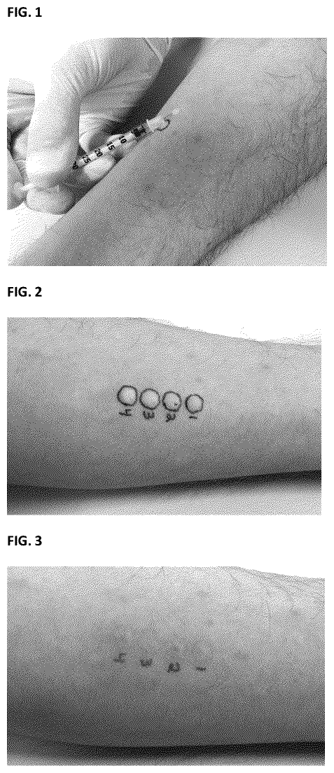

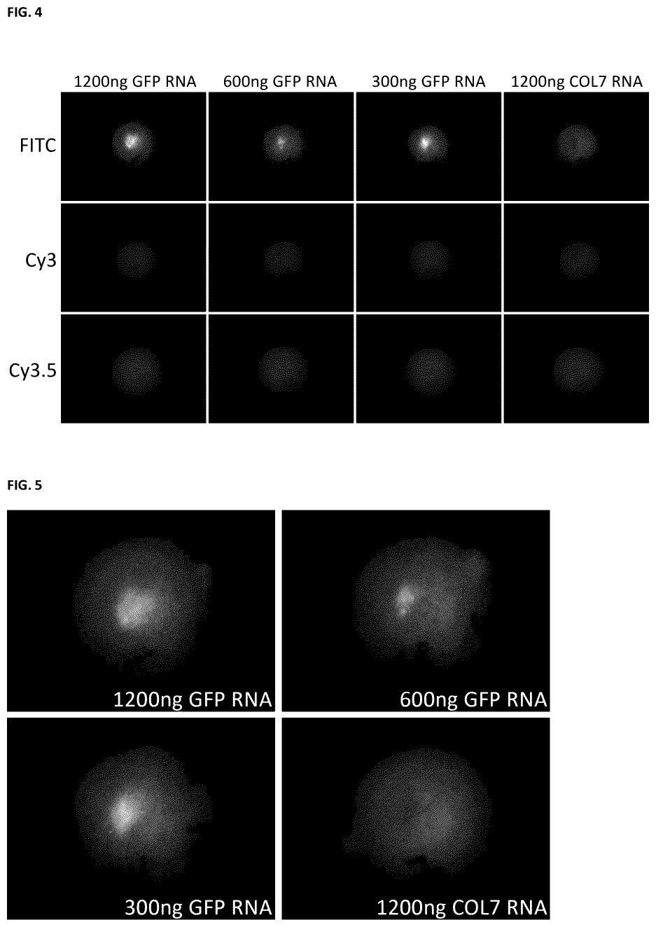

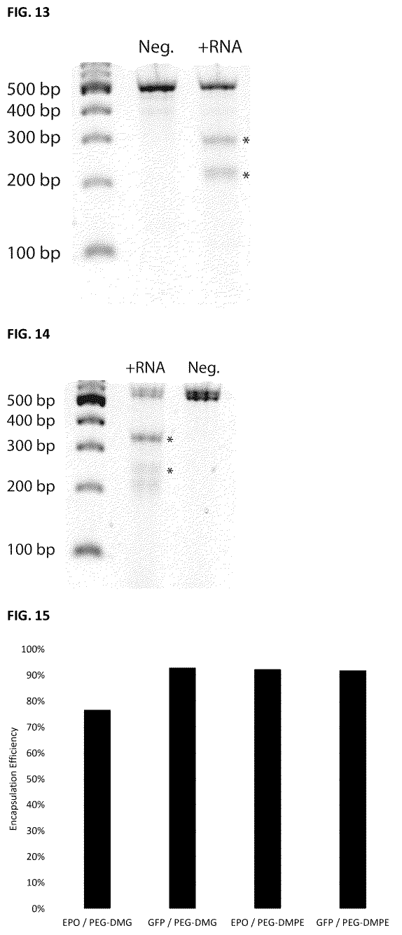

Transthyretin (TTR), (SEQ ID NOs: 637 and 638), Gene ID: 7276
Endothelial Cell Specific Molecule 1, (SEQ ID NO: 784 and 785), Gene ID: 11082
Parathyroid hormone, P012701PTHY_HUMAN Parathyroid hormone, (SEQ ID NO: 508)
BMP-1 GeneSeq Accession P80618 WO8800205, P13497/BMP1_HUMAN Bone morphogenetic protein 1, (isoform BMP1-3), (SEQ ID NO: 169)
P13497-2|BMP1_HUMAN Isoform BMP1-1 of Bone morphogenetic protein 1, (isoform BMP1-1), (SEQ ID NO: 509)
P13497-3|BMP1_HUMAN Isoform BMP1-4 of Bone morphogenetic protein 1, (isoform BMP1-4), (SEQ ID NO: 510)
P13497-4|BMP1_HUMAN Isoform BMP1-5 of Bone morphogenetic protein 1, (isoform BMP1-5), (SEQ ID NO: 511)
P13497-5|BMP1_HUMAN Isoform BMP1-6 of Bone morphogenetic protein 1, (isoform BMP1-6), (SEQ ID NO: 512)
P13497-6|BMP1_HUMAN Isoform BMP1-7 of Bone morphogenetic protein 1, (isoform BMP1-7), (SEQ ID NO: 513)
BMP-2 GeneSeq Accession P80619 WO8800205, P12643/BMP2_HUMAN Bone morphogenetic protein 2, (SEQ ID NO: 170)
BMP-3, P12645|BMP3_HUMAN Bone morphogenetic protein 3, (SEQ ID NO: 514)
BMP-2B GeneSeq Accession W24850 U.S. Pat. No. 5,631,142, P12644/BMP4_HUMAN Bone morphogenetic protein 4, (SEQ ID NO: 171)
BMP-4 GeneSeq Accession B02796 WO0020591, P12644/BMP4_HUMAN Bone morphogenetic protein 4, (SEQ ID NO: 172)
BMP-5 GeneSeq Accession B02797 WO0020591, P22003/BMP5_HUMAN Bone morphogenetic protein 5, (isoform 1), (SEQ ID NO: 173)
P22003-2|BMP5_HUMAN Isoform 2 of Bone morphogenetic protein 5, (isoform 2), (SEQ ID NO: 515)
BMP-6 GeneSeq Accession R32904 U.S. Pat. No. 5,187,076, P22004/BMP6_HUMAN Bone morphogenetic protein 6, (SEQ ID NO: 174)
Osteogenic Protein-1; OP-1; BMP-7 GeneSeq Accession W34783 WO973462, P18075/BMP7_HUMAN Bone morphogenetic protein 7,
(SEQ ID NO: 175)
BMP7 Variant A, (SEQ ID NO: 579)
BMP7 Variant B, (SEQ ID NO: 580)
BMP7 Variant C, (SEQ ID NO: 581)
Osteogenic Protein-2 GeneSeq Accession R57973 WO9406399, P34820/BMP8B_HUMAN Bone morphogenetic protein 8B, (SEQ ID NO: 176)
GDF-1 GeneSeq Accession R60961 WO9406449, P27539/GDF1_HUMAN Embryonic growth/differentiation factor 1, (SEQ ID NO: 177)
BMP-9 GeneSeq Accession R86903 WO9533830, Q9UK05/GDF2_HUMAN Growth/differentiation factor 2, (SEQ ID NO: 178)
BMP-10 GeneSeq Accession R66202 WO9426893, Q95393/BMP10_HUMAN Bone morphogenetic protein 10, (SEQ ID NO: 179)
BMP-12 GeneSeq Accession R78734 WO9516035, Q7Z4P5/GDF7_HUMAN Growth/differentiation factor 7, (SEQ ID NO: 180)
BMP-15 GeneSeq Accession W11261 WO9636710, O95972/BMP15_HUMAN Bone morphogenetic protein 15, (SEQ ID NO: 181)
BMP-17 GeneSeq Accession Y17870 WO9929718, SEQ ID NO: 2 from U.S. Pat. No. 7,151,086, (SEQ ID NO: 182)
BMP-18 GeneSeq Accession Y17871 WO9929718, SEQ ID NO: 4 from U.S. Pat. No. 7,151,086, (SEQ ID NO: 183)
Inhibin alpha GeneSeq Accession B02806 WO0020591, P05111/INHA_HUMAN Inhibin alpha chain, (SEQ ID NO: 184)
Inhibin beta GeneSeq Accession H02808 WO0020591, P08476/INHBA_HUMAN Inhibin beta A chain, (SEQ ID NO: 185)
P09529/INHBB_HUMAN Inhibin beta B chain, (SEQ ID NO: 186)
Cerberus Protein GeneSeq Accession W86032 WO9849296, O95813/CER1_HUMAN Cerberus, (SEQ ID NO: 187)
Soluble BMP Receptor Kinase Protein-3 GeneSeq Accession R95227 WO9614579, Q13873/BMPR2_HUMAN Bone morphogenetic
protein receptor type-2, (SEQ ID NO: 188)
BMP Processing Enzyme Furin GeneSeq Accession W36099 WO9741250, P09958/FURIN_HUMAN Furin, (SEQ ID NO: 189)
TGF-beta 1 GeneSeq Accession R29657 WO9216228, P01137/TGFB1_HUMAN Transforming growth factor beta-1, (SEQ ID NO: 190)
TGF-beta 2 GeneSeq Accession R39659 EP542679, P61812/TGFB2_HUMAN Transforming growth factor beta-2, (SEQ ID NO: 191)
ZTGF-beta 9 GeneSeq Accession Y70654 WO0015798, SEQ ID NO: 2 of WO0015798, (SEQ ID NO: 192)
Anti-TGF beta family antibodies GB2305921
Latent TGF beta binding protein II GeneSeq Accession Y70552 WO0012551, Q14767/LTBP2_HUMAN Latent-transforming growth factor
beta-binding protein 2, (SEQ ID NO: 193)
MP52 GeneSeq Accession W36100 WO9741250, P43026/GDF5_HUMAN Growth/differentiation factor 5, (SEQ ID NO: 194)
b57 Protein GeneSeq Accession W69293 WO9837195, SEQ ID NO: 2 of WO9837195, (SEQ ID NO: 195)
Resistin GeneSeq Accession W69293 WO0064920, Q9HD89/RETN_HUMAN Resistin, (isoform 1), (SEQ ID NO: 196)
Galectin-4 GeneSeq Accession W11841 WO9703190, P56470/LEG4_HUMAN Galectin-4, (SEQ ID NO: 197)
APM-I; ACRP-30; Famoxin GeneSeq Accession Y71035 WO0026363, Q15848/ADIPO_HUMAN Adiponectin, (SEQ ID NO: 198)
ACRP-30 Homologue; Complement Component Clq C GeneSeq Accession B30234 WO0063376, P02747/C1QC_HUMAN Complement
C1q subcomponent subunit C, (SEQ ID NO: 199)
Calpain-10a GeneSeq Accession Y79567 WO0023603, Q9HC96/CAN10_HUMAN Calpain-10, (Isoform A), (SEQ ID NO: 200)
Calpain-10b GeneSeq Accession Y79568 WO0023603, Q9HC96-2/CAN10_HUMAN Isoform B of Calpain-10, (SEQ ID NO: 201)
Calpain-10c GeneSeq Accession Y79569 WO0023603, Q9HC96-3/CAN10_HUMAN Isoform C of Calpain-10, (SEQ ID NO: 202)
PDGF-D GeneSeq Accession Y71130 WO0027879, Q9GZP0/PDGFD_HUMAN Platelet-derived growth factor D, (isoform 1), (SEQ ID NO: 203)
FasL GeneSeq Accession Y28594 WO9936079, P48023/TNFL6_HUMAN Tumor necrosis factor ligand superfamily member 6, (isoform 1),
(SEQ ID NO: 204)
Chondro modulin-like protein GeneSeq Accession Y71262 WO0029579, SEQ ID NO: 2 from WO0029579, (SEQ ID NO: 370)
Patched GeneSeq Accession W72969 U.S. Pat. No. 5,837,538, Q13635/PTC1_HUMAN Protein patched homolog 1, (isoform L), (SEQ ID NO: 205)
Patched-2 GeneSeq Accession Y43261 WO9953058, Q9Y6C5/PTC2_HUMAN Protein patched homolog 2, (isoform 1), (SEQ ID NO: 206)
Maspin; Protease Inhibitor 5 GeneSeq Accession R50938 WO9405804, P36952/SPB5_HUMAN Serpin B5, (isoform 1), (SEQ ID NO: 207)
Endostatin GeneSeq Accession B28399 WO0064946, P39060/COIA1_HUMAN Collagen alpha-1(XVIII) chain, (isoform 1), (SEQ ID NO: 208)
aFGF; FGF-1 GeneSeq Accession P94037 EP298723, P05230/FGF1_HUMAN Fibroblast growth factor 1, (isoform 1), (SEQ ID NO: 209)
bFGF; FGF-2 GeneSeq Accession R06685 FR2642086, P09038/FGF2_HUMAN Fibroblast growth factor 2, (isoform 1), (SEQ ID NO: 210)
FGF-3; INT-2 GeneSeq Accession R07824 WO9503831, P11487/FGF3_HUMAN Fibroblast growth factor 3, (SEQ ID NO: 211)
FGF-4; HST-1; HBGF-4 GeneSeq Accession R07825 WO9503831, P08620/FGF4_HUMAN Fibroblast growth factor 4, (isoform 1), (SEQ ID NO: 212)
FGF-5 GeneSeq Accession W22600 WO9730155, P12034/FGF5_HUMAN Fibroblast growth factor 5, (isoform long), (SEQ ID NO: 213)
FGF-6; Heparin binding secreted transforming factor-2 GeneSeq Accession R58555 EP613946, P10767/FGF6_HUMAN Fibroblast growth factor 6,
(SEQ ID NO: 214)
FGF-8 GeneSeq Accession R80783 WO9524928, P55075/FGF8_HUMAN Fibroblast growth factor 8, (isoform 8E), (SEQ ID NO: 215)
FGF-9; Gila activating factor GeneSeq Accession R70822 WO9503831, P31371/FGF9_HUMAN Fibroblast growth factor 9, (SEQ ID NO: 216)
FGF-12; Fibroblast growth factor homologous factor-1 GeneSeq Accession WO6309 WO9635708, P61328/FGF12_HUMAN Fibroblast growth
factor 12, (isoform 1), (SEQ ID NO: 217)
FGF-19 GeneSeq Accession Y08582 WO9927100, O95750/FGF19_HUMAN Fibroblast growth factor 19, (SEQ ID NO: 218)
FGF-16 GeneSeq Accession Y05474 WO9918128, O43320/FGF16_HUMAN Fibroblast growth factor 16, (SEQ ID NO: 219)
FGF-18 GeneSeq Accession Y08590 WO9927100, O76093/FGF18_HUMAN Fibroblast growth factor 18, (SEQ ID NO: 220)
fit-3 ligand GeneSeq Accession R67541 EP627487, P49771|FLT3L_HUMAN Fms-related tyrosine kinase 3 ligand, (isoform 1), (SEQ ID NO: 221)
VEGF-110 GeneSeq Accession Y69417 WO0013702, SEQ ID NO: 11 from WO0013702, (SEQ ID NO: 222)
VEGF-121 GeneSeq Accession B50432 WO0071713, SEQ ID NO: 2 from WO0071713, (SEQ ID NO: 223)
VEGF-138 GeneSeq Accession Y43483 WO9940197, SEQ ID NO: 4 of WO99/40197, (SEQ ID NO: 371)
VEGF-145 GeneSeq Accession Y69413 WO0013702, SEQ ID NO: 4 from WO0013702, (SEQ ID NO: 224)
VEGF-162 GeneSeq Accession Y43484 W09940197, SEQ ID NO: 8 of WO99/40197, (SEQ ID NO: 372)
VEGF-165 GeneSeq Accession Y69414 WO0013702, SEQ ID NO: 6 from WO0013702, (SEQ ID NO: 225)
VEGF-182 GeneSeq Accession Y43483 W09940197, SEQ ID NO: 6 of WO99/40197, (SEQ ID NO: 373)
VEGF-189 GeneSeq Accession Y69415 WO0013702, SEQ ID NO: 8 from WO0013702, (SEQ ID NO: 226)
VEGF-206 GeneSeq Accession Y69416 W00013702, SEQ ID NO: 10 from WO0013702, (SEQ ID NO: 227)
VEGF-D GeneSeq Accession W53240 WO9807832, O43915/VEGFD_HUMAN Vascular endothelial growth factor D, (SEQ ID NO: 374)
VEGF-E; VEGF-X GeneSeq Accession Y33679 WO9947677, SEQ ID NO: 2 from WO9947677, (SEQ ID NO: 228)
VEGF Receptor; KDR; flk-1 GeneSeq Accession W69679 WO9831794, P35968/VGFR2_HUMAN Vascular endothelial growth factor receptor 2,
(isoform 1), (SEQ ID NO: 229)
Soluble VEGF Receptor GeneSeq Accession W47037 U.S. Pat. No. 5,712,380; sVEGF-RI (FIG. 3) of U.S. Pat. No. 5,712,380, (SEQ ID NO: 442);
sVEGF-RII (FIG. 11) of U.S. Pat. No. 5,712,380, (SEQ ID NO: 443); sVEGF-RTMI (FIG. 15) of U.S. Pat. No. 5,712,380, (SEQ ID NO: 444);
sVEGF-RTMII (FIG. 13) of U.S. Pat. No. 5,712,380, (SEQ ID NO: 445)
fit-1 GeneSeq Accession Y70751 WO0021560, P17948/VGFR1_HUMAN Vascular endothelial growth factor receptor 1, (isoform 1), (SEQ ID NO: 230)
VEGF R-3; flt-4 GeneSeq Accession B29047 WO0058511, P35916/VGFR3_HUMAN Vascular endothelial growth factor receptor 3, (isoform 1),
(SEQ ID NO: 231)
Neuropilin-1 GeneSeq Accession Y06319 WO9929858, O14786/NRP1_HUMAN Neuropilin-1, (isoform 1), (SEQ ID NO: 232)
Neuropilin-2 GeneSeq Accession Y03618 WO9929858, O60462/NRP2_HUMAN Neuropilin-2, (isoform A22), (SEQ ID NO: 233)
Human fast twitch skeletal muscle troponin C GeneSeq Accession W22597 WO9730085, P02585/TNNC2_HUMAN Troponin C, skeletal muscle,
(SEQ ID NO: 234)
Human fast twitch skeletal muscle troponin I GeneSeq Accession W18054 WO9730085, P48788/TNNI2_HUMAN Troponin I, fast skeletal muscle,
(isoform 1), (SEQ ID NO: 235)
Human fast twitch skeletal muscle troponin T GeneSeq Accession W22599 WO9730085, SEQ ID NO: 3 of WO9730085, (SEQ ID NO: 236)
Fragment. myofibrillar protein troponin I GeneSeq Accession W18053 WO9719955, SEQ ID NO: 3 of WO9719955, (SEQ ID NO: 237)
myofibrillar protein troponin I GeneSeq Accession W18054 WO9719955, SEQ ID NO: 3 of WO9719955, (SEQ ID NO: 237)
Troponin peptides GeneSeq Accessions Y29581, Y29582, Y29583, Y29584, Y29585, and Y29586 WO9933874. Wildtype troponins provided as:
Human fast twitch skeletal muscle troponin C GeneSeq Accession W22597 W09730085, P02585/TNNC2_HUMAN Troponin C, skeletal muscle,
(SEQ ID NO: 234); Human fast twitch skeletal muscle troponin I GeneSeq Accession W18054 W09730085, P48788/TNNI2_HUMAN Troponin 1,
fast skeletal muscle, (isoform 1), (SEQ ID NO: 235); Human fast twitch skeletal musde troponin T GeneSeq Accession W22599 W09730085,
SEQ ID NO: 3 of WO9730085, (SEQ ID NO: 236); fragment. myofibrillar protein troponin I GeneSeq Accession W18053 W09719955,
SEQ ID NO: 3 of WO9719955, (SEQ ID NO: 237); Human fast twitch skeletal muscle Troponin subunit C GeneSeq Accession B00134
WO0054770, SEQ ID NO: 1 of WO0054770, (SEQ ID NO: 375); Human fast twitch skeletal muscle Troponin subunit I
Protein GeneSeq Accession B00135 WO0054770, SEQ ID NO: 2 of WO0054770, (SEQ ID NO: 376); Human fast twitch skeletal muscle
Troponin subunit T GeneSeq Accession B00136 WO0054770, SEQ ID NO: 3 of WO0054770, (SEQ ID NO: 377)
Human fast twitch skeletal muscle Troponin subunit C GeneSeq Accession B00134 WO0054770, SEQ ID NO: 1 of WO0054770, (SEQ ID NO: 375)
Human fast twitch skeletal muscle Troponin subunit I Protein GeneSeq Accession B00135 WO0054770, SEQ ID NO: 2 of WO0054770,
(SEQ ID NO: 376)
Human fast twitch skeletal muscle Troponin subunit T GeneSeq Accession B00136 WO0054770, SEQ ID NO: 3 of
WO0054770, (SEQ ID NO: 377)
Activator lnbibitor-1; PAI-1 GeneSeq Accession R08411 WO9013648, P05121/PAI1_HUMAN Plasminogen activator inhibitor 1, (isoform 1),
(SEQ ID NO: 238)
Plasminogen Activator Inhibitor-2; PAI-2 GeneSeq Accession P94160 DE3722673, P05120/PAI2_HUMAN Plasminogen activator inhibitor 2,
(SEQ ID NO: 239)
Activator Inhibitor-2; PAI-2 GeneSeq Accession R10921 WO9102057, P05120/PAI2_HUMAN Plasminogen activator inhibitor 2, (SEQ ID NO: 239)
Human PAI-1 mutants GeneSeq Accessions R11755, R11756, R11757, R11758, R11759, R11760, R11761, R11762 and R11763 WO9105048,
Wildtype PAI-1 is provided as P05121/PAILHUMAN Plasminogen activator inhibitor 1, (isoform 1), (SEQ ID NO: 238)
CXCR3; CXC GeneSeq Accession Y79372 WO0018431, P49682|CXCR3_HUMAN C-X-C chemokine receptor type 3, (isoform 1), (SEQ ID NO: 240)
Modified Rantes GeneSeq Accession W38129 WO9737005, Wildtype Rantes provided herein as P13501/CCL5_HUMAN C-C motif chemokine 5,
(SEQ ID NO: 241)
RANTES GeneSeq Accession Y05299 EP905240, P13501/CCL5_HUMAN C-C motif chemokine 5, (SEQ ID NO: 241)
MCP-Ia GeneSeq Accession R73914 WO9509232, MCP-1 provided as P13500/CCL2_HUMAN C-C motif chemokine 2, (SEQ ID NO: 337)
MCP-Ib GeneSeq Accession Y26176 WO9929728, MCP-1 provided as P13500/CCL2_HUMAN C-C motif chemokine 2, (SEQ ID NO: 337)
MCP-I receptor GeneSeq Accession R79165 WO9519436: MCP-1A,, SEQ ID NO: 2 of WO9519436, (SEQ ID NO: 446); MCP-1B,
SEQ ID NO: 4 of WO9519436, (SEQ ID NO: 447)
MCP-3 GeneSeq Accession R73915 W09509232, P80098/CCL7_HUMAN C-C motif chemokine 7, (SEQ ID NO: 336)
MCP-4 receptor GeneSeq Accession W56689 WO9809171, SEQ ID NO: 2 of WO9809171, (SEQ ID NO: 378)
RANTES receptor GeneSeq Accession W29588 U.S. Pat. No. 5,652,133, SEQ ID NO: 2 of U.S. Pat. No. 5,652,133, (SEQ ID NO: 379)
CCR5 variant GeneSeq Accession W88238 WO9854317, Variants of wildtype CCR5 which has the sequence, of: P51681|CCR5_HUMAN C-C
chemokine receptor type 5, (SEQ ID NO: 448)
CCR7 GeneSeq Accession B50859 U.S. Pat. No. 6,153,441, P32248/CCR7_HUMAN C-C chemokine receptor type 7, (SEQ ID NO: 243)
CXC3 GeneSeq Accession W23345 WO9727299, P78423/X3CD_HUMAN Fractalkine, (SEQ ID NO: 244)
Eotaxin GeneSeq Accession W10099 WO9700960, P51671/CCL11_HUMAN Eotaxin, (SEQ ID NO: 245)
Neurotactin GeneSeq Accessions Y77537, W34307, Y53259, and, Y77539 U.S. Pat. No. 6,013,257 WO9742224, P78423/X3CD_HUMAN
Fractalkine, (SEQ ID NO: 244)
Human CKbeta-9 GeneSeq Accession B50860 U.S. Pat. No. 6,153,441, SEQ ID NO: 2 of U.S. Pat. No. 6,153,441, (SEQ ID NO: 246)
Lymphotactin GeneSeq Accession B50052 WO0073320, P47992/XCL1_HUMAN Lymphotactin, (SEQ ID NO: 247)
MIP-3 alpha GeneSeq Accession W44398 WO9801557, P78556/CCL20_HUMAN C-C motif chemokine 20, (isoform 1), (SEQ ID NO: 248)
MIP-3 beta GeneSeq Accession W44399 WO9801557, Q99731/CCL19_HUMAN C-C motif chemokine 19, (SEQ ID NO: 249)
MIP-Gamma GeneSeq Accession R70798, WO2006135382, (SEQ ID NO: 457)
Stem Cell Inhibitory Factor GeneSeq Accession R11553 WO9104274, SCIF in Table I of WO9104274, (SEQ ID NO: 380); SCIF in Table II of
WO9104274, (SEQ ID NO: 381)
Thrombopoietin GeneSeq Accession R79905 WO9521920, P40225|TPO_HUMAN Thrombopoietin, (isoform 1), (SEQ ID NO: 250)
c-kit ligand; SCF; Mast cell growth factor; MGF; Fibrosarcoma-derived stem cell factor GeneSeq Accession Y53284, R83978 and
R83977 EP992579 and EP676470, P215831SCF_HUMAN Kit ligand, (isoform 1), (SEQ ID NO: 251)
Platelet derived growth factor GeneSeq Accession B48653 WO0066736, PDGF-A, P04085/PDGFA_HUMAN Platelet-derived growth factor
subunit A, (Isoform long), (SEQ ID NO: 257); PDGF-B, P01127/PDGFB_HUMAN Platelet-derived growth factor subunit B, (isoform 1),
(SEQ ID NO: 258)
Melanoma inhibiting protein GeneSeq Accession R69811 WO9503328, (SEQ ID NO: 458)
Glioma-derived growth factor GeneSeq Accession R08120 EP399816
Platelet derived growth factor precursor A GeneSeq Accession R84759 EP682110, PDGF-A precursor (variant D1), (SEQ ID NO: 382);
PDGF-A precursor (variant 13-1), (SEQ ID NO: 383)
Platelet derived growth factor precursor B GeneSeq Accession R84760 EP682110, FIG. 1 or FIG. 2, Wildtype PDGF-B provided as:,
PDGF-B, P01127/PDGFB_HUMAN Platelet-derived growth factor subunit B, (isoform 1), (SEQ ID NO: 258)
Platelet derived growth factor Bvsis GeneSeq Accession P80595 and P80596 EP282317, FIG. 1 of EP282317, (SEQ ID NO: 384)
Placental Growth Factor GeneSeq Accessions R23059 and R23060 WO9206194, P49763-2/PLGF_HUMAN Isoform PIGF-1 of
Placenta growth factor, (isoform PIGF-1), (SEQ ID NO: 252)
Placental Growth Factor-2 GeneSeq Accession Y08289 DE19748734, P49763-3/PLGF_HUMAN Isoform PIGF-2 of Placenta
growth factor, (isoform PIGF-2), (SEQ ID NO: 253)
Thrombopoietin derivative1 GeneSeq Accession Y77244 WO0000612 (e.g. Table 3), Wildtype thrombopoietin provided as:,
P40225|TPO_HUMAN Thrombopoietin, (isoform 1), (SEQ ID NO: 250)
Thrombopoietin derivative2 GeneSeq Accession Y77255 WO0000612 (e.g. Table 3), Wildtype thrombopoietin provided as:,
P40225|TPO_HUMAN Thrombopoietin, (isoform 1), (SEQ ID NO: 250)
Thrombopoietin derivative 3 GeneSeq Accession Y77262, WO0000612 (e.g. Table 3), Wildtype thrombopoietin provided as:,
P40225|TPO_HUMAN Thrombopoietin, (isoform 1), (SEQ ID NO: 250)
Thrombopoietin derivative 4 GeneSeq Accession Y77267, WO0000612 (e.g. Table 3), Wildtype thrombopoietin provided as:,
P40225|TPO_HUMAN Thrombopoietin, (isoform 1), (SEQ ID NO: 250)
Thrombopoietin derivative 5 GeneSeq Accession Y77246, WO0000612 (e.g. Table 3), Wildtype thrombopoietin provided as:,
P40225|TPO_HUMAN Thrombopoietin, (isoform 1), (SEQ ID NO: 250)
Thrombopoietin derivative 6 GeneSeq Accession Y77253, WO0000612 (e.g. Table 3), Wildtype thrombopoietin provided as:,
P40225|TPO_HUMAN Thrombopoietin, (isoform 1), (SEQ ID NO: 250)
Thrombopoietin derivative, 7 GeneSeq Accession Y77256, WO0000612 (e.g. Table 3), Wildtype thrombopoietin provided as:,
P40225|TPO_HUMAN Thrombopoietin, (isoform 1), (SEQ ID NO: 250)
Fractalkine GeneSeq Accession Y53255 U.S. Pat. No. 6,043,086, P78423/X3CL1_HUMAN Fractalkine, (SEQ ID NO: 244)
CXC3 GeneSeq Accession W23345 WO9757599, P78423/X3CD_HUMAN Fractalkine, (SEQ ID NO: 244)
CCR7 GeneSeq Accession B50859 U.S. Pat. No. 6,153,441, P32248/CCR7_HUMAN C-C chemokine receptor type 7, (SEQ ID NO: 243)
Nerve Growth Factor-beta GeneSeq Accession R11474 EP414151, P01138/NGF_HUMAN Beta-nerve growth factor, (SEQ ID NO: 254)
Nerve Growth Factor-beta2 GeneSeq Accession W69725 EP859056, Fig 1 of EP859056, (SEQ ID NO: 465)
Neurotrophin-3 GeneSeq Accession W8889 WO9821234, P20783/NTF3_HUMAN Neurotrophin-3, (isoform 1), (SEQ ID NO: 255)
Neurotrophin-4 GeneSeq Accession R47100 WO9325684, P34130/NTF4_HUMAN Neurotrophin-4, (SEQ ID NO: 256)
Neurotrophin-4a GeneSeq Accession R47101 WO9325684, Wildtype neurotrophin provided as:, P34130/NTF4_HUMAN Neurotrophin-4,
(SEQ ID NO: 256)
Neurotrophin-4b GeneSeq Accession R47102 WO9325684, P34130/NTF4_HUMAN Neurotrophin-4, (SEQ ID NO: 256)
Neurotrophin-4c GeneSeq Accession R47103 WO9325684, P34130/NTF4_HUMAN Neurotrophin-4, (SEQ ID NO: 256)
Neurotrophin-4d GeneSeq Accession R47102 WO9325684, P34130/NTF4_HUMAN Neurotrophin-4, (SEQ ID NO: 256)
Platelet-Derived Growth Factor A chain GeneSeq Accession R38918 U.S. Pat. No. 5,219,739, P04085/PDGFA_HUMAN
Platelet-derived growth factor subunit A, (Isoform long), (SEQ ID NO: 257)
Platelet-Derived Growth Factor B chain GeneSeq Accession R38919 U.S. Pat. No. 5,219,739, P01127/PDGFB_HUMAN
Platelet-derived growth factor subunit B, (isoform 1), (SEQ ID NO: 258)
Stromal Derived Factor-1 alpha GeneSeq Accession Y39995 WO9948528, P48061-2/SDF1_HUMAN Isoform Alpha of
Stromal cell-derived factor 1, (isoform alpha), (SEQ ID NO: 259)
Stromal Derived Factor-1 beta GeneSeq Accession R75420 CA2117953, P48061/SDF1_HUMAN Stromal cell-derived factor 1,
(isoform beta), (SEQ ID NO: 260)
Tarc GeneSeq Accession W14917 WO9711969, Q92583/CCL17_HUMAN C-C motif chemokine 17, (SEQ ID NO: 261)
Prolactin GeneSeq Accession R78691 WO9521625, P01236/PRL_HUMAN Prolactin, (SEQ ID NO: 262)
Prolactin2 GeneSeq Accession Y31764 U.S. Pat. No. 5,955,346
Follicle stimulating hormone Alpha subunit GeneSeq Accession Y54160 EP974359, P01215/GLHA_HUMAN Glycoprotein hormones
alpha chain, (SEQ ID NO: 263)
Follicle stimulating hormone Beta subunit GeneSeq Accession Y54161 EP974359, P01225/FSHB_HUMAN Follitropin subunit beta,
(SEQ ID NO: 264)
Substance P (tachykinin) GeneSeq Accession B23027 WO0054053, (SEQ ID NO: 385)
Oxytocin (Neurophysin 1) GeneSeq Accession B24085 and B24086 WO0053755, P01178/NEU1_HUMAN Oxytocin-
neurophysin 1, (SEQ ID NO: 265)
Vasopressin (Neurophysin II) GeneSeq Accession B24085 and B24086 WO0053755, P01185/NEU2_HUMAN Vasopressin-
neurophysin 2-copeptin, (SEQ ID NO: 266)
IL-1 GeneSeq Accession P60326 EP165654, IL-1 alpha, P01583|IL1A_HUMAN Interleukin-1 alpha, (SEQ ID NO: 269); IL-1 beta,
P0158411L1B_HUMAN Interleukin-1 beta, (SEQ ID NO: 267)
IL-1 mature GeneSeq Accession R14855 EP456332, (mature truncated form wherein the precursor is cleaved between amino acids
116-117), (SEQ ID NO: 386)
IL-1 beta GeneSeq Accession Y08322 WO9922763, P01584|IL1B_HUMAN Interleukin-1 beta, (SEQ ID NO: 267)
IL-3 variants GeneSeq Accession P80382, P80383, P80384, and P80381 WO8806161, Variants of wildtype IL-3 which has the sequence:,
P0870011L3_HUMAN Interleukin-3, (SEQ ID NO: 449)
IL-4 GeneSeq Accession P70615 WO8702990, P05112/IL4_HUMAN Interleukin-4, (isoform 1), (SEQ ID NO: 268)
IL-4 muteins GeneSeq Accession W52151 W52152 W52153 W52154 W52155 W52156 W52157 W52158 W52159 W52160 W52161
W52162 W52163 W52164 and W52165 WO9747744, Variants of wildtype IL-4 which has the sequence:, P05112/IL4_HUMAN
Interleukin-4, (isoform 1), (SEQ ID NO: 268)
IL-1 alpha GeneSeq Accession P90108 EP324447, P01583|IL1A_HUMAN Interleukin-1 alpha, (SEQ ID NO: 269)
IL-3 variants GeneSeq Accession R38561, R38562, R38563, R38564, R38565, R38566, R38567, R38568, R38569, R38570, R38571, and
R38572 WO9307171, Variants of wildtype IL-3 which has the sequence:, P0870011L3_HUMAN Interleukin-3, (SEQ ID NO: 449)
IL-6 GeneSeq Accession R45717 and R45718 WO9402512, P05231/IL6_HUMAN Interleukin-6, (SEQ ID NO: 270)
IL-13 GeneSeq Accession R48624 WO9404680, P35225/IL13_HUMAN Interleukin-13, (SEQ ID NO: 271)
IL-4 mutein GeneSeq Accession R47182 DE4137333, Variants of wildtype IL-4 which has the sequence:, P05112/IL4_HUMAN
Interleukin-4, (isoform 1), (SEQ ID NO: 268)
IL-4 mutein Y124X GeneSeq Accession R47183 DE4137333, Variants of wildtype IL-4 which has the sequence:, P05112/IL4_HUMAN
Interleukin-4, (isoform 1), (SEQ ID NO: 268))
IL-4 mutein Yl 24G GeneSeq Accession R47184 DE4137333, Variants of wildtype IL-4 which has the sequence:, P05112/IL4_HUMAN
Interleukin-4, (isoform 1), (SEQ ID NO: 268)
Human Interleukin-10 (precursor) GeneSeq Accession R41664 WO9317698, P22301/IL10_HUMAN Interleukin-10, (precursor form is processed
into a truncated mature form), (SEQ ID NO: 272)
Human Interleukin-10 GeneSeq Accession R42642 WO9318783-A, SEQ ID NO: 3 of WO9318783-A, (mature IL-10), (SEQ ID NO: 273)
Human interleukin-1 beta precursor. GeneSeq Accession R42447 EP569042, P01584/IL1B_HUMAN Interleukin-1 beta, (SEQ ID NO: 274)
Interleukin-1alpha GeneSeq Accession R45364 EP578278, P01583|IL1A_HUMAN Interleukin-1 alpha, (SEQ ID NO: 269)
Human interleukin-3 variant GeneSeq Accession R22814 JP04063595, Variants of wildtype IL-3 which has the sequence:, P0870011L3_HUMAN
Interleukin-3, (SEQ ID NO: 449)
IL-1i fragments GeneSeq Accession R35484 and R35485 EP541920
IL-1 inhibitor IL-Ii) GeneSeq Accession R35486 and R35484 EP5541920
ICE 22 kD subunit. GeneSeq Accession R33780 EP533350, SEQ ID NO: 16 of EP533350, (SEQ ID NO: 450)
ICE 20 kD subunit. GeneSeq Accession R33781 EP533350, SEQ ID NO: 17 of EP533350, (SEQ ID NO: 451)
ICE 10 kD subunit GeneSeq Accession R33782 EP533350, SEQ ID NO: 18 of EP533350, (SEQ ID NO: 452)
Human Interleukin-10 (precursor) GeneSeq Accession R41664 WO9317698, P22301/IL10_HUMAN Interleukin-10, (precursor form is processed into a
truncated mature form), (SEQ ID NO: 272)
Human Interleukin-10 GeneSeq Accession R42642 WO9318783, SEQ ID NO: 3 of WO9318783-A, (mature IL-10), (SEQ ID NO: 273)
Human Interleukin-1 beta precursor GeneSeq Accession R42447 EP569042, P01584/IL1B_HUMAN Interleukin-1 beta, (SEQ ID NO: 274)
Human interleukin-6 GeneSeq Accession R49041 WO9403492, P05231/IL6_HUMAN Interleukin-6, (SEQ ID NO: 270)
Mutant Interleukin 6 S176R GeneSeq Accession R54990 WO9411402, 5176R variant of wildtype IL-6 which has the sequence:, P05231/IL6_
HUMAN Interleukin-6, (SEQ ID NO: 270)
Interleukin 6 GeneSeq Accession R55256 JP06145063, P05231/IL6_HUMAN Interleukin-6, (SEQ ID NO: 270)
Interleukin 8 (IL-8) receptor GeneSeq Accession R53932 JP06100595, GenBank: AAA59159.1, (SEQ ID NO: 275)
Human interleukin-7 GeneSeq Accession R59919 U.S. Pat. No. 5,328,988, P13232/IL7_HUMAN Interleukin-7, (isoform 1), (SEQ ID NO: 276)
IL-3 containing fusion protein. GeneSeq Accession R79342 and R79344 WO9521254, Fusions of wildtype IL-3 which has the sequence:,
P08700|1L3_HUMAN Interleukin-3, (SEQ ID NO: 449)
IL-3 mutant proteins GeneSeq Accession R79254, R79255, R79256, R79257, R79258, R79259, R79260, R79261, R79262, R79263, R79264,
R79265, R79266, R79267, R79268, R79269, R79270, R79271, R79272, R79273, R79274, R79275, R79276, R79277, R79278, R79279, R79280,
R79281, R79282, R79283, R79284, and R79285 ZA9402636, Variants of wildtype IL-3 which has the sequence:,
P08700|IL3_HUMAN Interleukin-3, (SEQ ID NO: 449)
IL-12 p40 subunit. GeneSeq Accession R63018 AU9466072, P2946/|IL12B_HUMAN Interleukin-12 subunit beta, (SEQ ID NO: 277)
AGF GeneSeq Accession R64240 WO9429344, Q8NI99/ANGL6_HUMAN Angiopoietin-related protein 6, (SEQ ID NO: 278)
Human interlaukin-12 40 kD subunit GeneSeq Accession R79187 WO9519786, P2946/|IL12B_HUMAN Interleukin-12 subunit beta,
(SEQ ID NO: 277)
Human interleukin-15 receptor from clone Pb GeneSeq Accession R90843 WO9530695, Q13261|I15RA_HUMAN Interleukin-15 receptor
subunit alpha, Isoform 1), (SEQ ID NO: 453)
Human interleukin-7 GeneSeq Accession R92796 WO9604306, P13232/IL7_HUMAN Interleukin-7, (isoform 1), (SEQ ID NO: 276)
interleukin-9 GeneSeq Accession R92797 WO9604306, P15248/IL9_HUMAN Interleukin-9, (SEQ ID NO: 279)
interleukin-3 GeneSeq Accession R92801 WO9604306, P08700|IL3_HUMAN Interleukin-3, (SEQ ID NO: 280)
Human interleukin-5 GeneSeq Accession R92802 WO9604306, P05113/IL5_HUMAN Interleukin-5, (SEQ ID NO: 281)
Recombinant interleukin-16 GeneSeq Accession W33373 DE19617202, Q14005/IL16_HUMAN Pro-interleukin-16, (isoform 1), (SEQ ID NO: 282)
Human IL-16 protein GeneSeq Accession W33234 DE19617202, Q14005/IL16_HUMAN Pro-interleukin-16, (isoform 1), (SEQ ID NO: 282)
Thrl 17 human interleukin 9 GeneSeq Accession W27521 WO9708321, P15248|IL9_HUMAN Interleukin-9, (SEQ ID NO: 387)
Metl 17 human interleukin 9 GeneSeq Accession W27522 WO9708321, (SEQ ID NO: 388)
Human intracellular IL-1 receptor antagonist. GeneSeq Accession W77158 EP864585 (e.g. SEQ ID NOs: 12 to 19, or 22 to 25 of this publication).
Human interleukin-18 protein (IL-18) GeneSeq Accession W77158 EP864585, Q14116/IL18_HUMAN Interleukin-18, (isoform 1), (SEQ ID NO: 283)
Human interleukin-18 GeneSeq Accession W77077 EP861663, Q14116/IL18_HUMAN Interleukin-18, (isoform 1), (SEQ ID NO: 283)
Human interleukin 18 derivatives GeneSeq Accessions W77083, W77084, W77085, W77086, W77087, W77088, and W77089 EP861663,
Variants of wildtype IL18 which is provided as:, Q14116/IL18_HUMAN Interleukin-18, (isoform 1), (SEQ ID NO: 283)
Interleukin-9 (IL-9) mature protein (Thr117 version). GeneSeq Accession W68158 WO9827997, FIG. 2 of WO9827997, (SEQ ID NO: 389)
IL-9 mature protein variant (Met117 version) GenSeq Accession W68157 WO9827997, FIG. 3 of WO9827997, (SEQ ID NO: 390)
Human IL-9 receptor protein variant #3. GeneSeq Accession W64058 WO9824904, Wildtype IL-9R is provided as:, Q01113/IL9R_
HUMAN Interleukin-9 receptor, (isoform 1), SEQ ID NO: 303)
Human IL-9 receptor protein variant fragment GenSeq Accession W64060 WO9824904, Wildtype IL-9R is provided as:, Q01113/IL9R_HUMAN
Interleukin-9 receptor, (isoform 1), (SEQ ID NO: 303)
Human IL-9 receptor protein variant #3. GeneSeq Accession W64061 WO9824904, Wildtype IL-9R is provided as:, Q01113/IL9R_
HUMAN Interleukin-9 receptor, (isoform 1), (SEQ ID NO: 303)
Human Interleukin-12 p40 protein GeneSeq Accession W51311 WO9817689, P2946/|IL12B_HUMAN Interleukin-12 subunit beta, (SEQ ID NO: 277)
Human Interleukin-12 p35 protein GeneSeq Accession W51312 WO9817689, P29459/IL12A_HUMAN Interleukin-12 subunit alpha, (SEQ ID NO: 284)
Human protein with IL-16 activity GeneSeq Accession W63753 DE19649233-
Human protein with IL-16 activity GeneSeq Accession W59425 DE19649233-
Human interleukin-15 GeneSeq Accession W53878 U.S. Pat. No. 5,747,024, P40933/IL15_HUMAN Interleukin-15, (isoformIL15-S48AA),
(SEQ ID NO: 285)
Human wild-type interleukin-4 (hIL-4) protein GeneSeq Accession W52149 WO9747744, P05112/IL4_HUMAN Interleukin-4, (isoform 1),
(SEQ ID NO: 286)
interleukin-4 muteins GeneSeq Accessions W52150, W52151, W52153, W52154, W52155, W52156, W52157, W52158, W52159,
W52160, W52161, W52162, W52163, W52164, W52165, W52166, and W52167 WO9747744, Variants of wildtype IL-4
which has the sequence:, P05112/IL4_HUMAN Interleukin-4, (isoform 1), (SEQ ID NO: 268)
Human interleukin 1 delta GeneSeq Accession Y28408 WO9935268, SEQ ID NO: 4 of WO9935268, (SEQ ID NO: 287)
Human interleukin-1 receptor antagonist beta GeneSeq Accession Y24395 WO9935268,
Human EDIRF II protein sequence GeneSeq Accession Y22199 WO9932632, SEQ ID NO: 6 of WO9932632, (SEQ ID NO: 391)
Human EDIRF I protein sequence GeneSeq Accession Y22197 WO9932632, SEQ ID NO: 2 of WO9932632, (SEQ ID NO: 392)
Human IL-1RD10 protein sequence GeneSeq Accession Y14131 WO9919480, SEQ ID NO: 20 of WO9919480
Human IL-1RD9 GeneSeq Accession Y14122 WO9919480, SEQ ID NOS: 6, 8, 10 of WO9919480
Human DNAX interleukin-40 GeneSeq Accession Y09196 WO9919491, SEQ ID NO: 2 or 4 of, WO9919491, (SEQ ID NO: 454)
(DIL-40) alternative sequence GeneSeq Accession Y09197 WO9919491, SEQ ID NO: 4 of, WO9919491, (SEQ ID NO: 455)
IL-11 GeneSeq Accession R50176 WO9405318, P2080/|IL11_HUMAN Interleukin-11, (isoform 1), (SEQ ID NO: 288)
Human adipogenesis inhibitory factor GeneSeq Accession R43260 EP566410, (aIso known as IL-11), P2080/|IL11_HUMAN
Interleukin-11, (isoform 1), (SEQ ID NO: 288)
IL-11 GeneSeq Accession W02202 JP08127539, P2080/|IL11_HUMAN Interleukin-11, (isoform 1), (SEQ ID NO: 288)
IL-14 GeneSeq Accession R55800 WO9416074, P40222/TXLNA_HUMAN Alpha-taxilin, (SEQ ID NO: 289)
IL-17 receptor GeneSeq Accession B03807 U.S. Pat. No. 6,072,033, Q96F46/I17RA_HUMAN Interleukin-17 receptor A, (SEQ ID NO: 290)
IL-17 GeneSeq Accession R76573 WO9518826, Q16552/IL17_HUMAN Interleukin-17A, (SEQ ID NO: 291)
CTLA-8 GeneSeq Accession W13651 WO9704097, (also known as IL-17), Q16552/IL17_HUMAN Interleukin-17A, (SEQ ID NO: 291)
IL-19 GeneSeq Accession W37935 WO9808870, Q9UHD0|IL19_HUMAN Interleukin-19, (isoform 1), (SEQ ID NO: 292)
IL-21 (TIF) GeneSeq Accession Y92879 WO0024758, Q9HBE4/IL21_HUMAN Interleukin-21, (isoform 1), (SEQ ID NO: 293)
IL-8 receptor GeneSeq Accession R33420 WO9306229, IL-8RA, P25024/CXCR1_HUMAN C-X-C chemokine receptor type 1,
(SEQ ID NO: 294), IL-8RB, P25025/CXCR2_HUMAN C-X-C chemokine receptor type 2, (SEQ ID NO: 295)
Human type II interleukin-1 receptor GeneSeq Accession R85480 U.S. Pat. No. 5,464,937, P27930/IL1R2_HUMAN Interleukin-1
receptor type 2, (SEQ ID NO: 296)
Human interleukin-12 receptor GeneSeq Accession R69632 EP638644, IL-12 receptor B1, P42701|I12R1_HUMAN Interleukin-12
receptor subunit beta-1, (isoform 1), (SEQ ID NO: 393), IL-12 receptor B2, Q99665|I12R2_HUMAN
Interleukin-12 receptor subunit beta-2, (isoform 1), (SEQ ID NO: 394)
Interleukin 8 receptor B GeneSeq Accession R80758 U.S. Pat. No. 5,440,021, IL-8RB, P25025/CXCR2_HUMAN C-X-C chemokine
receptor type 2, (SEQ ID NO: 295)
Human IL-8 receptor protein hIL8RA GeneSeq Accession B09989 JP08103276, IL-8RA, P25024/CXCR1_HUMAN C-X-C chemokine receptor type 1,
(SEQ ID NO: 294)
Human IL-8 receptor protein hIL8R GeneSeq Accession B09990 JP08103276, IL-8RA, P25024/CXCR1_HUMAN C-X-C chemokine receptor type 1,
(SEQ ID NO: 294); IL-8RB, P25025/CXCR2_HUMAN C-X-C chemokine receptor type 2, (SEQ ID NO: 295)
Interleukin-2 receptor associated protein p43 GeneSeq Accession R97569 WO9621732-, SEQ ID NO: 2 of WO9621732, (SEQ ID NO: 395)
Human interleukin-17 receptor GeneSeq Accession W04185 WO9629408, Q96F46/I17RA_HUMAN Interleukin-17 receptor A, (SEQ ID NO: 290)
Human interleukin-11 receptor GeneSeq Accession R99090 WO9619574, Q14626/I11RA_HUMAN Interleukin-11 receptor subunit alpha,
(SEQ ID NO: 297)
Human interleukin-1 receptor accessory protein GeneSeq Accession W01911 WO9623067, Human IL1R Acp, SEQ ID NO: 3 of WO9623067,
(SEQ ID NO: 396); Soluble Human IL1R Acp, SEQ ID NO: 9 of WO9623067, (SEQ ID NO: 397)
AGF Protein GeneSeq Accession R92749 U.S. Pat. No. 5,488,032, Q8NI99/ANGL6_HUMAN Angiopoietin-related protein 6, (SEQ ID NO: 278)
Human interleukin-1 type-3 receptor GeneSeq Accession R91064 WO9607739, SEQ ID NO: 2 and 4 of WO9607739, (SEQ ID NO: 398 and
SEQ ID NO: 399, respectively)
Human interleukin-13 beta receptor GeneSeq Accession W24972 WO9720926, SEQ ID NO: 2 from WO9720926, (SEQ ID NO: 400)
Human interleukin-13 alpha receptor GeneSeq Accession W24973 WO9720926, IL-13RA1, P78552/I13R1_HUMAN Interleukin-13 receptor subunit
alpha-1, (isoform 1), (SEQ ID NO: 298); IL-13RA2, Q14627/I13R2_HUMAN Interleukin-13 receptor subunit alpha-2, (SEQ ID NO: 299)
Human interleukin-4 receptor GeneSeq Accession W13499 U.S. Pat. No. 5,599,905, P24394/IL4RA_HUMAN Interleukin-4 receptor subunit alpha,
(isoform 1), (SEQ ID NO: 300)
Human interleukin-12 beta-2 receptor GeneSeq Accession W12771 EP759466, Q9966/I12R2_HUMAN Interleukin-12 receptor subunit beta-2,
(isoform 1), (SEQ ID NO: 301)
Human interleukin-12 beta-1 receptor. GeneSeq Accession W12772 EP759466, P4270/I12R1_HUMAN Interleukin-12 receptor subunit beta-1,
(isoform 1), (SEQ ID NO: 302)
Human IL-9 receptor protein GeneSeq Accessions W64055, W64056, and W64057 WO9824904, Q01113/IL9R_HUMAN Interleukin-9 receptor,
(isoform 1), (SEQ ID NO: 303)
IL-10 receptor GeneSeq Accession W41804 U.S. Pat. No. 5,716,804, IL-10RA, Q13651/I10R1_HUMAN Interleukin-10 receptor subunit alpha,
(SEQ ID NO: 304); IL-10RB, Q0833/I10R2_HUMAN Interleukin-10 receptor subunit beta, (SEQ ID NO: 305)
Human IL-6 receptor GeneSeq Accession Y30938 JP11196867, P08887/IL6RA_HUMAN Interleukin-6 receptor subunit alpha, (isoform 1),
(SEQ ID NO: 306)
II-17 receptor GeneSeq Accession Y97181 U.S. Pat. No. 6,096,305, Q96F46/I17RA_HUMAN Interleukin-17 receptor A, (SEQ ID NO: 290)
II-17 receptor GeneSeq Accession Y97131 U.S. Pat. No. 6,100,235, Q96F46/I17RA_HUMAN Interleukin-17 receptor A, (SEQ ID NO: 290)
Human interleukin-3 receptor GeneSeq Accession R25300 EP509826, P26951/IL3RA_HUMAN Interleukin-3 receptor subunit alpha, (isoform 1),
(SEQ ID NO: 307)
Human GM-CSF receptor GeneSeq Accession R10919 WO9102063, GM-CSF receptor A, P15509/CSF2R_HUMAN Granulocyte-macrophage colony-
stimulating factor receptor subunit alpha, (isoform 1), (SEQ ID NO: 308); GM-CSF receptor B, P32927/IL3RB_HUMAN Cytokine receptor
common subunit beta, (isoform 1), (SEQ ID NO: 309)
Human IL-5 receptor alpha chain GeneSeq Accession R25064 EP492214, Q01344/IL5RA_HUMAN Interleukin-5 receptor subunit alpha, (isoform 1),
(SEQ ID NO: 310)
II-5 receptor GeneSeq Accession W82842 WO9847923, Q01344/IL5RA_HUMAN Interleukin-5 receptor subunit alpha, (isoform 1), (SEQ ID NO: 310)
II-6 receptor GeneSeq Accession R37215 JP05091892, P08887/IL6RA_HUMAN Interleukin-6 receptor subunit alpha, (isoform 1), (SEQ ID NO: 306)
Human B cell stimulating factor-2 receptor GeneSeq Accession P90525 AU8928720, P08887/IL6RA_HUMAN Interleukin-6 receptor subunit alpha,
(isoform 1), (SEQ ID NO: 306)
IL-7 receptor clone GeneSeq Accession R08330 EP403114, P1687/|IL7RA_HUMAN Interleukin-7 receptor subunit alpha, (isoform 1),
(SEQ ID NO: 311)
EPO receptor; EPOR GeneSeq Accession R06512 WO9008822, P19235/EPOR_HUMAN Erythropoietin receptor, (isoform EPOR-F),
(SEQ ID NO: 312)
IL-15 receptor GeneSeq Accession R90843 WO9530695, Q1326/|I15RA_HUMAN Interleukin-15 receptor subunit alpha, (isoform 1),
(SEQ ID NO: 313)
CD137; 4-1BB Receptor Protein GeneSeq Accession R70977 WO9507984, Q07011/TNR9_HUMAN Tumor necrosis factor receptor superfamily
member 9, (SEQ ID NO: 314)
BCMA GeneSeq Accession Y71979 WO0068378, Q02223/TNR17_HUMAN Tumor necrosis factor receptor superfamily member 17, (isoform 1),
(SEQ ID NO: 315)
CD27 GeneSeq Accession R20814 WO9201049, P26842/CD27_HUMAN CD27 antigen, (SEQ ID NO: 316)
CD30 GeneSeq Accession R35478 DE4200043, P28908/TNR8_HUMAN Tumor necrosis factor receptor superfamily member 8, (isoform 1),
(SEQ ID NO: 317)
CD40 GeneSeq Accession Y33499 WO9945944, P25942/TNR5_HUMAN Tumor necrosis factor receptor superfamily member 5, (isoform 1),
(SEQ ID NO: 318)
EDAR Genbank Accession AAD50077, Q9UNE0|EDAR_HUMAN Tumor necrosis factor receptor superfamily member EDAR, (isoform 1),
(SEQ ID NO: 319)
OX40; ACT-4 GeneSeq Accession R74737 WO9512673, P43489/TNR4_HUMAN Tumor necrosis factor receptor superfamily member 4,
(SEQ ID NO: 320)
TACI GeneSeq Accession W75783 WO9839361, O14836/TR13B_HUMAN Tumor necrosis factor receptor superfamily member 13B, (isoform 1),
(SEQ ID NO: 321)
TNF-R GeneSeq Accession R10986 AU9058976, P19438/TNR1A_HUMAN Tumor necrosis factor receptor superfamily member 1A, (isoform 1),
(SEQ ID NO: 322)
TNF-RII; TNF p75 receptor; Death Receptor GeneSeq Accession R11141 EP418014, P20333/TNR1B_HUMAN Tumor necrosis factor receptor
superfamily member 1B, (isoform 1), (SEQ ID NO: 323)
hAPO-4; TROY GeneSeq Accession W93581 WO9911791, Q9N568/TNR19_HUMAN Tumor necrosis factor receptor superfamily member 19,
(isoform 1), (SEQ ID NO: 324)
TNF-alpha precursor GeneSeq Accession P60074 EP205038
Human TNF-alpha GeneSeq Accession R62463 EP619372, P01375/TNFA_HUMAN Tumor necrosis factor, (SEQ ID NO: 325)
Human TNF-alpha GeneSeq Accession R42679 EP563714, P01375/TNFA_HUMAN Tumor necrosis factor, (SEQ ID NO: 325)
Human TNF-beta (LT-alpha) GeneSeq Accession B37799 WO0064479, P01374/TNFB_HUMAN Lymphotoxin-alpha, (SEQ ID NO: 326)
LT-alpha GeneSeq Accession P70107 EP250000, P01374/TNFB_HUMAN Lymphotoxin-alpha, (SEQ ID NO: 326)
LT-beta GeneSeq Accession R56869 WO9413808, Q06643/TNFC_HUMAN Lymphotoxin-beta, (isoform 1), (SEQ ID NO: 327)
OPGL GeneSeq Accession W83195 WO9846751, O14788/TNF11_HUMAN Tumor necrosis factor ligand superfamily member 11, (isoform 1),
(SEQ ID NO: 328)
FasL GeneSeq Accession W98071 WO9903999, P48023/TNFL6_HUMAN Tumor necrosis factor ligand superfamily member 6, (isoform 1),
(SEQ ID NO: 329)
FasL GeneSeq Accession W95041 WO9903998, P48023/TNFL6_HUMAN Tumor necrosis factor ligand superfamily member 6, (isoform 1),
(SEQ ID NO: 329)
CD27L GeneSeq Accession R50121 WO9405691, P32970/CD70_HUMAN CD70 antigen, (isoform 1), (SEQ ID NO: 330)
CD30 ligand GeneSeq Accession R45007 WO9324135, P32971/TNFL8_HUMAN Tumor necrosis factor ligand superfamily member 8,
(SEQ ID NO: 331)
CD40L GeneSeq Accession R85486 WO9529935, P29965/CD40L_HUMAN CD40 ligand, (SEQ ID NO: 332)
4-1BB ligand GeneSeq Accession W26657 U.S. Pat. No. 5,674,704, P41273/TNFL9_HUMAN Tumor necrosis factor ligand superfamily member 9,
(SEQ ID NO: 333)
FAS Ligand Inhibitory Protein (DcR3) GeneSeq Accession B19335 WO0058465, O95407/TNF6B_HUMAN Tumor necrosis factor receptor
superfamily member 6B, (SEQ ID NO: 334)
OX40L GeneSeq Accession R79903 WO9521915, P23510/TNFL4_HUMAN Tumor necrosis factor ligand superfamily member 4,
(isoform 1), (SEQ ID NO: 335)
Protease inhibitor peptides GeneSeq Accessions R12435, R12436, R12437, R12438, R12439, R12440, and R1244 WO9106561
Retroviral protease inhibitors GeneSeq Accessions R06660, R06661, R06662, R06663, R06664, R06665, R06666, R06667, R06668, R06669,
R06670, R06671, R06672, R06673, R06674, R06675, and R06676 EP387231
HIV protease inhibiting peptides GeneSeq Accessions R59293, R59294, R59295, R59296, R59297, R59298, R59299, R592300, R59301,
R59302, R59301, R59302, R59303, R59304, R59305, R59306, R59307, R59308, R59309, R59310, R59311, R59312, R59313, R59314,
R59315, R59316, R59317 R59318, R59319, R59320, R59321, R59322, R59323, R59324, R59325, R59326, R59327, R59328, R59329,
R59330, R59331, R59332, R59333, R59334, R59335, R59336, R59337, R59338, R59339, R59340, R59341, R59342, R59343, R59344,
R59345, R59346, R59347, R59348, R59349, and R59350 WO9301828
HIV-1 protease inhibitors GeneSeq Accessions R86326, R86327, R86328, R86329, R86330, R86331, R86332, R86333, R86334, R86335,
R86336, R86337, R86338, R86339, R86340, R86341, R86342, R86343, R86344, R86345, R86346, R86347, R86348, R86349, R86350,
R86351, R86352, R86353, R86354, R86355, R86356, R86357, R86358, R86359, R86360, R86361, R86362, R86363, R86364, R86365,
R86366, R86367, R86368, R86369, R86370, and R86371 DE4412174
HIV Inhibitor Peptide GeneSeq Accession Y89687 WO9959615
HIV Inhibitor Peptide GenSeq Accession Y31955 WO9948513
HIV Inhibitor Peptide Science 291, 884 (2001); Published online 12 Jan. 2001; 10.1126/science.1 057453
Human monocyte chemoattractant factor hMCP-3 GeneSeq Accession R73915 WO9509232, P80098/CCL7_HUMAN C-C motif chemokine 7,
(SEQ ID NO: 336)
Human monocyte chemoattractant factor hMCP-1 GeneSeq Accession R73914 WO9509232, P13500/CCL2_HUMAN C-C motif chemokine 2,
(SEQ ID NO: 337)
Human gro-beta chemokine GeneSeq Accessions R66699 and W17671 WO9429341, P19875/CXCL2_HUMAN C-X-C motif chemokine 2,
(SEQ ID NO: 338)
Human gro-gamma chemokine GeneSeq Accessions R66700 and W17672 WO9429341, P19876/CXCL3_HUMAN C-X-C motif chemokine 3,
(SEQ ID NO: 339)
Human gro-alpha chemokine GeneSeq Accessions R66698 and W18024 WO9429341, P09341/GROA_HUMAN Growth-regulated alpha protein,
(SEQ ID NO: 340)
Human eosinophil-expressed chemokine (EEC) GeneSeq Accession WO5186 WO9632481, SEQ ID NO: 2 of WO9632481, (SEQ ID NO: 401)
Chemokine-like protein PF4-414 Full-Length and Mature GeneSeq Accessions R92318 and R99809 WO9613587, FIG. 3C of WO9613587,
(SEQ ID NO: 402)
Chemokine-like protein IL-8M3 GeneSeq Accession R99812 WO9613587
Human interleukin-8 (IL-8) GeneSeq Accession R99814 WO9613587, P10145/IL8_HUMAN Interleukin-8, (isoform 1), (SEQ ID NO: 341)
Chemokine-like protein IL-8M1 Full-Length and Mature GeneSeq Accessions R99815 and R99803 WO9613587, FIG. 4B of
WO9613587, (SEQ ID NO: 403)
Chemokine-like protein IL-8M8 Full-Length and Mature GeneSeq Accessions R99816 and R99805 WO9613587, FIG. 4C of
WO9613587, (SEQ ID NO: 404)
Chemokine-like protein IL-8M8 Full-Length and Mature GeneSeq Accessions R99817 and R99806 WO9613587, FIG. 4C of
WO9613587, (SEQ ID NO: 404)
Chemokine-like protein IL-8M8 Full-Length and Mature GeneSeq Accessions R99818 and R99804 WO9613587, FIG. 4C of
WO9613587, (SEQ ID NO: 404)
Chemokine-like protein IL-8M8 Full-Length and Mature GeneSeq Accessions R99819 and R99807 WO9613587, FIG. 4C of
WO9613587, (SEQ ID NO: 404)
Chemokine-like protein IL-8M8 Full-Length and Mature GeneSeq Accessions R99822 and R9807 WO9613587, FIG. 4C of
WO9613587, (SEQ ID NO: 404)
Human foetal spleen ex-pressed chemo-kine, FSEC GeneSeq Accession R98499 WO9622374, SEQ ID NO: 2 of, WO9622374, (SEQ ID NO: 405)
Liver expressed chemokine-1(LVEC-1) GeneSeq Accession R95689 WO9616979, SEQ ID NO: 2 of, WO9616979, (SEQ ID NO: 406)
Liver expressed chemokine-2(LVEC-2) GeneSeq Accession R95690 WO9616979, SEQ ID NO: 4 of, WO9616979, (SEQ ID NO: 407)
Pituitary expressed chemokine (PGEC) GeneSeq Accession R95691 WO9616979, SEQ ID NO: 6 of, WO9616979, (SEQ ID NO: 408)
Adenoid-expressed chemokine (ADEC) GeneSeq Accession R97664 WO9617868, SEQ ID NO: 2 of, WO9617868, (SEQ ID NO: 409)
Human chemokine CC-2 GeneSeq Accession W38170 WO9741230, Q16663/CCL15_HUMAN C-C motif chemokine 15, (SEQ ID NO: 342)
Human chemokine HCC-1 GeneSeq Accession W38171 WO9741230, Q16627/CCL14_HUMAN C-C motif chemokine 14, (SEQ ID NO: 343)
Human chemokine CC-3 GeneSeq Accession W38172 WO9741230, Q16627/CCL14_HUMAN C-C motif chemokine 14, (SEQ ID NO: 343)
Novel betachemokine designated PTEC GeneSeq Accession W27271 WO9739126, SEQ ID NO: 2 of WO9739126, (SEQ ID NO: 410)
Human CX3C 111 amino acid chemokine GeneSeq Accession W23344 WO9727299, SEQ ID NO: 2 of WO9727299, (SEQ ID NO: 411)
Human CCF18 chemokine GeneSeq Accession W25942 WO9721812, SEQ ID NO: 4 of WO9721812, (SEQ ID NO: 412)
Human beta-chemokine H1305 (MCP-2) GeneSeq Accession W26655 WO9725427, P80075/CCL8_HUMAN C-C motif chemokine 8,
(SEQ ID NO: 344)
Human eosinocyte CC type chemokine eotaxin GeneSeq Accession W14990 WO9712914, P51671/CCL11_HUMAN Eotaxin, (SEQ ID NO: 245)
Human thymus and activation regulated cytokine (TARC) GeneSeq Accession W14018 WO9711969, Q92583/CCL17_HUMAN C-C motif
chemokine 17, (SEQ ID NO: 261)
Human chemokine beta-8 short forms GeneSeq Accession W16315 WO9712041, Wildtype chemokine beta-8 provided as:, P55773|CCL23_
HUMAN C-C motif chemokine 23, (SEQ ID NO: 459)
Microphage derived chemokine, MDC GeneSeq Accession W20058 WO9640923, O00626/CCL22_HUMAN C-C motif chemokine 22,
(SEQ ID NO: 345)
Human chemokine ZSIG-35 GeneSeq Accession W30565 WO9844117, SEQ ID NO: 2 of WO WO9844117, (SEQ ID NO: 413)
Primate CC chemokine “ILINCK” GeneSeq Accesssion W69990 WO98328658, SEQ ID NO: 4 from WO9832858, (SEQ ID NO: 414)
Primate CXC chemokine “IBICK” GeneSeq Accession W69989 WO9832858, SEQ ID NO: 2 from WO9832858, (SEQ ID NO: 415)
Human CC-type chemokine protein designated SLC (secondary lymphoid chemokine) GeneSeq Accession W69163 WO9831809, O00585/CCL21_
HUMAN C-C motif chemokine 21, (SEQ ID NO: 346)
Human CC chemokine ELC protein GeneSeq Accession W62542 WO9826071, Q99731/CCL19_HUMAN C-C motif chemokine 19,
(SEQ ID NO: 249)
Human DVic-1 C-C chemokine GeneSeq Accession W60649 WO9823750, SEQ ID NO: 2 of WO9823750, (SEQ ID NO: 416)
Human C-C chemokine DGWCC GeneSeq Accession W60650 WO9823750, SEQ ID NO: 6 of WO9823750, (SEQ ID NO: 417)
Human STCP-1 GeneSeq Accession W62783 WO9824907, O00626/CCL22_HUMAN C-C motif chemokine 22, (SEQ ID NO: 345)
Exodus protein GeneSeq Accession W61279 WO9821330, P78556/CCL20_HUMAN C-C motif chemokine 20, (isoform 1), (SEQ ID NO: 248)
Human Chr19kine protein GeneSeq Acession W50887 WO9814581, SEQ ID NO: 10 of WO9814581, (SEQ ID NO: 418)
Human T cell mixed lymphocyte reaction expressed chemokine (TMEC) GeneSeq Accession W58703 U.S. Pat. No. 5,780,268, SEQ ID NO: 2 of
U.S. Pat. No. 5,780,268, (SEQ ID NO: 460)
Human 6CKine protein GeneSeq Accession W50885 WO9814581, SEQ ID NO: 8 of WO9814581, (SEQ ID NO: 419)
human liver and activation regulated chemokine (LARC) GeneSeq Accession W57475 WO9817800, P78556/CCL20_HUMAN C-C motif chemokine
20, (isoform 1), (SEQ ID NO: 248)
RANTES peptide GeneSeq Accession W29538 WO9744462, Wildtype Rantes provided herien as P13501/CCL5_HUMAN C-C motif chemokine 5,
(SEQ ID NO: 241)
RANTES 8-68 GeneSeq Accession W29529 WO9744462, Wildtype Rantes provided herien as P13501/CCL5_HUMAN C-C motif chemokine 5,
(SEQ ID NO: 241)
RANTES 9-68 GeneSeq Accession W29528 WO9744462, Wildtype Rantes provided herien as P13501/CCL5_HUMAN C-C motif chemokine 5,
(SEQ ID NO: 241)
Human chemokine protein 331D5 GeneSeq Accession W59433 WO9811226, SEQ ID NO: 12 of WO9811226, (SEQ ID NO: 420)
Human chemokine protein 61164 GeneSeq Accession W59430 WO9811226, SEQ ID NO: 6 of WO9811226, (SEQ ID NO: 421)
Chemokine MCP-4 GeneSeq Accession W56690 WO9809171, Q99616/CCL13_HUMAN C-C motif chemokine 13, (SEQ ID NO: 347)
Human stromal cell-derived chemokine, SDF-1 GeneSeq Accession W50766 FR2751658, P48061/SDF1_HUMAN Stromal cell-derived factor 1,
(isoform beta), (SEQ ID NO: 260)
Thymus expressed chemokine (TECK) GeneSeq Accession W44397 WO9801557, O15444/CCL25_HUMAN C-C motif chemokine 25,
(SEQ ID NO: 348)
Human chemokine MIP-3alpha GeneSeq Accession W44398 WO9801557, P78556/CCL20_HUMAN C-C motif chemokine 20, (isoform 1),
(SEQ ID NO: 248)
Human chemokine MIP-3beta GeneSeq Accession W44399 WO9801557, Q99731/CCL19_HUMAN C-C motif chemokine 19, (SEQ ID NO: 249)
Human monocyte chemotactic proprotein (MCPP) sequence GeneSeq Accession W42072 WO9802459, SEQ ID NO: 1 of WO9802459,
(SEQ ID NO: 456)
Macrophage-derived chemokine (MDC) GeneSeq Accessions W40811 and Y24414 US Pat No. 5,688,927/U.S. Pat. No. 5,932,703,
O00626/CCL22_HUMAN C-C motif chemokine 22, (SEQ ID NO: 345)
Macrophage derived chemokine analogue MDC-eyfy GeneSeq Accession Y24416 U.S. Pat. No. 5,932,703 (“eyfy” disclosed as SEQ ID NO:
546), Wildtype MDC is SEQ ID NO: 2 of 5,932,703, (SEQ ID NO: 422)
Macrophage derived chemokine analogue MDC (n + 1) GeneSeq Accession Y24413 U.S. Pat. No. 5,932,703
Macrophage derived chemokine analogue MDC-yl GeneSeq Accession Y24415 U.S. Pat. No. 5,932,703
Human type CC chemokine eotaxin 3 protein sequence GeneSeq Accession Y43178 JP11243960, Q9Y258/CCL26_HUMAN C-C
motif chemokine 26, (SEQ ID NO: 349)
Human MCP-3 and human Muc-1 core epitope (VNT) fusion protein GeneSeq Acession Y29893 WO9946392, Wildtype MCP-3
has the sequence:, P80098/CCL7_HUMAN C-C motif chemokine 7, (SEQ ID NO: 336); Wildtype Muc-1 has the sequence:,
P15941|MUC1_HUMAN Mucin-1, (isoform 1), (SEQ ID NO: 461)
Human IP-10 and human Muc-1 core epitope (VNT) fusion protein GeneSeq Accession Y29894 WO9946392, Wildtype
IP10 has the sequence:, P02778/CXL10_HUMAN C-X-C motif chemokine 10, (SEQ ID NO: 242); Wildtype Muc-1 has the sequence:,
P15941|MUC1_HUMAN Mucin-1, (isoform 1), (SEQ ID NO: 461)
Human IP-10 and HIV-1 gp120 hyper-variable region fusion protein GeneSeq Accession Y29897 WO9946392, Wildtype
IP10 has the sequence:, P02778/CXL10_HUMAN C-X-C motif chemokine 10, (SEQ ID NO: 242); Wildtype gp120 has the sequence:,
P03378|32-509, (cleaved product of gp160), (SEQ ID NO: 462)
Human mammary associated chemokine (MACK) protein Full-Length and Mature GeneSeq Accessions Y29092 and Y29093 WO9936540,
Full-length: SEQ ID NO: 1 of WO9936540, (SEQ ID NO: 423); Mature Form: SEQ ID NO: 2 of WO9936540, (SEQ ID NO: 424)
Tim-1 protein GeneSeq Accession Y28290 WO9933990, SEQ ID NO: 2 of, WO9933990, (SEQ ID NO: 350)
Human Lkn-1 Full-Length and Mature protein GeneSeq Accessions Y17280, Y17274, Y17281, and Y17275 WO9928473 and WO9928472,
Q16663/CCL15_HUMAN C-C motif chemokine 15, (SEQ ID NO: 342)
N-terminal modified chemokine met-hSDF-1 alpha GeneSeq Accession Y05818 WO9920759, SEQ ID NO: 10 of WO9920759, (SEQ ID NO: 425)
N-terminal modified chemokine met-hSDF-1 beta GeneSeq Accession Y05819 WO9920759, SEQ ID NO: 11 of WO9920759, (SEQ ID NO: 426)
N-terminal modified chemokine GroHEK/hSDF-1alpha GeneSeq Accession Y05820 WO9920759, SEQ ID NO: 12 of WO9920759, (SEQ ID NO: 427)
N-terminal modified chemokine GroHEK/hSDF-1beta. GeneSeq Accession Y05821 WO9920759, SEQ ID NO: 13 of WO9920759, (SEQ ID NO: 428)
Chemokine Eotaxin GeneSeq Accession Y14230 WO9912968, P51671/CCL11_HUMAN Eotaxin, (SEQ ID NO: 245)
Chemokine hMCP1a GeneSeq Accession Y14225 WO9912968
Chemokine hMCP1b GeneSeq Accession Y14226 WO9912968
Chemokine hSDF1b GeneSeq Accession Y14228 WO9912968, P48061/SDF1_HUMAN Stromal cell-derived factor 1, (isoform beta),
(SEQ ID NO: 260)
Chemokine hIL-8 GeneSeq Accession Y14229 WO9912968, P10145/IL8_HUMAN Interleukin-8, (isoform 1), (SEQ ID NO: 341)
Chemokine hMCP1 GeneSeq Accession Y14222 WO9912968, P13500/CCL2_HUMAN C-C motif chemokine 2, (SEQ ID NO: 337)
Chemokine hMCP2 GeneSeq Accession Y14223 WO9912968, P80075/CCL8_HUMAN C-C motif chemokine 8, (SEQ ID NO: 344)
Chemokine hMCP3 GeneSeq Accession Y14224 WO9912968, P80098/CCL7_HUMAN C-C motif chemokine 7, (SEQ ID NO: 336)
C-C chemokine, MCP2 GeneSeq Accession Y05300 EP905240, P80075/CCL8_HUMAN C-C motif chemokine 8, (SEQ ID NO: 344)
Wild type monocyte chemotactic protein 2 GeneSeq Accession Y07233 EP906954, P80075/CCL8_HUMAN C-C motif chemokine 8, (SEQ ID NO: 344)
Truncated monocyte chemotactic protein 2 (6-76) GeneSeq Accession Y07234 EP906954, FIG. 1 of EP905241 and EP906954, (SEQ ID NO: 429)
Truncated RANTES protein (3-68) GeneSeq Accessions Y07236 and Y07232 EP905241; EP906954, FIG. 1 of EP906954, (SEQ ID NO: 430)
Wild type monocyte chemotactic protein 2 GeneSeq Accession Y07237 EP905241, P80075/CCL8_HUMAN C-C motif chemokine 8, (SEQ ID NO: 344)
Truncated monocyte chemotactic protein 2 (6-76) GeneSeq Accession Y07238 EP905241, FIG. 1 of EP905241 and EP906954, (SEQ ID NO: 429)
A partial CXCR4B protein GeneSeq Accession W97363 EP897980, SEQ ID NO: 2 of EP897980, (SEQ ID NO: 431)
Interferon gamma-inducible protein (IP-10) GeneSeq Accession W96709 U.S. Pat. No. 5,871,723, P02778/CXL10_HUMAN C-X-C motif
chemokine 10, (SEQ ID NO: 242)
A monokine induced by gamma-interferon (MIG) GeneSeq Accession W96710 U.S. Pat. No. 5,871,723, Q07325/CXCL9_HUMAN C-X-C motif
chemokine 9, (SEQ ID NO: 351)
Interleukin-8 (IL-8) protein. GeneSeq Accession W96711 U.S. Pat. No. 5,871,723, P10145/IL8_HUMAN Interleukin-8, (isoform 1), (SEQ ID NO: 341)
Epithelial neutrophil activating protein-78 (ENA-78) GeneSeq Accession W96712 U.S. Pat. No. 5,871,723, P42830/CXCL5_HUMAN C-X-C motif
chemokine 5, (SEQ ID NO: 352)
Growth related oncogene-alpha (GRO-alpha). GeneSeq Accession W96713 U.S. Pat. No. 5,871,723, P09341/GROA_HUMAN Growth-regulated
alpha protein, (SEQ ID NO: 340)
Growth related oncogene-beta (GRO-beta). GeneSeq Accession W96714 U.S. Pat. No. 5,871,723, P19875/CXCL2_HUMAN C-X-C motif
chemokine 2, (SEQ ID NO: 338)
Growth related oncogene-gamma (GRO-gamma) GeneSeq Accession W96715 U.S. Pat. No. 5,871,723, P19876/CXCL3_HUMAN C-X-C
motif chemokine 3, (SEQ ID NO: 339)
A platelet basic protein (PBP) GeneSeq Accession W96716 U.S. Pat. No. 5,871,723, P02775/CXCL7_HUMAN Platelet basic protein, (SEQ ID NO:
353)
Connective tissue activating protein-III (CTAP-III) GeneSeqAc-cession S96717 U.S. Pat. No. 5,871,723, SEQ ID NO: 9 of U.S. Patent No. 5,871,723,
(SEQ ID NO: 354)
Beta-thrombo-globulin protein (beta-TG) GeneSeq Accession W96718 U.S. Pat. No. 5,871,723, SEQ ID NO: 10 of U.S. Patent No. 5,871,723,
(SEQ ID NO: 355)
Neutrophil activating peptide-2 (NAP-2) GeneSeq Accession W96719 U.S. Pat. No. 5,871,723, SEQ ID NO: 11 of U.S. Patent No.
5,871,723, (SEQ ID NO: 356)
Granulocyte chemotactic protein-2 (GCP-2) GeneSeq Accession W96720 U.S. Pat. No. 5,871,723, P80162/CXCL6_HUMAN C-X-C
motif chemokine 6, (SEQ ID NO: 357)
Human chemokine MIG-beta protein GeneSeq Accession W90124 EP887409, (SEQ ID NO: 463)
Human ZCHEMO-8 GeneSeq Accession W82716 WO9854326, SEQ ID NO: 2 of WO9854326, (SEQ ID NO: 432)
Human Act-2 protein GeneSeq Accession W82717 WO9854326, P13236/CCL4_HUMAN C-C motif chemokine 4, (SEQ ID NO: 358)
Human SISD protein GeneSeq Acession W82720 WO9854326, P13501/CCL5_HUMAN C-C motif chemokine 5, (SEQ ID NO: 241)
Human MI10 protein GeneSeq Accession W82721 WO9854326, SEQ ID NO: 37 of WO9854326, (SEQ ID NO: 433)
Human MI1A protein GeneSeq Accession W82722 WO9854326, SEQ ID NO: 38 of WO9854326, (SEQ ID NO: 434)
Human CCC3 protein GeneSeq Accession W82723 WO9854326, SEQ ID NO: 39 of WO9854326, (SEQ ID NO: 435)
A human L105 chemokine designated huL105_3. GeneSeq Accession W87588 WO9856818, SEQ ID NO: 2 of WO9856818, (SEQ ID NO: 436)
A human L105 chemokine designated huL105_7. GeneSeq Accession W87589 WO9856818, SEQ ID NO: 4 of WO9856818, (SEQ ID NO: 437)
Human mature gro-alpha polypeptide used to treat sepsis GeneSeq Accession W81498 WO9848828, P09341/GROA_HUMAN Growth-
regulated alpha protein, (SEQ ID NO: 340)
Human mature gro-gamma polypeptide used to treat sepsis GeneSeq Accession W81500 WO9848828, P19876/CXCL3_HUMAN C-X-C
motif chemokine 3, (SEQ ID NO: 339)
Human thymus expressed chemokine TECK and TECK variant GeneSeq Accessions B19607 and B19608 WO0053635, Wildtype TECK provided as:,
O15444/CCL25_HUMAN C-C motif chemokine 25, (SEQ ID NO: 348)
Human chemokine SDF1alpha GeneSeq Accession B15791 WO0042071, P48061-2/SDF1_HUMAN Isoform Alpha of Stromal cell-derived factor 1,
(isoform alpha), (SEQ ID NO: 259),
Human chemokine GRO-alpha GeneSeq Accession B15793 WO0042071, P09341/GROA_HUMAN Growth-regulated alpha protein,
(SEQ ID NO: 340)
Human chemokine eotaxin GeneSeq Accession B15794 WO0042071, P51671/CCL11_HUMAN Eotaxin, (SEQ ID NO: 245)
Human chemokine MIG GeneSeq Accession B15803 WO0042071, Q07325/CXCL9_HUMAN C-X-C motif chemokine 9, (SEQ ID NO: 351)
Human chemokine PF4 GeneSeq Accession B15804 WO0042071, P02776/PLF4_HUMAN Platelet factor 4, (SEQ ID NO: 359)
Human chemokine I-309 GeneSeq Accession B15805 WO0042071, P22362/CCD_HUMAN C-C motif chemokine 1, (SEQ ID NO: 360)
Human chemokine HCC-1 GeneSeq Accession B15806 WO0042071, Q16627/CCL14_HUMAN C-C motif chemokine 14, (SEQ ID NO: 361)
Human chemokine C10 GeneSeq Accession B15807 WO0042071, SEQ ID NO: 49 of WO0042071, (SEQ ID NO: 438)
Human chemokine CCR-2 GeneSeq Accession B15808 WO0042071, P41597/CCR2_HUMAN C-C chemokine receptor type 2,
(isoform A), (SEQ ID NO: 362)
Human chemokine ENA-78 GeneSeq Accession B15809 WO0042071, P42830/CXCL5_HUMAN C-X-C motif chemokine 5, (SEQ ID NO: 352)
Human chemokine GRObeta GeneSeq Accession B15810 WO0042071, P19875/CXCL2_HUMAN C-X-C motif chemokine 2, (SEQ ID NO: 338)
Human chemokine IP-10 GeneSeq Accession B15811 WO0042071, P02778/CXL10_HUMAN C-X-C motif chemokine 10, (SEQ ID NO: 242)
Human chemokine SDF1beta GeneSeq Accession B15812 WO0042071, P48061/SDF1_HUMAN Stromal cell-derived factor 1, (isoform beta),
(SEQ ID NO: 260)
Human chemokine GRO alpha GeneSeq Accession B15813 WO0042071, P09341/GROA_HUMAN Growth-regulated alpha protein, (SEQ ID NO: 340),
Human chemokine MIP1beta GeneSeq Accession B15831 WO0042071, P13236/CCL4_HUMAN C-C motif chemokine 4, (SEQ ID NO: 358)
A human C-C chemokine designated exodus GeneSeq Accession B07939 U.S. Pat. No. 6,096,300, P78556/CCL20_HUMAN C-C motif chemokine 20,
(isoform 1), (SEQ ID NO: 248)
Human chemokine L105_7 GeneSeq Accession Y96922 U.S. Pat. No. 6,084,071, SEQ ID NO: 4 of WO9856818, (SEQ ID NO: 437)
Human chemokine L105_3 GeneSeq Accession Y96923 U.S. Pat. No. 6,084,071, SEQ ID NO: 2 of WO9856818, (SEQ ID NO: 436)
Human secondary lymphoid chemokine (SLC) GeneSeq Accession B01434 WO0038706, O00585/CCL21_HUMAN C-C motif chemokine 21,
(SEQ ID NO: 346)
Human non-ELR CXC chemokine H174 GeneSeq Accession Y96310 WO0029439, O14625/CXL11_HUMAN C-X-C motif chemokine 11,
(SEQ ID NO: 363)
Human non-ELR CXC chemokine IP10 GeneSeq Accession Y96311 WO0029439, P02778/CXL10_HUMAN C-X-C motif chemokine 10,
(SEQ ID NO: 242)
Human non-ELR CXC chemokine Mig GeneSeq Accession Y96313 WO0029439, Q07325/CXCL9_HUMAN C-X-C motif chemokine 9,
(SEQ ID NO: 351)
Human chemokine Ckbeta-7 GeneSeq Accession Y96280 WO0028035, FIG. 1 of WO0028035, (SEQ ID NO: 439)
Human chemokine MIP-1alpha GeneSeq Accession Y96281 WO0028035, P10147/CCL3_HUMAN C-C motif chemokine 3, (SEQ ID NO: 364)
Human mature chemokine Ckbeta-7 (optionally truncated) GenSeq Accession Y96282 WO0028035, FIG. 1 of WO0028035, (SEQ ID NO: 440)
Human chemokine receptor CXCR3 GeneSeq Accession Y79372 WO0018431, P49682|CXCR3_HUMAN C-X-C chemokine
receptor type 3, (isoform 1), (SEQ ID NO: 240)
Human neurotactin chemokine like domain GeneSeq Accession Y53259 U.S. Pat. No. 6,043,086, P78423/X3CD_HUMAN Fractalkine,
(SEQ ID NO: 244)
Human CC type chemokine interieukin C GeneSeq Accession Y57771 JP11302298
Human CKbeta-9 GeneSeq Accession B50860 U.S. Pat. No. 6,153,441, O00585/CCL21_HUMAN C-C motif chemokine 21, (SEQ ID NO: 346)
Preproapolipo-protein “paris” variant GeneSeq Accession WO8602 WO9637608, (SEQ ID NO: 466)
Preproapolipo-protein “milano” variant 5,721,114, SEQ ID NO: 6 of U.S. Pat. No. 5,721,114, (SEQ ID NO: 441)
Glycodelin-A; Progesterone-associated endometrial protein GeneSeq Accession WO0289 WO9628169, P09466/PAEP_HUMAN Glycodelin,
(SEQ ID NO: 365)
NOGO-A Genbank Accession CAB99248, (SEQ ID NO: 366)
NOGO-B Genbank Accession CAB99249, (SEQ ID NO: 367)
NOGO-C Genbank Accession CAB99250, (SEQ ID NO: 368),
NOGO-66 Receptor Genbank Accession AAG53612, (SEQ ID NO: 369)
Antibodies specific for collapsin U.S. Pat. No. 5,416,197, Wildtype collapsin has the sequence:, SEQ ID NO: 2 of 5,416,197, (SEQ ID NO: 464)
Humanized Anti-VEGF Antibodies, and fragments thereof WO9845331
Humanized Anti-VEGF Antibodies, and fragments thereof WO0029584
Membrane bound proteins GeneSeq. Accession Y66631-Y66765 WO9963088
Secreted and Transmembrane polypeptides GeneSeq Accession B44241-B44334 WO0053756
Secreted and Transmembrane polypeptides GeneSeq Accession Y41685-Y41774 WO9946281
Interleukin 2 (IL-2), (SEQ ID NO: 548)
Interleukin 15_vA, (IL-15_vA), (SEQ ID NO: 549)
Interleukin 15_vB, (IL-15_vB), (SEQ ID NO: 550)
Interleukin 15_vC, (IL-15_vC), (SEQ ID NO: 551)
Interleukin 15_vD, (IL15_vD), (SEQ ID NO: 552)
Interleukin 15_vE, (IL15_vE), (SEQ ID NO: 553)
Interleukin 15_vF, (IL15_vF), (SEQ ID NO: 565)
Interleukin 22, (IL22), (SEQ ID NO: 554)
Fibroblast Growth Factor 1 (FGF1), (SEQ ID NO: 555)
Fibroblast Growth Factor 1_vA, (FGF1_vA), (SEQ ID NO: 556)
Fibroblast Growth Factor 1_vB, (FGF1_vB), (SEQ ID NO: 557)
Fibroblast Growth Factor 1_vC, (FGF1_vC), (SEQ ID NO: 566)
Fibroblast Growth Factor 19_vA, (FGF19_vA), (SEQ ID NO: 558)
Fibroblast Growth Factor 21, (FGF21), (SEQ ID NO: 559)
Fibroblast Growth Factor 23, (FGF23), (SEQ ID NO: 560)
Brain-Derived Neurotrophic Factor (BDNF), (SEQ ID NO: 561)
Serpin Family A Member 1, (SERPINA1), ((SEQ ID NO: 584) and ((SEQ ID NO: 585)
Serpin Peptidase Inhibitor, Clade B (Ovalbumin), Member 1, (SERPINB1), (SEQ ID NO: 562)
CASPASE1, (SEQ ID NO: 563)
Leukemia Inhibitory Factor, (LIF), (SEQ ID NO: 564)
Proprotein Convertase Subtilisin/Kexin Type 1, (PCSK1), (SEQ ID NO: 567)
Proprotein Convertase Subtilisin/Kexin Type 2 (PCSK2), (SEQ ID NO: 568)
Proprotein Convertase Subtilisin/Kexin Type 3, (PCSK3), (SEQ ID NO: 569)
Proprotein Convertase Subtilisin/Kexin Type 3 Sol, (PCSK3_SOL), (SEQ ID NO: 570)
Proprotein Convertase Subtilisin/Kexin Type 4, (PCSK4), (SEQ ID NO: 571)
Proprotein Convertase Subtilisin/Kexin Type 5, (PCSK5), (SEQ ID NO: 572)
Proprotein Convertase Subtilisin/Kexin Type 6 (PCSK6), (SEQ ID NO: 573)
Proprotein Convertase Subtilisin/Kexin Type, (PCSK7), (SEQ ID NO: 574)
Proprotein Convertase Subtilisin/Kexin Type 8, (PCSK8), (SEQ ID NO: 575)
Proprotein Convertase Subtilisin/Kexin Type 9, (PCSK9), (SEQ ID NO: 576)
Membrane-Bound Transcription Factor Peptidase, Site 2, (MBTPS2), (SEQ ID NO: 577)
Carboxypeptidase E, (CPE), (SEQ ID NO: 578)

In various embodiments, the present methods and compositions find use in treating or preventing one or more of diseases or disorders in the table below. In various embodiments, the present methods and compositions find use in treating or preventing one or more of diseases or disorders in the table below for instance by modulating the genes associated with the diseases in the table below. In some embodiments, the present methods and compositions find use in gene-editing the genes described in the below Table using the present compositions.

TABLE 3C

| Category | Disease | Genes | Entrez ID |
|---|---|---|---|
| Disorders of | Galactosemia | GALT, | 2592, 2584, |
| carbohydrate | | GALK1, GALE | 2582 |
| metabolism | Essential fructosuria | KHK | 3795 |
| | Hereditary fructose | ALDOB | 229 |
| | intolerance | | |
| | Glycogen storage disease | G6PC, | 2538, 2542, |
| | type I | SLC37A4, | 10786 |
| | | SLC17A3 | |
| | Glycogen storage disease | GAA | 2548 |
| | type II | | |
| | Glycogen storage disease | AGL | 178 |
| | type III | | |
| | Glycogen storage disease | GBE1 | 2632 |
| | type IV | | |
| | Glycogen storage disease | PYGM | 5837 |
| | type V | | |
| | Glycogen storage disease | PYGL | 5836 |
| | type VI | | |
| | Glycogen storage disease | PYGM | 5837 |
| | type VII | | |
| | Glycogen storage disease | PHKA1, | 5255, 5256, |
| | type IX | PHKA2, | 5257, 5260, |
| | | PHKB, | 5261 |
| | | PHKG1, | |
| | | PHKG2 | |
| | Glycogen storage disease | SLC2A2 | 6514 |
| | type XI | | |
| | Glycogen storage disease | ALDOA | 226 |
| | type XII | | |
| | Glycogen storage disease | ENO1, ENO2, | 2023, 2026, |
| | type XIII | ENO3 | 2027 |
| | Glycogen storage disease | GYS1, GYS2 | 2997, 2998 |
| | type 0 | | |
| | Pyruvate carboxylase | PC | 5091 |
| | deficiency | | |
| | Pyruvate kinase deficiency | PKLR | 5313 |
| | Transaldolase deficiency | TALDO1 | 6888 |
| | Triosephosphate isomerase | TPI1 | 7167 |
| | deficiency | | |
| | Fructose bisphosphatase | FBP1 | 2203 |
| | deficiency | | |
| | Hyperoxaluria | AGXT, | 189, 9380 |
| | | GRHPR | |
| | Hexokinase deficiency | HK1 | 3098 |
| | Glucose-galactose | SLC5A1 | 6523 |
| | malabsorption | | |
| | Glucose-6-phosphate | G6PD | 2539 |
| | dehydrogenase deficiency | | |
| Disorders of | Alkaptonuria | HGD | 3081 |
| amino acid | Aspartylglucosaminuria | AGA | 175 |
| metabolism | Methylmalonic acidemia | MUT, MCEE, | 4594, 84693, |
| | | MMAA, | 166785, |
| | | MMAB, | 326625, |
| | | MMACHC, | 25974, 27249, |
| | | MMADHC, | 55788 |
| | | LMBRD1 | |
| | Maple syrup urine disease | BCKDHA, | 593, 594, |
| | | BCKDHB, | 1629, 1738 |
| | | DBT, DLD | |
| | Homocystinuria | CBS | 875 |
| | Tyrosinemia | FAH, TAT, | 2184, 6898, |
| | | HPD | 3242 |
| | Trimethylaminuria | FMO3 | 2328 |
| | Hartnup disease | SLC6A19 | 340024 |
| | Biotinidase deficiency | BTD | 686 |
| | Ornithine | OTC | 5009 |
| | carbamoyltransferase | | |
| | deficiency | | |
| | Carbamoyl-phosphate | CPS1 | 1373 |
| | synthase I deficiency disease | | |
| | Citrullinemia | ASS, | 445, 10165 |
| | | SLC25A13 | |
| | Hyperargininemia | ARG1 | 383 |
| | Hyperhomocysteinemia | MTHFR | 4524 |
| | Hypermethioninemia | MAT1A, | 4143, 27232, |
| | | GNMT, AHCY | 191 |
| | Hyperlysinemias | AASS | 10157 |
| | Nonketotic hyperglycinemia | GLDC, AMT, | 2731, 275, |
| | | GCSH | 2653 |
| | Propionic acidemia | PCCA, PCCB | 5095, 5096 |
| | Hyperprolinemia | ALDH4A1, | 8659, 5625 |
| | | PRODH | |
| | Cystinuria | SLC3A1, | 6519, 11136 |
| | | SLC7A9 | |
| | Dicarboxylic aminoaciduria | SLC1A1 | 6505 |
| | Glutaric acidemia type 2 | ETFA, ETFB, | 2108, 2109, |
| | | ETFDH | 2110 |
| | Isovaleric acidemia | IVD | 3712 |
| | 2-Hydroxyglutaric aciduria | L2HGDH, | 79944, |
| | | D2HGDH | 728294 |
| Disorders of | N-Acetylglutamate synthase | NAGS | 162417 |
| the urea | deficiency | | |
| cycle | Argininosuccinic aciduria | ASL | 435 |
| | Argininemia | ARG1 | 383 |
| Disorders of | Very long-chain | ACADVL | 37 |
| fatty acid | acyl-coenzyme A | | |
| metabolism | dehydrogenase deficiency | | |
| | Long-chain 3-hydroxyacyl- | HADHA | 3030 |
| | coenzyme A dehydrogenase | | |
| | deficiency | | |
| | Medium-chain | ACADM | 34 |
| | acyl-coenzyme A | | |
| | dehydrogenase deficiency | | |
| | Short-chain acyl-coenzyme | ACADS | 35 |
| | A dehydrogenase deficiency | | |
| | 3-hydroxyacyl-coenzyme A | HADH | 3033 |
| | dehydrogenase deficiency | | |
| | 2,4 Dienoyl-CoA reductase | NADK2 | 133686 |
| | deficiency | | |
| | 3-Hydroxy-3- | HMGCL | 3155 |
| | methylglutaryl-CoA | | |
| | lyase deficiency | | |
| | Malonyl-CoA decarboxylase | MLYCD | 23417 |
| | deficiency | | |
| | Systemic primary carnitine | SLC22A5 | 6584 |
| | deficiency | | |
| | Carnitine-acylcarnitine | SLC25A20 | 788 |
| | translocase deficiency | | |
| | Carnitine | CPT1A | 1374 |
| | palmitoyltransferase I | | |
| | deficiency | | |
| | Carnitine | CPT2 | 1376 |
| | palmitoyltransferase II | | |
| | deficiency | | |
| | Lysosomal acid lipase | LIPA | 3988 |
| | deficiency | | |
| | Gaucher's disease | GBA | 2629 |
| Disorders of | Acute intermittent porphyria | HMBS | 3145 |
| porphyrin | Gunther disease | UROS | 7390 |
| metabolism | Porphyria cutanea tarda | UROD | 7389 |
| | Hepatoerythropoietic | UROD | 7389 |
| | porphyria | | |
| | Hereditary coproporphyria | CPDX | 1371 |
| | Variegate porphyria | PPDX | 5498 |
| | Erythropoietic | FECH | 2235 |
| | protoporphyria | | |
| | Aminolevulinic acid | ALAD | 210 |
| | dehydratase deficiency | | |
| | porphyria | | |
| Lysosomal | Farber disease | ASAH1 | 427 |
| storage | Krabbe disease | GALC | 2581 |
| disorders | Galactosialidosis | CTSA | 5476 |
| | Fabry disease | GLA | 2717 |
| | Schindler disease | NAGA | 4668 |
| | GM1 gangliosidosis | GLB1 | 2720 |
| | Tay-Sachs disease | HEXA | 3073 |
| | Sandhoff disease | HEXB | 3074 |
| | GM2-gangliosidosis, AB | GM2A | 2760 |
| | variant | | |
| | Niemann-Pick disease | SMPD1, NPC1, | 6609, 4864, |
| | | NPC2 | 10577 |
| | Metachromatic | ARSA, PSAP | 410, 5660 |
| | leukodystrophy | | |
| | Multiple sulfatase deficiency | SUMF1 | 285362 |
| | Hurler syndrome | IDUA | 3425 |
| | Hunter syndrome | IDS | 3423 |
| | Sanfilippo syndrome | SGSH, | 6448, 4669, |
| | | NAGLU, | 138050, 2799 |
| | | HGSNAT, | |
| | | GNS | |
| | Morquio syndrome | GALNS, GLB1 | 2588, 2720 |
| | Maroteaux-Lamy syndrome | ARSB | 411 |
| | Sly syndrome | GUSB | 2990 |
| | Sialidosis | NEU1, NEU2, | 4758, 4759, |
| | | NEU3, NEU4 | 10825, |
| | | | 129807 |
| | I-cell disease | GNPTAB, | 79158, 84572 |
| | | GNPTG | |
| | Mucolipidosis type IV | MCOLN1 | 57192 |
| | Infantile neuronal ceroid | PPT1, PPT2 | 5538, 9374 |
| | lipofuscinosis | | |
| | Jansky-Bielschowsky | TPP1 | 1200 |
| | disease | | |
| | Batten disease | CLN1, CLN2, | 5538, 1200, |
| | | CLN3, CLN5, | 1201, 1203, |
| | | CLN6, MFSD8, | 54982, |
| | | CLN8, CTSD | 256471, 2055, |
| | | | 1509 |
| | Kufs disease, Type A | CLN6, PPT1 | 54982, 5538 |
| | Kufs disease, Type B | DNAJC5, | 80331, 8722 |
| | | CTSF | |
| | Alpha-mannosidosis | MAN2B1, | 4125, 23324, |
| | | MAN2B2, | 4123 |
| | | MAN2C1 | |
| | Beta-mannosidosis | MANBA | 4226 |
| | Fucosidosis | FUCA1 | 2517 |
| | Cystinosis | CTNS | 1497 |
| | Pycnodysostosis | CTSK | 1513 |
| | Salla disease | SLC17A5 | 26503 |
| | Infantile free sialic acid | SLC17A5 | 26503 |
| | storage disease | | |
| | Danon disease | LAMP2 | 3920 |
| Peroxisome | Zellweger syndrome | PEX1, PEX2, | 5189, 5828, |
| biogenesis | | PEX3, PEX5, | 8504, 5830, |
| disorders | | PEX6, PEX12, | 5190, 5193, |
| | | PEX14, PEX26 | 5195, 55670 |
| | Infantile Refsum disease | PEX1, PEX2, | 5189, 5828, |
| | | PEX26 | 55670 |
| | Neonatal | PEX5, PEX1, | 5830, 5189, |
| | adrenoleukodystrophy | PEX10, | 5192, 5194, |
| | | PEX13, PEX26 | 55670 |
| | RCDP Type 1 | PEX7 | 5191 |
| | Pipecolic acidemia | PAHX | 5264 |
| | Acatalasia | CAT | 847 |
| | Hyperoxaluria type 1 | AGXT | 189 |
| | Acyl-CoA oxidase | ACOX1 | 51 |
| | deficiency | | |
| | D-bifunctional protein | HSD17B4 | 3295 |
| | deficiency | | |
| | Dihydroxyacetonephosphate | GNPAT | 8443 |
| | acyltransferase deficiency | | |
| | X-linked | ABCD1 | 215 |
| | adrenoleukodystrophy | | |
| | α-Methylacyl-CoA racemase | AMACR | 23600 |
| | deficiency | | |
| | RCDP Type 2 | DHAPAT | 8443 |
| | RCDP Type 3 | AGPS | 8540 |
| | Adult Refsum disease-1 | PHYH | 5264 |
| | Mulibrey nanism | TRIM37 | 4591 |
| Disorders of | Lesch-Nyhan syndrome | HPRT | 3251 |
| purine or | Adenine | APRT | 353 |
| pyrimidine | phosphoribosyltransferase | | |
| metabolism | deficiency | | |
| | Adenosine deaminase | ADA | 100 |
| | deficiency | | |
| | Adenosine monophosphate | AMPD1 | 270 |
| | deaminase deficiency type 1 | | |
| | Adenylosuccinate lyase | ADSL | 158 |
| | deficiency | | |
| | Dihydropyrimidine | DPYD | 1806 |
| | dehydrogenase deficiency | | |
| | Miller syndrome | DHODH | 1723 |
| | Orotic aciduria | UMPS | 7372 |
| | Purine nucleoside | PNP | 4860 |
| | phosphorylase deficiency | | |
| | Xanthinuria | XDH, MOCS1, | 7498, 4337, |
| | | MOCS2, GEPH | 4338, 10243 |

The Entrez entries listed in the table above are hereby incorporated by reference in their entireties.

Additional illustrative targets of the present invention include the cosmetic targets listed in Table 6 of International Patent Publication No. WO 2013/151671, the contents of which are hereby incorporated by reference in their entirety.

Further, in some embodiments, the present methods and compositions find use in targeting any of the proteins or in treatment of any of the diseases or disorders of Table 3A, Table 3B, and/or Table 3C. In various embodiments, the present invention contemplates the targeting of the full-length and/or truncated forms of any of the proteins disclosed in Table 3B. In various embodiments, the present invention contemplates the targeting of the precursor forms and/or mature forms and/or isoforms of any of the proteins disclosed in Table 3A, Table 3B, and/or Table 3C.

In various embodiments, the present invention contemplates the targeting of a protein having about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the protein sequences disclosed herein (e.g. in Table 3A, Table 3B, and/or Table 3C).

In various embodiments, the present invention contemplates the targeting of a protein comprising an amino acid sequence having one or more amino acid mutations relative to any of the protein sequences described herein (e.g. in Table 3A, Table 3B, and/or Table 3C). For example, the present invention contemplates the targeting of a protein comprising an amino acid sequence having 1, or 2, or 3, or 4, or 5, or 6, or 7, or 8, or 9, or 10, or 11, or 12 amino acid mutations relative to any of the protein sequences described herein (e.g. in Table 3A, Table 3B, and/or Table 3C). In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In various embodiments, the nucleic acid drug, including synthetic RNA, is administered is a manner that it effects one or more of keratinocytes and fibroblasts (e.g. causes these cells to express one or more therapeutic proteins). For example, present methods allow for methods in which a patient's cells are used to generate a therapeutic protein and the levels of such protein are tailored by synthetic RNA dosing.

The dose of a nucleic acid drug is disclosed herein. In general, the dose of any additional agent that is useful is known to those in the art. For example, doses may be determined with reference Physicians' Desk Reference, 66th Edition, PDR Network; 2012 Edition (Dec. 27, 2011), the contents of which are incorporated by reference in its entirety. In some embodiments, the present invention allows a patient to receive doses that exceed those determined with reference Physicians' Desk Reference. The dosage of any additional agent described herein can depend on several factors including the severity of the condition, whether the condition is to be treated or prevented, and the age, weight, and health of the human patient to be treated. Additionally, pharmacogenomic (the effect of genotype on the pharmacokinetic, pharmacodynamic or efficacy profile of a therapeutic) information about a particular human patient may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

Cells, tissues, organs, and organisms, including, but not limited to, humans, have several characteristics that can inhibit or prevent the delivery of nucleic acids, including, for example, the stratum corneum, which can serve as a barrier to foreign organisms and nucleic acids. These characteristics can thus inhibit the effects of therapeutics and cosmetics comprising nucleic acids. It has now been discovered that many of these characteristics can be circumvented or overcome using a patch comprising a flexible membrane and a plurality of needles, and that such a patch can serve as an effective and safe article for the delivery of nucleic acids. Certain embodiments are therefore directed to a nucleic acid delivery patch. In one embodiment, the nucleic acid delivery patch comprises a flexible membrane. In another embodiment, the nucleic acid delivery patch comprises a plurality of needles. In yet another embodiment, the plurality of needles is attached to the flexible membrane. In some embodiments, the patch comprises a nucleic acid. In one embodiment, the nucleic acid is present in solution. In one embodiment, the plurality of needles includes one or more needles having a lumen. In another embodiment, the patch further comprises a second flexible membrane. In yet another embodiment, the flexible membrane and the second flexible membrane are arranged to form a cavity. In a further embodiment, the cavity contains a nucleic acid. In a still further embodiment, the membrane comprises one or more holes through which a nucleic acid can pass. In a still further embodiment, one or more holes and one or more needles having alumen are arranged to allow the passage of a solution containing a nucleic acid through at least one of the one or more holes and through at least one of the one or more needles having a lumen. In some embodiments, the patch is configured to deliver a solution to the skin. In one embodiment, the solution comprises a nucleic acid. In another embodiment, the solution comprises a vehicle. In yet another embodiment, the vehicle is a lipid or lipidoid. In a still further embodiment, the vehicle is a lipid-based transfection reagent.

The cell membrane can serve as a barrier to foreign nucleic acids. It has now been discovered that combining the patch of the present invention with an electric field can increase the efficiency of nucleic acid delivery. Certain embodiments are therefore directed to a nucleic acid delivery patch comprising a plurality of needles, wherein at least two needles form part of a high-voltage circuit. Certain embodiments are directed to an implantable “tattoo” for microneedle delivery (see, e.g. Nature Materials 12, pp 367-376 (2013), the contents of which are hereby incorporated by reference in their entirety). In one embodiment, the high-voltage circuit generates a voltage greater than about 10V. In another embodiment, the high-voltage circuit generates a voltage greater than about 20V. In yet another embodiment, an electric field is produced between two of the needles. In a further embodiment, the magnitude of the electric field is at least about 100V/cm. In a still further embodiment, the magnitude of the electric field is at least about 200V/cm. In some embodiments, the patch is configured to deliver a nucleic acid to the epidermis. In other embodiments, the patch is configured to deliver a nucleic acid to the dermis. In still other embodiments, the patch is configured to deliver a nucleic acid to sub-dermal tissue. In still other embodiments, the patch is configured to deliver a nucleic acid to muscle. Certain embodiments are directed to a nucleic acid delivery patch comprising a plurality of electrodes. In one embodiment, the plurality of electrodes is attached to a flexible membrane. Other embodiments are directed to a nucleic acid delivery patch comprising a rigid structure. In one embodiment, a plurality of electrodes is attached to the rigid structure.

In some embodiments, the compositions described herein are administered using an array of needles covering an affected area of the subject. In some embodiments, the treatment area is mechanically massaged after administration. In some embodiments, the treatment area is exposed to electric pulses after administration. In some embodiments, the electric pulses are between about 10V and about 200V for from about 50 microseconds to about 1 second. In some embodiments, the electric pulses are generated around the treatment area by a multielectrode array.

In some embodiments, the present invention provides a patch delivery system, comprising a non-viral RNA transfection composition enclosed within a membrane, and an array of delivery needles delivering from about 10 ng to about 2000 ng of RNA per treatment area of about 100 cm² or less, or about 50 cm² or less, or about 10 cm² or less, or about 5 cm² or less, or about 1 cm² or less, or about 0.5 cm² or less, or about 0.2 cm² or less. In some embodiments, the non-viral transfection composition contains from about 10 ng to about 2000 ng per injection volume of about 20 μL to about 1 ml. In some embodiments, each needle delivers an injection volume of between 1 μL and 500 μL.

In some embodiments, the delivery patch comprises an acrylic reservoir that holds the nucleic acid drug. In some embodiments, a silicon adhesive is added to create a semisolid suspension of microscopic, concentrated drug cells. Further, some embodiments pride a patch that is associated with one or more enhancers (these include, without limitation, iontophoresis, ultrasound, chemicals including gels, microneedles, sonophoresis, lasers, and electroporatic methods).

In some embodiments, the delivery is effected via a gel, optionally a hydro alcoholic gel containing a combination of enhancers (e.g. COMBIGEL (ANTARES PHARMA)).

In various embodiments, the RNA is delivered using needle arrays. Illustrative needle arrays include, but are not limited to AdminPen 600 and those described in U.S. Pat. Nos. 7,658,728, 7,785,301, and 8,414,548, the entire disclosure of which are hereby incorporated by reference. Other examples of needles include, for example, the 3M™ Hollow Microstructured Transdermal System and the 3M Solid Microstructured Transdermal Systems (sMTS). See, e.g. U.S. Pat. Nos. 3,034,507 and 3,675,766; Microneedles for Transdermal Drug Delivery. Advanced Drug Delivery Reviews. 56: 581-587 (2004); Pharm Res. 2011 January; 28(1): 31-40, the entire contents of which are hereby incorporated by reference in their entireties.

In some embodiments, microneedles and/or microneedle arrays may be used. In various embodiments, the microneedles and/or microneedle arrays may be, without limitation, solid, RNA-coated, dissolving, biodegradable, and/or hollow. In some embodiments, the delivery is effected via a microneedle system, optionally combined with an electronically controlled micropump that delivers the drug at specific times or upon demand. For example, the MACROFLUX (Alza) system may be used.

In another embodiment, the method further comprises contacting the cell with one or more nucleic acid molecules.

In yet another embodiment, at least one of the one or more nucleic acid molecules encodes a protein of interest.

In a further embodiment, the method results in the cell expressing the protein of interest. In a still further embodiment, the method results in the cell expressing a therapeutically or cosmetically effective amount of the protein of interest.

In another embodiment, the cell is contacted with a nucleic acid molecule. In yet another embodiment, the method results in the cell internalizing the nucleic acid molecule. In a further embodiment, the method results in the cell internalizing a therapeutically or cosmetically effective amount of the nucleic acid molecule. In one embodiment, the nucleic acid encodes a protein of interest. In one embodiment, the nucleic acid molecule comprises a member of the group: a dsDNA molecule, a ssDNA molecule, a RNA molecule, a dsRNA molecule, a ssRNA molecule, a plasmid, an oligonucleotide, a synthetic RNA molecule, a miRNA molecule, an mRNA molecule, and an siRNA molecule. In various embodiments, the RNA comprises one or more non-canonical nucleotides.

In some embodiments, the present invention relates to one or more administration techniques described in U.S. Pat. Nos. 5,711,964; 5,891,468; 6,316,260; 6,413,544; 6,770,291; and 7,390,780, the entire contents of which are hereby incorporated by reference in their entireties.

The invention also provides kits that can simplify the administration of the nucleic acid drugs described herein and/or any additional agent described herein. An illustrative kit of the invention comprises a nucleic acid drug and/or any additional agent described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit or one or more components of the kit may be stored at room temperature, about 4° C., about −20° C., about −80° C., or about −196° C. The kit may also include a lid speculum, topical anesthetic, and a cleaning agent for the administration location. The kit can also further comprise one or more additional agent described herein. In one embodiment, the kit comprises a container containing an effective amount of a nucleic acid drug as disclosed herein and an effective amount of another composition, such as an additional agent as described herein. In some embodiments, the unit dosage form is a pre-loaded (a.k.a. pre-dosed or pre-filled) syringe or a pen needle injector (injection pen)). Such unit dosage forms may comprise the effective doses of nucleic acid drug described herein, e.g. about 10 ng to about 2000 ng, e.g. about 10 ng, or about 20 ng, or about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng.

Some embodiments are directed to synthetic RNA molecules with low toxicity and high translation efficiency. Other embodiments are directed to a cell-culture medium for high-efficiency in vivo transfection, reprogramming, and gene editing of cells. Other embodiments pertain to methods for producing synthetic RNA molecules encoding reprogramming proteins. Still further embodiments pertain to methods for producing synthetic RNA molecules encoding gene-editing proteins.

Some embodiments are directed to methods of gene-editing and/or gene correction. Some embodiments encompass synthetic RNA-based gene-editing and/or gene correction, e.g. with RNA comprising non-canonical nucleotides, e.g. RNA encoding one or more of a nuclease, a transcription activator-like effector nuclease (TALEN), a zinc-finger nuclease, a meganuclease, a nickase, a clustered regularly interspaced short palindromic repeat (CRISPR)-associated protein a DNA-repair protein, a DNA-modification protein, a base-modification protein, a DNA methyltransferase, an protein that causes DNA demethylation, an enzyme for which DNA is a substrate or a natural or engineered variant, family-member, orthologue, fragment or fusion construct thereof. In some embodiments, the efficiency of the gene-editing and/or gene correction is high, for example, higher than DNA-based gene editing and/or gene correction. In some embodiments, the present methods of gene-editing and/or gene correction are efficient enough for in vivo application. In some embodiments, the present methods of gene-editing and/or gene correction are efficient enough to not require cellular selection (e.g. selection of cells that have been edited). In various embodiments, the efficiency of gene-editing of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100%. In various embodiments, the efficiency of gene-correction of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100% Some embodiments are directed to high-efficiency gene-editing proteins comprising engineered nuclease cleavage or DNA-modification domains. Other embodiments are directed to high-fidelity gene-editing proteins comprising engineered nuclease cleavage or DNA-modification domains. Various embodiments are directed to high-efficiency gene-editing proteins comprising engineered DNA-binding domains. Other embodiments are directed to high-fidelity gene-editing proteins comprising engineered DNA-binding domains. Still other embodiments are directed to gene-editing proteins comprising engineered repeat sequences. Some embodiments are directed to gene-editing proteins comprising one or more CRISPR associated family members. Some embodiments are directed to methods for altering the DNA sequence of a cell by transfecting the cell with or inducing the cell to express a gene-editing protein. Other embodiments are directed to methods for altering the DNA sequence of a cell that is present in an in vitro culture. Still further embodiments are directed to methods for altering the DNA sequence of a cell that is present in vivo.

It has been further discovered that in certain situations, including one or more steroids and/or one or more antioxidants in the transfection medium can increase in vivo transfection efficiency, in vivo reprogramming efficiency, and in vivo gene-editing efficiency. Certain embodiments are therefore directed to contacting a cell or patient with a glucocorticoid, such as hydrocortisone, prednisone, prednisolone, methylprednisolone, dexamethasone or betamethasone. Other embodiments are directed to a method for inducing a cell to express a protein of interest by contacting a cell with a medium containing a steroid and contacting the cell with one or more nucleic acid molecules. In one embodiment, the nucleic acid molecule comprises synthetic RNA. In another embodiment, the steroid is hydrocortisone. In yet another embodiment, the hydrocortisone is present in the medium at a concentration of between about 0.1 uM and about 10 uM, or about 1 uM. Other embodiments are directed to a method for inducing a cell in vivo to express a protein of interest by contacting the cell with a medium containing an antioxidant and contacting the cell with one or more nucleic acid molecules. In one embodiment, the antioxidant is ascorbic acid or ascorbic-acid-2-phosphate. In another embodiment, the ascorbic acid or ascorbic-acid-2-phosphate is present in the medium at a concentration of between about 0.5 mg/L and about 500 mg/L, including about 50 mg/L. Still other embodiments are directed to a method for reprogramming and/or gene-editing a cell in vivo by contacting the cell with a medium containing a steroid and/or an antioxidant and contacting the cell with one or more nucleic acid molecules, wherein the one or more nucleic acid molecules encodes one or more reprogramming and/or gene-editing proteins. In certain embodiments, the cell is present in an organism, and the steroid and/or antioxidant are delivered to the organism.

Adding transferrin to the complexation medium has been reported to increase the efficiency of plasmid transfection in certain situations. It has now been discovered that adding transferrin to the complexation medium can also increase the efficiency of in vivo transfection with synthetic RNA molecules. Certain embodiments are therefore directed to a method for inducing a cell in vivo to express a protein of interest by adding one or more synthetic RNA molecules and a transfection reagent to a solution containing transferrin. In one embodiment, the transferrin is present in the solution at a concentration of between about 1 mg/L and about 100 mg/L, such as about 5 mg/L. In another embodiment, the transferrin is recombinant.

In certain situations, including pertaining to culturing, it may be desirable to replace animal-derived components with non-animal-derived and/or recombinant components, in part because non-animal-derived and/or recombinant components can be produced with a higher degree of consistency than animal-derived components, and in part because non-animal-derived and/or recombinant components carry less risk of contamination with toxic and/or pathogenic substances than do animal-derived components. Certain embodiments are therefore directed to a protein that is non-animal-derived and/or recombinant. Other embodiments are directed to a medium, wherein some or all of the components of the medium are non-animal-derived and/or recombinant.

Other embodiments are directed to a method for transfecting a cell in vivo. In one embodiment, a cell in vivo is transfected with one or more nucleic acids, and the transfection is performed using a transfection reagent, such as a lipid-based transfection reagent. In one embodiment, the one or more nucleic acids includes at least one RNA molecule. In another embodiment, the cell is transfected repeatedly, such as at least about 2 times during about 10 consecutive days, or at least about 3 times during about 7 consecutive days, or at least about 4 times during about 6 consecutive days. Some embodiments are directed to a method for increasing expression of telomerase in one of a fibroblast, a hematopoietic stem cell, a mesenchymal stem cells, a cardiac stem cell, a hair follicle stem cell, a neural stem cell, an intestinal stem cell, an endothelial stem cell, an olfactory stem cell, a neural crest stem cell, a testicular cell, and a keratinocyte. Some embodiments are directed to a method for increasing the length of telomeres in one of a fibroblast, a hematopoietic stem cell, a mesenchymal stem cells, a cardiac stem cell, a hair follicle stem cell, a neural stem cell, an intestinal stem cell, an endothelial stem cell, an olfactory stem cell, a neural crest stem cell, a testicular cell, and a keratinocyte. Other embodiments are directed to a method for isolating a cell from a patient, contacting the cell with a nucleic acid drug encoding a component of telomerase (e.g., TERT), and reintroducing the cell to the patient. Various embodiments are directed to a method for increasing the replicative potential of a cell.

### Reprogramming

Cells can be reprogrammed by exposing them to specific extracellular cues and/or by ectopic expression of specific proteins, microRNAs, etc. While several reprogramming methods have been previously described, most that rely on ectopic expression require the introduction of exogenous DNA, which can carry mutation risks. DNA-free reprogramming methods based on direct delivery of reprogramming proteins have been reported. However, these methods are too inefficient and unreliable for commercial use. In addition, RNA-based reprogramming methods have been described (see, e.g., Angel. MIT Thesis. 2008. 1-56; Angel et al. PLoS ONE. 2010. 5, 107; Warren et al. Cell Stem Cell. 2010. 7, 618-630; Angel. MIT Thesis. 2011. 1-89; and Lee et al., Cell. 2012. 151, 547-558; the contents of all of which are hereby incorporated by reference). However, existing RNA-based reprogramming methods are slow, unreliable, and inefficient when performed on adult cells, require many transfections (resulting in significant expense and opportunity for error), can reprogram only a limited number of cell types, can reprogram cells to only a limited number of cell types, require the use of immunosuppressants, and require the use of multiple human-derived components, including blood-derived HSA and human fibroblast feeders. The many drawbacks of previously disclosed RNA-based reprogramming methods make them undesirable for research, therapeutic or cosmetic use.

Reprogramming can be performed by transfecting cells with one or more nucleic acids encoding one or more reprogramming factors. Examples of reprogramming factors include, but are not limited to Oct4 protein, Sox2 protein, Klf4 protein, c-Myc protein, I-Myc protein, TERT protein, Nanog protein, Lin28 protein, Utf1 protein, Aicda protein, miR200 micro-RNA, miR302 micro-RNA, miR367 micro-RNA, miR369 micro-RNA and biologically active fragments, analogues, variants and family-members thereof. Certain embodiments are therefore directed to a method for reprogramming a cell in vivo. In one embodiment, the cell in vivo is reprogrammed by transfecting the cell with one or more nucleic acids encoding one or more reprogramming factors. In one embodiment, the one or more nucleic acids includes an RNA molecule that encodes Oct4 protein. In another embodiment, the one or more nucleic acids also includes one or more RNA molecules that encodes Sox2 protein, Klf4 protein, and c-Myc protein. In yet another embodiment, the one or more nucleic acids also includes an RNA molecule that encodes Lin28 protein. In one embodiment, the cell is a human skin cell, and the human skin cell is reprogrammed to a pluripotent stem cell. In another embodiment, the cell is a human skin cell, and the human skin cell is reprogrammed to a glucose-responsive insulin-producing cell. Examples of other cells that can be reprogrammed and other cells to which a cell can be reprogrammed include, but are not limited to skin cells, pluripotent stem cells, mesenchymal stem cells, β-cells, retinal pigmented epithelial cells, hematopoietic cells, cardiac cells, airway epithelial cells, neural stem cells, neurons, glial cells, bone cells, blood cells, and dental pulp stem cells. In one embodiment, the cell is contacted with a medium that supports the reprogrammed cell. In one embodiment, the medium also supports the cell.

Importantly, infecting skin cells with viruses encoding Oct4, Sox2, Klf4, and c-Myc, combined with culturing the cells in a medium that supports the growth of cardiomyocytes, has been reported to cause reprogramming of the skin cells to cardiomyocytes, without first reprogramming the skin cells to pluripotent stem cells (See Efs et al Nat Cell Biol. 2011; 13:215-22, the contents of which are hereby incorporated by reference). In certain situations, direct reprogramming (reprogramming one somatic cell to another somatic cell without first reprogramming the somatic cell to a pluripotent stem cell, also known as “transdifferentiation”) may be desirable, in part because culturing pluripotent stem cells can be time-consuming and expensive, the additional handling involved in establishing and characterizing a stable pluripotent stem cell line can carry an increased risk of contamination, and the additional time in culture associated with first producing pluripotent stem cells can carry an increased risk of genomic instability and the acquisition of mutations, including point mutations, copy-number variations, and karyotypic abnormalities. Certain embodiments are therefore directed to a method for reprogramming a somatic cell in vivo, wherein the cell is reprogrammed to a somatic cell, and wherein a characterized pluripotent stem-cell line is not produced.

It has been further discovered that, in certain situations, fewer total transfections may be required to reprogram a cell according to the methods of the present invention than according to other methods. Certain embodiments are therefore directed to a method for reprogramming a cell in vivo, wherein between about 1 and about 12 transfections are performed during about 20 consecutive days, or between about 4 and about 10 transfections are performed during about 15 consecutive days, or between about 4 and about 8 transfections are performed during about 10 consecutive days. It is recognized that when a cell is contacted with a medium containing nucleic acid molecules, the cell may likely come into contact with and/or internalize more than one nucleic acid molecule either simultaneously or at different times. A cell can therefore be contacted with a nucleic acid more than once, e.g. repeatedly, even when a cell is contacted only once with a medium containing nucleic acids.

Of note, nucleic acids can contain one or more non-canonical or “modified” residues as described herein. For instance, any of the non-canonical nucleotides described herein can be used in the present reprogramming methods. In one embodiment, pseudouridine-5′-triphosphate can be substituted for uridine-5′-triphosphate in an in vitro-transcription reaction to yield synthetic RNA, wherein up to 100% of the uridine residues of the synthetic RNA may be replaced with pseudouridine residues. In vitro-transcription can yield RNA with residual immunogenicity, even when pseudouridine and 5-methylcytidine are completely substituted for uridine and cytidine, respectively (see, e.g., Angel. Reprogramming Human Somatic Cells to Pluripotency Using RNA [Doctoral Thesis]. Cambridge, MA: MIT; 2011, the contents of which are hereby incorporated by reference). For this reason, it is common to add an immunosuppressant to the transfection medium when transfecting cells with RNA. In certain situations, adding an immunosuppressant to the transfection medium may not be desirable, in part because the recombinant immunosuppressant most commonly used for this purpose, B18R, can be expensive and difficult to manufacture. It has now been discovered that cells in vivo can be transfected and/or reprogrammed according to the methods of the present invention, without using B18R or any other immunosuppressant. It has been further discovered that reprogramming cells in vivo according to the methods of the present invention without using immunosuppressants can be rapid, efficient, and reliable. Certain embodiments are therefore directed to a method for transfecting a cell in vivo, wherein the transfection medium does not contain an immunosuppressant. Other embodiments are directed to a method for reprogramming a cell in vivo, wherein the transfection medium does not contain an immunosuppressant. In certain situations, for example when using a high cell density, it may be beneficial to add an immunosuppressant to the transfection medium. Certain embodiments are therefore directed to a method for transfecting a cell in vivo, wherein the transfection medium contains an immunosuppressant. Other embodiments are directed to a method for reprogramming a cell in vivo, wherein the transfection medium contains an immunosuppressant. In one embodiment, the immunosuppressant is B18R or a biologically active fragment, analogue, variant or family-member thereof or dexamethasone or a derivative thereof. In one embodiment, the transfection medium does not contain an immunosuppressant, and the nucleic-acid dose is chosen to prevent excessive toxicity. In another embodiment, the nucleic-acid dose is less than about 1 mg/cm² of tissue or less than about 1 mg/100,000 cells or less than about 10 mg/kg.

Reprogrammed cells produced according to certain embodiments of the present invention are suitable for therapeutic and/or cosmetic applications as they do not contain undesirable exogenous DNA sequences, and they are not exposed to animal-derived or human-derived products, which may be undefined, and which may contain toxic and/or pathogenic contaminants. Furthermore, the high speed, efficiency, and reliability of certain embodiments of the present invention may reduce the risk of acquisition and accumulation of mutations and other chromosomal abnormalities. Certain embodiments of the present invention can thus be used to generate cells that have a safety profile adequate for use in therapeutic and/or cosmetic applications. For example, reprogramming cells using RNA and the medium of the present invention, wherein the medium does not contain animal or human-derived components, can yield cells that have not been exposed to allogeneic material. Certain embodiments are therefore directed to a reprogrammed cell that has a desirable safety profile. In one embodiment, the reprogrammed cell has a normal karyotype. In another embodiment, the reprogrammed cell has fewer than about 5 copy-number variations (CNVs) relative to the patient genome, such as fewer than about 3 copy-number variations relative to the patient genome, or no copy-number variations relative to the patient genome. In yet another embodiment, the reprogrammed cell has a normal karyotype and fewer than about 100 single nucleotide variants in coding regions relative to the patient genome, or fewer than about 50 single nucleotide variants in coding regions relative to the patient genome, or fewer than about 10 single nucleotide variants in coding regions relative to the patient genome.

Endotoxins and nucleases can co-purify and/or become associated with other proteins, such as serum albumin. Recombinant proteins, in particular, can often have high levels of associated endotoxins and nucleases, due in part to the lysis of cells that can take place during their production. Endotoxins and nucleases can be reduced, removed, replaced or otherwise inactivated by many of the methods of the present invention, including, for example, by acetylation, by addition of a stabilizer such as sodium octanoate, followed by heat treatment, by the addition of nuclease inhibitors to the albumin solution and/or medium, by crystallization, by contacting with one or more ion-exchange resins, by contacting with charcoal, by preparative electrophoresis or by affinity chromatography. It has now been discovered that partially or completely reducing, removing, replacing, or otherwise inactivating endotoxins and/or nucleases from a medium and/or from one or more components of a medium can increase the efficiency with which cells can be transfected and reprogrammed. Certain embodiments are therefore directed to a method for transfecting a cell in vivo with one or more nucleic acids, wherein the transfection medium is treated to partially or completely reduce, remove, replace or otherwise inactivate one or more endotoxins and/or nucleases.

Other embodiments are directed to a medium that causes minimal degradation of nucleic acids. In one embodiment, the medium contains less than about 1 EU/mL, or less than about 0.1 EU/mL, or less than about 0.01 EU/mL.

In certain situations, protein-based lipid carriers such as serum albumin can be replaced with non-protein-based lipid carriers such as methyl-beta-cyclodextrin. The medium of the present invention can also be used without a lipid carrier, for example, when transfection is performed using a method that may not require or may not benefit from the presence of a lipid carrier, for example, using one or more lipid-based transfection reagents, polymer-based transfection reagents or peptide-based transfection reagents or using electroporation. Many protein-associated molecules, such as metals, can be highly toxic to cells in vivo. This toxicity can cause decreased viability, as well as the acquisition of mutations. Certain embodiments thus have the additional benefit of producing cells that are free from toxic molecules.

The associated-molecule component of a protein can be measured by suspending the protein in solution and measuring the conductivity of the solution. Certain embodiments are therefore directed to a medium that contains a protein, wherein about a 10% solution of the protein in water has a conductivity of less than about 500 μmho/cm. In one embodiment, the solution has a conductivity of less than about 50 μmho/cm. In another embodiment, less than about 0.65% of the dry weight of the protein comprises lipids and/or less than about 0.35% of the dry weight of the protein comprises free fatty acids.

The amount of nucleic acid delivered to cells in vivo can be increased to increase the desired effect of the nucleic acid. However, increasing the amount of nucleic acid delivered to cells in vivo beyond a certain point can cause a decrease in the viability of the cells, due in part to toxicity of the transfection reagent. It has now been discovered that when a nucleic acid is delivered to a population of cells in vivo in a fixed volume (for example, cells in a region of tissue), the amount of nucleic acid delivered to each cell can depend on the total amount of nucleic acid delivered to the population of cells and to the density of the cells, with a higher cell density resulting in less nucleic acid being delivered to each cell. In certain embodiments, a cell in vivo is transfected with one or more nucleic acids more than once. Under certain conditions, for example when the cells are proliferating, the cell density may change from one transfection to the next. Certain embodiments are therefore directed to a method for transfecting a cell in vivo with a nucleic acid, wherein the cell is transfected more than once, and wherein the amount of nucleic acid delivered to the cell is different for two of the transfections. In one embodiment, the cell proliferates between two of the transfections, and the amount of nucleic acid delivered to the cell is greater for the second of the two transfections than for the first of the two transfections. In another embodiment, the cell is transfected more than twice, and the amount of nucleic acid delivered to the cell is greater for the second of three transfections than for the first of the same three transfections, and the amount of nucleic acid delivered to the cells is greater for the third of the same three transfections than for the second of the same three transfections. In yet another embodiment, the cell is transfected more than once, and the maximum amount of nucleic acid delivered to the cell during each transfection is sufficiently low to yield at least about 80% viability for at least two consecutive transfections.

It has now been discovered that modulating the amount of nucleic acid delivered to a population of proliferating cells in vivo in a series of transfections can result in both an increased effect of the nucleic acid and increased viability of the cells. It has been further discovered that, in certain situations, when cells in vivo are contacted with one or more nucleic acids encoding one or more reprogramming factors in a series of transfections, the efficiency of reprogramming can be increased when the amount of nucleic acid delivered in later transfections is greater than the amount of nucleic acid delivered in earlier transfections, for at least part of the series of transfections. Certain embodiments are therefore directed to a method for reprogramming a cell in vivo, wherein one or more nucleic acids is repeatedly delivered to the cell in a series of transfections, and the amount of the nucleic acid delivered to the cell is greater for at least one later transfection than for at least one earlier transfection. In one embodiment, the cell is transfected between about 2 and about 10 times, or between about 3 and about 8 times, or between about 4 and about 6 times. In another embodiment, the one or more nucleic acids includes at least one RNA molecule, the cell is transfected between about 2 and about 10 times, and the amount of nucleic acid delivered to the cell in each transfection is the same as or greater than the amount of nucleic acid delivered to the cell in the most recent previous transfection. In yet another embodiment, the amount of nucleic acid delivered to the cell in the first transfection is between about 20 ng/cm² and about 250 ng/cm², or between 100 ng/cm² and 600 ng/cm². In yet another embodiment, the cell is transfected about 5 times at intervals of between about 12 and about 48 hours, and the amount of nucleic acid delivered to the cell is about 25 ng/cm² for the first transfection, about 50 ng/cm² for the second transfection, about 100 ng/cm² for the third transfection, about 200 ng/cm² for the fourth transfection, and about 400 ng/cm² for the fifth transfection. In yet another embodiment, the cell is further transfected at least once after the fifth transfection, and the amount of nucleic acid delivered to the cell is about 400 ng/cm².

Certain embodiments are directed to a method for transfecting a cell in vivo with a nucleic acid, wherein the amount of nucleic acid is determined by measuring the cell density, and choosing the amount of nucleic acid to transfect based on the measurement of cell density. In one embodiment, the cell density is measured by optical means. In another embodiment, the cell is transfected repeatedly, the cell density increases between two transfections, and the amount of nucleic acid transfected is greater for the second of the two transfections than for the first of the two transfections.

It has now been discovered that, in certain situations, the amount of a circulating protein that is produced in a patient can be increased by administering to a patient a nucleic acid at a plurality of administration sites. In certain embodiments, the amount of a circulating protein is increased relative to the amount of the circulating protein that is produced in a patient by administering to the patient the nucleic acid at a single injection site. In one embodiment, the administering is by injection. In another embodiment, the injection is intradermal injection. In still another embodiment, the injection is subcutaneous or intramuscular injection. In some embodiments, the plurality of administration sites comprises administration sites in the skin. In other embodiments, the plurality of administration sites is at least about 1 or at least about 2 or at least about 5 or at least about 10 or at least about 20 or at least about 50 or at least about 100 administration sites. In one embodiment, the administering is performed within at least about 5 minutes or at least about 10 minutes or at least about 30 minutes or at least about 1 hour or at least about 2 hours or at least about 5 hours or at least about 12 hours or at least about 1 day. In certain embodiments, the amount of a circulating protein is increased by at least about 10 percent or at least about 20 percent or at least about 50 percent or at least about 100 percent or at least about 3-fold or at least about 5-fold or at least about 10-fold or at least about 20-fold or at least about 50-fold or at least about 100-fold or at least about 500-fold or at least about 1000-fold or greater than 1000-fold.

It has now been discovered that, in certain situations, the in vivo transfection efficiency and viability of cells contacted with the medium of the present invention can be improved by conditioning the medium. Certain embodiments are therefore directed to a method for conditioning a medium. Other embodiments are directed to a medium that is conditioned. In one embodiment, the feeders are fibroblasts, and the medium is conditioned for approximately 24 hours. Other embodiments are directed to a method for transfecting a cell in vivo, wherein the transfection medium is conditioned. Other embodiments are directed to a method for reprogramming and/or gene-editing a cell in vivo, wherein the medium is conditioned. In one embodiment, the feeders are mitotically inactivated, for example, by exposure to a chemical such as mitomycin-C or by exposure to gamma radiation. In certain embodiments, it may be beneficial to use only autologous materials, in part to, for example and not wishing to be bound by theory, avoid the risk of disease transmission from the feeders to the cell or the patient. Certain embodiments are therefore directed to a method for transfecting a cell in vivo, wherein the transfection medium is conditioned, and wherein the feeders are derived from the same individual as the cell being transfected. Other embodiments are directed to a method for reprogramming and/or gene-editing a cell in vivo, wherein the medium is conditioned, and wherein the feeders are derived from the same individual as the cell being reprogrammed and/or gene-edited.

Several molecules can be added to media by conditioning. Certain embodiments are therefore directed to a medium that is supplemented with one or more molecules that are present in a conditioned medium. In one embodiment, the medium is supplemented with Wnt1, Wnt2, Wnt3, Wnt3a or a biologically active fragment, analogue, variant, agonist, or family-member thereof. In another embodiment, the medium is supplemented with TGF-β or a biologically active fragment, analogue, variant, agonist, or family-member thereof. In yet another embodiment, a cell in vivo is reprogrammed according to the method of the present invention, wherein the medium is not supplemented with TGF-β for between about 1 and about 5 days, and is then supplemented with TGF-β for at least about 2 days. In yet another embodiment, the medium is supplemented with IL-6, IL-6R or a biologically active fragment, analogue, variant, agonist, or family-member thereof. In yet another embodiment, the medium is supplemented with a sphingolipid or a fatty acid. In still another embodiment, the sphingolipid is lysophosphatidic acid, lysosphingomyelin, sphingosine-1-phosphate or a biologically active analogue, variant or derivative thereof.

In addition to mitotically inactivating cells, under certain conditions, irradiation can change the gene expression of cells, causing cells to produce less of certain proteins and more of certain other proteins that non-irradiated cells, for example, members of the Wnt family of proteins. In addition, certain members of the Wnt family of proteins can promote the growth and transformation of cells. It has now been discovered that, in certain situations, the efficiency of reprogramming can be greatly increased by contacting a cell in vivo with a medium that is conditioned using irradiated feeders instead of mitomycin-c-treated feeders. It has been further discovered that the increase in reprogramming efficiency observed when using irradiated feeders is caused in part by Wnt proteins that are secreted by the feeders. Certain embodiments are therefore directed to a method for reprogramming a cell in vivo, wherein the cell is contacted with Wnt1, Wnt2, Wnt3, Wnt3a or a biologically active fragment, analogue, variant, family-member or agonist thereof, including agonists of downstream targets of Wnt proteins, and/or agents that mimic one or more of the biological effects of Wnt proteins, for example, 2-amino-4-[3,4-(methylenedioxy)benzylamino]-6-(3-methoxyphenyl)pyrimidine.

Because of the low efficiency of many DNA-based reprogramming methods, these methods may be difficult or impossible to use with cells derived from patient samples, which may contain only a small number of cells. In contrast, the high efficiency of certain embodiments of the present invention can allow reliable reprogramming of a small number of cells, including single cells. Certain embodiments are directed to a method for reprogramming a small number of cells. Other embodiments are directed to a method for reprogramming a single cell. In one embodiment, the cell is contacted with one or more enzymes. In another embodiment, the enzyme is collagenase. In yet another embodiment, the collagenase is animal-component free. In one embodiment, the collagenase is present at a concentration of between about 0.1 mg/mL and about 10 mg/mL, or between about 0.5 mg/mL and about 5 mg/mL. In another embodiment, the cell is a blood cell. In yet another embodiment, the cell is contacted with a medium containing one or more proteins that is derived from the patient's blood. In still another embodiment, the cell is contacted with a medium comprising: DMEM/F12+2 mM L-alanyl-L-glutamine+between about 5% and about 25% patient-derived serum, or between about 10% and about 20% patient-derived serum, or about 20% patient-derived serum.

It has now been discovered that, in certain situations, transfecting cells in vivo with a mixture of RNA encoding Oct4, Sox2, Klf4, and c-Myc using the medium of the present invention can cause the rate of proliferation of the cells to increase. When the amount of RNA delivered to the cells is too low to ensure that all of the cells are transfected, only afraction of the cells may show an increased proliferation rate. In certain situations, such as when generating a personalized therapeutic, increasing the proliferation rate of cells may be desirable, in part because doing so can reduce the time necessary to generate the therapeutic, and therefore can reduce the cost of the therapeutic. Certain embodiments are therefore directed to a method for transfecting a cell in vivo with a mixture of RNA encoding Oct4, Sox2, Klf4, and c-Myc. In one embodiment, the cell exhibits an increased proliferation rate. In another embodiment, the cell is reprogrammed.

Many diseases are associated with one or more mutations. Mutations can be corrected by contacting a cell with a nucleic acid that encodes a protein that, either alone or in combination with other molecules, corrects the mutation (an example of gene-editing). Examples of such proteins include: a nuclease, a transcription activator-like effector nuclease (TALEN), a zinc-finger nuclease, a meganuclease, a nickase, a clustered regularly interspaced short palindromic repeat (CRISPR)-associated protein a DNA-repair protein, a DNA-modification protein, a base-modification protein, a DNA methyltransferase, an protein that causes DNA demethylation, an enzyme for which DNA is a substrate or a natural or engineered variant, family-member, orthologue, fragment or fusion construct thereof. Certain embodiments are therefore directed to a method for transfecting a cell in vivo with a nucleic acid, wherein the nucleic acid encodes a protein that, either alone or in combination with other molecules, creates a single-strand or double-strand break in a DNA molecule. In a one embodiment, the protein is a zinc finger nuclease or a TALEN. In another embodiment, the nucleic acid is an RNA molecule. In yet another embodiment, the single-strand or double-strand break is within about 5,000,000 bases of the transcription start site of a gene selected from the group: SERPINA1, CCR5, CXCR4, GAD1, GAD2, CFTR, HBA1, HBA2, HBB, HBD, FANCA, XPA, XPB, XPC, ERCC2, POLH, HTT, DMD, SOD1, APOE, PRNP, BRCA1, and BRCA2 or an analogue, variant or family-member thereof. In one embodiment, the present invention relates to gene-editing of the MYC protein (e.g. correcting one or more mutations that may be linked to cancer), optionally with a TALEN. In yet another embodiment, the cell is transfected with a nucleic acid that acts as a repair template by either causing the insertion of a DNA sequence in the region of the single-strand or double-strand break or by causing the DNA sequence in the region of the single-strand or double-strand break to otherwise change. In yet another embodiment, the gene-editing protein contains a DNA modification domain. In yet another embodiment, the gene-editing protein corrects a mutation without creating a single-strand break. In yet another embodiment, the gene-editing protein corrects a mutation without creating a double-strand break. In yet another embodiment, the gene-editing protein corrects a mutation by causing the replacement of one base with another base. In one embodiment, adenine is replaced by cytosine. In another embodiment, adenine is replaced by guanine. In yet another embodiment, adenine is replaced by thymine. In yet another embodiment, cytosine is replaced by adenine. In yet another embodiment, cytosine is replaced by guanine. In yet another embodiment, cytosine is replaced by thymine. In yet another embodiment, guanine is replaced by adenine. In yet another embodiment, guanine is replaced by cytosine. In yet another embodiment, guanine is replaced by thymine. In yet another embodiment, thymine is replaced by adenine. In yet another embodiment, thymine is replaced by cytosine. In yet another embodiment, thymine is replaced by guanine. In one embodiment, the replacement of one base with another base is a one-step process. In another embodiment, the replacement of one base with another base is a multi-step process. In some embodiments, one base is replaced by more than one base, for example, by two bases. In other embodiments, more than one base is replaced by one base. In still other embodiments, more than one base is replaced by more than one base. In some embodiments, the gene-editing protein contains a deaminase domain. In one embodiment, the deaminase domain comprises a cytidine deaminase domain. In another embodiment, the deaminase domain comprises an adenosine deaminase domain. In yet another embodiment, the deaminase domain comprises a guanosine deaminase domain. In yet another embodiment, the gene-editing protein comprises a sequence that is at least about 50% or at least about 60% or at least about 70% or at least about 80% or at least about 90% or at least about 95% or at least about 99% homologous to one or more of SEQ ID NOs: 587, 588, 589, 590, 591, 592, and 593. In yet another embodiment, the gene-editing protein comprises a linker. In one embodiment, the linker is a flexible linker. In another embodiment, the linker positions the deaminase domain in proximity to a target base. In yet another embodiment, the gene-editing protein deaminates the target base. In yet another embodiment, the gene-editing protein comprises a glycosylase-inhibitor domain. In yet another embodiment, the gene-editing protein comprises glycosylase-inhibitor activity. In one embodiment, the glycosylase inhibitor is a uracil glycosylase inhibitor. In another embodiment, the glycosylase inhibitor is a N-methylpurine DNA glycosylase inhibitor. In yet another embodiment, the cell is reprogrammed, and subsequently, the cell is gene-edited. In yet another embodiment, the cell is gene-edited, and subsequently, the cell is reprogrammed. In yet another embodiment, the gene-editing and reprogramming are performed within about 7 days of each other. In yet another embodiment, the gene-editing and reprogramming occur simultaneously or on the same day. In yet another embodiment, the cell is a skin cell, the skin cell is gene-edited to disrupt the CCR5 gene, the skin cell is reprogrammed to a hematopoietic stem cell, thus producing a therapeutic for HIV/AIDS, and the therapeutic is used to treat a patient with HIV/AIDS. In yet another embodiment, the skin cell is derived from the same patient whom the therapeutic is used to treat.

Certain embodiments are directed to methods and compositions for the treatment of rare diseases. In some embodiments, the rare disease is one or more of a rare metabolic disease, a rare cardiovascular disease, a rare dermatologic disease, a rare neurologic disease, a rare developmental disease, a rare genetic disease, a rare pulmonary disease, a rare liver disease, a rare kidney disease, a rare psychiatric disease, a rare reproductive disease, a rare musculoskeletal disease, a rare orthopedic disease, an inborn error of metabolism, a lysosomal storage disease, and a rare ophthalmologic disease. In one embodiment, the disease is alpha-1-antitrypsin deficiency. Some embodiments are directed to a treatment comprising a nucleic acid encoding a gene-editing protein that is capable of causing a deletion in a gene that is associated with one or more of a gain-of-function mutation, a loss-of-function mutation, a recessive mutation, a dominant mutation or a dominant negative mutation. Other embodiments are directed to a treatment comprising a nucleic acid encoding a gene-editing protein that is capable of correcting one or more of a gain-of-function mutation, a loss-of-function mutation, a recessive mutation, a dominant mutation, or a dominant negative mutation. In one embodiment, the treatment ameliorates one or more of the symptoms in a subject. In another embodiment, the subject is a human subject. In yet another embodiment, the subject is a veterinary subject. Some embodiments are directed to a treatment for alpha-1-antitrypsin deficiency comprising administering to a subject a nucleic acid comprising a gene-editing protein that is capable of causing a deletion in or near the SERPINA1 gene. Other embodiments are directed to a treatment for alpha-1-antitrypsin deficiency comprising administering to a subject a nucleic acid encoding a gene-editing protein that is capable of correcting a mutation in or near the SERPINA1 gene. In one embodiment, the mutation is the Z mutation. In one embodiment, the deletion or correction reduces the accumulation of polymerized alpha-1-antitrypsin protein in the subject's cells and/or increases the secretion of alpha-1-antitrypsin from the subject's cells. In another embodiment, the treated cells regenerate a diseased organ. In yet another embodiment, the diseased organ is the liver. In yet another embodiment, the diseased organ is the lung. In yet another embodiment, the treatment delays or eliminates the subject's need for a liver and/or lung transplant. Other embodiments are directed to a treatment for epidermolysis bullosa. In one embodiment, the epidermolysis bullosa is dystrophic epidermolysis bullosa. In another embodiment, the epidermolysis bullosa is epidermolysis bullosa simplex. In yet another embodiment, the dystrophic epidermolysis bullosa is recessive dystrophic epidermolysis bullosa. In some embodiments, the treatment comprises administering to a subject a nucleic acid encoding a gene-editing protein that is capable of correcting a mutation in or near the COL7A1 gene. In one embodiment, the correction increases the amount of functional collagen VII produced by the subject's cells. In another embodiment, the treatment reduces the size, severity, and/or frequency of recurrence of skin lesions and/or blisters. Still other embodiments are directed to a treatment for primary hyperoxaluria. In one embodiment the primary hyperoxaluria is type I primary hyperoxaluria. In another embodiment, the treatment comprises administering to a subject a nucleic acid encoding a gene-editing protein that is capable of correcting a mutation in or near the AGXT gene. In yet another embodiment, the treatment delays or eliminates the subject's need for a kidney and/or liver transplant.

Genes that can be edited according to the methods of the present invention to produce therapeutics of the present invention include genes that can be edited to restore normal function, as well as genes that can be edited to reduce or eliminate function. Such genes include, but are not limited to alpha-1-antitrypsin (SERPINA1), mutations in which can cause alpha-1-antitrypsin deficiency, beta globin (HBB), mutations in which can cause sickle cell disease (SCD) and β-thalassemia, breast cancer 1, early onset (BRCA1) and breast cancer 2, early onset (BRCA2), mutations in which can increase susceptibility to breast cancer, C-C chemokine receptor type 5 (CCR5) and C-X-C chemokine receptor type 4 (CXCR4), mutations in which can confer resistance to HIV infection, cystic fibrosis transmembrane conductance regulator (CFTR), mutations in which can cause cystic fibrosis, dystrophin (DMD), mutations in which can cause muscular dystrophy, including Duchenne muscular dystrophy and Becker's muscular dystrophy, glutamate decarboxylase 1 and glutamate decarboxylase 2 (GAD1, GAD2), mutations in which can prevent autoimmune destruction of β-cells, hemoglobin alpha 1, hemoglobin alpha 2, and hemoglobin delta (HBA1, HBA2, and HBD), mutations in which can cause thalassemia, desmoplakin, keratin 5, keratin 14, plectin, integrin alpha-6, integrin beta-4, laminin subunit alpha-3, laminin subunit beta-3, laminin subunit gamma-2, collagen type VII alpha 1, collagen type XVII alpha 1, and matrix metalloproteinase-1 (DSP, KRT5, KRT14, PLEC1, ITGA6, ITGB4, LAMA3, LAMB3, LAMC2, COL7A1, COL17A1, and MMP1), mutations in which can cause epidermolysis bullosa, Huntington (HTT), mutations in which can cause Huntington's disease, superoxide dismutase 1 (SOD1), mutations in which can cause amyotrophic lateral sclerosis (ALS), XPA, XPB, XPC, XPD (ERCC6) and polymerase (DNA directed), eta (POLH), mutations in which can cause xeroderma pigmentosum, leucine-rich repeat kinase 2 (LRRK2), mutations in which can cause Parkinson's disease, and Fanconi anemia, complementation groups A, B, C, D1, D2, E, F, G, I, J, L, M, N, P (FANCA, FANCB, FANCC, FANCD1, FANCD2, FANCE, FANCF, FANCG, FANCI, FANCJ, FANCL, FANCM, FANCN, FANCP), and RAD51 homolog C (S. cerevisiae) (RAD51C), mutations in which can cause Fanconi anemia.

Certain embodiments are directed to a therapeutic comprising a nucleic acid. In one embodiment, the nucleic acid encodes one or more gene-editing proteins. Other embodiments are directed to a therapeutic comprising one or more cells that are transfected, reprogrammed, and/or gene-edited in vivo according to the methods of the present invention. In one embodiment, a cell is transfected, reprogrammed, and/or gene-edited, and the transfected, reprogrammed, and/or gene-edited cell is introduced into a patient. In another embodiment, the cell is harvested from the same patient into whom the transfected, reprogrammed and/or gene-edited cell is introduced. Examples of diseases that can be treated with therapeutics of the present invention include, but are not limited to Alzheimer's disease, spinal cord injury, amyotrophic lateral sclerosis, cystic fibrosis, heart disease, including ischemic and dilated cardiomyopathy, macular degeneration, Parkinson's disease, Huntington's disease, diabetes, sickle-cell anemia, thalassemia, Fanconi anemia, xeroderma pigmentosum, muscular dystrophy, severe combined immunodeficiency, hereditary sensory neuropathy, cancer, and HIV/AIDS. In certain embodiments, the therapeutic comprises a cosmetic. In one embodiment, a cell is harvested from a patient, the cell is reprogrammed and expanded to a large number of adipose cells to produce a cosmetic, and the cosmetic is introduced into the patient. In still another embodiment, the cosmetic is used for tissue reconstruction.

While detailed examples are provided herein for the production of specific types of cells and for the production of therapeutics comprising specific types of cells, it is recognized that the methods of the present invention can be used to produce many other types of cells, and to produce therapeutics comprising one or more of many other types of cells, for example, by reprogramming a cell according to the methods of the present invention, and culturing the cell under conditions that mimic one or more aspects of development by providing conditions that resemble the conditions present in the cellular microenvironment during development.

Certain embodiments are directed to a library of cells with a variety of human leukocyte antigen (HLA) types (“HLA-matched libraries”). An HLA-matched library may be beneficial in part because it can provide for the rapid production and/or distribution of therapeutics without the patient having to wait for a therapeutic to be produced from the patient's cells. Such a library may be particularly beneficial for the production of cosmetics and for the treatment of heart disease and diseases of the blood and/or immune system for which patients may benefit from the immediate availability of a therapeutic or cosmetic.

The DNA sequence of a cell can be altered by contacting the cell with a gene-editing protein or by inducing the cell to express a gene-editing protein. However, previously disclosed gene-editing proteins suffer from low binding efficiency and excessive off-target activity, which can introduce undesired mutations in the DNA of the cell, severely limiting their use in vivo, for example in therapeutic and cosmetic applications, in which the introduction of undesired mutations in a patient's cells could lead to the development of cancer. It has now been discovered that gene-editing proteins that comprise the Stsl endonuclease cleavage domain (SEQ ID NO: 1) can exhibit substantially lower off-target activity in vivo than previously disclosed gene-editing proteins, while maintaining a high level of on-target activity in vivo. Other novel engineered proteins have also been discovered that can exhibit high on-target activity in vivo, low off-target activity in vivo, small size, solubility, and other desirable characteristics when they are used as the nuclease domain of a gene-editing protein: Stsl-HA (SEQ ID NO: 2), Stsl-HA2 (SEQ ID NO: 3), Stsl-UHA (SEQ ID NO: 4), Stsl-UHA2 (SEQ ID NO: 5), Stsl-HF (SEQ ID NO: 6), and Stsl-UHF (SEQ ID NO: 7). Stsl-HA, Stsl-HA2 (high activity), Stsl-UHA, and Stsl-UHA2 (ultra-high activity) can exhibit higher on-target activity in vivo than both wild-type Stsl and wild-type FokI, due in part to specific amino-acid substitutions within the N-terminal region at the 34 and 61 positions, while Stsl-HF (high fidelity) and Stsl-UHF (ultra-high fidelity) can exhibit lower off-target activity in vivo than both wild-type Stsl and wild-type FokI, due in part to specific amino-acid substitutions within the C-terminal region at the 141 and 152 positions.

Certain embodiments are therefore directed to a protein. In some embodiments, the protein is present in vivo. In other embodiments, the protein comprises a nuclease domain. In one embodiment, the nuclease domain comprises one or more of the cleavage domain of FokI endonuclease (SEQ ID NO: 53), the cleavage domain of Stsl endonuclease (SEQ ID NO: 1), Stsl-HA (SEQ ID NO: 2), Stsl-HA2 (SEQ ID NO: 3), Stsl-UHA (SEQ ID NO: 4), Stsl-UHA2 (SEQ ID NO: 5), Stsl-HF (SEQ ID NO: 6), and Stsl-UHF (SEQ ID NO: 7) or a biologically active fragment or variant thereof.

It has also been discovered that engineered gene-editing proteins that comprise DNA-binding domains comprising certain novel repeat sequences can exhibit lower off-target activity in vivo than previously disclosed gene-editing proteins, while maintaining a high level of on-target activity in vivo. Certain of these engineered gene-editing proteins can provide several advantages over previously disclosed gene-editing proteins, including, for example, increased flexibility of the linker region connecting repeat sequences, which can result in increased binding efficiency. Certain embodiments are therefore directed to a protein comprising a plurality of repeat sequences. In one embodiment, at least one of the repeat sequences contains the amino-acid sequence: GabG, where “a” and “b” each represent any amino acid. In one embodiment, the protein is a gene-editing protein. In another embodiment, one or more of the repeat sequences are present in a DNA-binding domain. In a further embodiment, “a” and “b” are each independently selected from the group: H and G. In a still further embodiment, “a” and “b” are H and G, respectively. In one embodiment, the amino-acid sequence is present within about 5 amino acids of the C-terminus of the repeat sequence. In another embodiment, the amino-acid sequence is present at the C-terminus of the repeat sequence. In some embodiments, one or more G in the amino-acid sequence GabG is replaced with one or more amino acids other than G, for example A, H or GG. In one embodiment, the repeat sequence has a length of between about 32 and about 40 amino acids or between about 33 and about 39 amino acids or between about 34 and 38 amino acids or between about 35 and about 37 amino acids or about 36 amino acids or greater than about 32 amino acids or greater than about 33 amino acids or greater than about 34 amino acids or greater than about 35 amino acids. Other embodiments are directed to a protein comprising one or more transcription activator-like effector domains. In one embodiment, at least one of the transcription activator-like effector domains comprises a repeat sequence. Other embodiments are directed to a protein comprising a plurality of repeat sequences generated by inserting one or more amino acids between at least two of the repeat sequences of a transcription activator-like effector domain. In one embodiment, one or more amino acids is inserted about 1 or about 2 or about 3 or about 4 or about 5 amino acids from the C-terminus of at least one repeat sequence. Still other embodiments are directed to a protein comprising a plurality of repeat sequences, wherein about every other repeat sequence has a different length than the repeat sequence immediately preceding or following the repeat sequence. In one embodiment, every other repeat sequence is about 36 amino acids long. In another embodiment, every other repeat sequence is 36 amino acids long. Still other embodiments are directed to a protein comprising a plurality of repeat sequences, wherein the plurality of repeat sequences comprises at least two repeat sequences that are each at least 36 amino acids long, and wherein at least two of the repeat sequences that are at least 36 amino acids long are separated by at least one repeat sequence that is less than 36 amino acids long. Some embodiments are directed to a protein that comprises one or more sequences selected from, for example, SEQ ID NO: 54, SEQ ID NO: 55, SEQ ID NO: 56, SEQ ID NO: 57, SEQ ID NO: 58, SEQ ID NO: 59, and SEQ ID NO: 60.

Other embodiments are directed to a protein that comprises a DNA-binding domain. In some embodiments, the DNA-binding domain comprises a plurality of repeat sequences. In one embodiment, the plurality of repeat sequences enables high-specificity recognition of a binding site in a target DNA molecule. In another embodiment, at least two of the repeat sequences have at least about 50%, or about 60%, or about 70%, or about 80%, or about 90%, or about 95%, or about 98%, or about 99% homology to each other. In a further embodiment, at least one of the repeat sequences comprises one or more regions capable of binding to a binding site in a target DNA molecule. In a still further embodiment, the binding site comprises a defined sequence of between about 1 to about 5 bases in length. In one embodiment, the DNA-binding domain comprises a zinc finger. In another embodiment, the DNA-binding domain comprises a transcription activator-like effector (TALE). In a further embodiment, the plurality of repeat sequences includes at least one repeat sequence having at least about 50% or about 60% or about 70% or about 80% or about 90% or about 95% or about 98%, or about 99% homology to a TALE. In a still further embodiment, the gene-editing protein comprises a clustered regularly interspaced short palindromic repeat (CRISPR)-associated protein. In one embodiment, the gene-editing protein comprises a nuclear-localization sequence. In another embodiment, the nuclear-localization sequence comprises the amino-acid sequence: PKKKRKV (SEQ ID NO: 471). In one embodiment, the gene-editing protein comprises a mitochondrial-localization sequence. In another embodiment, the mitochondrial-localization sequence comprises the amino-acid sequence: LGRVIPRKIASRASLM (SEQ ID NO: 472). In one embodiment, the gene-editing protein comprises a linker. In another embodiment, the linker connects a DNA-binding domain to a nuclease domain. In a further embodiment, the linker is between about 1 and about 10 amino acids long. In some embodiments, the linker is about 1, about 2, or about 3, or about 4, or about 5, or about 6, or about 7, or about 8, or about 9, or about 10 amino acids long. In one embodiment, the gene-editing protein is capable of generating a nick or a double-strand break in a target DNA molecule.

Certain embodiments are directed to a method for modifying the genome of a cell in vivo, the method comprising introducing into a cell in vivo a nucleic acid molecule encoding a non-naturally occurring fusion protein comprising an artificial transcription activator-like (TAL) effector repeat domain comprising one or more repeat units 36 amino acids in length and an endonuclease domain, wherein the repeat domain is engineered for recognition of a predetermined nucleotide sequence, and wherein the fusion protein recognizes the predetermined nucleotide sequence. In one embodiment, the cell is a eukaryotic cell. In another embodiment, the cell is an animal cell. In a further embodiment, the cell is a mammalian cell. In a still further embodiment, the cell is a human cell. In one embodiment, the cell is a plant cell. In another embodiment, the cell is a prokaryotic cell. In some embodiments, the fusion protein introduces an endonucleolytic cleavage in a nucleic acid of the cell, whereby the genome of the cell is modified.

Certain embodiments are directed to a composition for altering the DNA sequence of a cell in vivo comprising a nucleic acid, wherein the nucleic acid encodes a gene-editing protein. Other embodiments are directed to a composition for altering the DNA sequence of a cell in vivo comprising a nucleic-acid mixture, wherein the nucleic-acid mixture comprises: a first nucleic acid that encodes a first gene-editing protein, and a second nucleic acid that encodes a second gene-editing protein. In one embodiment, the binding site of the first gene-editing protein and the binding site of the second gene-editing protein are present in the same target DNA molecule. In another embodiment, the binding site of the first gene-editing protein and the binding site of the second gene-editing protein are separated by less than about 50 bases, or less than about 40 bases, or less than about 30 bases or less than about 20 bases, or less than about 10 bases, or between about 10 bases and about 25 bases or about 15 bases. In one embodiment, the nuclease domain of the first gene-editing protein and the nuclease domain of the second gene-editing protein are capable of forming a dimer. In another embodiment, the dimer is capable of generating a nick or double-strand break in a target DNA molecule.

Certain embodiments are directed to a therapeutic composition. Other embodiments are directed to a cosmetic composition. In some embodiments, the composition comprises a repair template. In a further embodiment, the repair template is a single-stranded DNA molecule or a double-stranded DNA molecule.

Other embodiments are directed to an article of manufacture for synthesizing a protein or a nucleic acid encoding a protein. In one embodiment, the article is a nucleic acid. In another embodiment, the protein comprises a DNA-binding domain. In a further embodiment, the nucleic acid comprises a nucleotide sequence encoding a DNA-binding domain. In one embodiment, the protein comprises a nuclease domain. In another embodiment, the nucleic acid comprises a nucleotide sequence encoding a nuclease domain. In one embodiment, the protein comprises a plurality of repeat sequences. In another embodiment, the nucleic acid encodes a plurality of repeat sequences. In a further embodiment, the nuclease domain is selected from FokI, Stsl, Stsl-HA, Stsl-HA2, Stsl-UHA, Stsl-UHA2, Stsl-HF, and Stsl-UHF or a natural or engineered variant or biologically active fragment thereof. In one embodiment, the nucleic acid comprises an RNA-polymerase promoter. In another embodiment, the RNA-polymerase promoter is a T7 promoter or a SP6 promoter. In a further embodiment, the nucleic acid comprises a viral promoter. In one embodiment, the nucleic acid comprises an untranslated region. In another embodiment, the nucleic acid is an in vitro-transcription template.

Certain embodiments are directed to a method for inducing a cell to express a protein in vivo. Other embodiments are directed to a method for altering the DNA sequence of a cell in vivo comprising transfecting the cell in vivo with a gene-editing protein or inducing the cell to express a gene-editing protein in vivo. Still other embodiments are directed to a method for reducing the expression of a protein of interest in a cell in vivo. In one embodiment, the cell is induced to express a gene-editing protein, wherein the gene-editing protein is capable of creating a nick or a double-strand break in a target DNA molecule. In another embodiment, the nick or double-strand break results in inactivation of a gene. Still other embodiments are directed to a method for generating an inactive, reduced-activity or dominant-negative form of a protein in vivo. In one embodiment, the protein is survivin. Still other embodiments are directed to a method for repairing one or more mutations in a cell in vivo. In one embodiment, the cell is contacted with a repair template. In another embodiment, the repair template is a DNA molecule. In a further embodiment, the repair template does not contain a binding site of the gene-editing protein. In a still further embodiment, the repair template encodes an amino-acid sequence that is encoded by a DNA sequence that comprises a binding site of the gene-editing protein.

In various embodiments, the repair template is about 20 nucleotides, or about 30 nucleotides, or about 40 nucleotides, or about 50 nucleotides, or about 60 nucleotides, or about 70 nucleotides, or about 80 nucleotides, or about 90 nucleotides, or about 100 nucleotides, or about 150 nucleotides, or about 200 nucleotides, or about 300 nucleotides, or about 400 nucleotides, or about 500 nucleotides, or about 750 nucleotides, or about 1000 nucleotides. In various embodiments, the repair template is about 20-1000 nucleotides, or about 20-500 nucleotides, or about 20-400 nucleotides, or about 20-200 nucleotides, or about 20-100 nucleotides, or about 80-100 nucleotides, or about 50-100 nucleotides.

In various embodiments, the mass ratio of RNA (e.g. synthetic RNA encoding gene-editing protein) to repair template is about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

In various embodiments, the molar ratio of RNA (e.g. synthetic RNA encoding gene-editing protein) to repair template is about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

In various embodiments, the repair template has a dual function, causing a repair to a gene-edited target sequence and preventing further binding of a gene-editing protein, thereby reducing or eliminating further gene-editing (e.g. via the repair template causing a repair that renders what was the gene-editing protein binding site no longer suitable for gene-editing protein binding). Accordingly, in some embodiments, the present gene-editing methods are tunable to ensure a single gene-edit per target site.

Other embodiments are directed to a method for treating a patient comprising administering to the patient a therapeutically effective amount of a protein or a nucleic acid encoding a protein. In one embodiment, the treatment results in one or more of the patient's symptoms being ameliorated. Certain embodiments are directed to a method for treating a patient comprising: a. inducing a cell to express a protein of interest by transfecting the cell in vivo with a nucleic acid encoding the protein of interest and/or b. reprogramming the cell in vivo. In one embodiment, the cell is reprogrammed to a less differentiated state. In another embodiment, the cell is reprogrammed by transfecting the cell with one or more synthetic RNA molecules encoding one or more reprogramming proteins. In a further embodiment, the cell is differentiated. In a still further embodiment, the cell is differentiated into one of a skin cell, a glucose-responsive insulin-producing cell, a hematopoietic cell, a cardiac cell, a retinal cell, a renal cell, a neural cell, a stromal cell, a fat cell, a bone cell, a muscle cell, an oocyte, and a sperm cell. Other embodiments are directed to a method for treating a patient comprising: a. inducing a cell to express a gene-editing protein by transfecting the cell in vivo with a nucleic acid encoding a gene-editing protein and/or b. reprogramming the cell in vivo.

Other embodiments are directed to a complexation medium. In one embodiment, the complexation medium has a pH greater than about 7, or greater than about 7.2, or greater than about 7.4, or greater than about 7.6, or greater than about 7.8, or greater than about 8.0, or greater than about 8.2, or greater than about 8.4, or greater than about 8.6, or greater than about 8.8, or greater than about 9.0. In another embodiment, the complexation medium comprises transferrin. In a further embodiment, the complexation medium comprises DMEM. In a still further embodiment, the complexation medium comprises DMEM/F12. Still other embodiments are directed to a method for forming nucleic-acid-transfection-reagent complexes. In one embodiment, the transfection reagent is incubated with a complexation medium. In another embodiment, the incubation occurs before a mixing step. In a further embodiment, the incubation step is between about 5 seconds and about 5 minutes or between about 10 seconds and about 2 minutes or between about 15 seconds and about 1 minute or between about 30 seconds and about 45 seconds. In one embodiment, the transfection reagent is selected from Table 1. In another embodiment, the transfection reagent is a lipid or lipidoid. In a further embodiment, the transfection reagent comprises a cation. In a still further embodiment, the cation is a multivalent cation. In a still further embodiment, the transfection reagent is N1-[2-((1S)-1-[(3-aminopropyl)amino]-4-[di(3-amino-propyl)amino]butylcarboxamido)ethyl]-3,4-di[oleyloxy]-benzamide (a.k.a. MVL5) or a derivative thereof.

Certain embodiments are directed to a method for inducing a cell to express a protein by contacting the cell with a nucleic acid in vivo. In one embodiment, the cell is a mammalian cell. In another embodiment, the cell is a human cell or a rodent cell. Other embodiments are directed to a cell produced using one or more of the methods of the present invention. In one embodiment, the cell is present in a patient. In another embodiment, the cell is isolated from a patient. Other embodiments are directed to a screening library comprising a cell produced using one or more of the methods of the present invention. In one embodiment, the screening library is used for at least one of toxicity screening, including: cardiotoxicity screening, neurotoxicity screening, and hepatotoxicity screening, efficacy screening, high-throughput screening, high-content screening, and other screening.

Other embodiments are directed to a kit containing a nucleic acid. In one embodiment, the kit contains a delivery reagent (a.k.a. “transfection reagent”). In another embodiment, the kit is a reprogramming kit. In a further embodiment, the kit is a gene-editing kit. Other embodiments are directed to a kit for producing nucleic acids. In one embodiment, the kit contains at least two of pseudouridine-triphosphate, 5-methyluridine triphosphate, 5-methylcytidine triphosphate, 5-hydroxymethylcytidine triphosphate, N4-methylcytidine triphosphate, N4-acetylcytidine triphosphate, and 7-deazaguanosine triphosphate or one or more derivatives thereof. Other embodiments are directed to a therapeutic or cosmetic comprising a nucleic acid. In one embodiment, the therapeutic or cosmetic is a pharmaceutical composition. In another embodiment, the pharmaceutical composition is formulated. In a further embodiment, the formulation comprises an aqueous suspension of liposomes. Example liposome components are set forth in Table 1, and are given by way of example, and not by way of limitation. In one embodiment, the liposomes include one or more polyethylene glycol (PEG) chains. In another embodiment, the PEG is PEG2000. In a further embodiment, the liposomes include 1,2-distearoyl-sn-glycero-3-phosphoethanolamine (DSPE) or a derivative thereof. In one embodiment, the therapeutic comprises one or more ligands. In another embodiment, the therapeutic comprises at least one of androgen, CD30 (TNFRSF8), a cell-penetrating peptide, CXCR, estrogen, epidermal growth factor, EGFR, HER2, folate, insulin, insulin-like growth factor-I, interleukin-13, integrin, progesterone, stromal-derived-factor-1, thrombin, vitamin D, and transferrin or a biologically active fragment or variant thereof. Still other embodiments are directed to a therapeutic or cosmetic comprising a cell generated using one or more of the methods of the present invention. In one embodiment, the therapeutic is administered to a patient for the treatment of any of the diseases or disorders described herein, including by way of non-limitation, type 1 diabetes, heart disease, including ischemic and dilated cardiomyopathy, macular degeneration, Parkinson's disease, cystic fibrosis, sickle-cell anemia, thalassemia, Fanconi anemia, severe combined immunodeficiency, hereditary sensory neuropathy, xeroderma pigmentosum, Huntington's disease, muscular dystrophy, amyotrophic lateral sclerosis, Alzheimer's disease, cancer, and infectious diseases including: hepatitis and HIV/AIDS.

Other embodiments are directed to a method for reprogramming a cell in vivo. In one embodiment, the cell is reprogrammed by contacting the cell with one or more nucleic acids. In one embodiment, the cell is contacted with a plurality of nucleic acids encoding at least one of Oct4 protein, Sox2 protein, Klf4 protein, c-Myc protein, Lin28 protein or a biologically active fragment, variant or derivative thereof. In another embodiment, the cell is contacted with a plurality of nucleic acids encoding a plurality of proteins including: Oct4 protein, Sox2 protein, Klf4 protein, and c-Myc protein or one or more biologically active fragments, variants or derivatives thereof. Still other embodiments are directed to a method for gene editing a cell in vivo. In one embodiment, the cell is gene-edited by contacting the cell with one or more nucleic acids.

Certain embodiments are directed to a method for inducing a cell in vivo to express a protein of interest comprising contacting a cell in vivo with a solution comprising albumin that is treated with an ion-exchange resin or charcoal and one or more nucleic acid molecules, wherein at least one of the one or more nucleic acid molecules encodes a protein of interest. In one embodiment, the method results in the cell expressing the protein of interest. In another embodiment, the one or more nucleic acid molecules comprise a synthetic RNA molecule. In one embodiment, the cell is a skin cell. In another embodiment, the cell is a muscle cell. In yet another embodiment, the cell is a dermal fibroblast. In yet another embodiment, the cell is a myoblast. In one embodiment, the protein of interest is an extracellular matrix protein. In another embodiment, the protein of interest is selected from elastin, collagen, laminin, fibronectin, vitronectin, lysyl oxidase, elastin binding protein, a growth factor, fibroblast growth factor, transforming growth factor beta, granulocyte colony-stimulating factor, a matrix metalloproteinase, an actin, fibrillin, microfibril-associated glycoprotein, a lysyl-oxidase-like protein, and platelet-derived growth factor. In one embodiment, the solution is delivered to the dermis. In another embodiment, the delivering is by injection. In yet another embodiment, the delivering is by injection using a microneedle array. In one embodiment, the solution further comprises a growth factor. In another embodiment, the growth factor is selected from fibroblast growth factor and transforming growth factor beta. In yet another embodiment, the solution further comprises cholesterol. Other embodiments are directed a method for inducing a cell in vivo to express a protein of interest comprising contacting a cell in vivo with a solution comprising cholesterol and one or more nucleic acid molecules, wherein at least one of the one or more nucleic acid molecules encodes a protein of interest. In one embodiment, the method results in the cell expressing the protein of interest. Still other embodiments are directed to a method for transfecting a cell in vivo with a nucleic acid molecule comprising contacting a cell in vivo with a solution comprising albumin that is treated with an ion-exchange resin or charcoal and a nucleic acid molecule. In one embodiment, the method results in the cell being transfected with the nucleic acid molecule. In another embodiment, the nucleic acid molecule is one of a dsDNA molecule, a ssDNA molecule, a dsRNA molecule, a ssRNA molecule, a plasmid, an oligonucleotide, a synthetic RNA molecule, a miRNA molecule, an mRNA molecule, an siRNA molecule. Still other embodiments are directed to a method for treating a patient comprising delivering to a patient a composition comprising albumin that is treated with an ion-exchange resin or charcoal and one or more nucleic acid molecules, wherein at least one of the one or more nucleic acid molecules encodes a protein of interest. In one embodiment, the method results in the expression of the protein of interest in the patient. In another embodiment, the method results in the expression of the protein of interest in the dermis of the patient.

Despite the tendency of transfection reagent nucleic acid complexes to precipitate, form clumps or otherwise degrade when stored for more than a few minutes, the present inventors have surprisingly discovered that transfection reagent nucleic acid complexes produced according to some embodiments of the present invention can be frozen and/or can be stored at various temperatures, including room temperature, about 4° C., about −20° C., about −80° C., and about −196° C. for an extended period of time, for example, for several hours, about 1 day, about 1 week, about 1 month, about 1 year, and longer than about 1 year. Some embodiments are therefore directed to a pharmaceutical formulation comprising synthetic RNA and a transfection reagent, wherein the pharmaceutical formulation is provided in solid form. Other embodiments are directed to a pharmaceutical formulation comprising synthetic RNA transfection reagent complexes, wherein the synthetic RNA transfection reagent complexes are provided in solid form. In various embodiments, the synthetic RNA transfection reagent complexes are provided in frozen form. Various embodiments are directed to a method for stabilizing nucleic acid transfection reagent complexes comprising forming nucleic acid transfection reagent complexes and contacting the nucleic acid transfection reagent complexes or vessel in which such are contained with a cryogenic liquid to produce stabilized nucleic acid transfection reagent complexes. In one embodiment, the nucleic acid transfection reagent complexes are stabilized for shipment or storage.

Illustrative subjects or patients refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

### Definitions

By “molecule” is meant a molecular entity (molecule, ion, complex, etc.).

By “RNA molecule” is meant a molecule that comprises RNA.

By “synthetic RNA molecule” is meant an RNA molecule that is produced outside of a cell or that is produced inside of a cell using bioengineering, by way of non-limiting example, an RNA molecule that is produced in an in vitro-transcription reaction, an RNA molecule that is produced by direct chemical synthesis or an RNA molecule that is produced in a genetically-engineered E. coli cell.

By “transfection” is meant contacting a cell with a molecule, wherein the molecule is internalized by the cell.

By “upon transfection” is meant during or after transfection.

By “transfection reagent” is meant a substance or mixture of substances that associates with a molecule and facilitates the delivery of the molecule to and/or internalization of the molecule by a cell, by way of non-limiting example, a cationic lipid, a charged polymer or a cell-penetrating peptide.

By “reagent-based transfection” is meant transfection using a transfection reagent.

By “medium” is meant a solvent or a solution comprising a solvent and a solute, by way of non-limiting example, Dulbecco's Modified Eagle's Medium (DMEM), DMEM+10% fetal bovine serum (FBS), saline or water.

By “complexation medium” is meant a medium to which a transfection reagent and a molecule to be transfected are added and in which the transfection reagent associates with the molecule to be transfected.

By “transfection medium” is meant a medium that can be used for transfection, by way of non-limiting example, Dulbecco's Modified Eagle's Medium (DMEM), DMEM/F12, saline or water.

By “recombinant protein” is meant a protein or peptide that is not produced in animals or humans. Non-limiting examples include human transferrin that is produced in bacteria, human fibronectin that is produced in an in vitro culture of mouse cells, and human serum albumin that is produced in a rice plant.

By “Oct4 protein” is meant a protein that is encoded by the POU5F1 gene, or a natural or engineered variant, family-member, orthologue, fragment or fusion construct thereof, by way of non-limiting example, human Oct4 protein (SEQ ID NO: 8), mouse Oct4 protein, Oct1 protein, a protein encoded by POU5F1 pseudogene 2, a DNA-binding domain of Oct4 protein or an Oct4-GFP fusion protein. In some embodiments the Oct4 protein comprises an amino acid sequence that has at least 70% identity with SEQ ID NO: 8, or in other embodiments, at least 75%, 80%, 85%, 90%, or 95% identity with SEQ ID NO: 8. In some embodiments, the Oct4 protein comprises an amino acid sequence having from 1 to 20 amino acid insertions, deletions, or substitutions (collectively) with respect to SEQ ID NO: 8. Or in other embodiments, the Oct4 protein comprises an amino acid sequence having from 1 to 15 or from 1 to 10 amino acid insertions, deletions, or substitutions (collectively) with respect to SEQ ID NO: 8.

By “Sox2 protein” is meant a protein that is encoded by the SOX2 gene, or a natural or engineered variant, family-member, orthologue, fragment or fusion construct thereof, by way of non-limiting example, human Sox2 protein (SEQ ID NO: 9), mouse Sox2 protein, a DNA-binding domain of Sox2 protein or a Sox2-GFP fusion protein. In some embodiments the Sox2 protein comprises an amino acid sequence that has at least 70% identity with SEQ ID NO: 9, or in other embodiments, at least 75%, 80%, 85%, 90%, or 95% identity with SEQ ID NO: 9. In some embodiments, the Sox2 protein comprises an amino acid sequence having from 1 to 20 amino acid insertions, deletions, or substitutions (collectively) with respect to SEQ ID NO: 9. Or in other embodiments, the Sox2 protein comprises an amino acid sequence having from 1 to 15 or from 1 to 10 amino acid insertions, deletions, or substitutions (collectively) with respect to SEQ ID NO: 9.

By “KIf4 protein” is meant a protein that is encoded by the KLF4 gene, or a natural or engineered variant, family-member, orthologue, fragment or fusion construct thereof, by way of non-limiting example, human Klf4 protein (SEQ ID NO: 10), mouse Klf4 protein, a DNA-binding domain of Klf4 protein or a Klf4-GFP fusion protein. In some embodiments the Klf4 protein comprises an amino acid sequence that has at least 70% identity with SEQ ID NO: 10, or in other embodiments, at least 75%, 80%, 85%, 90%, or 95% identity with SEQ ID NO: 10. In some embodiments, the Klf4 protein comprises an amino acid sequence having from 1 to 20 amino acid insertions, deletions, or substitutions (collectively) with respect to SEQ ID NO: 10. Or in other embodiments, the Klf4 protein comprises an amino acid sequence having from 1 to 15 or from 1 to 10 amino acid insertions, deletions, or substitutions (collectively) with respect to SEQ ID NO: 10.

By “c-Myc protein” is meant a protein that is encoded by the MYC gene, or a natural or engineered variant, family-member, orthologue, fragment or fusion construct thereof, by way of non-limiting example, human c-Myc protein (SEQ ID NO: 11), mouse c-Myc protein, I-Myc protein, c-Myc (T58A) protein, a DNA-binding domain of c-Myc protein or a c-Myc-GFP fusion protein. In some embodiments the c-Myc protein comprises an amino acid sequence that has at least 70% identity with SEQ ID NO: 11, or in other embodiments, at least 75%, 80%, 85%, 90%, or 95% identity with SEQ ID NO: 11. In some embodiments, the c-Myc protein comprises an amino acid having from 1 to 20 amino acid insertions, deletions, or substitutions (collectively) with respect to SEQ ID NO: 11. Or in other embodiments, the c-Myc protein comprises an amino acid sequence having from 1 to 15 or from 1 to 10 amino acid insertions, deletions, or substitutions (collectively) with respect to SEQ ID NO: 11.

By “erythropoietin” or “erythropoietin protein” is meant a protein that is encoded by the EPO gene, or a natural or engineered variant, family-member, orthologue, fragment or fusion construct thereof, by way of non-limiting example, human erythropoietin (SEQ ID NO: 164), mouse erythropoietin, darbepoetin, darbepoetin alfa, NOVEPOETIN, a binding domain of erythropoietin or an erythropoietin-GFP fusion protein. In some embodiments, the erythropoietin comprises an amino acid sequence that has at least 70% identity with SEQ ID NO: 164, or in other embodiments, at least 75%, 80%, 85%, 90%, or 95% identity with SEQ ID NO: 164. In some embodiments, the erythropoietin comprises an amino acid sequence having from 1 to 20 amino acid insertions, deletions, or substitutions (collectively) with respect to SEQ ID NO: 164. Or in other embodiments, the erythropoietin comprises an amino acid sequence having from 1 to 15 or from 1 to 10 amino acid insertions, deletions, or substitutions (collectively) with respect to SEQ ID NO: 164.

By “reprogramming” is meant causing a change in the phenotype of a cell, by way of non-limiting example, causing a β-cell progenitor to differentiate into a mature β-cell, causing a fibroblast to dedifferentiate into a pluripotent stem cell, causing a keratinocyte to transdifferentiate into a cardiac stem cell, causing the telomeres of a cell to lengthen or causing the axon of a neuron to grow.

By “reprogramming factor” is meant a molecule that, when a cell is contacted with the molecule and/or the cell expresses the molecule, can, either alone or in combination with other molecules, cause reprogramming, by way of non-limiting example, Oct4 protein, Tert protein, or erythropoietin.

By “germ cell” is meant a sperm cell or an egg cell.

By “pluripotent stem cell” is meant a cell that can differentiate into cells of all three germ layers (endoderm, mesoderm, and ectoderm) in vivo.

By “somatic cell” is meant a cell that is not a pluripotent stem cell or a germ cell, by way of non-limiting example, a skin cell.

By “hematopoietic cell” is meant a blood cell or a cell that can differentiate into a blood cell, by way of non-limiting example, a hematopoietic stem cell, or a white blood cell.

By “cardiac cell” is meant a heart cell or a cell that can differentiate into a heart cell, by way of non-limiting example, a cardiac stem cell, or a cardiomyocyte.

By “retinal cell” is meant a cell of the retina or a cell that can differentiate into a cell of the retina, by way of non-limiting example, a retinal pigmented epithelial cell.

By “skin cell” is meant a cell that is normally found in the skin, by way of non-limiting example, a fibroblast, a keratinocyte, a melanocyte, an adipocyte, a mesenchymal stem cell, an adipose stem cell or a blood cell.

By “immunosuppressant” is meant a substance that can suppress one or more aspects of an immune system, and that is not normally present in a mammal, by way of non-limiting example, B18R or dexamethasone.

By “single-strand break” is meant a region of single-stranded or double-stranded DNA in which one or more of the covalent bonds linking the nucleotides has been broken in one of the one or two strands.

By “double-strand break” is meant a region of double-stranded DNA in which one or more of the covalent bonds linking the nucleotides has been broken in each of the two strands.

By “nucleotide” is meant a nucleotide or a fragment or derivative thereof, by way of non-limiting example, a nucleobase, a nucleoside, a nucleotide-triphosphate, etc.

By “nucleoside” is meant a nucleotide or a fragment or derivative thereof, by way of non-limiting example, a nucleobase, a nucleoside, a nucleotide-triphosphate, etc.

By “gene editing” is meant altering the DNA sequence of a cell, by way of non-limiting example, by transfecting the cell with a protein that causes a mutation in the DNA of the cell or by transfecting the cell with a protein that causes a chemical change in the DNA of the cell.

By “gene-editing protein” is meant a protein that can, either alone or in combination with one or more other molecules, alter the DNA sequence of a cell, by way of non-limiting example, a nuclease, a transcription activator-like effector nuclease (TALEN), a zinc-finger nuclease, a meganuclease, a nickase, a clustered regularly interspaced short palindromic repeat (CRISPR)-associated protein, a DNA-repair protein, a DNA-modification protein, a base-modification protein, a DNA methyltransferase, an protein that causes DNA demethylation, an enzyme for which DNA is a substrate or a natural or engineered variant, family-member, orthologue, domain, fragment or fusion construct thereof.

By “repair template” is meant a nucleic acid containing a region of at least about 70% homology with a sequence that is within 10 kb of a target site of a gene-editing protein.

By “repeat sequence” is meant an amino-acid sequence that is present in more than one copy in a protein, to within at least about 10% homology, by way of non-limiting example, a monomer repeat of a transcription activator-like effector.

By “DNA-binding domain” is meant a region of a molecule that is capable of binding to a DNA molecule, by way of non-limiting example, a protein domain comprising one or more zinc fingers, a protein domain comprising one or more transcription activator-like (TAL) effector repeat sequences or a binding pocket of a small molecule that is capable of binding to a DNA molecule.

By “binding site” is meant a nucleic-acid sequence that is capable of being recognized by a gene-editing protein, DNA-binding protein, DNA-binding domain or a biologically active fragment or variant thereof or a nucleic-acid sequence for which a gene-editing protein, DNA-binding protein, DNA-binding domain or a biologically active fragment or variant thereof has high affinity, by way of non-limiting example, an about 20-base-pair sequence of DNA in exon 1 of the human BIRC5 gene.

By “target” is meant a nucleic acid that contains a binding site.

By “liposome” is meant an entity containing amphiphilic molecules, hydrophobic molecules, or a mixture thereof, that is at least transiently stable in an aqueous environment, by way of non-limiting example, a micelle, a unilamellar bilayer with aqueous interior, a multilamellar bilayer, a lipid nanoparticle, any of the foregoing complexed with one or more nucleic acids, or a stable nucleic acid lipid particle.

By “PEGylated” is meant covalently or otherwise stably bound to a poly(ethylene glycol) chain of any length or any molecular weight.

By “safe harbor locus” is meant a genomic site capable of accommodating the integration of new genetic material such that the integrated inserted genetic elements function predictably and/or do not cause alterations of the host genome which pose a risk to the host cell or organism.

Other relevant definitions, aspects, and embodiments are set forth in WO2013/086008, WO2014/071219, WO2015/117021, WO2016/131052, and WO2018/035377, the contents of which are hereby incorporated by reference in their entireties.

Selected Sequences

| SEQ ID NO | Description |
|---|---|
| 1 | Stsl |
| 2 | Stsl-HA |
| 3 | Stsl-HA2 |
| 4 | Stsl-UHA |
| 5 | Stsl-UHA2 |
| 6 | Stsl-HF |
| 7 | Stsl-UHF |
| 8 | Oct4 |
| 9 | Sox2 |
| 10 | Klf4 |
| 11 | c-Myc |
| 12 | BIRC5_exon1 |
| 13 | BIRC5_exon2 |
| 14 | BIRC5_exon3 |
| 15 | BIRC5_exon4 |
| 16 | BIRC5-1.1-L |
| 17 | BIRC5-1.1-R |
| 18 | BIRC5-1.2-L |
| 19 | BIRC5-1.2-R |
| 20 | BIRC5-1.3-L |
| 21 | BIRC5-1.3-R |
| 22 | BIRC5-2.1-L |
| 23 | BIRC5-2.1-R |
| 24 | BIRC5-2.2-L |
| 25 | BIRC5-2.2-R |
| 26 | BIRC5-3.1-L |
| 27 | BIRC5-3.1-R |
| 28 | CDK1 |
| 29 | CDK2 |
| 30 | CDK3 |
| 31 | CDK4 |
| 32 | CDK5 |
| 33 | CDK6 |
| 34 | BIRC5 |
| 35 | HIF1A |
| 36 | RRM2 |
| 37 | KRAS |
| 38 | EGFR |
| 39 | MYC |
| 40 | PKN3 |
| 41 | KIF11 |
| 42 | APC |
| 43 | BRCA1 |
| 44 | BRCA2 |
| 45 | TP53 |
| 46 | APP |
| 47 | HIT |
| 48 | IAPP |
| 49 | MAPT |
| 50 | PRNP |
| 51 | SNCA |
| 52 | SOD1 |
| 53 | Fokl |
| 54 | Repeat1 |
| 55 | Repeat2 |
| 56 | Repeat3 |
| 57 | EO-GHGG-Fokl (“GHGG” disclosed as SEQ ID NO: |
| | 547) |
| 58 | GHGG-Fokl (“GHGG” disclosed as SEQ ID NO: 547) |
| 59 | EO-GHGG-Stsl (“GHGG” disclosed as SEQ ID NO: |
| | 547) |
| 60 | GHGG-Stsl (“GHGG” disclosed as SEQ ID NO: 547) |
| 61 | collagen alpha-1(I) chain preproprotein |
| 62 | collagen alpha-2(I) chain precursor |
| 63 | collagen alpha-1(II) chain isoform 1 precursor |
| 64 | collagen alpha-1(II) chain isoform 2 precursor |
| 65 | collagen alpha-1(III) chain preproprotein |
| 66 | collagen alpha-1(IV) chain preproprotein |
| 67 | collagen alpha-2(IV) chain preproprotein |
| 68 | collagen alpha-3(IV) chain precursor |
| 69 | collagen alpha-4(IV) chain precursor |
| 70 | collagen alpha-5(IV) chain isoform 1 precursor |
| 71 | collagen alpha-6(IV) chain isoform A precursor |
| 72 | collagen alpha-1(V) chain isoform 1 preproprotein |
| 73 | collagen alpha-2(V) chain preproprotein |
| 74 | collagen alpha-3(V) chain preproprotein |
| 75 | collagen alpha-1(VI) chain precursor |
| 76 | collagen alpha-2(VI) chain isoform 2C2 precursor |
| 77 | collagen alpha-3(VI) chain isoform 1 precursor |
| 78 | collagen alpha-1(VII) chain precursor |
| 79 | elastin isoform a precursor |
| 80 | elastin isoform b precursor |
| 81 | elastin isoform c precursor |
| 82 | elastin isoform d precursor |
| 83 | elastin isoform e precursor |
| 84 | elastin isoform f precursor |
| 85 | elastin isoform g precursor |
| 86 | elastin isoform h precursor |
| 87 | elastin isoform i precursor |
| 88 | elastin isoform j precursor |
| 89 | elastin isoform k precursor |
| 90 | elastin isoform l precursor |
| 91 | elastin isoform m precursor |
| 92 | protein-lysine 6-oxidase isoform 1 preproprotein |
| 93 | protein-lysine 6-oxidase isoform 2 |
| 94 | telomerase reverse transcriptase isoform 1 |
| 95 | telomerase reverse transcriptase isoform 2 |
| 96 | fibronectin isoform 1 preproprotein |
| 97 | fibronectin isoform 3 preproprotein |
| 98 | fibronectin isoform 4 preproprotein |
| 99 | fibronectin isoform 5 preproprotein |
| 100 | fibronectin isoform 6 preproprotein |
| 101 | fibronectin isoform 7 preproprotein |
| 102 | vitronectin precursor |
| 103 | nidogen-1 precursor |
| 104 | laminin subunit alpha-1 precursor |
| 105 | insulin-like growth factor I isoform 1 preproprotein |
| 106 | fibroblast growth factor 1 isoform 1 precursor |
| 107 | fibroblast growth factor 2 |
| 108 | transforming growth factor beta-1 precursor |
| 109 | transforming growth factor beta-2 isoform 1 precursor |
| 110 | transforming growth factor beta-2 isoform 2 precursor |
| 111 | actin, alpha skeletal muscle |
| 112 | actin, aortic smooth muscle |
| 113 | actin, cytoplasmic 1 |
| 114 | actin, alpha cardiac muscle 1 proprotein |
| 115 | actin, cytoplasmic 2 |
| 116 | actin, gamma-enteric smooth muscle isoform 1 precursor |
| 117 | actin, gamma-enteric smooth muscle isoform 2 precursor |
| 118 | granulocyte colony-stimulating factor isoform a precursor |
| 119 | granulocyte colony-stimulating factor isoform b precursor |
| 120 | granulocyte colony-stimulating factor isoform c precursor |
| 121 | granulocyte colony-stimulating factor isoform d precursor |
| 122 | platelet-derived growth factor subunit A isoform 1 |
| | preproprotein |
| 123 | platelet-derived growth factor subunit A isoform 2 |
| | preproprotein |
| 124 | platelet-derived growth factor subunit B isoform 1 |
| | preproprotein |
| 125 | platelet-derived growth factor subunit B isoform 2 |
| | preproprotein |
| 126 | platelet-derived growth factor C precursor |
| 127 | platelet-derived growth factor D isoform 1 precursor |
| 128 | platelet-derived growth factor D isoform 2 precursor |
| 129 | interstitial collagenase isoform 1 preproprotein |
| 130 | interstitial collagenase isoform 2 |
| 131 | neutrophil collagenase preproprotein |
| 132 | stromelysin-2 preproprotein |
| 133 | macrophage metalloelastase preproprotein |
| 134 | fibrillin-1 precursor |
| 135 | fibrillin-2 precursor |
| 136 | lysyl oxidase homolog 1 preproprotein |
| 137 | lysyl oxidase homolog 2 precursor |
| 138 | lysyl oxidase homolog 3 isoform 1 precursor |
| 139 | lysyl oxidase homolog 3 isoform 2 precursor |
| 140 | lysyl oxidase homolog 3 isoform 3 |
| 141 | lysyl oxidase homolog 4 precursor |
| 142 | microfibrillar-associated protein 2 isoform a precursor |
| 143 | microfibrillar-associated protein 2 isoform b precursor |
| 144 | microfibrillar-associated protein 5 precursor |
| 145 | disintegrin and metalloproteinase domain-containing |
| | protein 17 preprotein |
| 146 | desmoglein-2 preproprotein |
| 147 | DNA polymerase eta isoform 1 |
| 148 | DNA polymerase eta isoform 2 |
| 149 | DNA polymerase eta isoform 3 |
| 150 | ferrochelatase, mitochondrial isoform a precursor |
| 151 | ferrochelatase, mitochondrial isoform b precursor |
| 152 | filaggrin |
| 153 | hyaluronan synthase 1 isoform 1 |
| 154 | hyaluronan synthase 1 isoform 2 |
| 155 | hyaluronan synthase 2 |
| 156 | hyaluronan synthase 3 isoform a |
| 157 | hyaluronan synthase 3 isoform b |
| 158 | proopiomelanocortin |
| 159 | plakophilin-1 isoform 1a |
| 160 | plakophilin-1 isoform 1b |
| 161 | retinol dehydrogenase 10 |
| 162 | mitochondrial brown fat uncoupling protein 1 |
| 163 | tyrosinase precursor |
| 164 | erythropoietin |
| 165 | epoetin alfa |
| 166 | darbepoetin alfa |
| 167 | NOVEPOETIN |
| 168 | NOVECRIT |

This invention is further illustrated by the following non-limiting examples.

### EXAMPLES

### Example 1 RNA Synthesis

RNA encoding green fluorescent protein (“GFP”), NOVEPOETIN (“EPO”), elastin (“ELN”), tyrosinase (“TYR”), melanocortin-1-receptor (“MC1R”), HAS1, HAS2, HAS3, COL3A1, COL7A1, COL1A1, COL1A2, hTERT, Holly GFP, Fresno RFP, mRFP, Blitzen Blue, RIBOSLICE gene-editing proteins, TALENs, Cas9, Oct4, Sox2, KIfM, c-Myc-2 (T58A), Lin28, IL12, IL6, IL15, IL22, BMP2, BMP7, BDNF, LIF, BMP6, IL15RA, FGF21, LIF, PTH, KRT5, KRT5-GFP, KRT14, KRT14-GFP, GDF15 and ESM, and comprising various combinations of canonical and non-canonical nucleotides, was synthesized from DNA templates using the T7 High Yield RNA Synthesis Kit and the Vaccinia Capping System kit with mRNA Cap 2′-O-Methyltransferase (all from New England Biolabs, Inc.), according to the manufacturer's instructions and the present inventors' previously disclosed inventions (U.S. application Ser. No. 13/465,490 (now U.S. Pat. No. 8,497,124), International Application No. PCT/US12/67966, U.S. application Ser. No. 13/931,251, International Application No. PCT/US13/68118, and International Application No. PCT/US2015/013949, the contents of all of which are hereby incorporated by reference in their entirety) (Table 4). The RNA was then diluted with nuclease-free water to between 100 ng/μL and 2000 ng/μL. For certain experiments, an RNase inhibitor (Superase-In, Life Technologies Corporation) was added at a concentration of 1 μL/100 μg of RNA. RNA solutions were stored at room temperature, 4° C., −20° C. or −80° C. For reprogramming experiments, RNA encoding Oct4, Sox2, KIf4, c-Myc-2 (T58A), and Lin28 was mixed at a molar ratio of 3:1:1:1:1.

TABLE 4
RNA Synthesis

| | | Reaction | ivT |
|---|---|---|---|
| Template | Nucleotides | Volume/μL | Yield/μg |
| ELN | A, 0.5 7dG, 0.4 5mU, | 20 | 34.1 |
| | 5mC | | |
| ELN | A, 0.5 7dG, 0.4 5mU, | 20 | 67.6 |
| | 5mC | | |
| GFP | A, 0.5 7dG, 0.4 5mU, | 10 | 60.5 |
| | 5mC | | |
| GFP | A, 0.5 7dG, 0.4 5mU, | 10 | 25.5 |
| | 5hmC | | |
| GFP | A, G, U, 5hmC | 10 | 58.3 |
| GFP | A, 0.5 7dG, U, 5hmC | 10 | 47.3 |
| GFP | A, 0.5 7dG, 0.4 5mU, | 10 | 33.8 |
| | 5cC | | |
| GFP | A, G, U, 5hmC | 15 | 30.3 |
| GFP | A, G, U, 5hmC | 15 | 44.6 |
| GFP | A, G, U, 5hmC | 15 | 24.7 |
| TYR | A, G, U, 5hmC | 15 | 45.4 |
| MC1R | A, G, U, 5hmC | 15 | 47.5 |
| TYR | A, G, U, C | 20 | 67.0 |
| TYR | A, G, psU, C | 20 | 93.7 |
| TYR | A, G, 0.4 5mU, C | 20 | 85.7 |
| TYR | A, G, U, 5mC | 20 | 73.4 |
| TYR | A, G, U, 5hmC | 20 | 72.7 |
| TYR | A, 0.5 7dG, U, C | 20 | 62.7 |
| TYR | A, G, psU, 5mC | 20 | 116.3 |
| TYR | A, G, psU, 5hmC | 20 | 102.4 |
| TYR | A, 0.5 7dG, psU, C | 20 | 87.3 |
| TYR | A, G, 0.4 5mU, 5mC | 20 | 106.5 |
| TYR | A, G, 0.4 5mU, 5hmC | 20 | 85.0 |
| TYR | A, 0.5 7dG, 0.4 5mU, | 20 | 70.9 |
| | C | | |
| TYR | A, 0.5 7dG, U, 5mC | 20 | 88.5 |
| TYR | A, 0.5 7dG, U, 5hmC | 20 | 59.1 |
| TYR | A, 0.5 7dG, psU, 5mC | 20 | 7.8 |
| TYR | A, 0.5 7dG, psU, | 20 | 98.0 |
| | 5hmC | | |
| TYR | A, 0.5 7dG, 0.4 5mU, | 20 | 106.5 |
| | 5mC | | |
| TYR | A, 0.5 7dG, 0.4 5mU, | 20 | 82.3 |
| | 5hmC | | |
| HAS1 | A, G, U, 5hmC | 20 | 178.4 |
| HAS2 | A, G, U, 5hmC | 20 | 59.3 |
| HAS3 | A, G, U, 5hmC | 20 | 102.6 |
| TYR | A, G, 0.4 5mU, 5hmC | 100 | 377.3 |
| COL3A1 | A, G, 0.4 5mU, 5hmC | 20 | 108.3 |
| COL7A1 | A, G, 0.4 5mU, 5hmC | 20 | 94.6 |
| COL1A1 (20 μL) | A, G, 0.4 5mU, 5hmC | 20 | 114.0 |
| COL1A2 (10 μL) | A, G, 0.4 5mU, 5hmC | 10 | 31.3 |
| TYR | A, G, 0.4 5mU, 5hmC | 100 | 249.9 |
| GFP | A, G, 0.4 5mU, 5hmC | 100 | 264.0 |
| hTERT | A, G, 0.4 5mU, 5hmC | 100 | 349.2 |
| GFP | A, G, U, 5hC | 20 | 81.7 |
| GFP | A, G, U, 0.5 5hC | 20 | 65.4 |
| GFP | A, sG, U, C | 20 | 34.7 |
| GFP | A, 0.5 sG, U, C | 20 | 47.5 |
| GFP | A, G, 5hmU, C | 20 | 22.1 |
| GFP | A, G, 0.5 5hmU, C | 20 | 28.4 |
| GFP | A, G, 5cU, C | 20 | 24.4 |
| GFP | A, G, 0.5 5cU, C | 20 | 28.4 |
| GFP | A, G, 5moU, C | 20 | 39.2 |
| GFP | A, G, 0.5 5moU, C | 20 | 34.2 |
| GFP | A, G, U, C | 20 | 42.0 |
| GFP | A, G, 5moU, C | 20 | 53.8 |
| GFP | A, G, 5moU, 5hmC | 20 | 101.5 |
| GFP | A, G, 0.4 5mU, 0.6 | 20 | 98.6 |
| | 5moU, C | | |
| GFP | A, G, 0.4 5mU, C | 20 | 99.6 |
| GFP | A, G, U, 5mC | 20 | 106.1 |
| GFP | A, G, U, C | 20 | 85.7 |
| GFP | A, G, 5moU, C | 100 | 398.4 |
| hTERT | A, G, 5moU, C | 20 | 82.6 |
| COL7A1 | A, G, 5moU, C | 20 | 34.9 |
| COL7A1 | A, G, 5moU, C | 100 | 342.0 |
| Holly GFP | A, G, 5moU, C | 20 | 36.7 |
| Fresno RFP | A, G, 5moU, C | 20 | 72.0 |
| Blitzen Blue | A, G, 5moU, C | 20 | 30.3 |
| hTERT | A, G, 5moU, C | 20 | 49.6 |
| Cas9 | A, G, 5moU, C | 20 | 31.6 |
| EPO | A, G, U, C | 20 | 101.0 |
| EPO | A, G, 5moU, C | 20 | 52.9 |
| EPO | A, G, psU, C | 20 | 106.0 |
| COL7A1 | A, G, 5moU, C | 20 | 80.2 |
| Oct4 (SEQ ID NO: 8) | A, G, 5moU, C | 300 | 1925.5 |
| Sox2 (SEQ ID NO: 9) | A, G, 5moU, C | 100 | 641.8 |
| Klf4 (SEQ ID NO: 10) | A, G, 5moU, C | 100 | 739.0 |
| c-Myc-2 (T58A) | A, G, 5moU, C | 100 | 574.0 |
| Lin28 | A, G, 5moU, C | 100 | 556.0 |
| IL2 | A, G, 5moU, C | 20 | 62.4 |
| IL6 | A, G, 5moU, C | 20 | 22.2 |
| IL15 | A, G, 5moU, C | 20 | 50.4 |
| IL22 | A, G, 5moU, C | 20 | 63.6 |
| BMP2 | A, G, 5moU, C | 20 | 83.2 |
| BDNF | A, G, 5moU, C | 20 | 45.0 |
| LIF | A, G, 5moU, C | 20 | 54.0 |
| BMP6 | A, G, 5moU, C | 20 | 92.2 |
| IL15RA | A, G, 5moU, C | 20 | 91.4 |
| FGF21 | A, G, 5moU, C | 20 | 79.2 |
| GFP | A, G, 5moU, C | 40 | 181.0 |
| IL2 | A, G, 5moU, C | 30 | 99.4 |
| IL6 | A, G, 5moU, C | 30 | 31.2 |
| IL15 | A, G, 5moU, C | 30 | 89.8 |
| IL22 | A, G, 5moU, C | 30 | 104.0 |
| BDNF | A, G, 5moU, C | 30 | 95.9 |
| BMP2 | A, G, 5moU, C | 30 | 112.0 |
| LIF | A, G, 5moU, C | 30 | 116.0 |
| PTH | A, G, 5moU, C | 30 | 88.4 |
| EPO | A, G, 5moU, C | 30 | 83.3 |
| KRT5 | A, G, 5moU, C | 15 | 66.6 |
| KRT5-GFP | A, G, 5moU, C | 15 | 81.1 |
| KRT14 | A, G, 5moU, C | 15 | 75.1 |
| KRT14-GFP | A, G, 5moU, C | 15 | 90.4 |
| GDF15 | A, G, 5moU, C | 15 | 71.1 |
| A1AT TALEN L | A, G, 5moU, C | 15 | 56.4 |
| A1AT TALEN R | A, G, 5moU, C | 15 | 57.3 |
| A1AT RIBOSLICE L_A | A, G, 5moU, C | 15 | 74.3 |
| A1AT RIBOSLICE L_B | A, G, 5moU, C | 15 | 56.4 |
| A1AT RIBOSLICE R_A | A, G, 5moU, C | 15 | 60.3 |
| A1AT RIBOSLICE R_B | A, G, 5moU, C | 15 | 35.7 |
| COL7A1 exon 73 | A, G, 5moU, C | 15 | 86.48 |
| TALEN L | | | |
| COL7A1 exon 73 | A, G, 5moU, C | 15 | 83.66 |
| TALEN R | | | |
| COL7A1 exon 73 rs3L | A, G, 5moU, C | 15 | 103.4 |
| 50A | | | |
| COL7A1 exon 73 rs3L | A, G, 5moU, C | 15 | 112.8 |
| 50B | | | |
| COL7A1 exon 73 rs3R | A, G, 5moU, C | 15 | 81.404 |
| 50A | | | |
| COL7A1 exon 73 rs3R | A, G, 5moU, C | 15 | 78.02 |
| 50B | | | |
| COL7A1 exon 73 | A, G, 5moU, C | 15 | 88.924 |
| TALEN L EA | | | |
| COL7A1 exon 73 | A, G, 5moU, C | 15 | 75.2 |
| TALEN L Het | | | |
| COL7A1 exon 73 | A, G, 5moU, C | 15 | 86.48 |
| TALEN R EA | | | |
| COL7A1 exon 73 | A, G, 5moU, C | 15 | 62.98 |
| TALEN R Het | | | |
| COL7A1 exon 73 | A, G, 5moU, C | 15 | 82.7 |
| TALEN L EA/Het | | | |
| COL7A1 exon 73 | A, G, 5moU, C | 15 | 69.7 |
| TALEN R EA/Het | | | |
| HBB exon 1 TALEN L | A, G, 5moU, C | 15 | 112.8 |
| HBB exon 1 TALEN R | A, G, 5moU, C | 15 | 108.1 |
| PD-1 exon 1 TALEN L | A, G, 5moU, C | 15 | 95.88 |
| PD-1 exon 1 TALEN R | A, G, 5moU, C | 15 | 101.52 |
| ESM1—transcript | A, G, 5moU, C | 15 | 61 |
| variant 1 | | | |
| ESM1—transcript | A, G, 5moU, C | 15 | 66 |
| variant 2 | | | |
| COL7A1 exon 73 | A, G, 5moU, C | 15 | 107.16 |
| spliceMod TALEN 1L | | | |
| COL7A1 exon 73 | A, G, 5moU, C | 15 | 95.88 |
| spliceMod TALEN 2L | | | |
| COL7A1 exon 73 | A, G, 5moU, C | 15 | 101.52 |
| spliceMod TALEN 3L | | | |
| COL7A1 exon 73 | A, G, 5moU, C | 15 | 99.64 |
| spliceMod TALEN 4L | | | |
| COL7A1 exon 73 | A, G, 5moU, C | 15 | 107.16 |
| spliceMod TALEN 1R | | | |
| COL7A1 exon 73 | A, G, 5moU, C | 15 | 103.4 |
| spliceMod TALEN 2R | | | |
| COL7A1 exon 73 | A, G, 5moU, C | 15 | 97.76 |
| spliceMod TALEN 3R | | | |
| COL7A1 exon 73 | A, G, 5moU, C | 15 | 95.88 |
| spliceMod RIBOSLICE | | | |
| L2A | | | |
| COL7A1 exon 73 | A, G, 5moU, C | 15 | 99.64 |
| spliceMod RIBOSLICE | | | |
| L2B | | | |
| COL7A1 exon 73 | A, G, 5moU, C | 15 | 103.4 |
| spliceMod RIBOSLICE | | | |
| R2A | | | |
| COL7A1 exon 73 | A, G, 5moU, C | 15 | 99.64 |
| spliceMod RIBOSLICE | | | |
| R2B | | | |

“A” refers to adenosine-5′-triphosphate,
“G” refers to guanosine-5′-triphosphate,
“U” refers to uridine-5′-triphosphate,
“C” refers to cytidine-5′-triphosphate,
“7dG” refers to 7-deazaguanosine-5′-triphosphate,
“sG” refers to thienoguanosine-5′-triphosphate,
“5mC” refers to 5-methylcytidine-5′-triphosphate,
“5hmC” refers to 5-hydroxymethylcytidine-5′-triphosphate,
“5cC” refers to 5-carboxycytidine-5′-triphosphate,
“5fC” refers to 5-formylcytidine-5′-triphosphate,
“5hC” refers to 5-hydroxycytidine-5′-triphosphate,
“psU” refers to 5-pseudouridine-5′-triphosphate,
“5mU” refers to 5-methyluridine-5′-triphosphate,
“5hmU” refers to 5-hydroxymethyluddine-5′-triphosphate,
“5cU” refers to 5-carboxyuridine-5′-triphosphate, and
“5moU” refers to 5-methoxyuridine-5′-triphosphate.

### Example 2 Preparation of RNA-Transfection-Reagent Complexes

For each microgram of RNA, 1 μg RNA and 1 μL transfection reagent (LIPOFECTAMINE 3000, Life Technologies Corporation) were first diluted separately in complexation medium (Opti-MEM, Life Technologies Corporation or DMEM/F12+10 μg/mL insulin+5.5 μg/mL transferrin+6.7 ng/mL sodium selenite+2 μg/mL ethanolamine) to a total volume of between 5 NL and 100 NL each. Diluted RNA and transfection reagent were then mixed and incubated for 10 min at room temperature, according to the transfection reagent-manufacturer's instructions.

### Example 3 Transfection of Cells with Synthetic RNA

Complexes were prepared according to Example 2, and were then added directly to cells in culture. For transfection in 6-well plates, between 10 μL and 250 μL of complexes were added to each well of the 6-well plate, which already contained 2 mL of transfection medium per well. Plates were shaken gently to distribute the complexes throughout the well. Cells were incubated with complexes for 4 hours to overnight, before replacing the medium with fresh transfection medium (2 mL/well). Alternatively, the medium was not replaced. Volumes were scaled for transfection in 24-well and 96-well plates.

### Example 4 Toxicity of and Protein Translation from Synthetic RNA Containing Non-Canonical Nucleotides

Primary human fibroblasts were transfected according to Example 2, using RNA synthesized according to Example 1. Cells were fixed and stained 20-24 h after transfection using an antibody against Oct4. The relative toxicity of the RNA was determined by assessing cell density at the time of fixation.

### Example 5 Delivery of Synthetic RNA to the Skin

The complexation reaction shown in Table 5 was prepared using RNA encoding green fluorescent protein (GFP) or collagen, type VII, alpha (COL7), synthesized according to Example 1. The concentration of the RNA stock solution was 500 μg/mL.

TABLE 5
RNA Complexation Reaction

| | Volume |
|---|---|
| RNA solution tube | |
| GFP or COL7 RNA | 8 μL |
| FactorPlex ™ complexation buffer | 42 μL |
| Transfection reagent solution tube | |
| LIPOFECTAMINE 3000 (LIFE TECHNOLOGIES) | 4 μL |
| FactorPlex ™ complexation buffer | 46 μL |

Each tube was mixed by pipetting, and the transfection reagent solution tube was incubated for 30 s at room temperature. The transfection reagent solution was then transferred to the RNA solution, and the contents were mixed by rapidly pipetting up and down 10 times. Following a 10 min incubation, dilutions were prepared according to

TABLE 6
Injection Solutions

| | | Complexation | FactorPlex ™ | RNA |
|---|---|---|---|---|
| Site | RNA | Volume | Volume | amount |
| 1 | GFP | 7.5 μL | 22.5 μL | 0.3 μg |
| 2 | GFP | 15 μL | 15 μL | 0.6 μg |
| 3 | GFP | 30 μL | 0 μL | 1.2 μg |
| 4 | COL7 | 30 μL | 0 μL | 1.2 μg |

For each injection, the corresponding solution was drawn into a 3 cc insulin syringe with an 8 mm, 31-gauge needle (Becton, Dickinson and Company, Part Number: 328291) and air bubbles were removed. A clear field was selected on the left forearm of a healthy 33-year-old male human subject, and was disinfected with 70% isopropanol and allowed to dry. The needle was positioned at an angle of approximately 100 to the anterior (palmar) forearm with bevel facing up, and was inserted until the bevel was just covered. 30 μL of the RNA solution was injected intradermally over the course of approximately 10 sec. A distinct wheal appeared during the injection process. The needle was withdrawn, the wheal remained for approximately 1 minute, and no fluid escaped from the injection site. A total of 4 injections were performed according to Table 6, and all of the injections were performed between 11 and 28 minutes following the preparation of the RNA complexation reaction. No swelling, redness, or soreness occurred as a result of the injections. A small amount of bleeding occurred when the needle was removed from sites 2 and 4, resulting in the appearance of a small red spot at these sites.

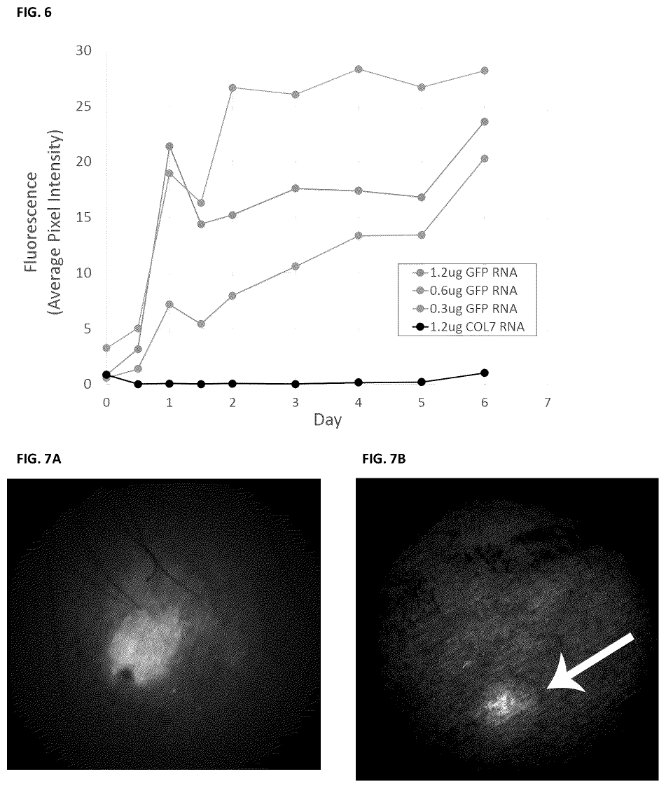

The injection sites were imaged according to the schedule of Table 7, and every 24 hours thereafter for 6 days. Fluorescence images were acquired using an inverted microscope (Nikon Eclipse TS100) equipped with an EXFO X-Cite™ 120 fluorescence illumination system and the filter sets shown in Table 7. Fluorescence images were captured using a Sony NEX-7 digital camera (FIG. 1 to FIG. 6).

TABLE 7
Measurement Parameters

| Site | Time | Image Type | Exposure Time |
|---|---|---|---|
| All | 0 h | Brightfield | Automatic |
| 1 | 0 h | FITC | 1/10 s |
| 2 | 0 h | FITC | 1/10 s |
| 3 | 0 h | FITC | 1/10 s |
| 4 | 0 h | FITC | 1/10 s |
| 1 | 12 h | FITC | 1/10 s |
| 2 | 12 h | FITC | 1/10 s |
| 3 | 12 h | FITC | 1/10 s |
| 4 | 12 h | FITC | 1/10 s |
| 1 | 12 h | FITC | 1/20 s |
| 2 | 12 h | FITC | 1/20 s |
| 3 | 12 h | FITC | 1/20 s |
| 4 | 12 h | FITC | 1/20 s |
| All | 24 h | Brightfield | Automatic |
| 1 | 24 h | FITC | 1/20 s |
| 2 | 24 h | FITC | 1/20 s |
| 3 | 24 h | FITC | 1/20 s |
| 4 | 24 h | FITC | 1/20 s |
| 1 | 24 h | Cy3.5 | 1/5 s |
| 2 | 24 h | Cy3.5 | 1/5 s |
| 3 | 24 h | Cy3.5 | 1/5 s |
| 4 | 24 h | Cy3.5 | 1/5 s |
| 1 | 24 h | Cy3 | 1/5 s |
| 2 | 24 h | Cy3 | 1/5 s |
| 3 | 24 h | Cy3 | 1/5 s |
| 4 | 24 h | Cy3 | 1/5 s |
| 1 | 36 h | FITC | 1/20 s |
| 2 | 36 h | FITC | 1/20 s |
| 3 | 36 h | FITC | 1/20 s |
| 4 | 36 h | FITC | 1/20 s |
| 1 | 48 h | FITC | 1/20 s |
| 2 | 48 h | FITC | 1/20 s |
| 3 | 48 h | FITC | 1/20 s |
| 4 | 48 h | FITC | 1/20 s |

An independent experiment was carried out using the 1.2 μg dose of GFP RNA, with similar results (FIG. 7A).

FIG. 7B depicts intradermal injection of RNA encoding GFP, formulated for intradermal injection, into the ventral forearm of the subject in FIG. 1, 48 months following the injection of FIG. 1. The arrow indicates an approximately 1 cm² area of GFP expression at the site of injection.

TABLE 8
Filter Sets

| Image Type | Filter Set |
|---|---|
| Cy3 | Chroma SP102V2 |
| Cy3.5 | Chroma SP103V2 |
| FITC | Chroma SP101 |

### Example 6 High-Efficiency Gene Editing by Repeated Transfection with RIBOSLICE

Primary human fibroblasts were plated in 6-well plates coated with recombinant human fibronectin and recombinant human vitronectin (each diluted in DMEM/F12 to a concentration of 1 μg/mL, 1 mL/well, and incubated at room temperature for 1 h) at a density of 10,000 cells/well in transfection medium. The following day, the cells were transfected as in Example 2 with RNA synthesized according to Example 1. The following day cells in one of the wells were transfected a second time. Two days after the second transfection, the efficiency of gene editing was measured using a mutation-specific nuclease assay.

### Example 7 Transfection of Cells with Synthetic RNA Containing Non-Canonical Nucleotides and DNA Encoding a Repair Template

For transfection in 6-well plates, 1 μg RNA encoding gene-editing proteins targeting exon 16 of the human APP gene, 1 μg single-stranded repair template DNA containing a Pstl restriction site that was not present in the target cells, and 6 μL transfection reagent (LIPOFECTAMINE RNAiMAX, Life Technologies Corporation) were first diluted separately in complexation medium (Opti-MEM, Life Technologies Corporation) to a total volume of 120 μL. Diluted RNA, repair template, and transfection reagent were then mixed and incubated for 15 min at room temperature, according to the transfection reagent-manufacturer's instructions. Complexes were added to cells in culture. Approximately 120 μL of complexes were added to each well of a 6-well plate, which already contained 2 mL of transfection medium per well. Plates were shaken gently to distribute the complexes throughout the well. Cells were incubated with complexes for 4 hours to overnight, before replacing the medium with fresh transfection medium (2 mL/well). The next day, the medium was changed to DMEM+10% FBS. Two days after transfection, genomic DNA was isolated and purified. A region within the APP gene was amplified by PCR, and the amplified product was digested with Pstl and analyzed by gel electrophoresis.

### Example 8 Reprogramming Human Fibroblasts Using Synthetic RNA Containing Non-Canonical Nucleotides

Primary human neonatal fibroblasts were plated in 6-well plates coated with recombinant human fibronectin and recombinant human vitronectin (each diluted in DMEM/F12 to a concentration of 11 μg/mL, 1 mL/well, and incubated at room temperature for 1 h) at a density of 10,000 cells/well in transfection medium. The following day, the cells were transfected as in Example 2, using RNA containing A, 0.5 7 dG, 0.5 5 mU, and 5mC, and an RNA dose of 0.5 μg/well on day 1, 0.5 μg/well on day 2, 2 μg/well on day 3, 2 μg/well on day 4, and 4 μg/well on day 5. Small colonies of cells exhibiting morphology consistent with reprogramming became visible as early as day 5. The medium was replaced with maintenance medium on day 6. Cells were stained using an antibody against Oct4. Oct4-positive colonies of cells exhibiting a morphology consistent with reprogramming were visible throughout the well (FIG. 8).

### Example 9 Screening of Reagents for Delivery of Nucleic Acids to Cells

Delivery reagents including polyethyleneimine (PEI), various commercial lipid-based transfection reagents, a peptide-based transfection reagent (N-TER, Sigma-Aldrich Co. LLC.), and several lipid-based and sterol-based delivery reagents were screened for transfection efficiency and toxicity in vitro. Delivery reagents were complexed with RIBOSLICE BIRC5-1.2, and complexes were delivered to HeLa cells in culture. Toxicity was assessed by analyzing cell density 24 h after transfection. Transfection efficiency was assessed by analyzing morphological changes. The tested reagents exhibited a wide range of toxicities and transfection efficiencies. Reagents containing a higher proportion of ester bonds exhibited lower toxicities than reagents containing a lower proportion of ester bonds or no ester bonds.

### Example 10 High-Concentration Liposomal RIBOSLICE

High-Concentration Liposomal RIBOSLICE was prepared by mixing 1 μg RNA at 500 ng/μL with 3 μL of complexation medium (Opti-MEM, Life Technologies Corporation), and 2.5 μL of transfection reagent (LIPOFECTAMINE 2000, Life Technologies Corporation) per μg of RNA with 2.5 μL of complexation medium. Diluted RNA and transfection reagent were then mixed and incubated for 10 min at room temperature to form High-Concentration Liposomal RIBOSLICE. Alternatively, a transfection reagent containing DOSPA or DOSPER is used.

### Example 11 Liposome Formulation and Nucleic-Acid Encapsulation

Liposomes are prepared using the following formulation: 3.2 mg/mL N-(carbonyl-ethoxypolyethylene glycol 2000)-1,2-distearoyl-sn-glycero-3-phosphoethanolamine (MPEG2000-DSPE), 9.6 mg/mL fully hydrogenated phosphatidylcholine, 3.2 mg/mL cholesterol, 2 mg/mL ammonium sulfate, and histidine as a buffer. pH is controlled using sodium hydroxide and isotonicity is maintained using sucrose. To form liposomes, lipids are mixed in an organic solvent, dried, hydrated with agitation, and sized by extrusion through a polycarbonate filter with a mean pore size of 800 nm. Nucleic acids are encapsulated by combining 10 g of the liposome formulation per 1 g of nucleic acid and incubating at room temperature for 5 minutes.

### Example 12 Folate-Targeted Liposome Formulation

Liposomes are prepared using the following formulation: 3.2 mg/mL N-(carbonyl-ethoxypolyethylene glycol 2000)-1,2-distearoyl-sn-glycero-3-phosphoethanolamine (MPEG2000-DSPE), 9.6 mg/mL fully hydrogenated phosphatidylcholine, 3.2 mg/mL cholesterol, 2 mg/mL ammonium sulfate, and histidine as a buffer, with 0.27 mg/mL 1,2-distearoyl-sn-glycero-3-phosphoethanolamine-N-[folate(polyethylene glycol)-5000](FA-MPEG5000-DSPE) added to the lipid mixture. pH is controlled using sodium hydroxide and isotonicity is maintained using sucrose. To form liposomes, lipids are mixed in an organic solvent, dried, hydrated with agitation, and sized by extrusion through a polycarbonate filter with a mean pore size of 800 nm. Nucleic acids are encapsulated by combining 10 g of the liposome formulation per 1 g of nucleic acid and incubating at room temperature for 5 minutes.

### Example 13 Therapy Comprising Liposomal Protein-Encoding RNA

Liposomes encapsulating synthetic RNA encoding a therapeutic protein, synthesized according to Example 1, are prepared according to Example 11 or Example 12. The liposomes are administered by injection or intravenous infusion.

### Example 14 Storage and Stability of Synthetic RNA Complexes

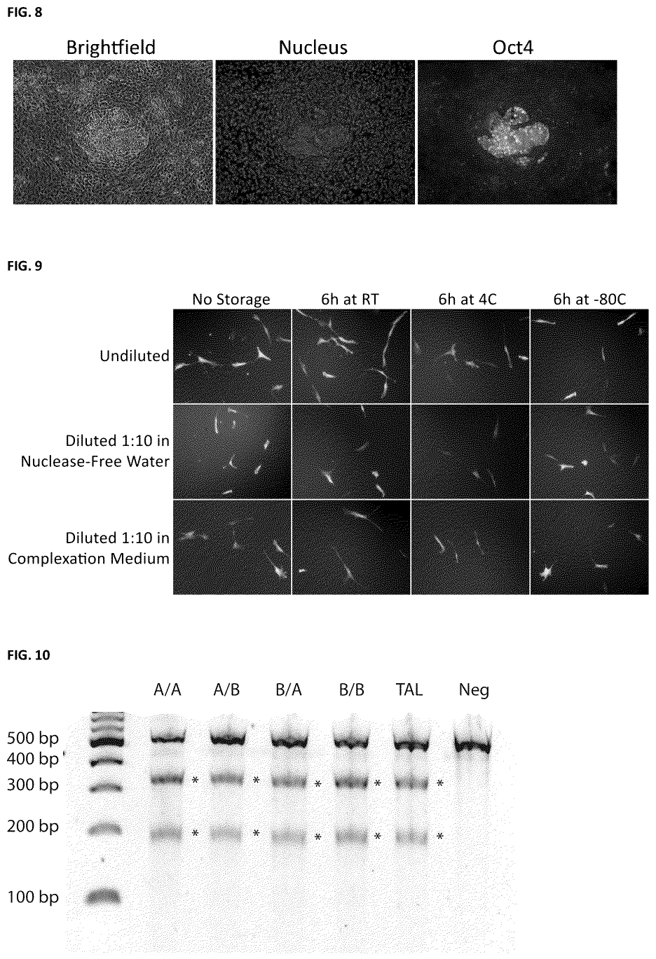

A complexation reaction using RNA encoding GFP was prepared according to Example 5. Following the 10 min incubation, the complexation reaction was divided into three equal parts, one of which was diluted 1:10 in FactorPlex™ complexation medium, one of which was diluted 1:10 in sterile, nuclease-free water, and one of which was left undiluted. Each of the three parts was then further divided into four equal parts, one of which was applied to primary human dermal fibroblasts according to Example 3, one of which was left at room temperature for six hours before applying to primary human dermal fibroblasts according to Example 3, one of which was placed at 4° C. for six hours before applying to primary human dermal fibroblasts according to Example 3, and one of which was snap frozen in liquid nitrogen and placed at −80° C. for six hours before applying to primary human dermal fibroblasts according to Example 3. The cells were imaged using a fluorescence microscope approximately 24 hours after the first transfection (FIG. 9). All wells contained GFP-positive cells, demonstrating that the synthetic RNA complexes were stable and maintained activity in all of the storage conditions tested.

### Example 15: Gene Editing of A1AT, COL7A1, HBB, and PD-1

With reference to Example 1, Table 4, the following templates and targets were used:

| | | SEQ |
|---|---|---|
| | | ID |
| Template | Target Sequence | NO: |
| A1AT TALEN L | TGCCTGGTCCCTGTCTCCCT | 615 |
| A1AT TALEN R | TGTCTTCTGGGCAGCATCTC | 616 |
| A1AT RIBOSLICE L_A | TGCCTGGTCCCTGTCTCCCT | 615 |
| A1AT RIBOSLICE L_B | TGCCTGGTCCCTGTCTCCCT | 615 |
| A1AT RIBOSLICE R_A | TGTCTTCTGGGCAGCATCTC | 616 |
| A1AT RIBOSLICE R_B | TGTCTTCTGGGCAGCATCTC | 616 |
| COL7A1 exon 73 TALEN L | TATTCCCGGGCTCCCAGGCA | 622 |
| COL7A1 exon 73 TALEN R | TCTCCTGGCCTTCCTGCCTC | 612 |
| COL7A1 exon 73 rs3L 50A | TATTCCCGGGCTCCCAGGCA | 622 |
| COL7A1 exon 73 rs3L 50B | TATTCCCGGGCTCCCAGGCA | 622 |
| COL7A1 exon 73 rs3R 50A | TCTCCTGGCCTTCCTGCCTC | 612 |
| COL7A1 exon 73 rs3R 50B | TCTCCTGGCCTTCCTGCCTC | 612 |
| COL7A1 exon 73 TALEN L | TATTCCCGGGCTCCCAGGCA | 622 |
| EA | | |
| COL7A1 exon 73 TALEN L | TATTCCCGGGCTCCCAGGCA | 622 |
| Het | | |
| COL7A1 exon 73 TALEN R | TCTCCTGGCCTTCCTGCCTC | 612 |
| EA | | |
| COL7A1 exon 73 TALEN R | TCTCCTGGCCTTCCTGCCTC | 612 |
| Het | | |
| COL7A1 exon 73 TALEN L | TATTCCCGGGCTCCCAGGCA | 622 |
| EA/Het | | |
| COL7A1 exon 73 TALEN R | TCTCCTGGCCTTCCTGCCTC | 612 |
| EA/Het | | |
| HBB exon 1 TALEN L | TGGTGCATCTGACTCCTGAG | 623 |
| HBB exon 1 TALEN R | TCACCTTGCCCCACAGGGCA | 624 |
| PD-1 exon 1 TALEN L | TCCAGGCATGCAGATCCCAC | 625 |
| PD-1 exon 1 TALEN R | TTGTAGCACCGCCCAGACGA | 626 |
| TTR (forward (top) and | TGCTGTCCGAGGCAGTCCTG | 639; |
| reverse (bottom) | TCAGCAGCCTTTCTGAACAC | 640 |
| TTR (forward (top) and | TGTCCGAGGCAGTCCTGCCA | 641; |
| reverse (bottom) | TCAGCAGCCTTTCTGAACAC | 642 |
| TTR (forward (top) and | TCCGAGGCAGTCCTGCCATC | 643; |
| reverse (bottom) | TCAGCAGCCTTTCTGAACAC | 644 |
| TTR (forward (top) and | TGTCCGAGGCAGTCCTGCCA | 645; |
| reverse (bottom) | TCATCAGCAGCCTTTCTGAA | 646 |
| TTR (forward (top) and | TCCGAGGCAGTCCTGCCATC | 647; |
| reverse (bottom) | TCATCAGCAGCCTTTCTGAA | 648 |
| TTR (forward (top) and | TCCGAGGCAGTCCTGCCATC | 649; |
| reverse (bottom) | TGTCATCAGCAGCCTTTCTG | 650 |

About 100,000 primary human neonatal epidermal keratinocytes were plated in EpiLife+Supplement S7 and transfected according to Example 3 with RNA encoding gene-editing proteins that target the sequences L: TGCCTGGTCCCTGTCTCCCT (SEQ ID NO: 615) and R: TGTCTTCTGGGCAGCATCTC (SEQ ID NO: 616), which were located approximately 75 bp from the Alpha-1-Antitrypsin (AlAT) start codon. Cells were gene edited, and gene-editing efficiency was measured as previously described. Results as shown in FIG. 10 demonstrate efficient gene-editing by the gene editing proteins.

About 100,000 primary human neonatal epidermal keratinocytes were plated in EpiLife+Supplement S7 and transfected according to Example 3 with RNA encoding gene-editing proteins that target the sequences L: TATTCCCGGGCTCCCAGGCA (SEQ ID NO: 622) and R: TCTCCTGGCCTTCCTGCCTC (SEQ ID NO: 612), which were located near the end of exon 73 of COL7A1. Cells were gene edited, and gene-editing efficiency was measured as previously described. Results as shown in FIG. 11 demonstrate efficient gene-editing by the gene editing proteins.

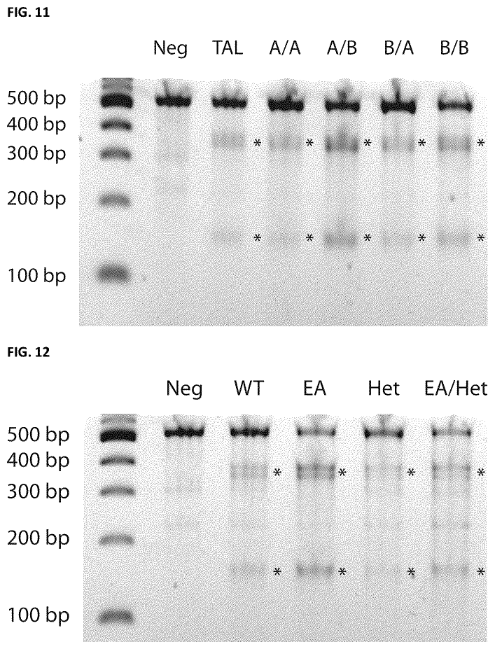

About 50,000 primary human neonatal epidermal keratinocytes were plated in EpiLife+Supplement S7 and transfected according to Example 3 with RNA encoding various gene-editing proteins that target the sequences L: TATTCCCGGGCTCCCAGGCA (SEQ ID NO: 622) and R: TCTCCTGGCCTTCCTGCCTC (SEQ ID NO: 612), which were located near the end of exon 73 of COL7A1. In particular, the gene-editing proteins comprised the endonuclease cleavage domain of FokI and variants thereof. The variants included a FokI variant with enhanced activity (S35P, K58E), a FokI heterodimer (i.e., L: Q103E, N113D, I116L; and R: E107K, H154R, I155K), and a combination thereof (i.e., L: S35P, K58E, Q103E, N113D, I116L; and R: S35P, K58E, E107K, H154R, I155K). Cells were gene edited, and gene-editing efficiency was measured as previously described. Results as shown in FIG. 12 demonstrate efficient gene-editing by the gene editing proteins.

About 100,000 primary human neonatal epidermal keratinocytes (animal-protein free) were plated in EpiLife+Supplement S7. Cells were transfected according to Example 3 with 2 μg RNA encoding the HBB exon 1 TALEN L and HBB exon 1 TALEN R gene-editing proteins (1 μg each). 48 hours after transfection, DNA was harvested and analyzed for gene editing (T7E1 assay; forward primer: gccaaggacaggtacggctgtcatc (SEQ ID NO: 627); reverse primer: cttgccatgagccttcaccttagggttg (SEQ ID NO: 628); product size: 518 nt; predicted band sizes: 300 nt, 218 nt). Results as shown in FIG. 13 demonstrate efficient gene-editing by the gene editing proteins.

About 100,000 primary human neonatal epidermal keratinocytes (animal-protein free) were plated in EpiLife+Supplement S7. Cells were transfected according to Example 3 with 2 μg RNA encoding the PD1 exon 1 TALEN L and PD1 exon 1 TALEN R gene-editing proteins (1 μg each). After 48 hours, DNA was harvested and analyzed for gene editing (T7E1 assay; forward primer: tcctctgtctccctgtctctgtctctctctc (SEQ ID NO: 594); reverse primer: ggacttgggccaggggaggag (SEQ ID NO: 595); product size: 612 nt; predicted band sizes: 349 nt, 263 nt). Results as shown in FIG. 14 demonstrate efficient gene-editing by the gene editing proteins.

### Example 16 Liposome Formulation Using Microfluidics

A liposome preparation used to encapsulate nucleic acids comprising 50 mol % (DLin-MC3-DMA), 38.5 mol % cholesterol, 10 mol % (DSPC), and 1.5 mol % (DMG-PEG) or 1.5 mol % (DMPE-PEG), was prepared as follows: Lipids were dissolved in absolute ethanol to a total lipid concentration of 12.5 mM. RNA prepared according to Example 1 was diluted to 228 ng/μL in 50 mM citrate buffer, pH 3.0 (Teknova). The solutions were applied to a Nanoassemblr Spark cartridge (Precision Nanosystems) according to the manufacturer's instructions, and were mixed through a microfluidic channel by the Spark at a 3:1 volume ratio of the aqueous to organic solutions. Dilution into phosphate-buffered saline (1×PBS, Ambion) was performed immediately after formulation.

Liposomal encapsulation efficiency was determined by fluorometric analysis. Formulated RNA was quantitated with a Qubit broad-range RNA kit (Invitrogen) in the presence (total RNA) or absence (unencapsulated RNA) of 1% Triton X-100. The encapsulation efficiency, calculated as encapsulated RNA as a percentage of total RNA, is shown in FIG. 15.

Liposome-formulated RNA was added dropwise to 20,000 human epidermal keratinocytes (HEKn) or fully-differentiated human adipocytes in each well of a 24-well plate, at 0.05 to 3.0 μg/well.

### Example 17 Liposome Formulation for in Vivo Injection

A liposome preparation comprising 30 mol % DODAP, 30 mol % DOPE, and 40 mol % cholesterol, was prepared as follows: Lipids were diluted from stocks in ethanol to a final concentration of 37.5 mM total lipid at the indicated molar ratios. RNA encoding GFP was diluted to 411 ng/μL in 50 mM citrate buffer, pH 3.0. Both solutions were transferred to syringes and assembled into a Nanoassemblr Benchtop (Precision Nanosystems) as directed by the manufacturer. The instrument was used to mix the aqueous and organic solutions at a 3:1 flowrate ratio (aqueous:organic) and a total flow rate of 20 mL/min. The resulting liposomes were diluted 3:1 in PBS, pH 7.4 (Ambion) and used without further modification.

### Example 18 Formulation of RIBOSLICE for in Vitro Gene Editing

Synthetic RNAs encoding RIBOSLICE were prepared according to the method of Example 1 and were mixed in 50 mM citrate buffer, pH 3.0 (Teknova) to a final concentration of 137 ng/μL. Lipid stocks in ethanol were diluted in ethanol to a final molar ratio of 30 mol % DODAP, 30 mol % DOPE, 39.75 mol % cholesterol, 0.25 mol % DMPE-PEG, and a total lipid concentration of 12.5 mM. The solutions were applied to a Nanoassemblr Spark cartridge (Precision Nanosystems) and liposomes were formed in the instrument per the manufacturer's directions. The resulting liposomes were collected and immediately diluted in PBS (Ambion).

Liposomes comprising 2 μg of RNA were applied dropwise to 50,000 HEKn in one well of a 6-well plate. After 48 hours, gene editing efficiency was assayed using a mutation-specific nuclease (T7E1). Results of this assay are depicted in FIG. 16.

### Example 19 in Vivo RIBOSLICE Safety Study

40 female NCr nu/nu mice were injected subcutaneously with 5×10⁶ MDA-MB-231 tumor cells in 50% Matrigel (BD Biosciences). Cell injection volume was 0.2 mL/mouse. The age of the mice at the start of the study was 8 to 12 weeks. A pair match was conducted, and animals were divided into 4 groups of 10 animals each when the tumors reached an average size of 100-150 mm³, and treatment was begun. Body weight was measured every day for the first 5 days, and then biweekly to the end of the study. Treatment consisted of RIBOSLICE BIRC5-1.2 complexed with a vehicle (LIPOFECTAMINE 2000, Life Technologies Corporation). To prepare the dosing solution for each group, 308 μL of complexation buffer (Opti-MEM, Life Technologies Corporation) was pipetted into each of two sterile, RNase-free 1.5 mL tubes. 22 μL of RIBOSLICE BIRC5-1.2 (500 ng/μL) was added to one of the two tubes, and the contents of the tube were mixed by pipetting. 22 μL of vehicle was added to the second tube. The contents of the second tube were mixed, and then transferred to the first tube, and mixed with the contents of the first tube by pipetting to form complexes. Complexes were incubated at room temperature for 10 min. During the incubation, syringes were loaded. Animals were injected either intravenously or intratumorally with a total dose of 1 μg RNA/animal in 60 μL total volume/animal. A total of 5 treatments were given, with injections performed every other day. Doses were not adjusted for body weight. Animals were followed for 17 days. No significant reduction in mean body weight was observed, demonstrating the in vivo safety of RIBOSLICE gene-editing RNA.

### Example 20 In Vivo RIBOSLICE Efficacy Study—Subcutaneous Glioma Model

40 female NCr nu/nu mice were injected subcutaneously with 1×10⁷ U-251 tumor cells. Cell injection volume was 0.2 mL/mouse. The age of the mice at the start of the study was 8 to 12 weeks. A pair match was conducted, and animals were divided into 4 groups of 10 animals each when the tumors reached an average size of 35-50 mm³, and treatment was begun. Body weight was measured every day for the first 5 days, and then biweekly to the end of the study. Caliper measurements were made biweekly, and tumor size was calculated. Treatment consisted of RIBOSLICE BIRC5-2.1 complexed with a vehicle (LIPOFECTAMINE 2000, Life Technologies Corporation). To prepare the dosing solution, 294 μL of complexation buffer (Opti-MEM, Life Technologies Corporation) was pipetted into a tube containing 196 μL of RIBOSLICE BIRC5-1.2 (500 ng/μL), and the contents of the tube were mixed by pipetting. 245 μL of complexation buffer was pipetted into a tube containing 245 μL of vehicle. The contents of the second tube were mixed, and then transferred to the first tube, and mixed with the contents of the first tube by pipetting to form complexes. Complexes were incubated at room temperature for 10 min. During the incubation, syringes were loaded. Animals were injected intratumorally with a total dose of either 2 μg or 5 μg RNA/animal in either 20 μL or 50 μL total volume/animal. A total of 5 treatments were given, with injections performed every other day. Doses were not adjusted for body weight. Animals were followed for 25 days.

### Example 21 Wound Healing Treatment

RNA encoding basic fibroblast growth factor or IL22 is prepared according to Example 1. The RNA is delivered by loading into a syringe and delivering the RNA by intradermal injection to the ventral forearm of a healthy 33-year-old male patient over the course of approximately 30 seconds or by exposing an area of skin to electrical pulses of between 10V and 155V and between approximately 10 milliseconds and approximately 1 second using a two-electrode array electrically connected to a capacitor, or by applying the RNA (with or without a liposome) directly to the skin, with or without disruption of the stratum corneum or injected intradermally or delivered by injection to the epidermis using a dose of one microgram or less per square centimeter. Optionally, an electric field is applied or a surface-contact patch to enhance delivery of the RNA is used.

### Example 22: Gene Editing of the COL7A1 Gene

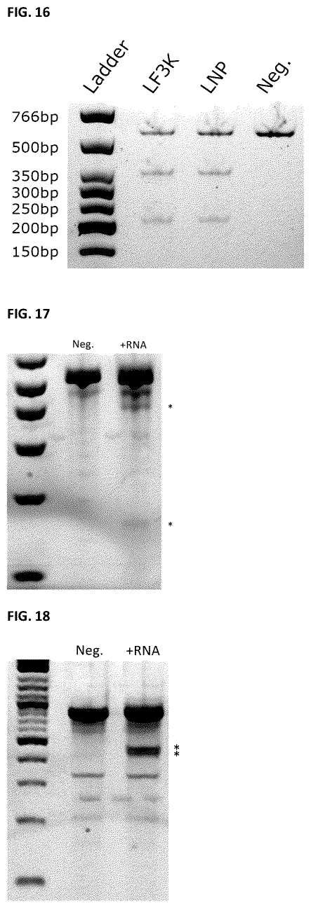

The present RNA-based gene editing approaches were applied to the COL7A1 gene. This gene is of interest because, inter alia, it is frequently involved in dystrophic epidermolysis bullosa. FIG. 17 depicts a SURVEYOR assay using the DNA of primary adult human dermal fibroblasts transfected with RNA TALENs targeting the sequence TGAGCAGAAGTGGCTCAGTG (SEQ ID NO: 467) and TGGCTGTACAGCTACACCCC (SEQ ID NO: 468), located within the COL7A1 gene. The bands present in the +RNA lane indicate editing of a region of the gene that is frequently involved in dystrophic epidermolysis bullosa. FIG. 18 depicts another SURVEYOR assay using the DNA of primary adult human dermal fibroblasts transfected with RNA TALENs, now targeting the sequence TTCCACTCCTGCAGGGCCCC (SEQ ID NO: 469) and TCGCCCTTCAGCCCGCGTTC (SEQ ID NO: 470), located within the COL7A1 gene. The bands present in the +RNA lane indicate editing of a region of the gene that is frequently involved in dystrophic epidermolysis bullosa. This data points to, among others, a gene editing approach to the treatment of certain genetic disorders such as dystrophic epidermolysis bullosa.

### Example 23: Gene-Editing of the MYC Gene Using a Synthetic RNA with Non-Canonical Nucleotides

Experiments were conducted with in vitro transcribed synthetic RNA molecules containing non-canonical nucleotides and encoding gene-editing proteins. The immunogenicity and the gene-editing efficiency of in vitro transcribed synthetic RNA molecules having (1) only pseudouridine (psU) as a non-canonical nucleotide; (2) only 5-methylcytidine (5mC) as a non-canonical nucleotide; and (3) both of pseudouridine and 5-methylcytidine as non-canonical nucleotides was evaluated (as well as controls).

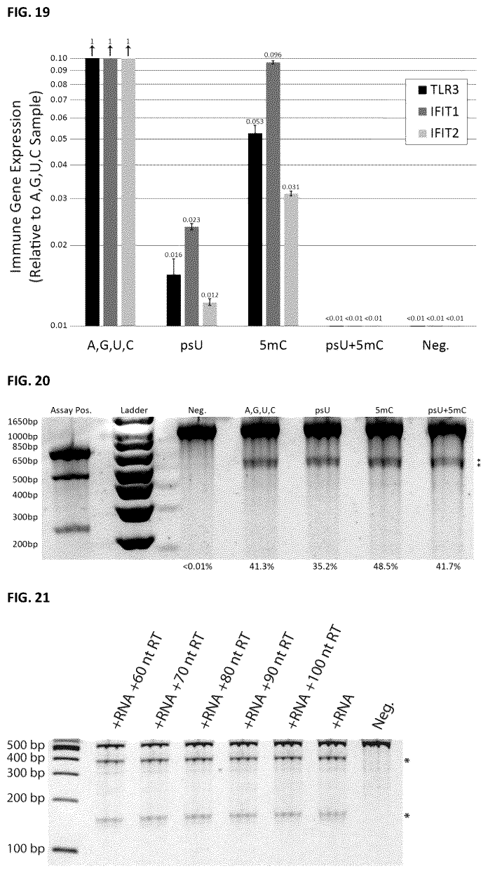

Specifically, RNA containing the following nucleotide combinations: (i) A,G,U,C, (ii) A,G,psU,C, (iii) A,G,U,5mC, and (iv) A,G,psU,5mC, and encoding TALEN pairs targeting the following DNA sequences, which can be found within the MYC gene: TCGGCCGCCGCCAAGCTCGT (SEQ ID NO: 474) and TGCGCGCAGCCTGGTAGGAG (SEQ ID NO: 475), were synthesized according to the methods described herein. Human dermal fibroblasts (MA001SK) were plated in 6-well and 24-well tissue culture plates in DMEM with 10% FBS at 100,000 and 10,000 cells per well, respectively. The next day, the cells were transfected in the 6-well plate with 2 μg of RNA (1 μg for each component of the TALEN pair) and the cells were transfected in the 24-well plate with 0.2 μg of RNA (0.1 μg for each component of the TALEN pair) according to the methods described herein. 24 hours after transfection, the total RNA from the cells in the 24-well culture plate was isolated using an RNeasy Mini Kit (74106; QIAGEN), including isolating the total RNA from a sample of cells that had not been transfected with RNA (negative control; “Neg.” in FIG. 19). The genomic DNA was removed by a 15-minute digestion with DNase I (RNase-Free) (M0303L; NEW ENGLAND BIOLABS) and the reaction purified using an RNeasy Mini Kit. 1 μL of total RNA was used to assess gene expression by real-time RT-PCR using TAQMAN gene-expression assays (APPLIED BIOSYSTEMS) designed to detect expression of the immunogenicity markers TLR3, IFIT1, and IFIT2 (FIG. 19). The data were normalized to both the positive experimental control sample (“A,G,U,C”) and to a loading control (GAPDH).

48 hours after transfection, the genomic DNA was isolated from the cells in the 6-well culture plate using a DNeasy Blood and Tissue Kit (69506; QIAGEN), including from a sample of cells that had not been transfected with RNA (negative control, “Neg.” in FIG. 20). A 970 bp region of the MYC gene surrounding the predicted TALEN cut location was amplified using a 35 cycle 2-step PCR reaction containing the following primers: TAACTCAAGACTGCCTCCCGCTTT (SEQ ID NO: 476) and AGCCCAAGGTTTCAGAGGTGATGA (SEQ ID NO: 477). 160 ng was hybridized in 5 μL of amplified sequence from RNA-treated cells to 160 ng in 5 μL of amplified sequences from untreated MA001SK cells by mixing the two sequences with 0.5 μL of 1M KCl and 0.5 μL of 25 mM MgCI₂ and running the following program in a thermocycler: 95° C. for 10 minutes; 95° C. to 85° C. at 0.625C/s; 85° C. to 25° C. at 0.125C/s. The SURVEYOR assay was performed by adding 0.5 μL of SURVEYOR nuclease and 0.5 μL of Enhancer from the SURVEYOR Mutation Detection Kit (7060201; INTEGRATED DNA TECHNOLOGIES) to the hybridized product, mixing, and incubating at 42° C. for 25 minutes. The protocol above was also used to process the positive control DNA sample provided with the SURVEYOR Mutation Detection Kit as a positive experimental control for the SURVEYOR Assay (“Assay Pos.” in FIG. 20). Samples were analyzed by agarose gel electrophoresis (FIG. 20). For each sample, gene-editing efficiency was calculated as a ratio of the intensity of the digested bands (indicated by “*” in FIG. 20) to that of the undigested band.

As shown in FIG. 19, the samples from cells transfected with the positive control RNA (A,G,U,C), and the samples from cells transfected with RNA containing either pseudouridine or 5-methylcytidine exhibited upregulation of all three of the immunogenicity markers TLR3, IFIT1, and IFIT2. The sample from cells transfected with RNA containing both pseudouridine and 5-methylcytidine exhibited negligible upregulation of the immunogenicity markers (less than 0.01-fold of the positive control), demonstrating that in vitro transcribed synthetic RNA with both pseudouridine and 5-methylcytidine and encoding a gene-editing protein can evade detection by the innate-immune system of mammalian cells.

Further, as shown in FIG. 20 below, the sample from cells transfected with RNA containing both pseudouridine and 5-methylcytidine exhibited highly efficient gene editing (41.7%), which was greater than the efficiency exhibited by samples from cells transfected with RNA containing pseudouridine alone (35.2%), demonstrating that in vitro transcribed synthetic RNA comprising both pseudouridine and 5-methylcytidine and encoding a gene-editing protein can both (i) gene-edit mammalian cells at high efficiency, and (ii) gene-edit mammalian cells at higher efficiency than in vitro transcribed synthetic RNA comprising pseudouridine and not comprising 5-methylcytidine.

### Example 24: COL7A1 Gene Editing and Repair in Human Cells

RNA encoding gene editing proteins targeting the following sequences in the COL7A1 gene was synthesized according to Example 1: TGAGCAGAAGTGGCTCAGTG (SEQ ID NO: 473) and TGGCTGTACAGCTACACCCC (SEQ ID NO: 468) (see also table below).

### RNA Synthesis

| | | Reaction | ivT |
|---|---|---|---|
| Template | Nucleotides | Volume/μL | Yield/μg |
| COL7A1 TALEN 1L | A, G, 5moU, C | 20 | 120.528 |
| COL7A1 TALEN 1R | A, G, 5moU, C | 20 | 120.204 |
| COL7A1 TALEN 1L | A, G, 5moU, C | 15 | 81.94 |
| COL7A1 TALEN 1R | A, G, 5moU, C | 15 | 61.88 |

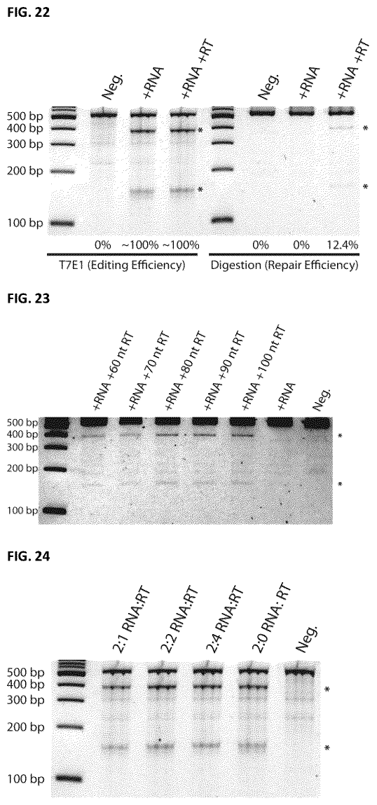

50,000 primary human epidermal keratinocytes (HEKn, Gibco) were plated in wells of 6-well plates in EpiLife+Supplement S7. The next day, cells were transfected according to Example 3 with 1 μg of RNA encoding each component of the gene editing pair and 2 μg of a single-stranded DNA repair template having a length of 60, 70, 80, 90 or 100 nucleotides (“nt”). 48 hours after transfection, genomic DNA was purified. A segment of the COL7A1 gene was amplified using the primers GCATCTGCCCTGCGGGAGATC (SEQ ID NO: 478) and CCACGTTCTCCTTTCTCTCCCCGTTC (SEQ ID NO: 479), which produce a 535 bp amplicon. The efficiency of gene editing was assessed using T7 Endonuclease I (“T7E1”, New England Biolabs) according to the manufacturer's instructions. Bands of approximately 385 bp and 150 bp indicate successful gene editing. FIG. 21 and FIG. 22 show the result of digestion with T7El, analyzed by agarose gel electrophoresis. FIG. 22 and FIG. 23 show the result of digestion with Mlul-HF, analyzed by agarose gel electrophoresis. Because the repair template contains the sequence ACGCGT (SEQ ID NO: 480), digestion of the amplified product with Mlul-HF (New England Biolabs) produces bands of approximately 385 bp and 150 bp in the case of successful gene repair.

RNA encoding gene editing proteins targeting the following sequences in the COL7A1 gene was synthesized according to Example 1: TGAGCAGAAGTGGCTCAGTG (SEQ ID NO: 473) and TGGCTGTACAGCTACACCCC (SEQ ID NO: 468). 50,000 primary human epidermal keratinocytes (HEKn, Gibco) were plated in wells of 6-well plates in EpiLife+Supplement S7. The next day, cells were transfected according to Example 3 with 1 μg of RNA encoding each component of the gene editing pair and 1-4 μg of an 80 nucleotide single-stranded DNA repair template. 48 hours after transfection, genomic DNA was purified. A segment of the COL7A1 gene was amplified using the primers GCATCTGCCCTGCGGGAGATC (SEQ ID NO: 481) and CCACGTTCTCCTTTCTCTCCCCGTTC (SEQ ID NO: 482), which produce a 535 bp amplicon. The efficiency of gene editing was assessed using T7 Endonuclease I (“T7E1”, New England Biolabs) according to the manufacturer's instructions. Bands of approximately 385 bp and 150 bp indicate successful gene editing. FIG. 24 show the result of digestion with T7El, analyzed by agarose gel electrophoresis. FIG. 25 show the result of digestion with Mlul-HF, analyzed by agarose gel electrophoresis. Because the repair template contains the sequence ACGCGT (SEQ ID NO: 480), digestion of the amplified product with Mlul-HF (New England Biolabs) produces bands of approximately 385 bp and 150 bp in the case of successful gene repair.

### Example 25: Pharmacokinetic Study Via Intradermal Injection in Rats

Studies were conducted to evaluate the responses of Sprague Dawley rats to intradermal administration of various RNAs. Specifically, 8-10 weeks old, female Sprague Dawley rats weighing about 200 g to about 350 g were used for this study. A total of 33 rats were tested, and the animals were assigned to study groups and treated as indicated in the Table below:

| | | | | | | Dose | |
|---|---|---|---|---|---|---|---|
| | | | | | Dose | Volume | Number of |
| | Group | Test | Dose | Dose Level | Concentration | (μL/per | Animals |
| Group | Color | Article | Route | (μg) | (μg/mL) | injection)^{a} | Females |
| 1 | White | Control | ID | 4.0 | 20.0 | 200 | 3^{b} |
| | | (NOVEPOEITIN) | | | | (4 × 50) | |
| 2 | Yellow | TA1 (IL2) | ID | 4.0 | 20.0 | 200 | 3^{b} |
| | | | | | | (4 × 50) | |
| 3 | Green | TA2 (IL6) | ID | 4.0 | 20.0 | 200 | 3^{b} |
| | | | | | | (4 × 50) | |
| 4 | Blue | TA3 (IL15) | ID | 4.0 | 10.0 | 200 | 3^{b} |
| | | | | | | (4 × 50) | |
| 5 | Red | TA4 | ID | 4.0 | 20.0 | 200 | 3^{b} |
| | | (IL15 + IL15RA) | | | (10.0 each) | (4 × 50) | |
| 6 | Dark Grey | TA5 (IL22) | ID | 4.0 | 20.0 | 200 | 3^{b} |
| | | | | | | (4 × 50) | |
| 7 | Purple | TA6 (BMP2) | ID | 4.0 | 20.0 | 200 | 3^{b} |
| | | | | | | (4 × 50) | |
| 8 | Black | TA7 (BDNF) | ID | 4.0 | 20.0 | 200 | 3^{b} |
| | | | | | | (4 × 50) | |
| 9 | White/ | TA8 (LIF) | ID | 4.0 | 20.0 | 200 | 3^{b} |
| | Yellow | | | | | (4 × 50) | |
| 10 | Green/ | TA9 (PTH) | ID | 4.0 | 20.0 | 200 | 3^{b} |
| | Blue | | | | | (4 × 50) | |
| 11 | Red/Dark | TA10 (FGF21) | ID | 4.0 | 20.0 | 200 | 3^{b} |
| | Grey | | | | | (4 × 50) | |

^{a}Total dose volume (μL/per animal) was constant. Each animal received four intradermal injections of 50 μL/per injection for a total of 200 μL per animal.
^{b}Animals, euthanized on Day 3 without examination or necropsy
Intradermal = ID

For the study, the animals were treated with 4 μg of RNA. All groups were dosed via intradermal injection. Each dose was administered in four intradermal injections of 50 NL/injection for a total of 200 NL per animal. Injections occurred into previously marked sites near the midline of the dorsal lumbar area (upper left, upper right, lower left and lower right quadrants). Dose time (after the last injection) was recorded. Additional markings were made as needed to allow for identification of the dose site. Animals were administered with the RNAs on day 1 and euthanized on day 3. Clinical observations were made on the rats twice daily. Food consumption and body weight were also monitored.

During the study, approximately 1 ml of blood samples was collected from the jugular vein for pharmacokinetic analysis as follows:

| Time Point | PK^{a} |
|---|---|
| Acclimation | — |
| Day 1 | 12 hours postdose |
| Day 2 | 24 hours postdose |
| Day 3 | 48 hours postdose |

^{a}Time points for blood collection (n = 3 animals/Group/Time point)
PK = Pharmacokinetics

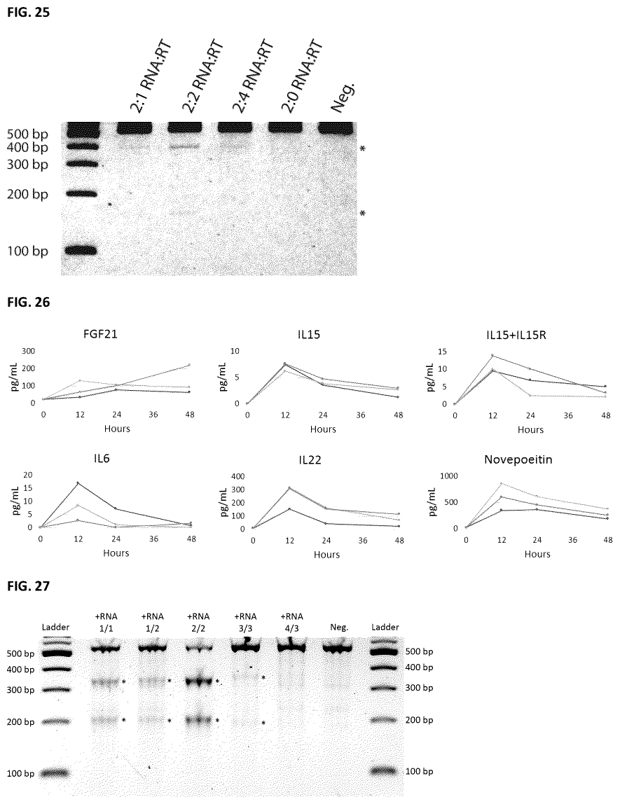

Results indicate that following administration of RNAs encoding FGF21, IL15, IL15 and IL15R, IL6, IL22, and NOVEPOEITIN, these proteins were readily detected in the blood with protein levels peaking at approximately 12 hours post injection (FIG. 26). Of note, the proteins tested in this study can be taken up by cells and tissues and/or can exert an effect near the site of expression without appreciable accumulation in systemic circulation.

### Example 60: Gene Editing of COL7A1

With reference to Example 1, Table 4, the following templates and targets were used:

| | | SEQ ID |
|---|---|---|
| Template | Target Sequence | NO: |
| COL7A1 exon 73 spliceMod | TACAGCCACCAGCATTCTCT | 651 |
| TALEN L1 | | |
| COL7A1 exon 73 spliceMod | TGTACAGCCACCAGCATTCT | 652 |
| TALEN L2 | | |
| COL7A1 exon 73 spliceMod | TGGGGTGTAGCTGTACAGCC | 653 |
| TALEN L3 | | |
| COL7A1 exon 73 spliceMod | TGGAGTGGGGTGTAGCTGTA | 654 |
| TALEN L4 | | |
| COL7A1 exon 73 spliceMod | TCTCCAGGAAAGCCGATGGG | 655 |
| TALEN R1 | | |
| COL7A1 exon 73 spliceMod | TCCAGGAAAGCCGATGGGGC | 656 |
| TALEN R2 | | |
| COL7A1 exon 73 spliceMod | TGGGGCCCTGCAGGAGTGGA | 657 |
| TALEN R3 | | |

About 50,000 primary human neonatal epidermal keratinocytes (animal-protein free) were plated in EpiLife+Supplement S7. Cells were transfected according to Example 3 with 2 μg RNA encoding the COL7A1 exon 73 spliceMod TALEN L1-4 and COL7A1 exon 73 spliceMod TALEN R1-3 gene-editing proteins (1 μg each), in the following combinations: L1/R1, L1/R2, L2/R2, L3/R3, or L4/R3. After 48 hours, DNA was harvested and analyzed for gene editing (T7E1 assay; forward primer: GCATCTGCCCTGCGGGAGATC (SEQ ID NO: 478); reverse primer: CCACGTTCTCCTTTCTCTCCCCGTTC (SEQ ID NO: 479); product size: 535 nt; predicted band sizes: 203 nt, 332 nt for L1/R1; 202 nt, 333 nt for L1/R2; 201 nt, 334 nt for L2/R2; 189 nt, 346 nt for L3/R3; and 186 nt, 349 nt for L4/R3). Results shown in FIG. 27 demonstrate efficient gene editing by the gene-editing proteins.

### Example 27: Gene Editing of COL7A1 Using RIBOSLICE

With reference to Example 1, Table 4, the following templates and targets were used:

| | | SEQ ID |
|---|---|---|
| Template | Target Sequence | NO: |
| COL7A1 exon 73 spliceMod | TGTACAGCCACCAGCATTCT | 652 |
| TALEN L2 | | |
| COL7A1 exon 73 spliceMod | TCCAGGAAAGCCGATGGGGC | 656 |
| TALEN R2 | | |
| COL7A1 exon 73 spliceMod | TGTACAGCCACCAGCATTCT | 652 |
| RIBOSLICE L2A | | |
| COL7A1 exon 73 spliceMod | TGTACAGCCACCAGCATTCT | 652 |
| RIBOSLICE L2B | | |
| COL7A1 exon 73 spliceMod | TCCAGGAAAGCCGATGGGGC | 656 |
| RIBOSLICE R2A | | |
| COL7A1 exon 73 spliceMod | TCCAGGAAAGCCGATGGGGC | 656 |
| RIBOSLICE R2B | | |

About 50,000 primary human neonatal epidermal keratinocytes (animal-protein free) were plated in EpiLife+Supplement S7. Cells were transfected according to Example 3 with 2 μg RNA encoding the COL7A1 exon 73 spliceMod TALEN or RIBOSLICE gene-editing proteins (1 μg each), in the following combinations: RIBOSLICE L2A/RIBOSLICE R2A, RIBOSLICE L2B/RIBOSLICE R2B, and TALEN L2/TALEN R2. After 48 hours, DNA was harvested and analyzed for gene editing (T7E1 assay; forward primer: GCATCTGCCCTGCGGGAGATC (SEQ ID NO: 478); reverse primer: CCACGTTCTCCTTTCTCTCCCCGTTC (SEQ ID NO: 479); product size: 535 nt; predicted band sizes: 202 nt, 333 nt). Results shown in FIG. 28 demonstrate efficient gene editing by the gene-editing proteins.

### Example 28: Gene Editing of COL7A1 for Altering RNA Splicing

With reference to Example 1, Table 4, the following templates and targets were used:

| | | SEQ ID |
|---|---|---|
| Template | Target Sequence | NO: |
| COL7A1 exon 73 spliceMod | TACAGCCACCAGCATTCTCT | 651 |
| TALEN L1 | | |
| COL7A1 exon 73 spliceMod | TGTACAGCCACCAGCATTCT | 652 |
| TALEN L2 | | |
| COL7A1 exon 73 spliceMod | TGGGGTGTAGCTGTACAGCC | 653 |
| TALEN L3 | | |
| COL7A1 exon 73 spliceMod | TGGAGTGGGGTGTAGCTGTA | 654 |
| TALEN L4 | | |
| COL7A1 exon 73 spliceMod | TCTCCAGGAAAGCCGATGGG | 655 |
| TALEN R1 | | |
| COL7A1 exon 73 spliceMod | TCCAGGAAAGCCGATGGGGC | 656 |
| TALEN R2 | | |
| COL7A1 exon 73 spliceMod | TGGGGCCCTGCAGGAGTGGA | 657 |
| TALEN R3 | | |

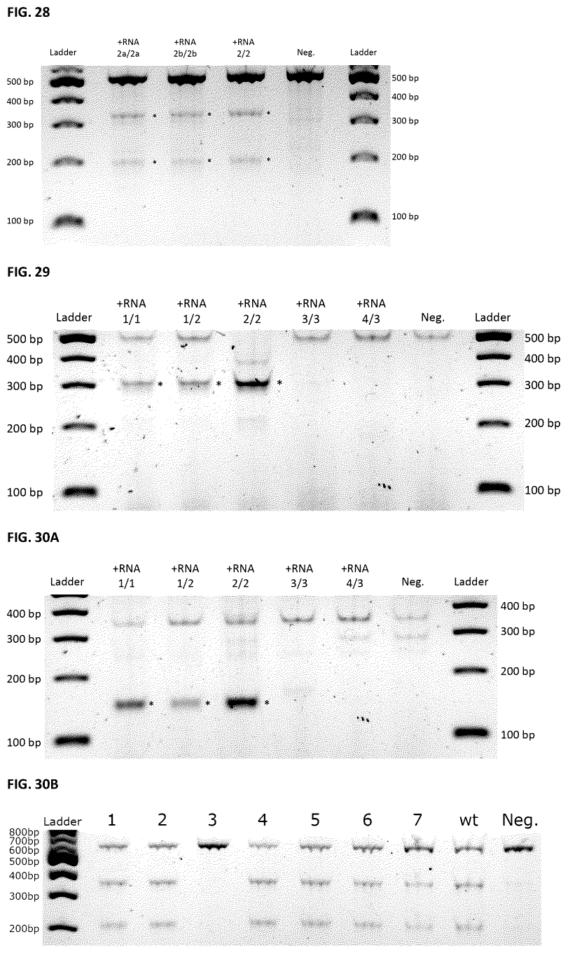

About 50,000 primary human neonatal epidermal keratinocytes (animal-protein free) were plated in EpiLife+Supplement S7. Cells were transfected according to Example 3 with 2 μg RNA encoding the COL7A1 exon 73 spliceMod TALEN L1-4 and COL7A1 exon 73 spliceMod TALEN R1-3 gene-editing proteins (1 μg each), in the following combinations: L1/R1, L1/R2, L2/R2, L3/R3, L4/R3. After 48 hours, RNA was harvested. mRNA was reverse transcribed using ROCKETSCRIPT reverse transcriptase and the primer GCTCTCCTGGTAGACCCGGGTTG (SEQ ID NO: 658). The resulting product was amplified by PCR using the primer pair GGTTGCTGGAAACTGCTGGCATCAAGGCATCTG (SEQ ID NO: 659) and CACCCTTGAGTCCAGGGGGTCCCTGTTCTC (SEQ ID NO: 661) (This pair produces a product size of 513 nt if exon 73 is present in the spliced mRNA and a product size of 312 nt if exon 73 is not present in the spliced mRNA) and analyzed by gel electrophoresis. Results as shown in FIG. 29 demonstrate efficient removal of exon 73. To confirm the result, the reverse-transcription product was amplified using the primer pair GCATCTGCCCTGCGGGAGATC (SEQ ID NO: 478) and CCACGTTCTCCTTTCTCTCCCCGTTC (SEQ ID NO: 479) (This pair is predicted to produce a product size of 353 nt if exon 73 is present in the spliced mRNA and a product size of 152 nt if exon 73 is not present in the spliced mRNA) and analyzed by gel electrophoresis. Results shown in FIG. 30A demonstrate efficient removal of exon 73.

FIG. 30B depicts the results of an experiment in which 50,000 primary human neonatal epidermal keratinocytes (animal-protein free) were transfected with 2 μg RNA encoding the COL7A1 exon 73 splice acceptor-targeting pairs (target sequences: TGTACAGCCACCAGCATTCT (SEQ ID NO: 652) and TCCAGGAAAGCCGATGGGGC (SEQ ID NO: 656)) (1 μg each individual pair component) with mutations in the N-terminus of the protein. 1: I56L, 2: K57R, 3: R61K, 4: A65G, 5: A70G, 6: K57E, 7. K57E and V60A. DNA was harvested after 48 h and analyzed for gene editing (T7E1 assay; forward primer: GCATCTGCCCTGCGGGAGATC (SEQ ID NO: 478), reverse primer: CCACGTTCTCCTTTCTCTCCCCGTTC (SEQ ID NO: 479), product size: 535 nt, predicted band sizes: 202 nt, 333 nt).

### Example 29: Time Course of Gene Editing of COL7A1 for Altering RNA Splicing

With reference to Example 1, Table 4, the following templates and targets were used:

| | | SEQ ID |
|---|---|---|
| Template | Target Sequence | NO: |
| COL7A1 exon 73 spliceMod | TGTACAGCCACCAGCATTCT | 652 |
| TALEN L2 | | |
| COL7A1 exon 73 spliceMod | TCCAGGAAAGCCGATGGGGC | 656 |
| TALEN R2 | | |

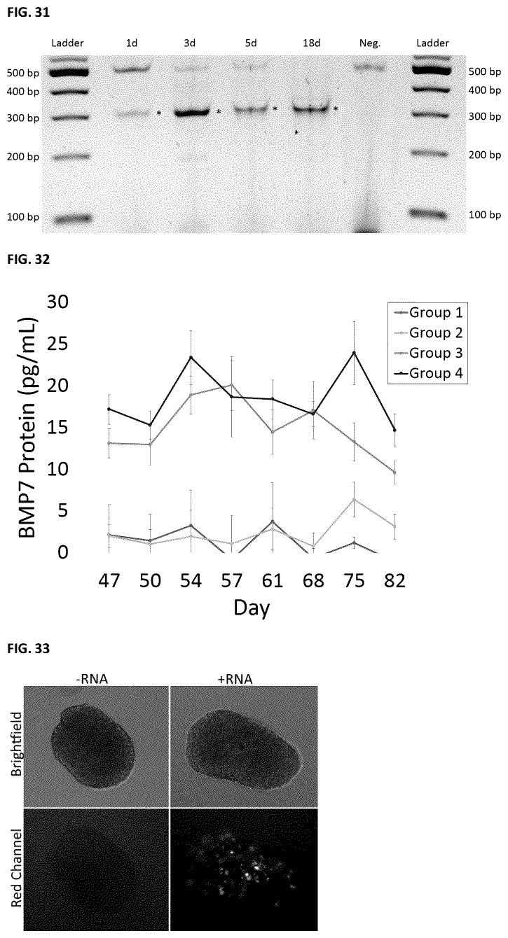

Four wells of about 50,000 primary human neonatal epidermal keratinocytes (animal-protein free) were plated in EpiLife+Supplement S7. Cells were transfected according to Example 3 with 2 μg RNA encoding the COL7A1 exon 73 spliceMod TALEN L2 and COL7A1 exon 73 spliceMod TALEN R2 gene-editing proteins (1 μg each). After 1, 3, and 5 days, RNA was harvested. The cells in the remaining well were cultured until near-senescence, 18 days after transfection, and RNA was harvested. mRNA was reverse transcribed using ROCKETSCRIPT reverse transcriptase and the primer GCTCTCCTGGTAGACCCGGGTTG (SEQ ID NO: 658). The resulting product was amplified by PCR using the primer pair GGTTGCTGGAAACTGCTGGCATCAAGGCATCTG (SEQ ID NO: 659) and CACCCTTGAGTCCAGGGGGTCCCTGTTCTC (SEQ ID NO: 661) (This pair is predicted to produce a product size of 513 nt if exon 73 is present in the spliced mRNA and a product size of 312 nt if exon 73 is not present in the spliced mRNA) and analyzed by gel electrophoresis. Results shown in FIG. 31 demonstrate efficient and persistent removal of exon 73.

### Example 30: Huntington Disease Therapy Comprising RNA Encoding Gene-Editing Proteins

RNA encoding one or more gene-editing proteins capable of creating one or more double-strand breaks in the following DNA sequence: TGCACTGCTGTCCTGCATTTCAGCATCTTCAGGATGCTGTGCAGCTGAAACATTTGATAACGGTGGAACTGT TCGTTATTTTGCAAGCCTGTGATTCCCTATTGAATGTTTTCTCTCGCCATTTGACAAATGAGTGTTTCTCTGTC TTCACCTCAGTGAAGGATGAGATCAGTGGAGAGCTGGCTGCTTCTTCAGGGGTTTCCACTCCAGGGTCAGC AGGTCATGACATCATCACAGAACAGCCACGGTC (SEQ ID NO: 708) are synthesized according to Example 1. RNA is formulated according to the methods of the present invention, and delivered by injection to the basal ganglia of a patient with Huntington's disease.

### Example 32: Gene-Editing Proteins Comprising DNA-Binding and Deaminase Activity

RNA encoding a gene-editing protein comprising two or more repeat sequences, followed by: BASE_EDIT_FRONT (SEQ ID NO: 587), followed by any of BASE_EDIT_ADA1 (SEQ ID NO: 588), BASE_EDIT_ADA2 (SEQ ID NO: 589), BASE_EDIT_ADA3 (SEQ ID NO: 590), BASE_EDIT_ADA4 (SEQ ID NO: 591), BASE_EDIT_CDA1 (SEQ ID NO: 592) and BASE_EDIT_CDA2 (SEQ ID NO: 593) is synthesized according to Example 1. The gene-editing protein is capable of correcting one or more mutations within 1 to 50 bases downstream of the target sequence.

### Example 33: Transfection of Human Keratinocytes with RNA Encoding BDNF, BMP-2, BMP-6, IL-2, IL-6, IL-15, IL-22, LIF or FGF-21

100,000 human epidermal keratinocytes (HEK, Gibco) were plated in EpiLife+Supplement S7. Cells were transfected according to Example 3 with 2 μg of RNA encoding BDNF, BMP-2, BMP-6, IL-2, IL-6, IL-15, IL-22, LIF or FGF-21. 24 hours after transfection, the medium was sampled and secreted protein levels were measured using a human ELISA kit (see Table below) according the manufacturer's instructions. Secreted protein levels were determined by measuring 450 nm absorbance using a microplate reader (EMax Plus, Molecular Devices). Secreted protein levels are shown in FIG. 32, panels A-I.

| Protein | Part Number | Vendor |
|---|---|---|
| BDNF | DBNT00 | R&D Systems |
| BMP-2 | DBP200 | R&D Systems |
| BMP-6 | ab99984 | Abcam |
| IL-2 | D2050 | R&D Systems |
| IL-6 | D6050 | R&D Systems |
| IL-15 | D1500 | R&D Systems |
| IL-22 | D2200 | R&D Systems |
| LIF | DLF00 | R&D Systems |
| FGF-21 | DF2100 | R&D Systems |

### Example 33: Friedrich's Ataxia Therapy Comprising RNA Encoding Gene-Editing Proteins

RNA encoding one or more gene-editing proteins capable of creating one or more double-strand breaks in FXNA (SEQ ID NO: 582) and RNA encoding one or more gene-editing proteins capable of creating one or more double-strand breaks in FXNB (SEQ ID NO: 583) are synthesized according to Example 1. RNA is formulated according to the methods of the present invention, and delivered to the heart of a patient with Friedrich's ataxia using a catheter. Target-sequence pairs are selected from Pair 1: TCCCACACGTGTTATTTGGC (SEQ ID NO: 618) and TGGCAACCAATCCCAAAGTT (SEQ ID NO: 619); Pair 2: TAATAAATAAAAATAAAAAA (SEQ ID NO: 620) and TTGCCTATTTTTCCAGAGAT (SEQ ID NO: 621).

### Example 34: Liposomal Transfection of CNS Tissue

Cortical tissue from whole brains of embryonic day 18 Sprague Dawley rat embryos (BRAINBITS LLC) was cut into sections of approximately 200 μm in thickness and placed onto 20 μL of gelled rat tail collagen in DMEM. RNA encoding mRFP was synthesized according to Example 1. RNA-transfection-reagent complexes with Opti-MEM and LIPOFECTAMINE 3000 were created according to Example 2. The sections were treated with a 5 μL volume containing 0.1 μg mRFP RNA-transfection-reagent complexes. The sections were covered with a second 20 μL layer of rat tail collagen in DMEM that was allowed to gel and then immersed in 500 μL of Neurobasal media with 2 mM GlutaMAX, 2% B-27 supplement and 1× antibiotic-antimycotic. Treated sections were incubated at 5% CO₂ and 37° C. for 16 h and then imaged by brightfield and fluorescent microscopy. Results shown in FIG. 33 demonstrate transfection of CNS tissue and expression of the encoded protein.

### Example 35: Liposomal Transfection of Cortical Neurons

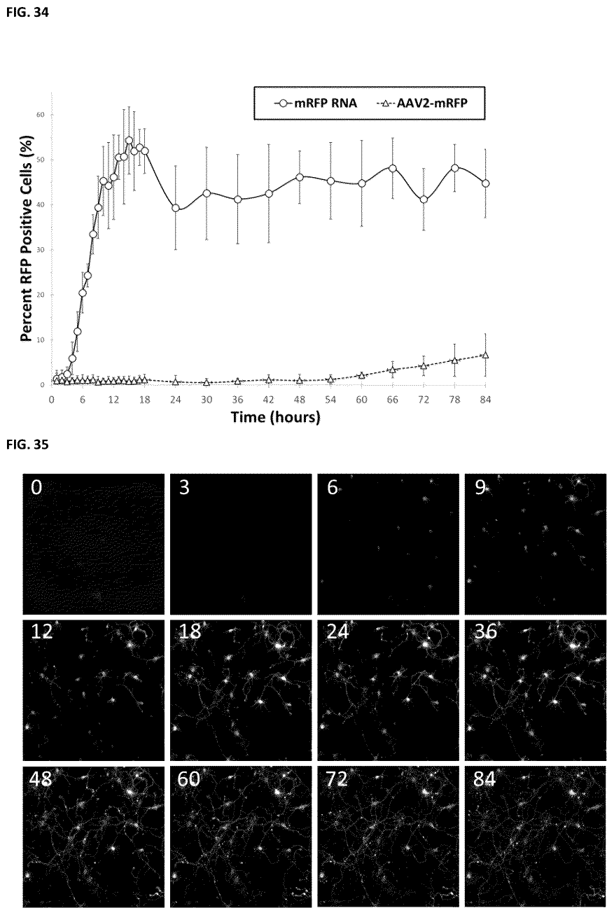

Embryonic day 18 Sprague Dawley rat embryo (BRAINBITS LLC) cortical tissue was dissociated. 50,000-100,000 cells were cultured on a poly-D-lysine coated 24-well in 500 μL of Neurobasal media with 2 mM GlutaMAX, 2% B-27 supplement and 1× antibiotic-antimycotic. After 6 days, the cells were transfected with 0.05 μg RNA encoding mRFP or 1-5×10¹⁰ viral genomes of adeno associated virus serotype 2 packed with mRFP under a CMV promoter (AAV2-mRFP purchased from SignaGen, product #SL100853). RNA encoding mRFP was synthesized according to Example 1. RNA-transfection-reagent complexes with Opti-MEM and LIPOFECTAMINE 3000 were prepared according to Example 2. The cells were imaged every hour for the first 18 h and then every 6 h by brightfield and fluorescent microscopy using a PerkinElmer Operetta CLS system at 5% CO₂ and 37° C. The percent RFP positive cells was measured using PerkinElmer Harmony 4.6 software. Results as shown in FIG. 34 and FIG. 35 demonstrate transfection of cortical neurons within 4 hours with mRFP RNA.

### Example 36: Gene Editing of HTT

About 20,000 human neuroblastoma cells (SH-SY5Y) are plated in DMEM/F-12 media with 10% fetal bovine serum. Cells are transfected according to Example 3 with 0.5 μg RNA encoding gene-editing proteins (0.25 μg each) selected from the table below; pairs 1 to 17 recognize exon 12 of the Huntingtin (HTT) gene. 48 hours after transfection, DNA is harvested and analyzed for gene editing.

| | | SEQ | | SEQ |
|---|---|---|---|---|
| | Forward Target | ID | Reverse Target | ID |
| Pair | Sequence | NO: | Sequence | NO: |
| 1 | TATTGAATGTTTTCTCTCGC | 674 | TGAGGTGAAGACAGAGAAAC | 675 |
| 2 | TATTGAATGTTTTCTCTCGC | 676 | TCACTGAGGTGAAGACAGAG | 677 |
| 3 | TGAATGTTTTCTCTCGCCAT | 678 | TCACTGAGGTGAAGACAGAG | 679 |
| 4 | TGAATGTTTTCTCTCGCCAT | 680 | TTCACTGAGGTGAAGACAGA | 681 |
| 5 | TGTTTTCTCTCGCCATTTGA | 682 | TCCTTCACTGAGGTGAAGAC | 683 |
| 6 | TTTTCTCTCGCCATTTGACA | 684 | TCCTTCACTGAGGTGAAGAC | 685 |
| 7 | TGTTTTCTCTCGCCATTTGA | 686 | TCATCCTTCACTGAGGTGAA | 687 |
| 8 | TTTCTCTCGCCATTTGACAA | 688 | TCATCCTTCACTGAGGTGAA | 689 |
| 9 | TCTCTCGCCATTTGACAAAT | 690 | TCATCCTTCACTGAGGTGAA | 691 |
| 10 | TCTCTCGCCATTTGACAAAT | 692 | TCTCATCCTTCACTGAGGTG | 693 |
| 11 | TCTCGCCATTTGACAAATGA | 694 | TCTCATCCTTCACTGAGGTG | 695 |
| 12 | TCTCTCGCCATTTGACAAAT | 696 | TGATCTCATCCTTCACTGAG | 697 |
| 13 | TCTCGCCATTTGACAAATGA | 698 | TGATCTCATCCTTCACTGAG | 699 |
| 14 | TCGCCATTTGACAAATGAGT | 700 | TGATCTCATCCTTCACTGAG | 701 |
| 15 | TTTGACAAATGAGTGTTTCT | 702 | TCCACTGATCTCATCCTTCA | 703 |
| 16 | TTTGACAAATGAGTGTTTCT | 704 | TCTCCACTGATCTCATCCTT | 705 |
| 17 | TGACAAATGAGTGTTTCTCT | 706 | TCTCCACTGATCTCATCCTT | 707 |

### Example 37: Gene Editing of HTT

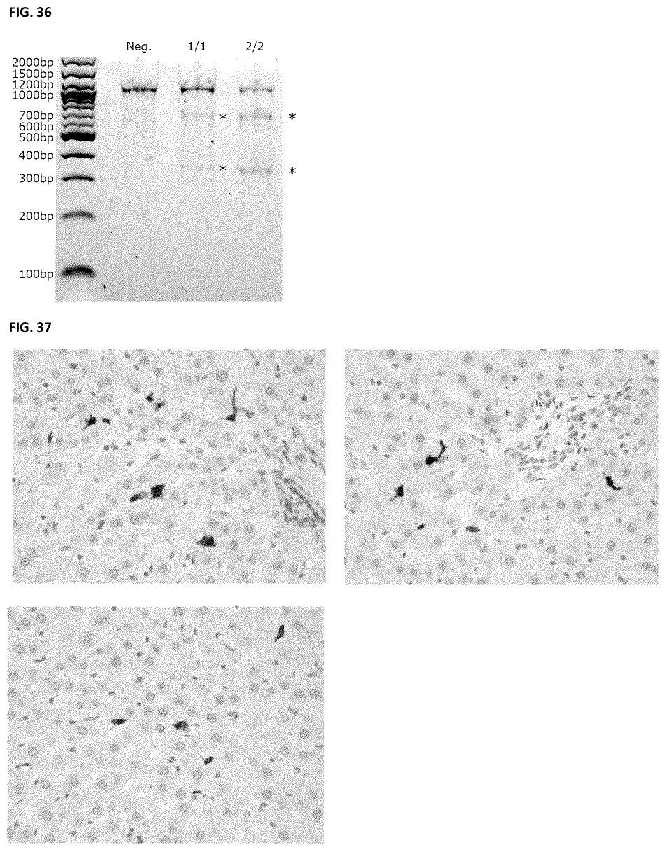

50,000 human neonatal epidermal keratinocytes (HEKn) were plated in EpiLife media supplemented with S7 (Gibco). Cells were transfected according to Example 3 with 2 μg RNA encoding gene-editing proteins (1 μg each) selected from the table below. 48 hours after transfection, DNA or RNA were harvested and analyzed for gene editing and splice modification using the T7E1 assay and RT-PCR, respectively. The appearance of 322 bp and 598 bp bands for pair 1/1 and 328 bp and 592 bp bands for pair 2/2 in the T7E1 assay indicated successful gene editing (FIG. 36). The appearance of a 236 bp band indicated successful splice modification by elimination of exon 12 from the HTT mRNA at both 3 days and 10 days.

| | Forward Target | Reverse Target |
|---|---|---|
| Pair | Sequence | Sequence |
| 1/1 | TTTGACAAATGAGTGTTTCT | TCTCCACTGATCTCATCCTT |
| | (SEQ ID NO: 710) | (SEQ ID NO: 711) |
| 2/2 | TCGCCATTTGACAAATGAGT | TGATCTCATCCTTCACTGAG |
| | (SEQ ID NO: 712) | (SEQ ID NO: 713) |

### Example 38: Localized in Vivo Protein Expression Using Directed Administration of mRNA

In this example, in vivo expression levels and expression patterns of an mRNA formulation delivered in vivo to the liver, hippocampus, intraventricular space, and intrathecal space was tested.

Two to three month old, male or female Sprague-Dawley rats were administered either a test article (of in vitro-transcribed mRNA encoding GFP, suspended in a buffer) or a control article (a control Buffer, comprising FactorPlex™ Buffer). Two days after administration, tissues were harvested and embedded in paraffin and sections were collected. Later, the sections were stained for the presence of GFP (via an anti-GFP antibody and visualized using horseradish peroxidase (HRP)/3,3′-Diaminobenzidine (DAB).

Rats in the liver expression experiments had a catheter inserted into their portal vein. Either the test article or control article was administration at 5 mL/kg IV via the indwelling cannula. GFP expression data is shown in FIG. 37.

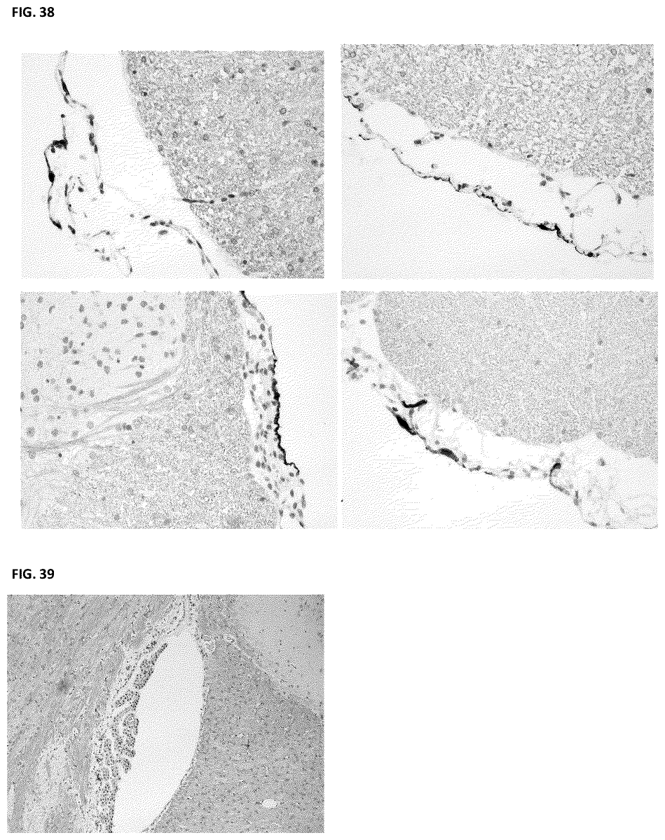

Rats in the spinal cord expression experiments had an intrathecal catheter inserted. Test article or control article administration was performed via the indwelling cannula with a bolus injection of 10 μL administered over a period of 15-20 seconds. GFP expression data is shown in FIG. 38.

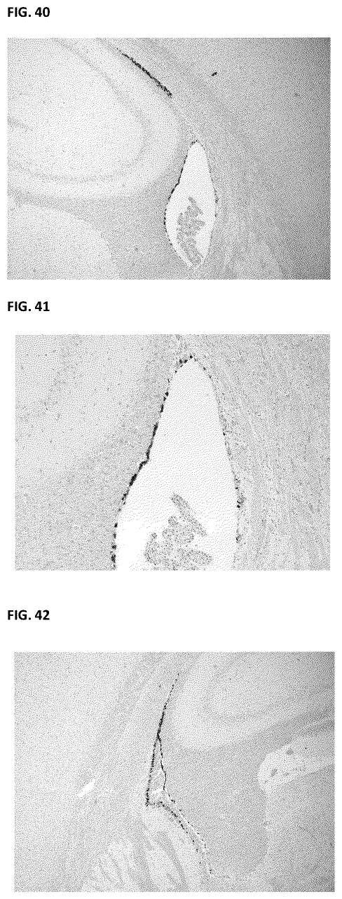

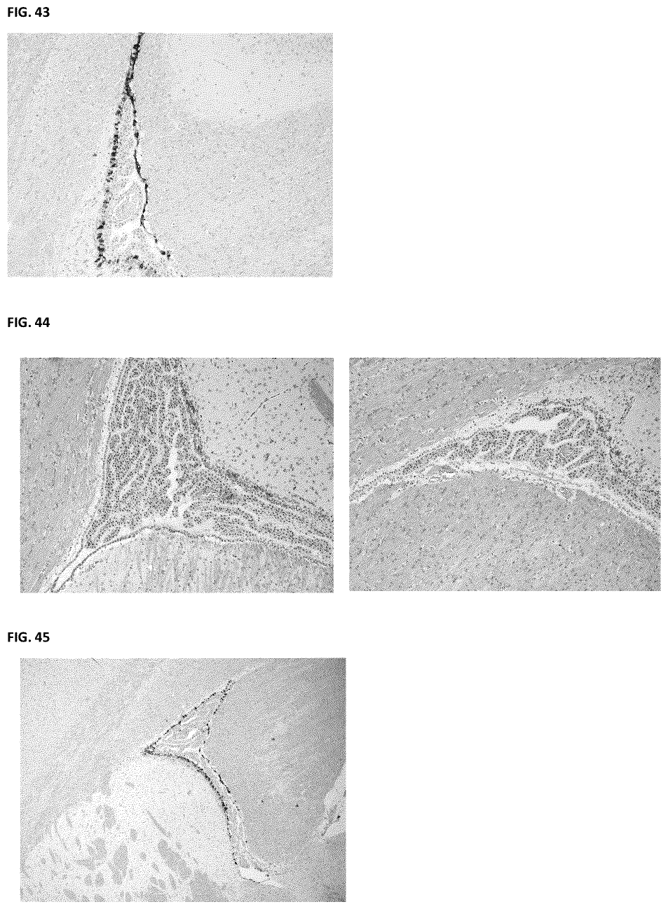

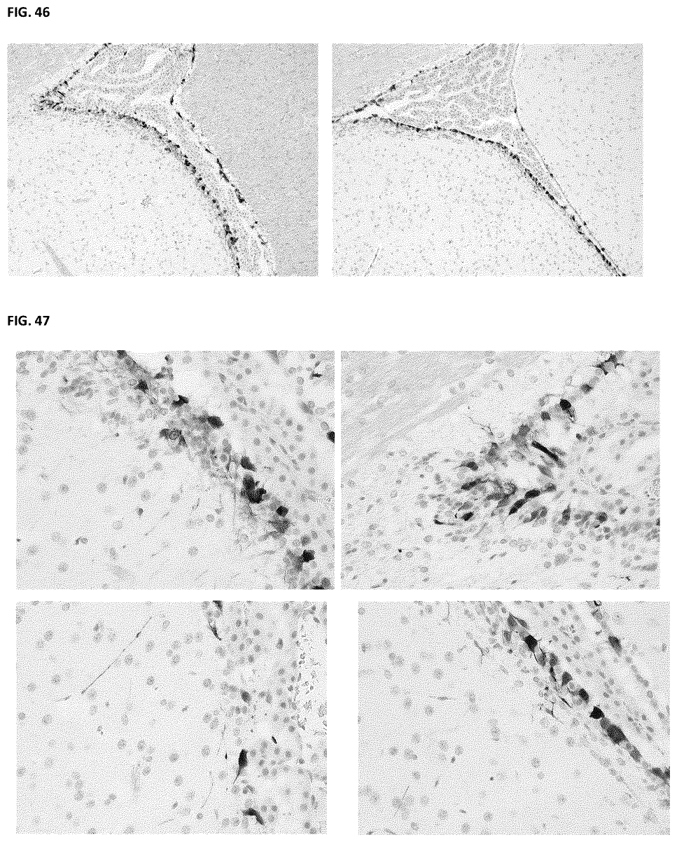

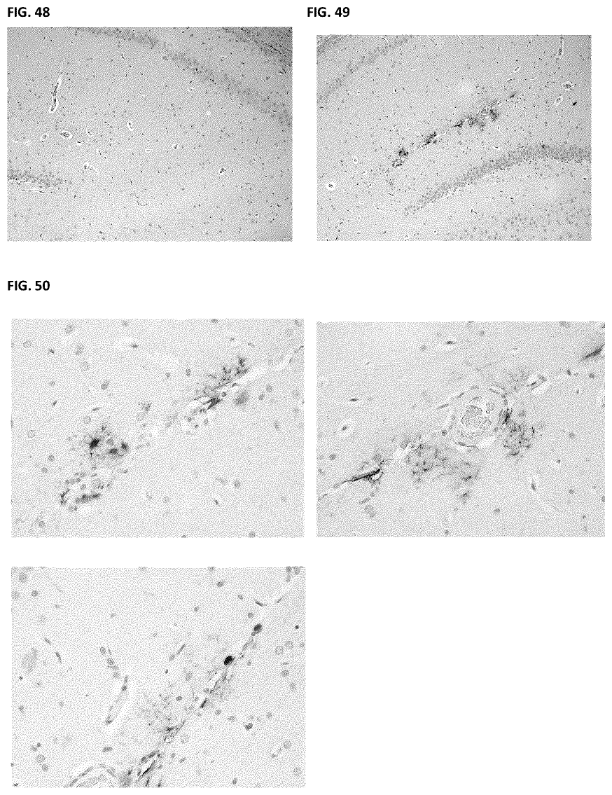

Rats in the lateral ventricle expression experiments and hippocampus expression experiments received a longitudinal, mid-scalp incision extending from the level of the eyes caudally for visualization of the bregma. The overlying tissue/muscle was retracted. The tip of the drill was placed in a position relative to the bregma for injections. The skull was drilled, taking care that only the bone was penetrated and the brain was uninjured. An appropriate syringe (Hamilton or equivalent) was be placed in position using the stereotactic frame. For administration to the lateral ventricle, the upper incisor bar was set at 3.3 mm below the interauricular line, with the bregma taken as A-P zero. The needle was placed at A −0.9, L −1.2 and V −3.6 mm. For administration to the Hippocampus, the needle was placed at AP −3.6, ML −2.2, and DV −3.2 mm from bregma. Test articles or control articles were injected slowly at 1 μL/minute for a total volume of 5 μL administered. Following the injection, the needle was left in place for 2 minutes before being raised to a depth of 1.5 mm. The needle was left in place for an additional minute at the 1.5 mm depth before complete removal. The burr hole was sealed with bone wax and the incision closed in layers using appropriate suture and materials (wound clips or equivalent). GFP expression data in the lateral ventricle is shown in FIG. 39 to FIG. 47. GFP expression data in the hippocampus is shown in FIG. 48 to FIG. 50.

These data show localized protein expression is achievable using directed administration of mRNA.

### Example 39: In Vivo Expression of Synthetic mRNA in Liver, Skin, Spinal Cord, and Brain

Experiments were conducted to examine in vivo expression levels and patterns of GFP expression from mRNA formulations delivered to the liver, neostriatum, intraventricular space, intrathecal space, and intradermal space.

Adult Sprague-Dawley rats were anesthetized and placed in an appropriate position (e.g., dorsal, lateral, or ventral recumbency) to facilitate the injection procedure. Warming devices were used to aid the animal in maintaining adequate body temperature while under anesthesia. The injection site(s) were aseptically prepared.

Animals were treated with mRNA (as lipid nanoparticles (LNPs) (mRNA-GFPA) or using LIPOFECTAMINE 3000 (mRNA-GFPB)) according to the parameters described in below the below table.

| | No. of | Description | | |
|---|---|---|---|---|
| Group | Animals | of Treatment | Dose | Dose Volume |
| 1 | 2 | Liver | 0.375 mg/kg | 5 mL/kg |
| | | infusion—mRNA- | | |
| | | GFP-A | | |
| 2 | 2 | Neostriatal | 0.375 μg/animal | 5 μL/animal |
| | | injection—mRNA- | | |
| | | GFP-A | | |
| 3 | 2 | Lateral ventricle | 0.375 μg/animal | 5 μL/animal |
| | | injection—mRNA- | | |
| | | GFP-A | | |
| 4 | 2 | Intrathecal | 0.75 μg/animal | 10 μL/animal |
| | | injection—mRNA- | | |
| | | GFP-A | | |
| 5 | 2 | Intradermal | 7.5 μg/animal | 100 μL/animal |
| | | Injection—mRNA- | | |
| | | GFP-A | | |
| 6 | 2 | Intradermal | 2 μg/animal | 100 μL/animal |
| | | Injection—mRNA- | | |
| | | GFP-B | | |

Animals in Group 1, which were treated with mRNA by liver infusion, had a portal vein catheter inserted. mRNA (5 mL/kg) was intravenously administered via the indwelling cannula.

Animals in Group 2 and 3, which were, respectively, treated with mRNA by injection into the Lateral Ventricle or Neostriatum had an incision made longitudinally in the mid-scalp extending from the level of the eyes caudally for visualization of the Bregma. The overlying tissue/muscle was be retracted. The tip of the drill was placed in a position relative to the Bregma, which is the anatomical point on the skull where the Coronal Suture intersects the Sagittal Suture. The skull was drilled, taking care that only the bone was penetrated and the brain was uninjured.

The drill was removed and an appropriate syringe (Hamilton or equivalent) was positioned using a stereotactic frame.

For treatments to the Lateral Ventricle (Group 2 rats): The upper incisor bar was set at 3.3 mm below the intraarticular line, and the bregma was taken as A-P zero. The needle was placed at A −0.9, L −1.2 and V −3.6 mm.

For treatments to the Neostriatum (Group 3 rats): AP 0.5-1, ML+2.5, DV 5.4-6 mm. mRNA was injected slowly at 1 μL/minute for a total volume of 5 μL.

Animals in Group 4, which were treated with mRNA by intrathecal injection, had an intrathecal catheter inserted. A bolus injection of 10 μL mRNA was administered via the indwelling cannula over a period of 15-20 seconds.

Animals in Groups 5 and 6, which were treated with mRNA by intradermal injection, were administered a bolus injection of 100 μL mRNA/animal with the injection site held off to prevent any leakage of material from the injection site. The injection site was marked with indelible marker to aid in necropsy tissue collections.

Rats were sacrificed on Day 2 (48±4 hours after treatment). Tissues were collected, paraffin embedded and sectioned. GFP protein was detected by immunocytochemistry.

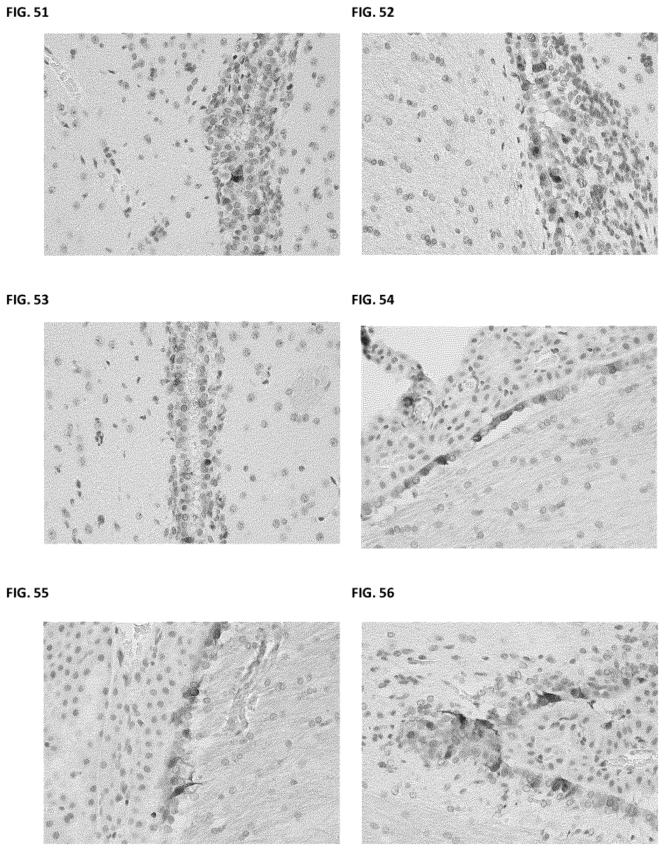

As shown in FIG. 51 to FIG. 56, Group 2 rats (which received GFP mRNA treatments to the Lateral Ventricle) has GFP protein expression in cells lining the lateral ventricle.

These data show successful in vivo expression of synthetic mRNA of the present invention.

### Example 40: In Vivo Expression of Synthetic mRNA in the Eye

Experiments will be conducted to examine in vivo expression levels and patterns of GFP expression following intravitreal injection, subretinal injection, or topical administration of mRNA formulations in rabbits or rats.

Adult rats or rabbits will be anesthetized and injected with a synthetic mRNA formulation as in Example 39, according the parameters described in below table. For each animal in each group, one eye will receive the mRNA formulation via the intended route.

| | | | No. of | |
|---|---|---|---|---|
| | No. of | Description of | Treatments | Interim |
| Group | Animals | Treatment | per animal | Procedures |
| 1 | 2 (rabbit) | Unilateral intravitreal | 1 | Ophthalmic |
| | | injection, control buffer | examinations | |
| 2 | 2 (rabbit) | Unilateral intravitreal | 1 | Ophthalmic |
| | | injection, mRNA GFP-A | examinations | |
| 3 | 2 (rabbit) | Unilateral intravitreal | 1 | Ophthalmic |
| | | injection, mRNA GFP-B | examinations | |
| 4 | 4 (rat) | Unilateral subretinal | 1 | Ophthalmic |
| | | injection, GFP-A | examinations | |
| 5 | 4 (rat) | Unilateral subretinal | 1 | Ophthalmic |
| | | injection, GFP-B | | examinations |
| 6 | 2 (rabbit) | Unilateral topical | 1 | Ophthalmic |
| | | application, GFP-A | | examinations |
| 7 | 2 (rabbit) | Unilateral topical | 1 | Ophthalmic |
| | | application, GFP-B | | examinations |

6 hours, 24 hours, and 48 hours after administration, eyes will be imaged using a fluorescent ophthalmic microscope.

Animals will be sacrificed on Day 2 (48±4 hours after treatment). Eye tissue will be collected, paraffin embedded and sectioned. GFP will be detected by immunocytochemistry.

### Example 41: Chronic Pain Therapy Comprising RNA Encoding Gene-Editing Proteins

RNA encoding one or more gene-editing proteins capable of creating one or more double-strand breaks in NaV1.3 (SCN3A, SEQ ID NO: 671), NaV1.7 (SC9N9A, SEQ ID NO: 662), NaV1.8 (SCN10A, SEQ ID NO: 672), and/or NaV1.9 (SCN11A, SEQ ID NO: 673) are synthesized according to Example 1. RNA is formulated according to the methods of the present invention, and delivered by injection to the dorsal root ganglia of a patient with chronic pain.

### Example 42: Gene Editing of NaV 1.7 in Human Cells

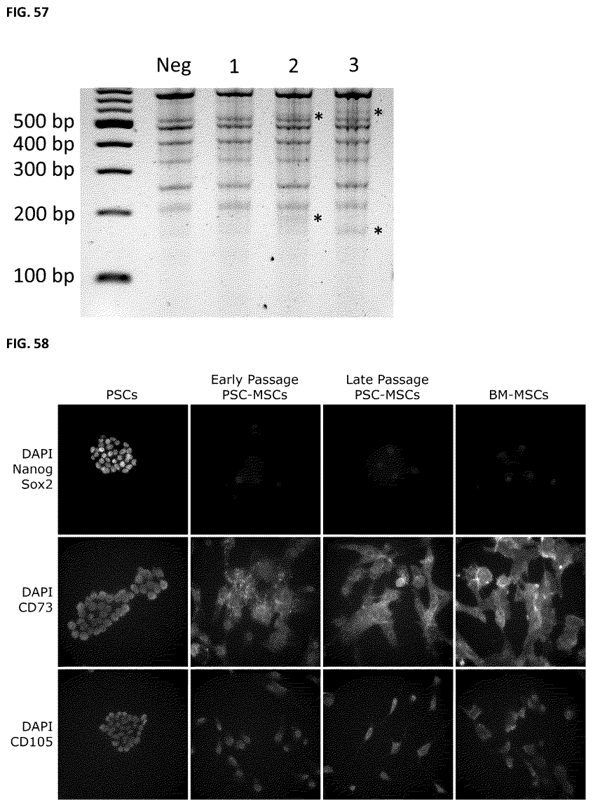

About 20,000 human neuroblastoma cells (SH-SY5Y) were plated in DMEM/F-12 media with 10% fetal bovine serum. Cells were transfected according to Example 3 with 0.5 μg RNA encoding the NaV 1.7 pairs 1, 2 or 3 gene-editing proteins (0.25 μg each): Pair 1: TCCATCCAGGCCTCTTATGT (SEQ ID NO: 663) and TCTTTTCATCCTGTATATTT (SEQ ID NO: 664); Pair 2: TGAAAAGATGGCAATGTTGC (SEQ ID NO: 665) and TGTGAAATGGACAAAGCTCT (SEQ ID NO: 666); Pair 3 TCCCCCAGGACCTCAGAGCT (SEQ ID NO: 667) and TTCAATGAGGGCAAGAGACT (SEQ ID NO: 668). 48 hours after transfection, DNA was harvested and analyzed for gene editing (T7E1 assay; forward primer: gatggaatcttctcctggtc, (SEQ ID NO: 669); reverse primer: aggaatgtccccatagatga, (SEQ ID NO: 670); product size: 725 nt; predicted band sizes: for pair 1: 495 nt and 230 nt, for pair 2: 544 nt and 181 nt, for pair 3: 567 nt and 158 nt). Results shown in FIG. 57 demonstrate efficient gene-editing by the pair 2 and pair 3 gene-editing proteins.

### Example 43: Generation and Characterization of Mesenchymal Stem Cells Differentiated from RNA-Reprogrammed Human Pluripotent Stem Cells

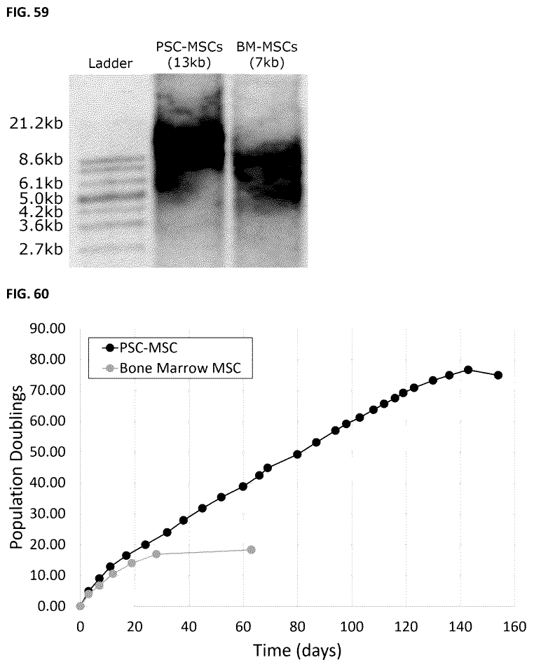

Primary human dermal fibroblasts were reprogrammed according to Example (Example 8) and differentiated using the STEMDIFF™ Mesenchymal Progenitor Kit (STEMCELL Technologies). Following differentiation, cells were stained with antibodies targeting Nanog, Sox2, CD73, and CD105 (FIG. 58). The differentiated cells and STEMPRO™ BM Mesenchymal Stem Cells (“MSC”; Thermo Fisher) were grown to approximately 90% confluency in 10 cm cell culture dishes and DNA was extracted. Telomere length was measured by Southern blot using the TELOTAGGG™ Telomere Length Assay Kit (Millipore-Sigma). The mean telomere length of the PSC-MSCs was observed to be much longer than that of the BM-MSCs (FIG. 59), and the proliferative capacity of the PSC-MSCs was observed to be much greater than that of the BM-MSCs (FIG. 60).

### Example 44: In Vivo Treatment of Experimental Autoimmune Encephalomyelitis, a Demyelinating Disease Model

Experiments will be conducted to examine in vivo consequences of Mesenchymal Stem Cells (MSC) cell transplantation for treating a model demyelinating disease.

Experimental autoimmune encephalomyelitis (EAE) is a central nervous system (CNS) autoimmune demyelinating disease that mimics many of the clinical and pathologic features of multiple sclerosis (MS). See, Gilgun-Sherki Y. et al., Neurosciences Research 47:201-207, 2003.

Here, in the so-called myelin oligodendrocyte glycoprotein (MOG) murine model, adult, female C57B1/6J mice undergo a sensitization period, induced by a subcutaneous (SC) injection of MOG 200 l/animal (at 2 mg/ml) emulsified in Complete Freund's adjuvant (“CFA”; containing heat-killed Mycobacterium tuberculosis H37 Ra) on Study Day 0, followed by intraperitoneal (IP) supplemental immunostimulation with pertussis toxin (“PT” of about 300 ng/mouse of a 2 μg/ml PT solution) on the first day of EAE induction (Study Day 0) and again 48 hours later (on Study Day 2). In these experiments, on Study Day 5 and/or Study Day 10, certain groups of mice (as described the below table), will be administered MSC (as described in Example 43) intravenously or intrathecally. Mice will be sacrificed on day 28 of the study.

| | Group | | MSC or PBS | Administration | Number of | Dosage of |
|---|---|---|---|---|---|---|
| | Size | Status | injected | route | Injections | PBS or MSC |
| Group 1 | N = 5 | Naive | N/A | N/A | 0 | N/A |
| Group 2 | N = 5 | Disease | PBS | Intravenous | 1 | 200 uL |
| | | Only | | | | |
| Group 3 | N = 5 | Disease | PBS | Intrathecal | 1 | 10 uL |
| | | Only | | | | |
| Group 4 | N = 5 | Disease | PBS | Intravenous | 2 | 200 uL |
| | | Only | | | | |
| Group 5 | N = 5 | Disease | PBS | Intrathecal | 2 | 10 uL |
| | | Only | | | | |
| Group 6 | N = 5 | Treated | MSC | Intravenous | 1 | 200 uL, |
| | | | | | | 500k cells |
| Group 7 | N = 5 | Treated | MSC | Intrathecal | 1 | 10 uL, |
| | | | | | | 100k cells |
| Group 8 | N = 5 | Treated | MSC | Intravenous | 2 | 200 uL, |
| | | | | | | 500k cells |
| Group 9 | N = 5 | Treated | MSC | Intrathecal | 2 | 10 uL, |
| | | | | | | 100k cells |
| Group 10 | N = 5 | Control | Dimethyl | Oral | 0 | 100 mg/kg |
| | | Treated | fumarate | | | |

Clinical signs will be assayed from Study Day 0 to termination day, on Study Day 28, and mice will be assigned a clinical score based on the criteria in the below table.

| Grade | Clinical sign | Comment |
|---|---|---|
| 0 | No clinical signs | Normal gait, tail moves and can be |
| | | raised, tail wraps around a round |
| | | object if mouse is held at the base |
| | | of the tail |
| 1 | Partially limp tail | Normal gait, tip of the tail droops |
| 2 | Paralyzed tail | Normal gait, tail droops |
| 3 | Hind limb paresis, | Uncoordinated gait, tail limps, hind |
| | uncoordinated | limbs respond to pinching |
| | movement | |
| 4 | One hind limb | Uncoordinated gait with one hind |
| | paralyzed | limb dragging, tail limps, one hind |
| | | limb does not respond to pinch |
| 5 | Both hind limbs | Uncoordinated gait with both hind |
| | paralyzed | limbs dragging, tail limps, both hind |
| | | limbs do not respond to pinch |
| 6 | Hind limbs paralyzed, | Uncoordinated gait with forelimbs |
| | weakness in forelimbs | struggle to pull body, forelimbs |
| | | reflex after pinching, tail limps |
| 7 | Hind limbs paralyzed, | Mouse cannot move, one forelimb |
| | one forelimb paralyzed | responds to toe pinch, tail limps |
| 8 | Hind limbs paralyzed, | Mouse cannot move, both forelimbs |
| | both forelimbs paralyzed | do not respond to toe pinch, tail |
| | | limps |
| 9 | Moribund | No movement, altered breathing |
| 10 | Death | |

After the mice are terminated on Study Day 28, tissues will be collected and assayed with Luxol fast blue stain (to stain myelin of spinal cord) and/or CD45 antibody stain.

### Example 45 Preparation of RNA with G-Substituted Poly(A) Tails

Single-stranded DNAoligomers comprising the desired tail sequence (see table below), flanked by EcoRI (5) and Spel (3′) restriction sites, and random sequences to promote proper annealing, were designed and synthesized (Integrated DNA Technologies), as were the reverse complement sequences. 4 nmol oligomer was dissolved in 100 μL nuclease-free water and hybridized with its reverse complement by slow cooling from 95° C. to 4° C. over a period of 2-3 hours. The hybridized oligomer was digested with EcoRI and Spel (New England Biolabs) and cloned into a GFP or NOVEPOIETIN DNA template immediately 3′ of the 3′ UTR.

| Tail | Sequence |
|---|---|
| A₁₅₀ 0% G | (A)₁₅₀ (SEQ ID NO: 736) |
| A₁₅₀ 2.5% G | (A₃₉G)₃(A)₃₀ (SEQ ID NO: 737) |
| A₁₅₀ 5% G | (A₁₉G)₇(A)₁₀ (SEQ ID NO: 738) |
| A₁₅₀ 10% G | (A₉G)₁₅ (SEQ ID NO: 739) |

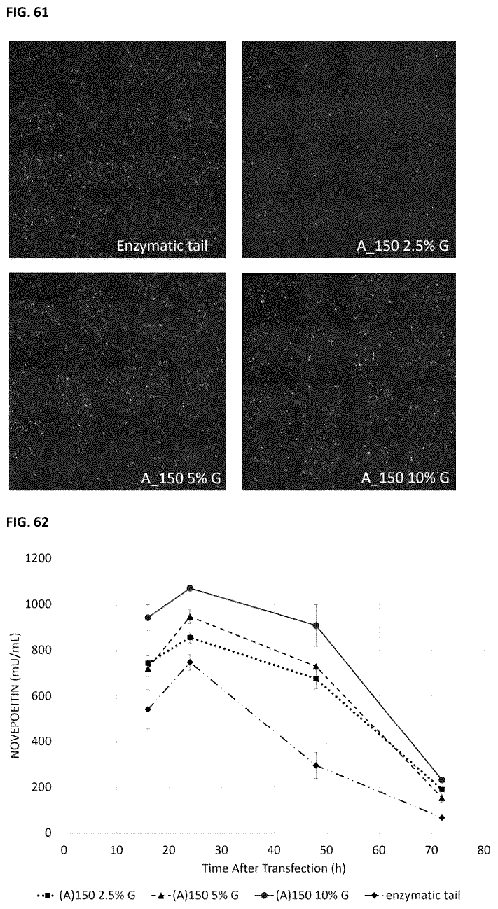

RNA was prepared by in vitro transcription from these templates according to Example 1, and was transfected into human epidermal keratinocytes (HEK) according to Example 3. Fluorescence data showing increased GFP expression in cells transfected with GFP with a poly(A) tail containing 5-10% G compared to those transfected with GFP tailed enzymatically (E. co/ipoly-A polymerase) are shown in FIG. 61. Expression of NOVEPOIETIN secreted into the culture medium was analyzed with an ELISA kit according to the manufacturer's instructions (01630, StemCell Technologies). Medium was changed every 24 hours. Expression levels are shown in FIG. 62.

### Example 46. Transfection of Keratinocytes with Synthetic RNA Containing Novel 3′-UTR Sequences

RNA encoding GFP and containing APOBEC3H, CD52, DMC1, EIF3E, GPR160, or RPS243′-UTRs (Respectively, SEQ ID NOs: 718, 720, 722, 724, 726, and 728) was synthesized according to Example 1. 50,000 or 100,000 neonatal human epidermal keratinocytes (HEKn) were plated in 6-well plates and were transfected with 1 μg RNA according to Example 2. After 6 hours, 24 hours, 48 hours, 72 hours, and 96 hours cells were evaluated for expression of GFP using fluorescence microscopy. At each time point, cells were trypsinized and evaluated for expression of GFP using flow cytometry. At each time point, samples of cells were lysed and mRNA was extracted and purified. GFP RNA was measured by rtPCR (Part Number 4331182, Thermo).

RNA encoding NOVEPOEITIN and containing APOBEC3H, CD52, DMC1, EIF3E, GPR160, or RPS24 3′-UTRs (Respectively, SEQ ID NOs: 718, 720, 722, 724, 726, and 728) was synthesized according to Example 1. 50,000 or 100,000 neonatal human epidermal keratinocytes (HEKn) were plated in 6-well plates and were transfected with 1 μg RNA according to Example 2. After 6 hours, 24 hours, 48 hours, 72 hours, and 96 hours cell media was evaluated for expression of NOVEPOEITIN using an ELISA kit (StemCell Technologies Part Number 01630)

### Example 47: Gene Editing HBB, PD-1, TRAC, and ADORA2A

RNA encoding gene editing proteins targeting the sequences TGGTGCATCTGACTCCTGAG (SEQ ID NO: 623) and TCACCTTGCCCCACAGGGCA (SEQ ID NO: 624) in the beta globin (HBB) gene were synthesized from DNA templates using the T7 High Yield RNA Synthesis Kit and the Vaccinia Capping System kit with mRNA Cap 2′-O-Methyltransferase (all from New England Biolabs, Inc.), according to the manufacturer's instructions and as previously described in, e.g., WO/2016/131052. The RNA was then diluted with nuclease-free water to between 100 ng/μL and 2000 ng/μL. RNA solutions were stored at room temperature, 4° C., −20° C., or −80° C.

200,000 Primary human cord blood CD34⁺ cells (70008.1, STEMCELL Technologies Inc.) were plated in a well of a 6-well plate in 1.8 mL of StemSpan SFEM 11(09605, STEMCELL Technologies Inc.)+0.2 mL of StemSpan 10× CD34⁺ Expansion Supplement (02691, STEMCELL Technologies Inc). Following expansion, 10⁶ cells were pelleted at 300×g for 5 minutes and resuspended in 100 μL of OptiMEM (Life Technologies). The RNA encoding gene editing proteins and an oligonucleotide repair template having the sequence: ACCTCAAACAGACACCATGGTGCATCTGACTCCTGAGGAGAAGCTTGCCGTTACTGCCCTGTGGGGCAAGG TGAACGTGGATGAAGTTGG (SEQ ID NO: 709) were added to the resuspended cells and gently mixed with a pipette tip. The mixture was transferred to an electroporation cuvette with a 2 mm gap (89047-208, VWR). The cells were electroporated in the cuvette using a pulse generator (ECM 830, BTX) set to deliver a square-wave pulse of 200V for 5 ms. The cells were then transferred from the cuvette to a new well of a 6-well plate with 1.98 mL of StemSpan SFEM II+20 μL of StemSpan CC110 100× expansion supplement (02697, STEMCELL Technologies Inc).

ICells were transfected with 5 μg of RNA encoding each component of the gene editing protein pair and 5 μg of the oligonucleotide repair template. 48 hours after transfection, the cells' genomic DNA was purified. A segment of the HBB gene was then amplified by PCR using the primers: GCCAAGGACAGGTACGGCTGTCATC (SEQ ID NO: 627) and CTTGCCATGAGCCTTCACCTTAGGGTTG (SEQ ID NO: 628), to produce a518 bp amplicon. Gene editing was assessed using T7 Endonuclease I (“T7E1”, New England Biolabs), which identifies and cleaves mismatches in double-stranded DNA. Briefly, the PCR product was denatured by heating approximately 200 ng of PCR product, 2 μL of NEBuffer 2 (New England Biolabs, Inc.), and nuclease-free water in a total volume of 19 μL to 95C for 5 minutes. The PCR product was then hybridized by cooling from 95C to 85C at −2C per second, then from 85C to 25C at −0.1C per second. The hybridized PCR product was then digested with T7 Endonuclease I. Bands of approximately 299 bp and 219 bp produced by T7E1 thus indicate successful gene editing. Gene repair was assessed using digestion with HindIII-HF, which identifies and cleaves DNA comprising the recognition sequence: AAGCTT, which is not present in the above-mentioned 518 bp amplicon generated from the wild-type HBB sequence, but is present in the repair template. Bands of approximately 301 bp and 217 bp produced by HindIII-HF thus indicate successful gene repair.

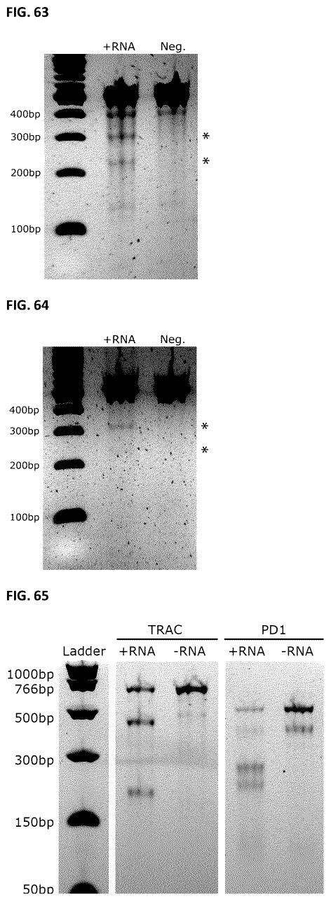

FIG. 63 shows that hematopoietic cells transfected with RNA encoding gene editing proteins and a repair template had T7E1-produced bands of approximately 299 bp and 219 bp. This data demonstrates successful generation of a single-strand or double-strand break in the DNA of hematopoietic cells. FIG. 64 shows that cells transfected with RNA encoding gene editing proteins and a repair template had HindIII-HF-produced bands of approximately 301 bp and 217 bp. This data demonstrates successful generation of a repair in the DNA of hematopoietic cells, the repair corresponding to the sequence of the nucleic acid that acts as a repair template. Together, the data demonstrate HBB gene editing and HBB gene repair in hematopoietic cells.

FIG. 65 depicts the results of an experiment in which 50,000 primary human neonatal epidermal keratinocytes (animal-protein free) were transfected with 2 μg RNA encoding the PD1 TALEN pairs 1/1 (target sequences: TGCTCCAGGCATGCAGATCC (SEQ ID NO: 794) and TAGCACCGCCCAGACGACTG (SEQ ID NO: 795)) or 1/2 (target sequences: TGCTCCAGGCATGCAGATCC (SEQ ID NO: 794) and TGTAGCACCGCCCAGACGAC (SEQ ID NO: 796)) (1 μg each individual pair component) or with 2 μg RNA encoding the TRAC TALEN pairs 2/2 (target sequences: TCACCGATTTTGATTCTCAA (SEQ ID NO: 797) and TATACACATCAGAATCCTTA (SEQ ID NO: 798)) or 2/3 (target sequences: TCACCGATTTTGATTCTCAA (SEQ ID NO: 797) and TGATATACACATCAGAATCC (SEQ ID NO: 799)) (1 μg each individual pair component). Cells were harvested and DNA was extracted after 48 and analyzed for gene editing (T7E1 assay; PD1: forward primer: AGTGACCACTGCCAACAGCTTCATGTC (SEQ ID NO: 800), reverse primer: GGGTAACAGCTGAATCAGGCCCTTCG (SEQ ID NO: 801), product size: 482 nt, predicted band sizes: 223 nt, 259 nt for pair 1/1, 219 nt, 263 nt for pair 1/2; TRAC: forward primer: GAATAAGCAGTATTATTAAGTAGCCCTGCATTTCAGGTTTC (SEQ ID NO: 802), reverse primer: CGAAGGCACCAAAGCTGCCCTTAC (SEQ ID NO: 803), product size: 606 nt, predicted band sizes: 197 nt, 409 nt for pair 2/2, 195 nt, 411 nt for pair 2/3).

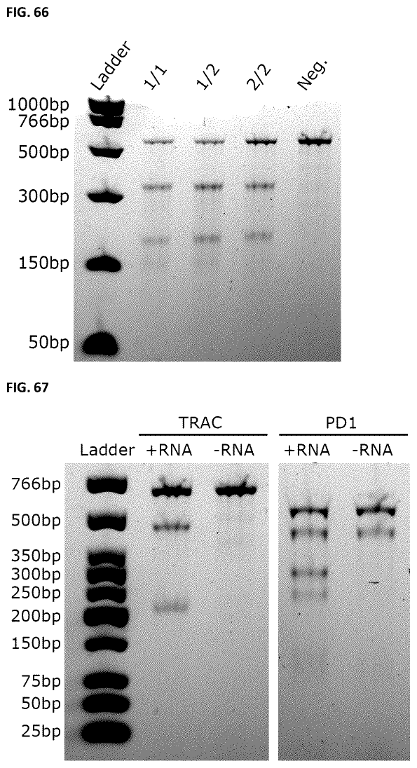

FIG. 66 depicts the results of an experiment in which 50,000 primary human neonatal epidermal keratinocytes (animal-protein free) were transfected with 2 μg RNA encoding the ADORA2A TALEN pairs 1/1 (target sequences: TGCTGAGCCTGCCTGTCGTC (SEQ ID NO: 804) and TGTACACCGAGGAGCCCATG (SEQ ID NO: 805)), 1/2 (target sequences: TGCTGAGCCTGCCTGTCGTC (SEQ ID NO: 804) and TGATGTACACCGAGGAGCCC (SEQ ID NO: 806)), or 2/2 (target sequences: TGAGCCTGCCTGTCGTCTGT (SEQ ID NO: 807) and TGATGTACACCGAGGAGCCC (SEQ ID NO: 806)) (1 μg each individual pair component). Cells were harvested and DNA was extracted after 48 and analyzed for gene editing (T7E1 assay; forward primer: CAGAGTCCTCTGTGAAAAAGCCCTTGGAG (SEQ ID NO: 808), reverse primer: ATGCGGATGGCAATGTAGCGGTCAATG (SEQ ID NO: 809), product size: 517 nt, predicted band sizes: 194 nt, 323 nt for pair 1/1, 196 nt, 321 nt for pair 1/2, 197 nt, 320 nt for pair 2/2).

FIG. 67 depicts the results of an experiment in which 50,000 RNA-reprogrammed human pluripotent stem cells were transfected with 2 μg RNA encoding a PD1 TALEN pair (target sequences: TGCTCCAGGCATGCAGATCC (SEQ ID NO: 794) and TGTAGCACCGCCCAGACGAC (SEQ ID NO: 796)) (1 μg each individual pair component) or with 2 μg RNA encoding a TRAC TALEN pair (target sequences: TCACCGATTTTGATTCTCAA (SEQ ID NO: 797) and TATACACATCAGAATCCTTA (SEQ ID NO: 798)) (1 μg each individual pair component). Cells were harvested and DNA was extracted after 48 and analyzed for gene editing (T7E1 assay; PD1: forward primer: AGTGACCACTGCCAACAGCTTCATGTC (SEQ ID NO: 800), reverse primer: GGGTAACAGCTGAATCAGGCCCTTCG (SEQ ID NO: 801), product size: 482 nt, predicted band sizes: 219 nt, 263 nt; TRAC: forward primer: GAATAAGCAGTATTATTAAGTAGCCCTGCATTTCAGGTTTC (SEQ ID NO: 802), reverse primer: CGAAGGCACCAAAGCTGCCCTTAC (SEQ ID NO: 803), product size: 606 nt, predicted band sizes: 197 nt, 409 nt).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1.-155. (canceled)

156. A synthetic RNA molecule comprising a 3′ tail sequence and a coding sequence for a peptide, wherein the 3′ tail sequence is downstream of the coding sequence and terminates at the 3′ terminus of the synthetic RNA molecule, wherein the 3′ tail sequence comprises at least 3 consecutive adenosine residues followed by at least one non-adenosine residue, and wherein the synthetic RNA molecule comprises one or more non-canonical nucleotides.

157. The synthetic RNA molecule of claim 156, wherein the 3′ tail sequence comprises at least two repeat sequences in tandem, and wherein each repeat sequence comprises at least 3 consecutive adenosine residues followed by at least one non-adenosine residue.

158. The synthetic RNA molecule of claim 156, wherein the 3′ tail sequence comprises more than 5 repeat sequences in tandem, and wherein each repeat sequence comprises at least 10 consecutive adenosine residues followed by at least one non-adenosine residue.

159. The synthetic RNA molecule of claim 156, wherein the at least one non-adenosine residue comprises a guanosine residue, a cytidine residue, or a uridine residue, or combinations thereof.

160. The synthetic RNA molecule of claim 156, wherein the one or more non-canonical nucleotides comprise one or more of pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

161. The synthetic RNA molecule of claim 156, wherein the synthetic RNA molecule comprises 100% 5-methoxyuridine in place of uridine.

162. The synthetic RNA molecule of claim 156, wherein the 3′ tail sequence comprises at least 2.5%, 5%, or 10% of one or more nucleotides other than adenosine.

163. The synthetic RNA molecule of claim 156, wherein the 3′ tail sequence comprises at least 80% adenosine.

164. The synthetic RNA molecule of claim 156, wherein the 3′ tail sequence comprises about 10 to about 250 nucleotides.

165. The synthetic RNA molecule of claim 156, wherein the 3′ tail sequence comprises (A)₁₄G (SEQ ID NO: 710), (A)₄G (SEQ ID NO: 712), (A)₁₃GG (SEQ ID NO: 713), (A)₁₂GGG (SEQ ID NO: 714), (A)₈GG (SEQ ID NO: 715), (A)₇GGG (SEQ ID NO: 716), (A)₃GG (SEQ ID NO: 717), (A39G)₃(A)₃₀ (SEQ ID NO: 737), (A19G)₇(A)₁₀ (SEQ ID NO: 738), (A9G)₁₅ (SEQ ID NO: 739), or at least two repeats of (A)₉G (SEQ ID NO: 711).

166. The synthetic RNA molecule of claim 156, wherein the 3′ tail sequence comprises 10 repeats of (A)₁₄G (SEQ ID NO: 710), or 15 repeats of (A)₉G (SEQ ID NO: 711), or 30 repeats of (A)₄G (SEQ ID NO: 712).

167. The synthetic RNA molecule of claim 156, wherein the 3′ tail sequence comprises 10 repeats of (A)₁₃GG (SEQ ID NO: 713), or 10 repeats of (A)₁₂GGG (SEQ ID NO: 714), or 15 repeats of (A)₈GG (SEQ ID NO: 715), or 15 repeats of (A)₇GGG (SEQ ID NO: 716), or 30 repeats of (A)₃GG (SEQ ID NO: 717).

168. The synthetic RNA molecule of claim 156, wherein the synthetic RNA molecule further comprises a 5′ untranslated region (UTR), a 3′ UTR, or both.

169. The synthetic RNA molecule of claim 156, wherein the synthetic RNA molecule is in vitro transcribed from a template.

170. The synthetic RNA molecule of claim 156, wherein the coding sequence for the peptide encodes interleukin (IL)-2, IL-4, IL-7, IL-10, IL-12, IL-15, or IL-18.

171. An in vitro composition comprising at least 1 nmol of synthetic RNA molecules, wherein each synthetic RNA molecule comprises a coding sequence for a peptide and a 3′ tail sequence that is downstream of the coding sequence and terminates at the 3′ terminus of the respective synthetic RNA molecule, and wherein the 3′ tail sequence comprises at least 3 consecutive adenosine residues followed by at least one non-adenosine residue.

172. The composition of claim 171, wherein the composition is in a solid form.

173. A nucleic acid template comprising a template sequence for synthesizing an RNA molecule, wherein the template sequence comprises: (a) a coding sequence for a peptide, and (b) downstream of the coding sequence, a sequence encoding a 3′ tail sequence comprising at least 3 deoxyadenosine nucleotides and one or more other nucleotides.

174. The nucleic acid template of claim 173, wherein the template sequence further comprises a restriction site located downstream of the sequence encoding the 3′ tail sequence.

175. The nucleic acid template of claim 173, wherein the nucleic acid template is a DNA molecule.

176. A synthetic RNA molecule, wherein the synthetic RNA molecule is synthesized from the nucleic acid template of claim 173.
